(19) United States
(12) Patent Application Publication
(10) Pub. No.: US 2003/0208067 A1
(43) Pub. Date: Nov. 6, 2003

(54) Inhibitors of protein kinase for the treatment of disease

(76) Inventors: Sheldon Xiaodong Cao, Carlsbad, CA (US); David Paul Dumas, San Diego, CA (US); Xiaohua Chen, San Diego, CA (US); Jae Young Yang, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/158,103

(22) Filed: May 28, 2002

Related U.S. Application Data
(60) Provisional application No. 60/294,792, filed on May 30, 2001.

(51) Int. Cl. C07D 279/12; C07D 333/32; C07D 265/30; C07D 237/02

(52) U.S. Cl. 544/59; 544/162; 544/181; 544/224; 544/335; 544/336; 546/332; 548/205; 548/247; 548/335.5; 548/375.1; 548/503; 549/66; 549/491; 564/36

(57) ABSTRACT

The present invention is directed in part towards methods of modulating the function of protein kinases with phenol- and hydroxynaphthalene-based compounds. The methods incorporate cells that express a protein kinase. In addition, the invention describes methods of preventing and treating protein kinase-related abnormal conditions in organisms with a compound identified by the invention. Furthermore, the invention pertains to phenol- and hydroxynaphthalene-based compounds and pharmaceutical compositions comprising these compounds.

### RELATED APPLICATIONS

The present application claims priority to the U.S. Provisional Patent Application Serial No. 60/294,792, filed May 30, 2001, by Cao et al., and entitled “INHIBITORS OF PROTEIN KINASE FOR THE TREATMENT OF DISEASE,” the disclosure of which is incorporated by reference herein in its entirety.

The present application is also related to the U.S. patent application Ser. No. 10/______, filed May 28, 2002, by Cao et al., and entitled “INHIBITORS OF PROTEIN KINASE FOR THE TREATMENT OF DISEASE,” (Attorney Docket No. LGBIOMD.002DV1) and U.S. patent application Ser. No. 10/______, filed May 28, 2002, by Cao et al., and entitled “INHIBITORS OF PROTEIN KINASE FOR THE TREATMENT OF DISEASE,” (Attorney Docket No. LGBIOMD.002DV2), both of whose disclosures are incorporated by reference herein in their entirety.

### BACKGROUND OF THE INVENTION

The following description of the background of the invention is provided to aid in understanding the invention, but is not admitted to describe or constitute prior art to the invention.

There are at least more than 400 enzymes identified as protein kinases, which catalyze the phosphoryl group transfer reaction from adenine triphosphate (ATP) to target protein substrates. The specific amino acid in the target substrate to which the phosphate group is transferred is a tyrosine, serine, or threonine, thereby protein kinase enzymes are commonly referred to as protein tyrosine kinases (PTKs) or serine/threonine kinases (STKs).

The protein kinases constitute a large family of structurally related enzymes that are necessary for the regulation of a wide variety of signaling pathways within the cell, including proliferation, differentiation, apoptosis, motility, transcription, translation, and many different signaling processes by phosphate groups transfer to target proteins. These phosphorylation processes act as molecular on/off switches that can regulate the biological function of the target proteins or protein complex. The appropriate function of the protein kinases in signaling pathways activate or inactivate metabolic enzymes, regulatory proteins, receptors, cytoskeletal proteins, ion channel and pump, transcription factors, etc. The inappropriately controlled signaling due to defective regulation of protein phosphorylation has been implicated in a number of diseases including inflammation, cancer, allergy/asthma, disease of the immune system, disease of the central nervous system, angiogenesis, etc.

Almost all kinases contain a similar 250-300 amino acid catalytic domain. The kinases may be categorized into families by the substrates they phosphorylate. Sequence motifs have been identified that generally correspond to each of these kinase families.

Protein tyrosine kinases (PTKs) are enzymes which catalyze the phosphorylation of specific tyrosine residues in cellular proteins. Aberrant or excess PTK activity has been observed in many diseases including proliferative disorders as well as diseases from inappropriate activation of the immune system, allograft rejection, and graft vs. host disease. PTKs can be of the receptor tyrosine kinases (RTKs) having extracellular, transmembrane and intracellular domains or the non-receptor tyrosine kinases (or cellular tyrosine kinases: CTKs) being wholly intracellular.

RTKs are growth factor receptors comprising a large family of transmembrane receptors with diverse biological activity. In fact, about twenty different subfamilies of RTKs have been identified. An example of these the subfamily designated the “HER” subfamily, which includes EGFR (epithelial growth factor receptor), HER2, HER3 and HER4. These RTKs consist of an extracellular glycosylated ligand. Another subfamily of theseRTKs is the insulin's, which includes INS-R, IGF-IR, and IR-R. The PDGF subfamily includes the PDGF-α and β receptors, CSFIR, c-kit and FLK-II. The FLK family includes the kinase insert domain receptor (KDR), fetal liver kinase-1 (FLK-1), fetal liver kinase-4 (FLK-4), and the fms-like tyrosine kinase-1 (flt-1). The PDGF and FLK families are usually considered together due to the similarities of the two groups.

A more complete listing of the known RTK subfamilies is described in Plowman et al., DN&P, 1994, 7(6):334-339 which is incorporated by reference, including any drawings, as if fully set forth herein.

The non-receptor type of tyrosine kinases or cellular tyrosine kinases (CTKs) is also comprised of numerous subfamilies, including Src, Frk, Btk, Csk, Abl, Zap70, Fes/Fps, Fak, Jak, Ack, and LIMK. Each of these subfamilies is sub-divided into various receptors. For instance, the Src subfamily is one of the largest including Src, Yes, Fyn, Lyn, Lck, Blk, Hck, Fgr, and Yrk. The Src subfamily of proteins has been linked to oncogenesis. Foe a more detailed discussion of CTKs, see Bolen, Oncogene, 1993, 8:2025-2031, which is incorpotated by reference, including any drawings, as if fully set forth herein.

Both receptor type and non-receptor type tyrosine kinases are implicated in cellular signaling pathways leading to numerous pathogenic conditions including cancer, psoriasis and hyperimmune responses.

The serine/threonine kinases (STKs) like the non-receptor tyrosine kinases, are predominantly intracellular although there are a few STK receptor kinases. STKs are the most common of the cytosolic kinases. The cytosol is the region within the cell where much of the cell's intermediary metabolic and biosynthetic activity occurs.

Cyclin dependent kinases (CDKs) have been shown to play important roles in cellular processes including cell cycle control, transcription, neuronal and muscular function, and apoptosis. Some enzymes for cell cycle control to be cyclinD/CDK4, cyclinD/CDK6, cyclinE/CDK2, cyclinE/CDK2 and cyclinB/CDK1 (also known as cyclinB/cdc2). CyclinD/CDK4, cyclinD/CDK6, and cyclinE/CDK2 control passage through the G1-phase and the G1 to S-phase transition by phosphorylation of the retinoblastoma phosphoprotein (pRb). CyclinA/CDK2 regulates passage through the S-phase, and cyclinB/CDK1 controls the G2 checkpoint and regulates entry into mitosis. Thus, specific inhibitors are expected to be useful in the treatment of proliferative diseases such as cancer, neurodegenerative diseases, cardiovascular diseases, and tissue damage as a result of trauma. Extensive search for specific inhibitors has been of particular interest for the treatment of various diseases.

Aurora kinases (AKs) have been shown to be protein kinases of a new family that regulate the structure and function of the mitotic spindle. There are three classes of aurora kinases containing aurora-A, aurora-B and aurora-C. Aurora-A includes AIRK1, DmAurora, HsAurora-2, HsAIK, HsSTK15, CeAIR-1, MmARK1, MmAYK1, MmIAK1 and XIEg2. Aurora-B includes AIRK-2, DmIAL-1, HsAurora-1, HsAIK2, HsAIM-1, HsSTK12, CeAIR-2, MmARK2 and XAIRK2. Aurora-C includes HsAIK3 (Adams, et al., Trends Cell Biol. 2001, 11, 49-54). Members of the Aurora family of mitotically regulated serine/threonine kinases are emerging as key regulators of chromosome segregation and cytokinesis. Deregulation of AKs has been implicated in oncogenesis as a consequence of chromosome missegregation (Hsu, et al., Cell, 2000, 102, 279-291).

RTKs, CTKs, CDKs, AKs, and STKs have all been implicated in a host of pathogenic conditions including cancer. Other pathogenic conditions include, without limitation, psoriasis, hepatic cirrhosis, diabetes, atherosclerosis, angiogenesis, restenosis, ocular diseases, rheumatoid arthritis and other inflammatory disorders, immunological disorders such as autoimmune disease, cardiovascular diseases such as atherosclerosis and a variety of renal disorders. With regard to cancer, two of the major hypotheses advanced to explain the excessive cellular proliferation that drives tumor development relate to functions known to be PK regulated. It has been suggested that malignant cell growth is the result of a breakdown in the mechanisms that control cell division and/or differentiation. It has been shown that the protein products of a number of proto-oncogenes are involved in the signal transduction pathways that regulate cell growth and differentiation. These protein products of proto-oncogenes include the extracellular growth factors, transmenbrane growth factor PTK receptors (RTKs), cytoplasmic PTKs (CTKs) and cytosolic STKs.

In view of the apparent link between PK-related cellular activities and a number of human disorders, it is no surprise that a great deal of effort is being spent to identify ways to modulate PK activity. Some of these efforts have been directed at biomimetic approaches using large molecules patterned on those involved in the actual cellular processes (e.g., mutant ligands U.S. application Ser. No. 4,966,849); soluble receptors and antibodies (App. No. WO 04/10202, Kendall and Thomas, Proc. Nat'l Acad Sci., 1994, 90:10705-09, Kim, et al., Nature, 1993, 3 62:841-844); RNA ligands (Jelinek, et al., Biochemistry, 33:10450-56); Takano, et al., Mol. Bio. Cell, 1993, 4:358A; Kinsella, et al., Exp. Cell Res., 1992, 199:56-62; Wright, et al., J Cellular Phys., 152:448-57) and tyrosine kinase inhibitors (WO 94/03427; WO 92/21660; WO 91/15495; WO 94/14808; U.S. Pat. No. 5,330,992; Mariani, et al., Proc. Am. Assoc. Cancer Res., 1994, 35:2268).

More recently, attempts have been made to identify small molecules that act as PTK inhibitors. For example, bis- monocylic, bicyclic and heterocyclic aryl compounds (PCT WO 92/20642; PCT WO00/43373; PCT WO01/19828 A2; PCT WO01/17995), vinylene-azaindole derivatives (PCT WO 15 94/14808) and 1-cyclopropyl-4-pyridylquinolones (U.S. Pat. No. 5,330,992) have been described as PTK inhibitors. Styryl compounds (U.S. Pat. No. 5,217,999), styryl-substituted pyridyl compounds (U.S. Pat. No. 5,302,606), quinazoline derivatives (EP App. No. 0 566 266 A1; Expert Opin. Ther. Pat., 1998, 8(4), 475-478), selenaindoles and selenides (PCT WO 94/03427), tricyclic polyhydroxylic compounds (PCT WO 92/21660) and benzylphosphonic acid compounds (PCT WO 91/15495) have all been described as PTK inhibitors useful in the treatment of cancer. Anilinocinnolines (PCT WO97/34876) and quinazoline derivative compounds (PCT WO97/22596; PCT WO97/42187) have been described as inhibitors of angiogenesis and vascular permeability.

In addition, attempts have been made to identify small molecules which act as STK inhibitors. For examples, bis(indolylmaleimide) compounds have been described as inhibiting particular PKC STK isoforms whose signal transducing function is associated with altered vascular permeability in VEGF-related diseases (PCT WO97/40830; PCT WO97/40831, both of which are incorporated herein by reference in their entirety).

### SUMMARY OF THE INVENTION

An aspect of the present invention relates to a compound of Formula I

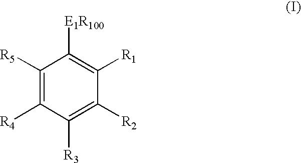

or a pharmaceutically acceptable salt, amide, ester, or prodrug thereof, where

a) R₁ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) an ancyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1; and

iii) a substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that R₁ is not —C₆H₅, —C(O)H, —C(O)CH₃, —C(O)—C₆H₅, —C(O)NH₂, or —C₆H₄CH₃;

b) R₂, R₃, and R₄ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or hetroaryl ring, optionally substituted with one or more substitutents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisitng of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkowy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

or R₂ and R₃, taken together along with the two ring carbons to which they are attached, or R₄ and R₃, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl

iv) optionally substituted heterocyle;

v) substituent of formula —C(X₄)═N—NX₅—C(═E)—NX₆X₇ or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroarylr;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1; and

c) R₅ is selected from the group consisting of hydrogen, lower alkyl, aryl, and heteroaryl, or R₄ and R₅, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

d) R₁₀₀ is selected from the group consisting of hydrogen, lower alkyl, lower alkene, lower alkyne, aryl, and heteroaryl;

e) E₁ is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

provided that at least one of R₁-R₅ is not selected from the group consisting of hydrogen, lower alkyl, aryl, and heteroaryl.

An aspect of the present invention relates to a compound of Formula II

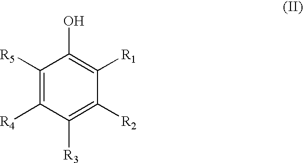

or a pharmaceutically acceptable salt, amide, ester, or prodrug thereof, where

a) R₁ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) an acyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1; and

iii) a substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that R₁ is not —C₆H₅, —C(O)H, —C(O)CH₃, —C(O)—C₆H₅, —C(O)NH₂, or —C₆H₄CH₃;

b) R₂, R₃, and R₄ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

or R₂ and R₃, taken together along with the two ring carbons to which they are attached, or R₄ and R₃, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1; and

c) R₅ is selected from the group consisting of hydrogen, lower alkyl, aryl, and heteroaryl, or R₄ and R₅, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

provided that at least one of R₁-R₅ is not selected from the group consisting of hydrogen, lower alkyl, aryl, and heteroaryl.

Another aspect of the invention relates to a compound of Formula III

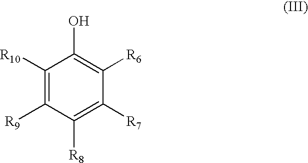

or a pharmaceutically acceptable salt, amide, ester, or prodrug thereof, where

a) R₆ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro; and

ii) acyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1;

provided that R₆ is not —C₆H₅, —C(O)H, —C(O)CH₃, —C(O)—C₆H₅, —C(O)NH₂, or —C₆H₄CH₃.

b) R₇, R₈, and R₉ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

where X₂₀ and X₂₁, are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl; and

c) R₁₀ is selected from the group consisting of hydrogen, lower alkyl, aryl, and heteroaryl;

provided that at least one of R₆-R₁₀ is not selected from the group consisting of hydrogen, lower alkyl, aryl, and heteroaryl.

A further aspect of the invention relates to a compound of Formula IV

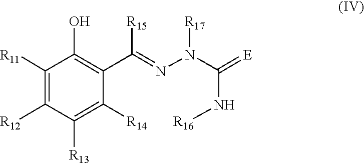

or a pharmaceutically acceptable salt, amide, ester, or prodrug thereof, where

a) R₁₁ is selected from the group consisting of hydrogen, lower alkyl, aryl, and heteroaryl;

b) R₁₂, R₁₃, and R₁₄, are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

c) R₁₅ is selected from the group consisting of hydrogen, lower alkyl, aryl, alkaryl, heteroaryl, and amino;

d) R₁₆ and R₁₇ are each independently selected from the group consisting of hydrogen, lower alkyl, aryl, and heteroaryl; and

e) E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl.

In another aspect the invention relates to a compound of Formula V or of

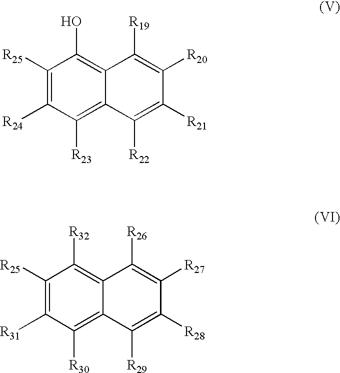

or a pharmaceutically acceptable salt, amide, ester, or prodrug thereof, where

a) R₁₉-R₂₂ and R₂₆-R₂₉ are each independently selected from the group consisting of:

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₁₉ is selected from the group consisting of hydrogen, lower alkyl, aryl, heteroaryl, amino, and —NX₂₀X₂₁,

where X₂₀ and X₂₁, are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n₂₂ is 0 or 1;

b) R₂₃ and R₃₀ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)n₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl; and

c) R₂₄, R₂₅, R₃₁ and R₃₂ are each independently selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) acyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1; and

iii) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that none of R₂₄, R₂₅, R₃₁ or R₃₂ is —C₆H₅, —C(O)H, —C(O)CH₃, —C(O)—C₆H₅, —C(O)NH₂, or —C₆H₄CH₃.

Certain aspects of the present invention also relate to a compound selected from the group consisting of the compounds set forth in Table 1, or a pharmaceutically acceptable salt, ester, amide, or prodrug thereof.

The compounds of the present invention are capable of inhibiting the catalytic activity of a protein kinase. The protein kinase may be selected from the group consisting of a receptor protein tyrosine kinase, a cellular tyrosine kinase, and a serine-threonine kinase.

In certain aspects, the invention relates to a method for the modulation of the catalytic activity of a protein kinase comprising contacting the protein kinase with any of the compounds of the invention.

The invention also relates to a method of modulating a signal transduction pathway in a cells comprising the step of contacting the cell with the compound with any of the compounds of the invention.

Another aspect of the present invention relates to a method of identifying an aromatic compound that modulates the function of protein kinase, comprising the following steps:

a) contacting cells expressing the protein kinase with a any of the compounds of the invention; and

b) monitoring an effect of the compound upon the cells.

In another aspect, the invention relates to method of regulating an unregulated protein kinase signal transduction comprising administering to a subject a therapeutically effective amount of any of the compounds of the invention. The unregulated protein kinase signal transduction may lead to a disease or an abnormal condition in an organism and the method may lead to the treatment or prevention of the disease or abnormal condition; where the disease or abnormal condition is associated with an aberration in a signal transduction pathway characterized by an interaction between a protein kinase and a binding partner, and where the method further comprises the steps of promoting or disrupting the abnormal interaction.

In the above method, the disease or abnormal condition may be selected from the group consisting of cell proliferative disease, cerebrovascular damage, autoimmune diseases, neurodegenerative disease, degenerative diseases of the musculoskeletal system.

In an additional aspect, the present invention relates to a pharmaceutical composition comprising i) a physiologically acceptable carrier, diluent, or excipient, or a combination thereof; and ii) a compound of the invention.

### DETAILED DESCRIPTION OF THE PREFERRED EMBODIMENTS

### I. Compounds of the Invention

A. General Description of the Compounds

An aspect of the present invention relates to a compound of Formula I

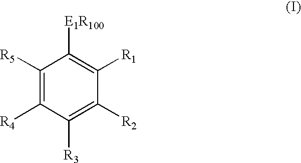

or a pharmaceutically acceptable salt, amide, ester, or prodrug thereof, where

a) R₁ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) an acyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1; and

iii) a substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that R₁ is not —C₆H₅, —C(O)H, —C(O)CH₃, —C(O)—C₆H₅, —C(O)NH₂, or —C₆H₄CH₃;

b) R₂, R₃, and R₄ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

or R₂ and R₃, taken together along with the two ring carbons to which they are attached, or R₄ and R₃, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1; and

c) R₅ is selected from the group consisting of hydrogen, lower alkyl, aryl, and heteroaryl, or R₄ and R₅, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

d) R₁₀₀ is selected from the group consisting of hydrogen, lower alkyl, lower alkene, lower alkyne, aryl, and heteroaryl;

e) E₁ is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

provided that at least one of R₁-R₅ is not selected from the group consisting of hydrogen, lower alkyl, aryl, and heteroaryl.

In another aspect, the present invention relates to a compound of Formula II

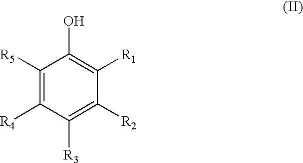

or a pharmaceutically acceptable salt, amide, ester, or prodrug thereof, where

a) R₁ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) an acyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1; and

iii) a substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that R₁ is not —C₆H₅, —C(O)H, —C(O)CH₃, —C(O)—C₆H₅, —C(O)NH₂, or —C₆H₄CH₃;

b) R₂, R₃, and R₄ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

or R₂ and R₃, taken together along with the two ring carbons to which they are attached, or R₄ and R₃, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1; and

c) R₅ is selected from the group consisting of hydrogen, lower alkyl, aryl, and heteroaryl, or R₄ and R₅, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

provided that at least one of R₁-R₅ is not selected from the group consisting of hydrogen, lower alkyl, aryl, and heteroaryl.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The term “aromatic” refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” refers to an aromatic group which contains at least one heterocyclic ring.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds of the invention may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethly, propyl, iso-propyl, n-butyl, iso-butyl, sec-butyl, and t-butyl.

The alkyl group may be substituted or unsubstituted. When substituted, the substituent group(s) is (are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substitutent may be substituted with one of the above substituents.

The substituent “R” appearing by itself and without a number designation refers to a substituent selected from the group consisting of of alkyl, cycloalkyl, aryl, heteroaryl (bonded through a ring carbon) and heteroalicyclic (bonded through a ring carbon).

An “O-carboxy” group refers to a RC(═O)O— group, where R is as defined herein.

A “C-carboxy” group refers to a —C(═O)OR groups where R is as defined herein.

An “acetyl” group refers to a —C(═O)CH₃, group.

A “trihalomethanesulfonyl” group refers to a X₃CS(═O)₂— group where X is a halogen.

A “cyano” group refers to a —CN group.

An “isocyanato” group refers to a —NCO group.

A “thiocyanato” group refers to a —CNS group.

An “isothiocyanato” group refers to a —NCS group.

A “sulfinyl” group refers to a —S(═O)—R group, with R as defined herein.

A “S-sulfonamido” group refers to a —S(═O)₂NR, group, with R as defined herein.

A “N-sulfonamido” group refers to a RS(═O)₂NH— group with R as defined herein.

A “trihalomethanesulfonarnido” group refers to a X₃CS(═O)₂NR— group with X and R as defined herein.

An “O-carbamyl” group refers to a —OC(═O)—NR, group-with R as defined herein.

An “N-carbamyl” group refers to a ROC(═O)NH— group, with R as defined herein.

An “O-thiocarbamyl” group refers to a —OC(═S)—NR, group with R as defined herein.

An “N-thiocarbamyl” group refers to an ROC(═S)NH— group, with R as defined herein.

A “C-amido” group refers to a —C(═O)—NR₂ group with R as defined herein.

An “N-amido” group refers to a RC(═O)NH— group, with R as defined herein.

The term “perhaloalkyl” refers to an alkyl group where all of the hydrogen atoms are replaced by halogen atoms.

When two substituents taken together along with the two ring carbons to which they are attached form a ring, it is meant that the following structure:

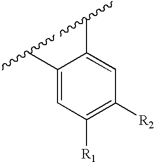

is representative of the following structure:

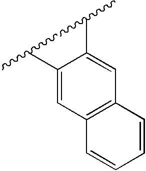

In the above example, R₁ and R₂, taken together along with the two ring carbons to which they are attached, form a six-membered aromatic ring.

Unless otherwise indicated, when a substituent is deemed to be “optionally subsituted,” it is meant that the subsitutent is a group that may be substituted with one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

In certain embodiments, the invention relates to a compound of Formula I or Formula II, where R₁ is selected from the group consisting of

i) hydrogen;

ii) a six-membered aromatic ring, optionally substituted with one or more substituents selected from the group consisting of lower alkyl, hydroxy, and amino;

iii) a six-membered heteroaromatic ring, optionally substituted with one or more substituents selected from the group consisting of lower alkyl, hydroxy, alkoxy, amino, and nitro;

iv) a five-membered heteroaromatic ring, optionally substituted with one or more substituents selected from the group consisting of lower alkyl, hydroxy, and amino;

v) acyl of formula —(X₁)ₙ₁—C(O)—X₂, where

X₁ is lower alkylene or lower alkenylene;

X₂ is selected from the group consisting of hydrogen, amino, hydroxy, and —NH—X₃,

where X₃ is selected from the group consisting of hydrogen, lower alkyl, amino, and amide; and

n1 is 0 or 1; and

vi) substituent of formula —C(X₄)═N—NH—C(═E)—NX₆X₇, or of formula —C(X₄)═N—N═C(EX₅)—NX₆X₇, where

X₄ is selected from the group consisting of hydrogen, lower alkyl, aryl, and alkaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen and lower alkyl.

In other embodiments, R₁ is selected from the group consisting of

i) hydrogen;

ii) a phenyl, optionally substituted with one or more of hydroxy or —NH₂;

iii) a six-membered heteroaromatic ring, selected from the group consisting of pyridine, pyrazine, pyridazine, pyrimidine, and 1,3,5-triazine, each independently and optionally substituted with one or more substituents selected from the group consisting of lower alkyl, hydroxy, alkoxy, and amino;

iv) a five-membered heteroaromatic ring, selected from the group consisting of pyrrole, imidazole, 1,2,3-triazole, 1,2,4-triazole, tetrazole, thiophene, thiazole, and thiadiazole, each independently and optionally substituted with one or more substituent selected from the group consisting of hydroxy, an amide of formula —(X₂₄)ₙ₂₄—NH—C(O)—X₂₅ or —(X₂₆)ₙ₂₆—C(O)—NH—X₂₇, and —NH₂;

v) acyl of formula —C(O)—X₂, where X₂ is hydrogen or lower alkyl;

vi) acyl of formula —X₁—C(O)—X₂, where

X₁ is lower alkylene or lower alkenylene; and

X₂ is —NH—X₃, where X₃ is selected from the group consisting of hydrogen, amino, and amide;

vi) substituent of formula —C(X₄)═N—NH—C(═E)—NH₂, where

X₄ is selected from the group consisting of hydrogen, methyl, ethyl, phenyl, and —CH₂CH₂—Ph; and

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

vii) substituent of formula —C(X₄)═N—N═C(SX₅)—NH₂, where

X₄ is selected from the group consisting of hydrogen, methyl, ethyl, phenyl, and —CH₂CH₂—Ph; and

X₅ is hydrogen or methyl.

The five- or six-membered heteroaryl ring in R₁ may be selected from the group consisting of optionally substituted

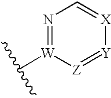

optionally substituted

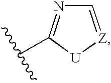

and optionally substituted

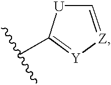

where W, X, Y, and Z are each independently CR or nitrogen, and U is selected from the group consisting of CR₂, oxygen, sulfur, and NR; where R is as defined herein.

Other embodiments of the present invention relate to a compound of Formula I or Formula II, where the above heteroaryl ring is selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, triazine,

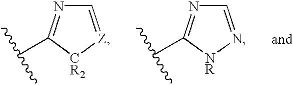

and

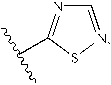

where R is as defined herein.

The heteroaryl ring may be selected from the group consisting of aminofuran, aminothiophene, aminopyrrole, aminopyrroline, aminopyrrolidine, aminooxazole, aminothiazole, aminoimidazole, aminoimidazoline, aminoimidazolidine, aminopyrazole, aminopyrazoline, aminopyrazolidine, aminoisoxazole, aminoisothiazole, aminotriazole, aminothiadiazole, aminopyran, aminopyridine, aminopiperidine, aminomorpholine, aminothiomorpholine, aminopyridazine, aminopyrimidine, aminopyrazine, aminopiperazine, aminotriazine,

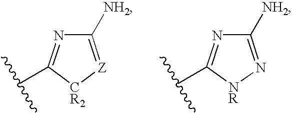

and

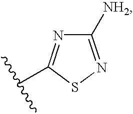

where R is defined as herein.

Embodiments of the invention relate to a compound of Formula I or Formula II, where R₁ is selected from the group consisting of hydrogen, —C(O)—CH₃, —C(O)—NH—CH₂—C(O)—NH₂, —CH═CH—C(O)—NH₂, —CH₂CH₂—C(O)—NH—NH₂, —C(H)═N—NH—C(O)—NH₂, —C(CH₃)═N—NH—C(O)—NH₂, —C(H)═N—NH—C(S)—NH₂, —C(CH₃)═N—NH—C(S)—NH₂, —C(Ph)═N—NH—C(S)—NH₂, —C(CH₂CH₂Ph)═N—NH—C(S)—NH₂, —C(H)═N—N═C(SCH₃)—NH₂,

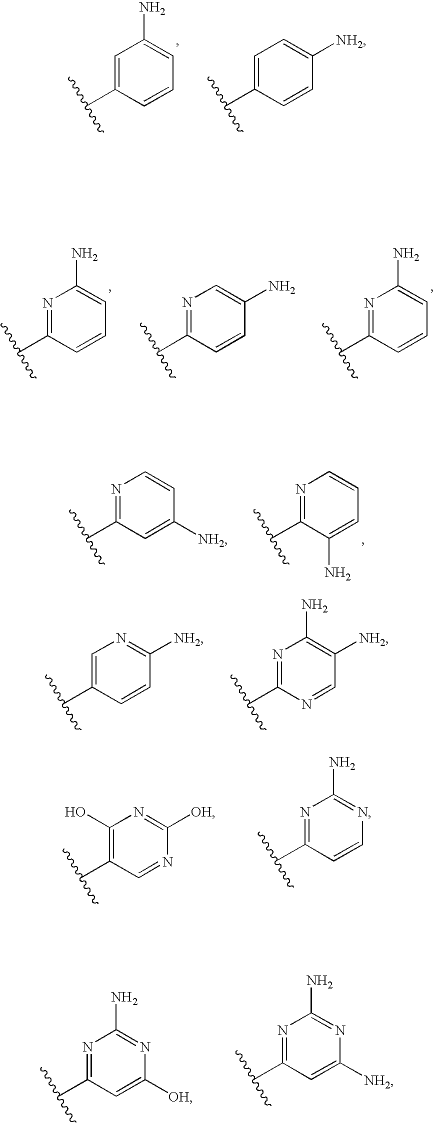

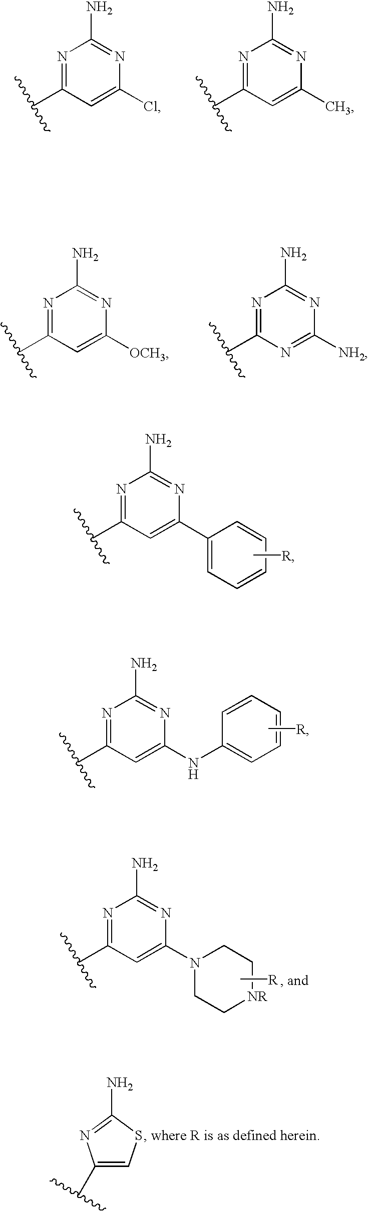

The substituent R₃ may in certain embodiments be selected from the group consisting of

i) hydrogen;

ii) C₂-C₆ alkenylene;

iii) halogen or perhaloalkyl;

iv) an alkoxy of formula —O—X₁₄, where

X₁₄ is selected from the group consisting of hydrogen, lower alkyl, and aryl; and

v) a five-membered or six-membered heteroaryl ring or a six-membered aryl or heteroaryl ring, optionally substituted with one or more substituents selected from the group consisting of

A) optionally substituted C₁-C₄ straight-chain, branched, or cyclic saturated or unsaturated alkyl;

B) an alkoxy of formula —O—X₁₄, where X₁₄ is hydrogen or lower alkyl; and

C) halogen or perhaloalkyl;

D) cyano;

E) nitro;

F) a substituent of formula —(X₁₈)ₙ₁₈—C(O)—X₁₉, where

X₁₈ is lower alkylene;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, and aryl; and

n18 is 0 or 1; and

G) a thioether or thiol of formula —S—X₂₃, where X₂₃ is selected from the group consisting of hydrogen, lower alkyl, aryl, and heteroaryl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, lower alkyl, aryl, hydroxy, and alkoxy; and

X₂₇ is selected from the group consisting of hydrogen, lower alkyl, aryl, and heteroaryl.

In other embodiments, R₃ may be selected from the group consisting of

i) hydrogen;

ii) C₂-C₆ alkenylene;

iii) halogen or perfluoroalkyl;

iv) an alkoxy of formula —O—X₁₄, where

X₁₄ is selected from the group consisting of hydrogen, methyl, ethyl, and propyl; and

v) a six-membered aryl ring, optionally substituted with one or more substituents selected from the group consisting of

A) methyl, ethyl, and propyl;

B) hydroxy, methoxy, ethoxy, phenoxy, hydroxymethyl, 2-hydroxyethyl, and 3-hydroxypropyl; and

C) halogen or perfluoroalkyl;

D) cyano;

E) nitro;

F) a substituent of formula —C(O)—X₁₉, where

X₁₉ is selected from the group consisting of hydrogen, methyl, ethyl, propyl, phenyl, hydroxy, methoxy, ethoxy, propoxy, amino, and —NX₂₀X₂₁,

where X₂₀ and X₂₁ are each independently selected from the group consisting of hydrogen, methyl, ethyl, propyl, and phenyl; and

G) a thioether or thiol of formula —S—X₂₃, where X₂₃ is selected from the group consisting of hydrogen, methyl, ethyl, propyl, and phenyl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, methyl, ethyl, propyl, phenyl, hydroxy, methoxy, and phenoxy; and

X₂₇ is selected from the group consisting of hydrogen, methyl, ethyl, propyl, and phenyl.

The five-membered or six-membered heteroaryl ring or the six-membered aryl or heteroaryl ring of R₃ may be selected from the group consisting of optionally substituted

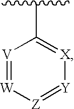

optionally substituted

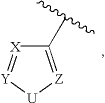

and optionally substituted

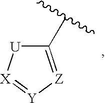

where V, W, X, Y and Z are each independently CR or nitrogen, and U is selected from the group consisting of CR₂, oxygen, sulfur, and NR; where R is as defined herein.

In further embodiments, the five-membered or six-membered heteroaryl ring or the six-membered aryl or heteroaryl ring of R₃ may be selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

The R₃ substituent may also be selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonylphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-

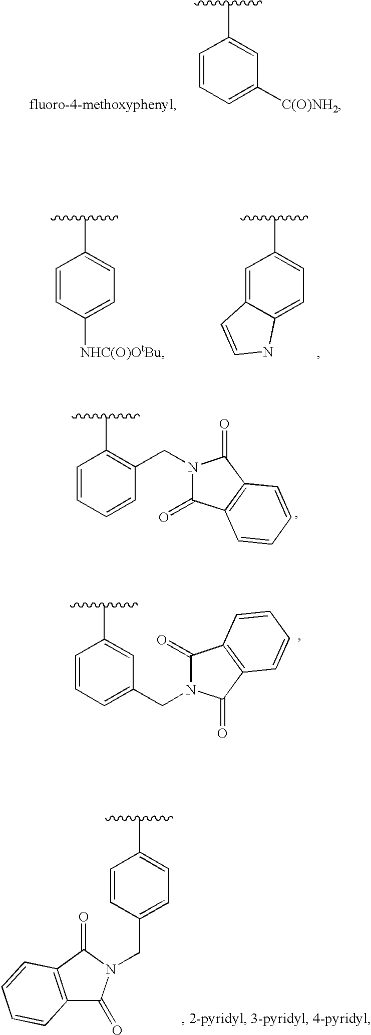

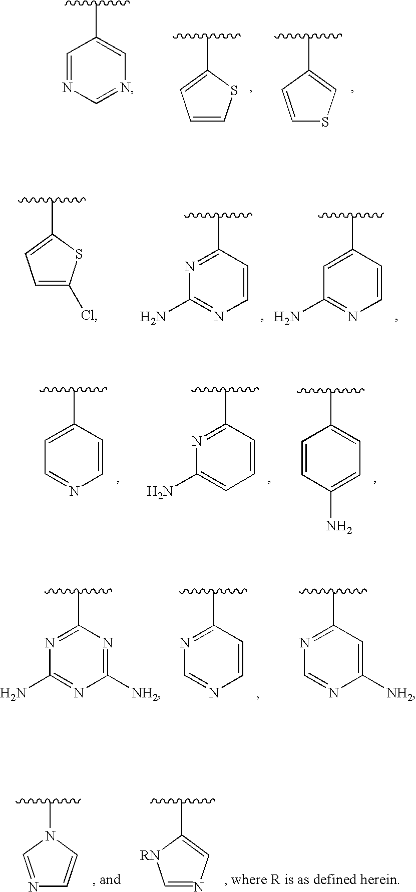

In certain embodiments, the R₂ and R₃ substituents of the compound of Formula I or Formula II, taken together along with the two ring carbons to which they are attached, or R₄ and R₃ substituents of the compound of Formula I or Formula II, taken together along with the two ring carbons to which they are attached, or R₄ and R₅ substituents of the compound of Formula I or Formula II, taken together along with the two ring carbons to which they are attached, form a six-membered aromatic or heteroaromatic, or a five- or six-membered heteroaromatic ring, optionally substituted with one or more substituents selected from the group consisting of hydrogen, hydroxy, halogens, cyano, nitro, amino, carbonyl, hydroxycarbonyl, aminocarbonyl, aminothiocarbonyl, lower alkoxy, phenoxy, amino-furan, amino-thiophene, amino-pyrrole, amino-pyrroline, amino-pyrrolidine, amino-oxazole, amino-thiazole, amino-imidazole, amino-imidazoline, amino-imidazolidine, amino-pyrazole, amino-pyrazoline, amino-pyrazolidine, amino-isoxazole, amino-isothiazole, aminotriazole, amino-thiadiazole, amino-pyran, amino-pyridine, amino-piperidine, amino-morpholine, amino-thiomorpholine, amino-pyridazine, amino-pyrimidine, amino-pyrazine, aminopiperazine, amino-triazine, semicarbazone, thiosemicarbazone, and amino guanidine.

In certain embodiments, R₂ and R₃, taken together along with the rest of the compound of Formula I or Formula II, or the R₄ and R₃, taken together along with the rest of the compound of Formula I, or the R₄ and R₅, taken together along with the rest of the compound of Formula I or Formula II, result in the formation of an optionally substituted naphthalene. The resulting naphthalene molecule may be substituted with a hydroxy.

B. The Phenol Derivatives

In another aspect, the invention relates to a compound of Formula III

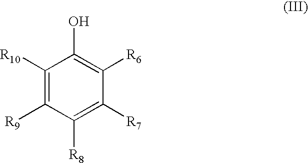

or a pharmaceutically acceptable salt, amide, ester, or prodrug thereof, where

a) R₆ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro; and

ii) acyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1;

provided that R₆ is not —C₆H₅, —C(O)H, —C(O)CH₃, —C(O)—C₆H₅, —C(O)NH₂, or —C₆H₄CH₃.

b) R₇, R₈, and R₉ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl; and

c) R₁₀ is selected from the group consisting of hydrogen, lower alkyl, aryl, and heteroaryl;

provided that at least one of R₆-R₁₀ is not selected from the group consisting of hydrogen, lower alkyl, aryl, and heteroaryl.

In certain embodiments, R₆ is selected from the group consisting of

i) hydrogen;

ii) a six-membered aromatic ring, optionally substituted with one or more substituents selected from the group consisting of lower alkyl, hydroxy, and amino;

iii) a six-membered heteroaromatic ring, optionally substituted with one or more substituents selected from the group consisting of lower alkyl, hydroxy, alkoxy, amino, and nitro;

iv) a five-membered heteroaromatic ring, optionally substituted with one or more substituents selected from the group consisting of lower alkyl, hydroxy, and amino; and

v) acyl of formula —(X₁)ₙ₁—C(O)—X₂, where

X₁ is lower alkylene or lower alkenylene;

X₂ is selected from the group consisting of hydrogen, amino, hydroxy, and —NH—X₃,

where X₃ is selected from the group consisting of hydrogen, lower alkyl, amino, and amide; and

n1 is 0 or 1.

The R₆ substituent may be selected from the group consisting of

i) hydrogen;

ii) a phenyl, optionally substituted with one or more of hydroxy or —NH₂;

iii) a six-membered heteroaromatic ring, selected from the group consisting of pyridine, pyrazine, pyridazine, pyrimidine, and 1,3,5-triazine, each independently and optionally substituted with one or more substituents selected from the group consisting of lower alkyl, hydroxy, alkoxy, and amino;

iv) a five-membered heteroaromatic ring, selected from the group consisting of pyrrole, imidazole, 1,2,3-triazole, 1,2,4-triazole, tetrazole, thiophene, thiazole, and thiadiazole, each independently and optionally substituted with one or more substituent selected from the group consisting of hydroxy, an amide of formula —(X₂₄)ₙ₂₄—NH—C(O)—X₂₅ or —(X₂₆)ₙ₂₆—C(O)—NH—X₂₇, and —NH₂;

v) acyl of formula —C(O)—X₂, where X₂ is hydrogen or lower alkyl; and

vi) acyl of formula —X₁—C(O)—X₂, where

X₁ is lower alkylene or lower alkenylene; and

X₂ is —NH—X₃, where X₃ is selected from the group consisting of hydrogen, amino, and amide.

It is conceivable that the five- or six-membered heteroaryl ring in R₆ may be selected from the group consisting of optionally substituted

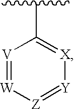

optionally substituted

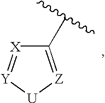

and optionally substituted

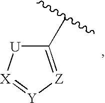

where V, W, X, Y and Z are each independently CR or nitrogen, and U is selected from the group consisting of CR₂, oxygen, sulfur, and NR; where R is as defined herein.

The above heteroaryl ring may also be selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, oxadiazole

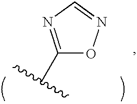

pyran, pyridine, piperidine, morpholine, thiomorpholine, pyridazine, pyrimidine, pyrazine, piperazine, and triazine. It is also possible that the above heteroaryl ring may be selected from the group consisting of aminofuran, aminothiophene, aminopyrrole, aminopyrroline, aminopyrrolidine, aminooxazole, aminothiazole, aminoimidazole, aminoimidazoline, aminoimidazolidine, aminopyrazole, aminopyrazoline, aminopyrazolidine, aminoisoxazole, aminoisothiazole, aminotriazole, aminothiadiazole, aminooxadiazole, aminopyran, aminopyridine, aminopiperidine, aminomorpholine, aminothiomorpholine, aminopyridazine, aminopyrimidine, aminopyrazine, aminopiperazine, and aminotriazine.

In certain embodiments, the R₆ substituent is selected from the group consisting of hydrogen, —C(O)—CH₃, —C(O)—NH—CH₂—C(O)—NH₂, —CH═CH—C(O)—NH₂, —CH₂CH₂—C(O)—NH—NH₂,

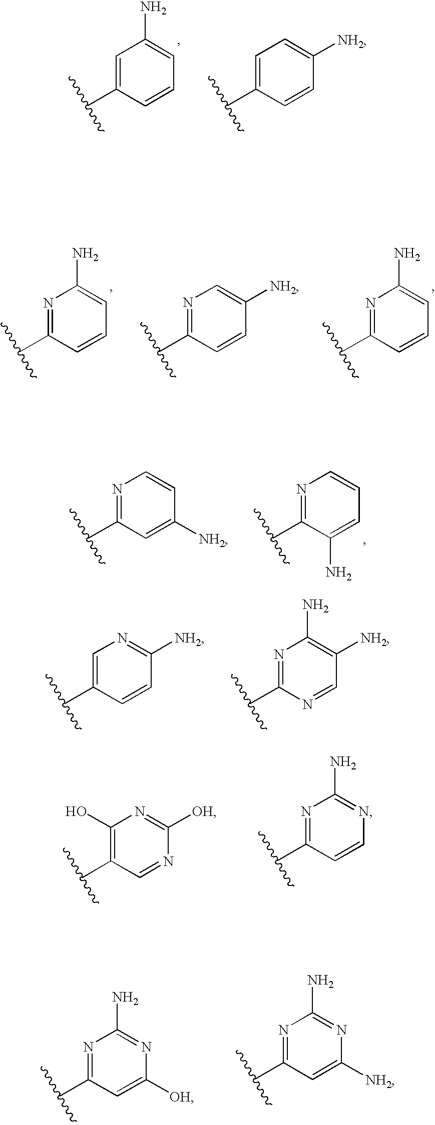

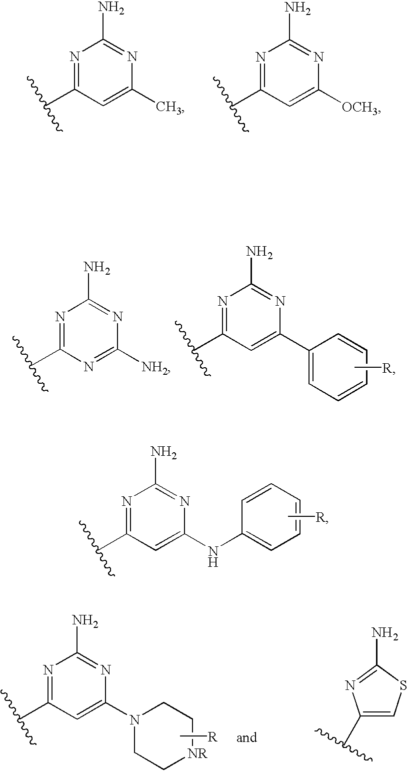

The R₈ substituent of the compounds of Formula III may be selected from the group consisting of

i) hydrogen;

ii) C₂-C₆ alkenylene;

iii) halogen or perhaloalkyl;

iv) an alkoxy of formula —O—X₁₄, where

X₁₄ is selected from the group consisting of hydrogen, lower alkyl, and aryl; and

v) a five-membered or six-membered heteroaryl ring or a six-membered aryl or heteroaryl ring, optionally substituted with one or more substituents selected from the group consisting of

A) optionally substituted C₁-C₄ straight-chain, branched, or cyclic saturated or unsaturated alkyl;

B) an alkoxy of formula —O—X₁₄, where X₁₄ is hydrogen or lower alkyl; and

C) halogen or perhaloalkyl;

D) cyano;

E) nitro;

F) a substituent of formula —(X₁₈)ₙ₁₈—C(O)—X₁₉, where

X₁₈ is lower alkylene;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, and aryl; and

n18 is 0 or 1; and

G) a thioether or thiol of formula —S—X₂₃, where X₂₃ is selected from the group consisting of hydrogen, lower alkyl, aryl, and heteroaryl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, lower alkyl, aryl, hydroxy, and alkoxy; and

X₂₇ is selected from the group consisting of hydrogen, lower alkyl, aryl, and heteroaryl.

In other embodiments, R₈ may be selected from the group consisting of

i) hydrogen;

ii) C₂-C₆ alkenylene;

iii) halogen or perfluoroalkyl;

iv) an alkoxy of formula —O—X₁₄, where

X₁₄ is selected from the group consisting of hydrogen, methyl, ethyl, and propyl; and

v) a six-membered aryl ring, optionally substituted with one or more substituents selected from the group consisting of

A) methyl, ethyl, and propyl;

B) hydroxy, methoxy, ethoxy, phenoxy, hydroxymethyl, 2-hydroxyethyl, and 3-hydroxypropyl; and

C) halogen or perfluoroalkyl;

D) cyano;

E) nitro;

F) a substituent of formula —C(O)—X₁₉, where

X₁₉ is selected from the group consisting of hydrogen, methyl, ethyl, propyl, phenyl, hydroxy, methoxy, ethoxy, propoxy, amino, and —NX₂₀X₂₁,

where X₂₀ and X₂₁ are each independently selected from the group consisting of hydrogen, methyl, ethyl, propyl, and phenyl; and

G) a thioether or thiol of formula —S—X₂₃, where X₂₃ is selected from the group consisting of hydrogen, methyl, ethyl, propyl, and phenyl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, methyl, ethyl, propyl, phenyl, hydroxy, methoxy, and phenoxy; and

X₂₇ is selected from the group consisting of hydrogen, methyl, ethyl, propyl, and phenyl.

The five-membered or six-membered heteroaryl ring or the six-membered aryl or heteroaryl ring of R₈ may be selected from the group consisting of optionally substituted

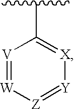

optionally substituted

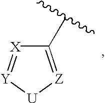

and optionally substituted

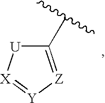

where V, W, X, Y and Z are each independently CR or nitrogen, and U is selected from the group consisting of CR₂, oxygen, sulfur, and NR; where R is as defined herein.

The five-membered or six-membered heteroaryl ring or the six-membered aryl or heteroaryl ring of R₈ is selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, triazine, and benzimidazole.

In other embodiments, the R₈ substituent of the compounds of Formula III is selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonylphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-fluoro-4-methoxyphenyl,

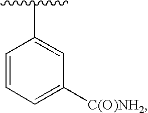

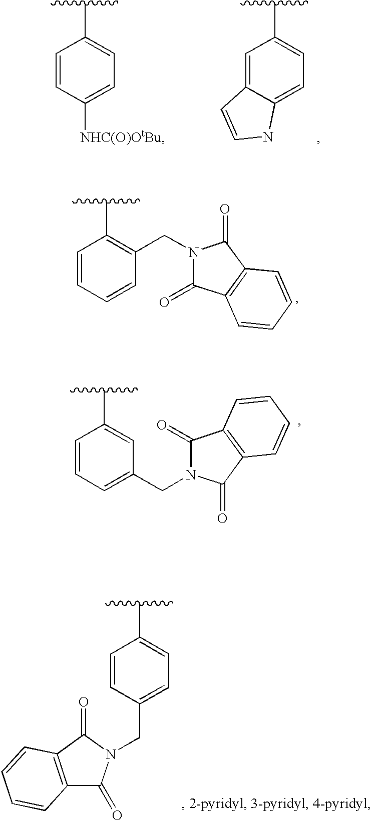

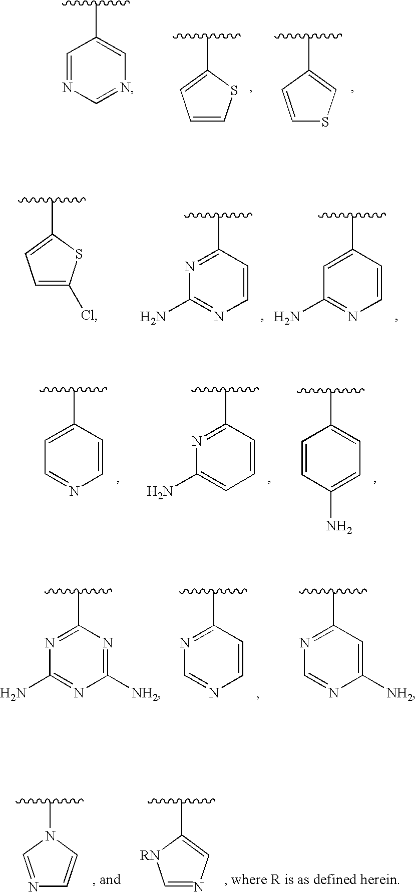

C. The Carbazone Derivatives

In another aspect, the invention relates to a compound of Formula IV

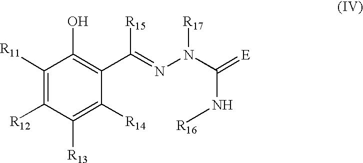

or a pharmaceutically acceptable salt, amide, ester, or prodrug thereof, where

a) R₁₁ is selected from the group consisting of hydrogen, lower alkyl, aryl, and heteroaryl;

b) R₁₂, R₁₃, and R₁₄, are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

c) R₁₅ is selected from the group consisting of hydrogen, lower alkyl, aryl, alkaryl, heteroaryl, and amino;

d) R₁₆ and R₁₇ are each independently selected from the group consisting of hydrogen, lower alkyl, aryl, and heteroaryl; and

e) E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl.

In certain embodiemtns, R₁₃ of the compound of Formula IV is selected from the group consisting of

i) hydrogen;

ii) C₂-C₆ alkenylene;

iii) halogen or perhaloalkyl;

iv) an alkoxy of formula —O—X₁₄, where

X₁₄ is selected from the group consisting of hydrogen, lower alkyl, and aryl; and

v) a five-membered or six-membered heteroaryl ring or a six-membered aryl or heteroaryl ring, optionally substituted with one or more substituents selected from the group consisting of

A) optionally substituted C₁-C₄ straight-chain, branched, or cyclic saturated or unsaturated alkyl;

B) an alkoxy of formula —O—X₁₄, where X₁₄ is hydrogen or lower alkyl; and

C) halogen or perhaloalkyl;

D) cyano;

E) nitro;

F) a substituent of formula —(X₁₈)ₙ₁₈—C(O)—X₁₉, where

X₁₈ is lower alkylene;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, and aryl; and

n18 is 0 or 1; and

G) a thioether or thiol of formula —S—X₂₃, where X₂₃ is selected from the group consisting of hydrogen, lower alkyl, aryl, and heteroaryl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, lower alkyl, aryl, hydroxy, and alkoxy; and

X₂₇ is selected from the group consisting of hydrogen, lower alkyl, aryl, and heteroaryl.

In certain other embodiments, R₁₃ is selected from the group consisting of

i) hydrogen;

ii) C₂-C₆ alkenylene;

iii) halogen or perfluoroalkyl;

iv) an alkoxy of formula —O—X₁₄, where

X₁₄ is selected from the group consisting of hydrogen, methyl, ethyl, and propyl; and

v) a six-membered aryl ring, optionally substituted with one or more substituents selected from the group consisting of

A) methyl, ethyl, and propyl;

B) hydroxy, methoxy, ethoxy, phenoxy, hydroxymethyl, 2-hydroxyethyl, and 3-hydroxypropyl; and

C) halogen or perfluoroalkyl;

D) cyano;

E) nitro;

F) a substituent of formula —C(O)—X₁₉, where

X₁₉ is selected from the group consisting of hydrogen, methyl, ethyl, propyl, phenyl, hydroxy, methoxy, ethoxy, propoxy, amino, and —NX₂₀X₂₁,

where X₂₀ and X₂₁ are each independently selected from the group consisting of hydrogen, methyl, ethyl, propyl, and phenyl; and

G) a thioether or thiol of formula —S—X₂₃, where X₂₃ is selected from the group consisting of hydrogen, methyl, ethyl, propyl, and phenyl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, methyl, ethyl, propyl, phenyl, hydroxy, methoxy, and phenoxy; and

X₂₇ is selected from the group consisting of hydrogen, methyl, ethyl, propyl, and phenyl.

The five-membered or six-membered heteroaryl ring or the six-membered aryl or heteroaryl ring of R₁₃ may in certain embodiments be selected from the group consisting of optionally substituted

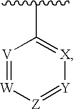

optionally substituted

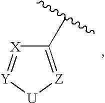

and optionally substituted

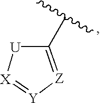

where V, W, X, Y and Z are each independently CR or nitrogen, and U is selected from the group consisting of CR₂, oxygen, sulfur, and NR; where R is as defined herein.

The five-membered or six-membered heteroaryl ring or the six-membered aryl or heteroaryl ring of R₁₃ may also be selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

In some embodiments, R₁₃ may be selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonylphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-

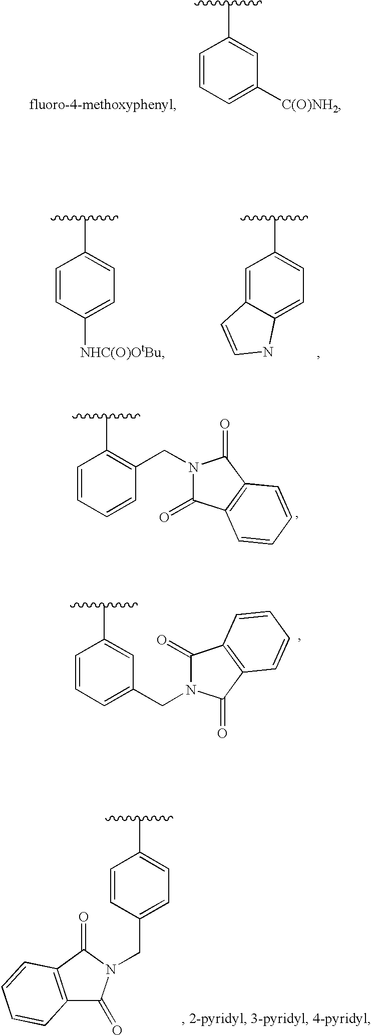

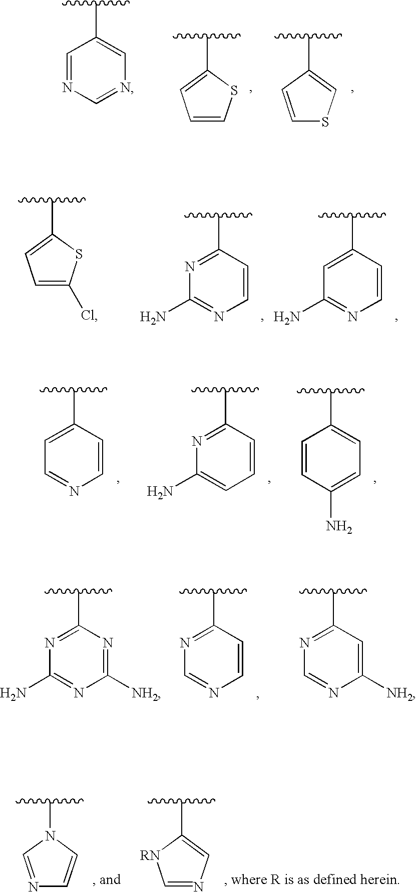

In certain embodiments, R₁₁, R₁₂, R₁₃, and R₁₄ of the compound of Formula IV may each be independently selected from the group consisting of (i) hydrogen, (ii) hydroxyl, (iii) halogens, (iv) cyano, (v) nitro, (vi) amino, (vii) hydroxycarbonyl, (viii) aminocarbonyl, (ix) aminothiocarbonyl, (x) lower alkoxy, (xi) phenoxy, (xii) (C₁-C₄)alkylamino, (xiii) arylamino, (xiv) C₁-C₈ straight-chain, branched, and cyclic saturated and unsaturated alkyl or alkenyl, (xv) optionally substituted aryl and (xvi) optionally substituted hereocycle.

Certain embodiments relate to a compound of Formula IV, in which R₁₅ may be selected from the group consisting of (i) hydrogen, (ii) cyano, (iii) amino, (iv) hydroxycarbonyl, (v) aminocarbonyl, (vi) aminothiocarbonyl, (vii) (C₁-C₄)alkylamino, (viii) arylamino, (ix) C₁-C₈ straight-chain, branched, and cyclic saturated and unsaturated alkyl or alkenyl, (x) optionally substituted aryl and (xi) optionally substituted hereocycle.

In some of the embodiments of the invention, R₁₆ of the compound of Formula IV may be selected from the group consisting of (i) hydrogen, (ii) amino, (iii) hydroxycarbonyl, (iv) aminocarbonyl, (v) aminothiocarbonyl, (vi) (C₁-C₄)alkylamino, (vii) arylamino, (viii) C₁-C₈ straight-chain, branched, and cyclic saturated and unsaturated alkyl or alkenyl, (ix) optionally substituted aryl and (x) optionally substituted hereocycle.

R17 of the compound of Formula IV may in certain embodiments be selected from the group consisting of (i) hydrogen, (ii) (C₁-C₄)alkylamino, (iii) arylamino, (iv) C₁-C₈ straight-chain, branched, and cyclic saturated and unsaturated alkyl, (v) optionally substituted aryl and (vi) optionally substituted hereocycle.

The heterocyle moiety mentioned in the description of the compound of Formula IV may be selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

D. The Naphthalene Derviatives

In another aspect, the invention relates to a compound of Formula V or of Formula VI

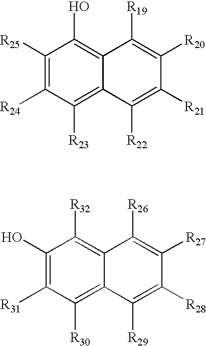

or a pharmaceutically acceptable salt, amide, ester, or prodrug thereof, where

a) R₁₉-R₂₂ and R₂₆-R₂₉ are each independently selected from the group consisting of:

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

b) R₂₃ and R₃₀ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; oxygen or sulfur;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl; and

c) R₂₄, R₂₅, R₃₁ and R₃₂ are each independently selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) acyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1; and

iii) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that none of R₂₄, R₂₅, R₃₁ or R₃₂ is —C₆H₅, —C(O)H, —C(O)CH₃, —C(O)—C₆H₅, —C(O)NH₂, or —C₆H₄CH₃.

In certain embodiments, R₂₄, R₂₅, R₃₁, and R₃₂ may each be independently selected from the group consisting of

i) hydrogen;

ii) a six-membered aromatic ring, optionally substituted with one or more substituents selected from the group consisting of lower alkyl, hydroxy, and amino;

iii) a six-membered heteroaromatic ring, optionally substituted with one or more substituents selected from the group consisting of lower alkyl, hydroxy, alkoxy, amino, and nitro;

iv) a five-membered heteroaromatic ring, optionally substituted with one or more substituents selected from the group consisting of lower alkyl, hydroxy, and amino;

v) acyl of formula —(X₁)ₙ₁—C(O)—X₂, where

X₁ is lower alkylene or lower alkenylene;

X₂ is selected from the group consisting of hydrogen, amino, hydroxy, and —NH—X₃,

where X₃ is selected from the group consisting of hydrogen, lower alkyl, amino, and amide; and

n1 is 0 or 1; and

vi) substituent of formula —C(X₄)═N—NH—C(═E)—NX₆X₇, or of formula —C(X₄)═N—N═C(EX₅)—NX₆X₇, where

X₄ is selected from the group consisting of hydrogen, lower alkyl, aryl, and alkaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen and lower alkyl;

R₂₄, R₂₅, R₃₁ and R₃₂ may also each be independently selected from the group consisting of

i) hydrogen;

ii) a phenyl, optionally substituted with one or more of hydroxy or —NH₂;

iii) a six-membered heteroaromatic ring, selected from the group consisting of pyridine, pyrazine, pyridazine, pyrimidine, and 1,3,5-triazine, each independently and optionally substituted with one or more substituents selected from the group consisting of lower alkyl, hydroxy, alkoxy, and amino;

iv) a five-membered heteroaromatic ring, selected from the group consisting of pyrrole, imidazole, 1,2,3-triazole, 1,2,4-triazole, tetrazole, thiophene, thiazole, and thiadiazole, each independently and optionally substituted with one or more substituent selected from the group consisting of hydroxy, an amide of formula —(X₂₄)ₙ₂₄—NH—C(O)—X₂₅ or —(X₂₆)ₙ₂₆—C(O)—NH—X₂₇, and —NH₂;

v) acyl of formula —C(O)—X₂, where X₂ is hydrogen or lower alkyl;

vi) acyl of formula —X₁—C(O)—X₂, where

X₁ is lower alkylene or lower alkenylene; and

X₂ is —NH—X₃, where X₃ is selected from the group consisting of hydrogen, amino, and amide;

vi) substituent of formula —C(X₄)═N—NH—C(═E)—NH₂, where

X₄ is selected from the group consisting of hydrogen, methyl, ethyl, phenyl, and —CH₂CH₂—Ph; and

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

vii) substituent of formula —C(X₄)═N—N═C(SX₅)—NH₂, where

X₄ is selected from the group consisting of hydrogen, methyl, ethyl, phenyl, and —CH₂CH₂—Ph; and

X₅ is hydrogen or methyl.

The five- or six-membered heteroaryl ring in R₂₄, R₂₅, R₃₁ and R₃₂ in certain embodiments may be selected from the group consisting of optionally substituted

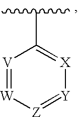

optionally substituted

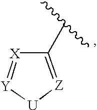

and optionally substituted

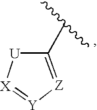

where V, W, X, Y and Z are each independently CR or nitrogen, and U is selected from the group consisting of CR₂, oxygen, sulfur, and NR; where R is as defined herein.

In certain other embodiments, the heteroaryl ring may be selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, oxadiazole, pyran, pyridine, piperidine, morpholine, thiomorpholine, pyridazine, pyrimidine, pyrazine, piperazine, and triazine.

More specifically, the heteroaryl ring may be selected from the group consisting of aminofuran, aminothiophene, aminopyrrole, aminopyrroline, aminopyrrolidine, aminooxazole, aminothiazole, aminoimidazole, aminoimidazoline, aminoimidazolidine, aminopyrazole, aminopyrazoline, aminopyrazolidine, aminoisoxazole, aminoisothiazole, aminotriazole, aminothiadiazole, aminooxadiazole, aminopyran, aminopyridine, aminopiperidine, aminomorpholine, aminothiomorpholine, aminopyridazine, aminopyrimidine, aminopyrazine, aminopiperazine, and aminotriazine.

Certain embodiments of the present invention relate to a compound fo Formula V or Formula VI, in which R₂₄, R₂₅, R₃₁ and R₃₂ may each be independently selected from the group consisting of hydrogen, —C(O)—CH₃, —C(O)—NH—CH₂—C(O)—NH₂, —CH═CH—C(O)—NH₂, —CH₂CH₂—C(O)—NH—NH₂, —C(H)═N—NH—C(O)—NH₂, —C(CH₃)═N—NH—C(O)—NH₂, —C(H)═N—NH—C(S)—NH₂, —C(CH₃)═N—NH—C(S)—NH₂, —C(Ph)═N—NH—C(S)—NH₂, —C(CH₂CH₂Ph)═N—NH—C(S)—NH₂, —C(H)═N—N═C(SCH₃)—NH₂,

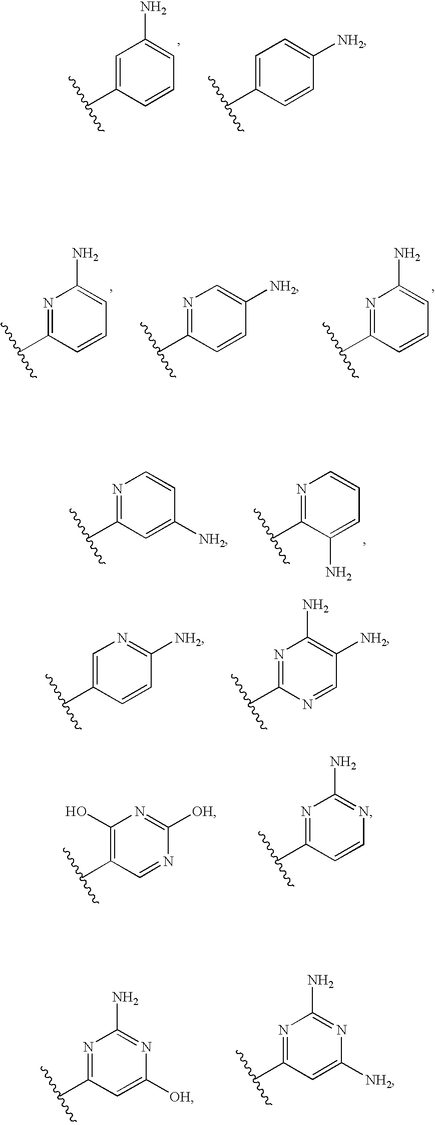

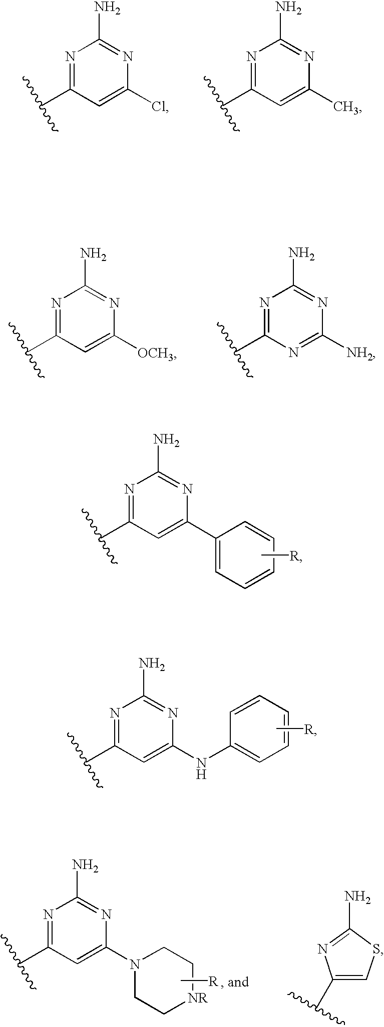

where R is as defined herein.

In some embodiments, R₂₃ and R₃₀ may each be independently selected from the group consisting of

i) hydrogen;

ii) C₂-C₆ alkenylene;

iii) halogen or perhaloalkyl;

iv) an alkoxy of formula —O—X₁₄, where

X₁₄ is selected from the group consisting of hydrogen, lower alkyl, and aryl; and

v) a five-membered or six-membered heteroaryl ring or a six-membered aryl or heteroaryl ring, optionally substituted with one or more substituents selected from the group consisting of

A) optionally substituted C₁-C₄ straight-chain, branched, or cyclic saturated or unsaturated alkyl;

B) an alkoxy of formula —O—X₁₄, where X₁₄ is hydrogen or lower alkyl; and

C) halogen or perhaloalkyl;

D) cyano;

E) nitro;

F) a substituent of formula —(X₁₈)ₙ₁₈—C(O)—X₁₉, where

X₁₈ is lower alkylene;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, and aryl; and

n18 is 0 or 1; and

G) a thioether or thiol of formula —S—X₂₃, where X₂₃ is selected from the group consisting of hydrogen, lower alkyl, aryl, and heteroaryl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, lower alkyl, aryl, hydroxy, and alkoxy; and

X₂₇ is selected from the group consisting of hydrogen, lower alkyl, aryl, and heteroaryl.

In other embodiments, R₂₃ and R₃₀ may each be independently selected from the group consisting of

i) hydrogen;

ii) C₂-C₆ alkenylene;

iii) halogen or perfluoroalkyl;

iv) an alkoxy of formula —O—X₁₄, where

X₁₄ is selected from the group consisting of hydrogen, methyl, ethyl, and propyl; and

v) a six-membered aryl ring, optionally substituted with one or more substituents selected from the group consisting of

A) methyl, ethyl, and propyl;

B) hydroxy, methoxy, ethoxy, phenoxy, hydroxymethyl, 2-hydroxyethyl, and 3-hydroxypropyl; and

C) halogen or perfluoroalkyl;

D) cyano;

E) nitro;

F) a substituent of formula —C(O)—X₁₉, where

X₁₉ is selected from the group consisting of hydrogen, methyl, ethyl, propyl, phenyl, hydroxy, methoxy, ethoxy, propoxy, amino, and —NX₂₀X₂₁,

where X₂₀ and X₂₁ are each independently selected from the group consisting of hydrogen, methyl, ethyl, propyl, and phenyl; and

G) a thioether or thiol of formula —S—X₂₃, where X₂₃ is selected from the group consisting of hydrogen, methyl, ethyl, propyl, and phenyl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, methyl, ethyl, propyl, phenyl, hydroxy, methoxy, and phenoxy; and

X₂₇ is selected from the group consisting of hydrogen, methyl, ethyl, propyl, and phenyl.

The five-membered or six-membered heteroaryl ring or the six-membered aryl or heteroaryl ring of R₂₃ and R₃₀ in some of the embodiments of the present invention may be selected from the group consisting of optionally substituted

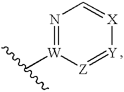

optionally substituted

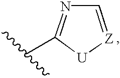

and optionally substituted

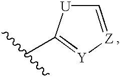

where W, X, Y, and Z are each independently CR or nitrogen, and U is selected from the group consisting of CR₂, oxygen, sulfur, and NR; where R is as defined herein.

The five-membered or six-membered heteroaryl ring or the six-membered aryl or heteroaryl ring of R₂₃ and R₃₀ may also be selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

In some of the embodiments, R₂₃ and R₃₀ may each be independently selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonylphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-fluoro-4-methoxyphenyl,

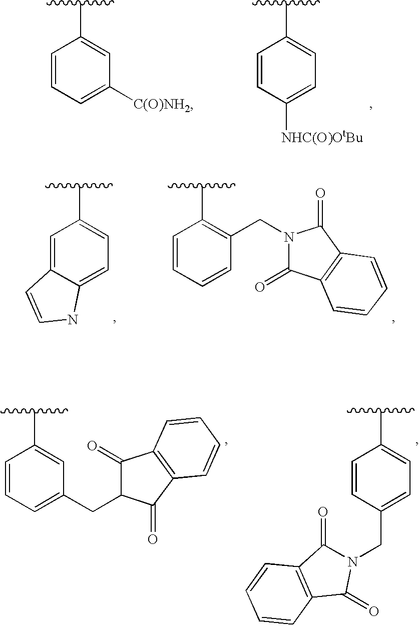

2-pyridyl, 3-pyridyl, 4-pyridyl,

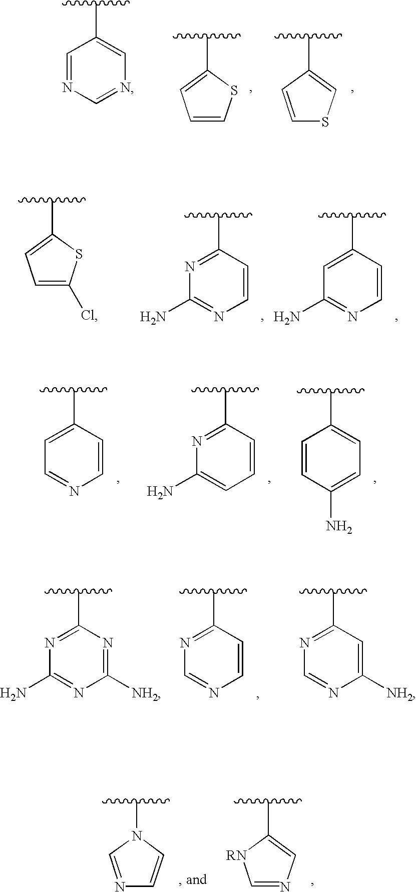

where R is as defined herein.

E. Compounds of the Invention

In another aspect, the invention relates to a compound selected from the group consisting of the compounds set forth in Table 1, or a pharmaceutically acceptable salt, ester, amide, or prodrug thereof.

| Example | Structure |
|---|---|
| 2 | 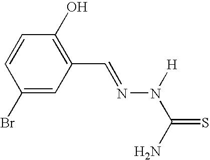 |
| 3 | 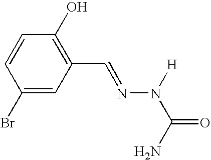 |
| 4 | 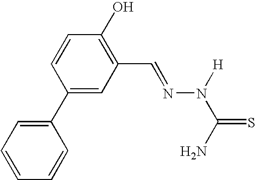 |
| 5 | 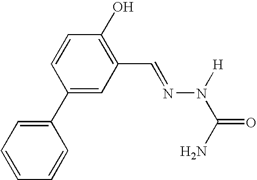 |
| 6 | 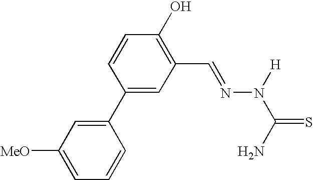 |
| 7 | 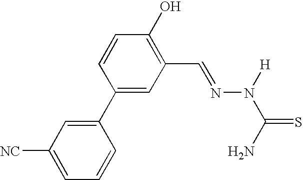 |
| 8 | 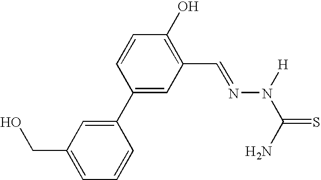 |
| 9 | 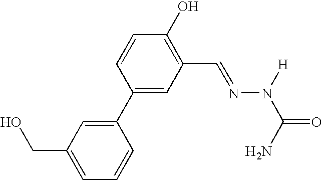 |
| 10 | 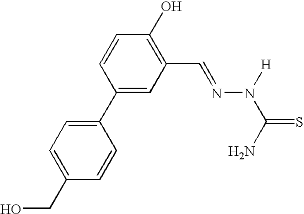 |
| 11 | 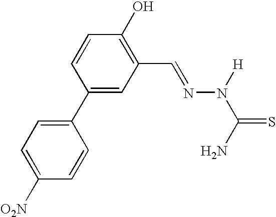 |
| 12 | 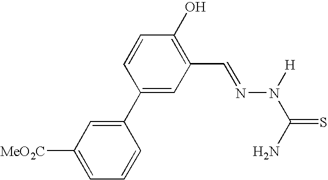 |
| 13 | 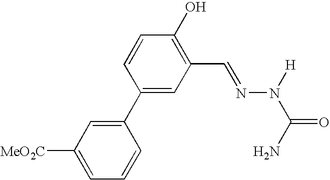 |
| 14 | 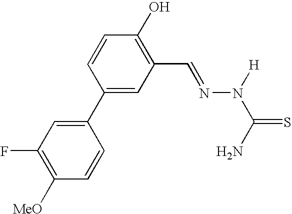 |
| 15 | 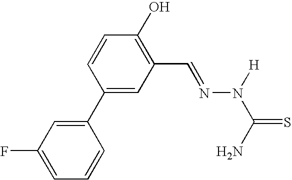 |
| 16 | 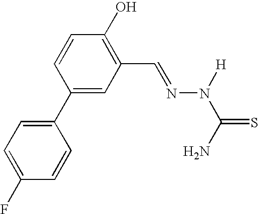 |
| 17 | 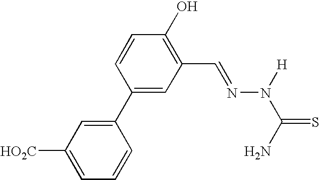 |
| 18 | 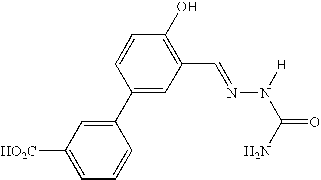 |
| 19 | 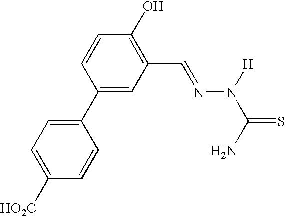 |
| 20 | 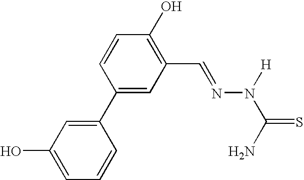 |
| 21 | 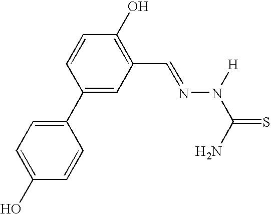 |
| 22 | 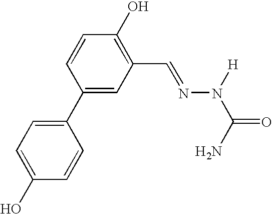 |
| 23 | 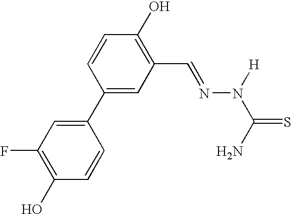 |
| 24 | 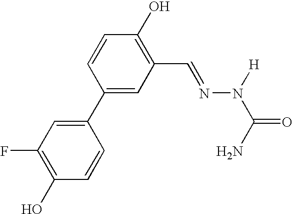 |
| 25 | 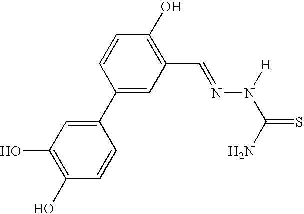 |
| 26 | 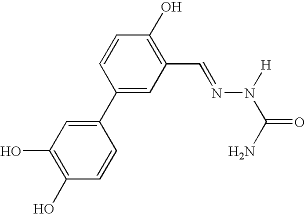 |
| 27 | 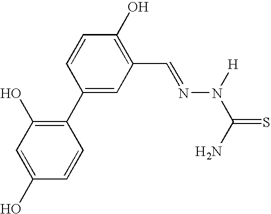 |
| 28 | 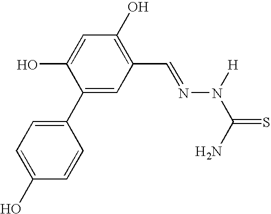 |
| 29 | 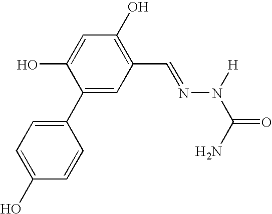 |
| 30 | 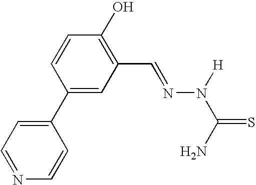 |
| 31 | 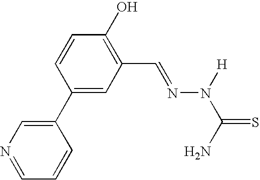 |
| 32 | 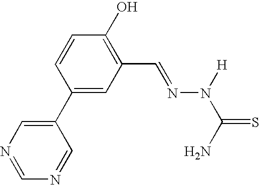 |
| 33 | 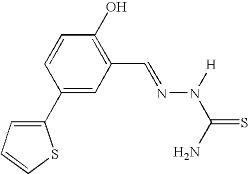 |
| 34 | 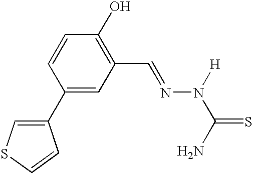 |
| 35 | 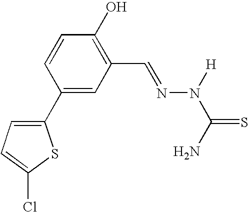 |
| 36 | 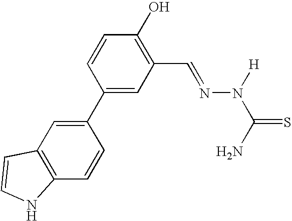 |
| 37 | 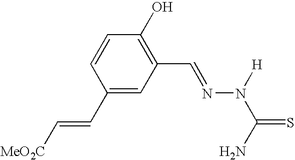 |
| 38 | 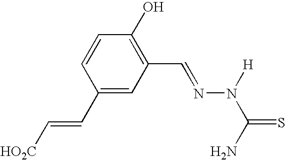 |
| 39 | 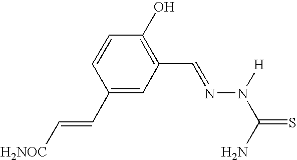 |
| 40 | 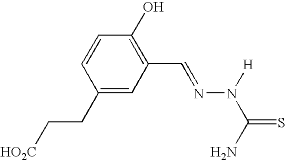 |
| 41 | 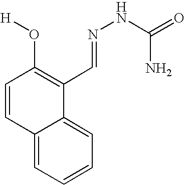 |
| 42 | 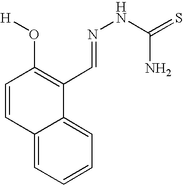 |
| 43 | 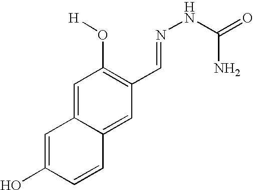 |
| 44 | 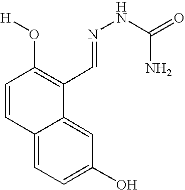 |
| 45 | 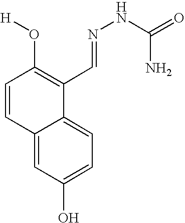 |
| 46 | 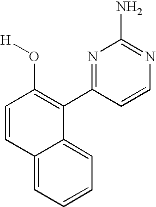 |
| 47 | 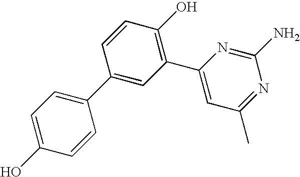 |
| 48 | 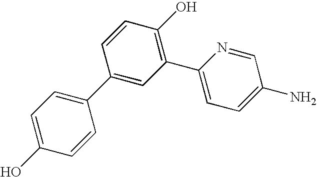 |
| 49 | 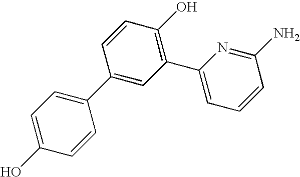 |
| 50 | 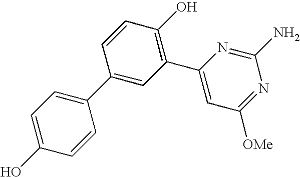 |
| 51 | 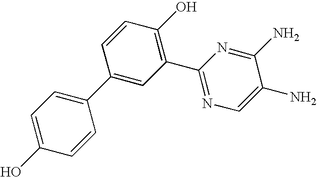 |
| 52 | 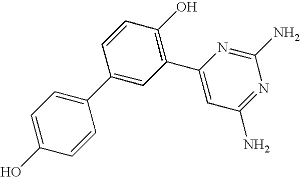 |
| 53 | 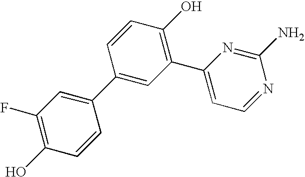 |
| 54 | 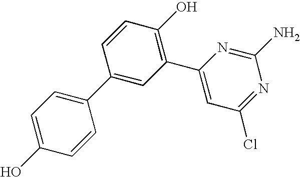 |
| 55 | 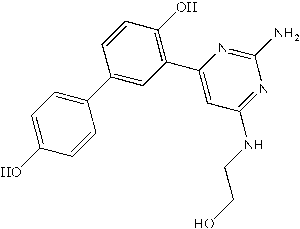 |
| 56 | 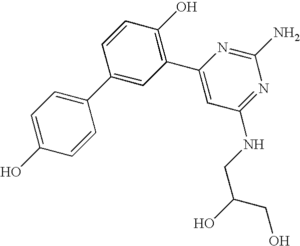 |
| 57 | 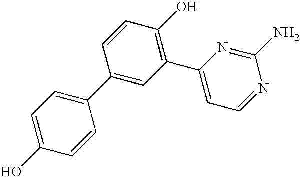 |
| 58 | 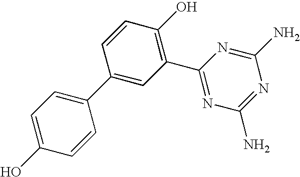 |
| 59 | 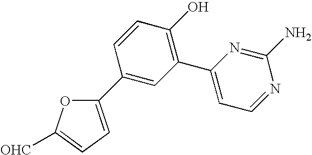 |
| 60 | 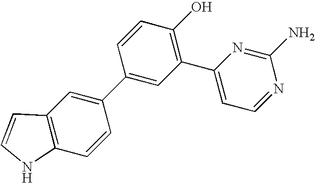 |
| 61 | 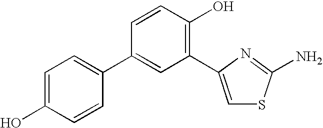 |
| 62 | 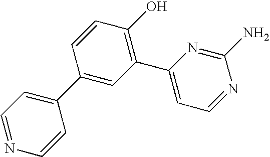 |
| 63 | 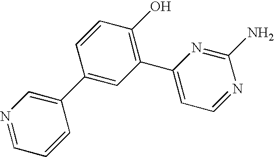 |
| 64 | 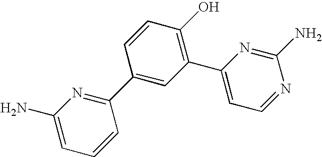 |
| 65 | 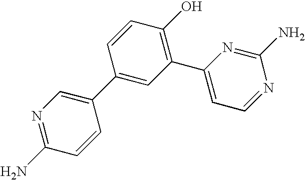 |
| 66 | 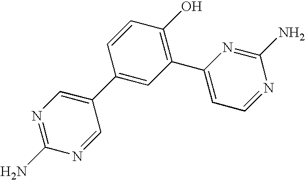 |
| 67 | 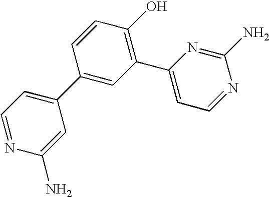 |
| 68 | 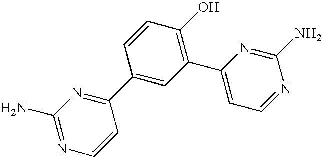 |
| 69 | 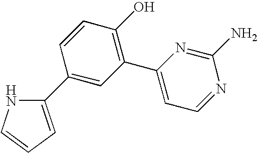 |
| 70 | 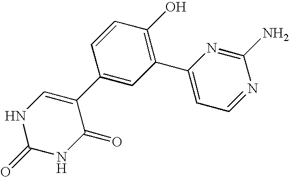 |
| 71 | 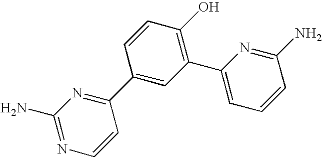 |
| 72 | 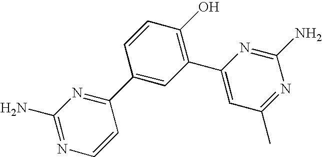 |
| 73 | 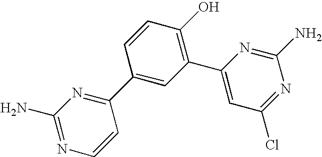 |
| 74 | 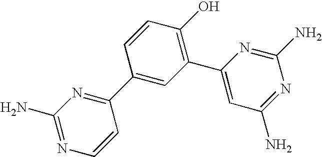 |
| 75 | 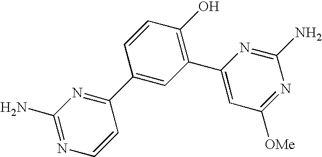 |
| 76 | 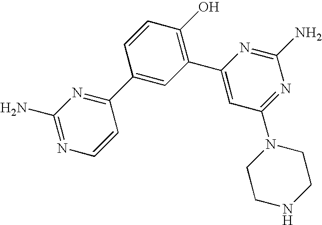 |
| 77 | 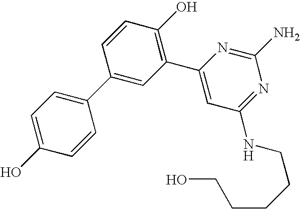 |
| 78 | 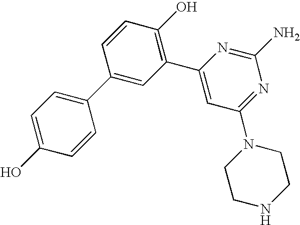 |
| 79 | 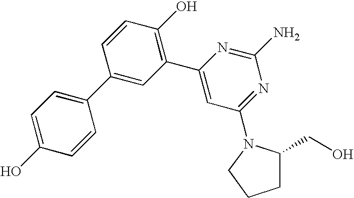 |
| 80 | 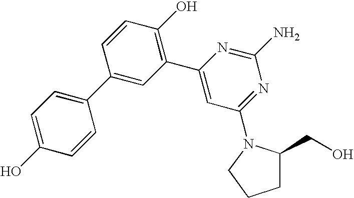 |
| 81 | 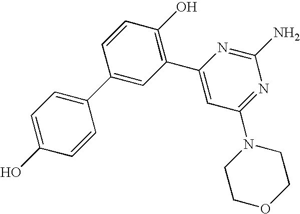 |
| 82 | 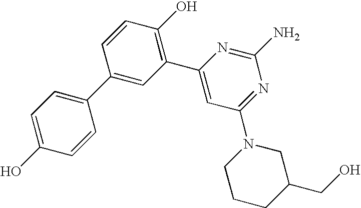 |
| 83 | 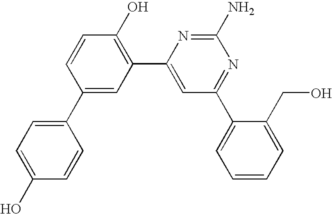 |
| 84 | 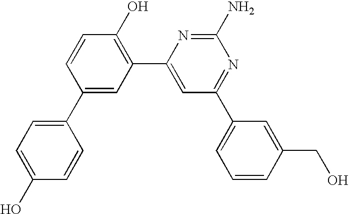 |
| 85 | 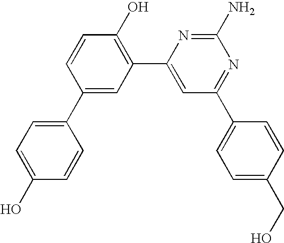 |
| 86 | 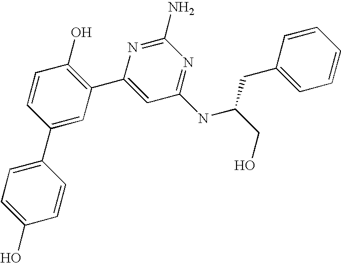 |
| 87 | 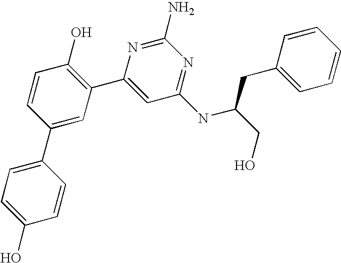 |
| 88 | 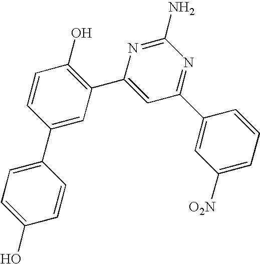 |
| 89 | 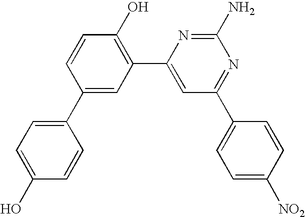 |
| 90 | 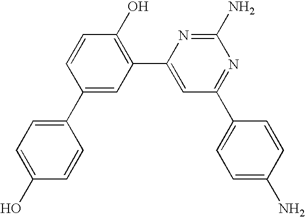 |
| 91 | 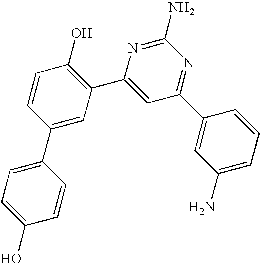 |
| 92 | 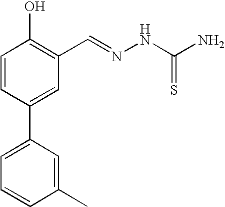 |
| 93 | 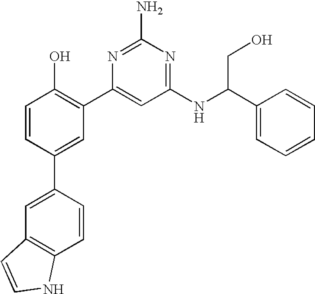 |
| 94 | 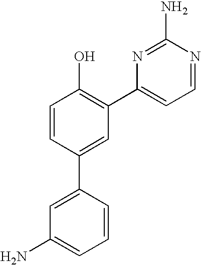 |
| 95 | 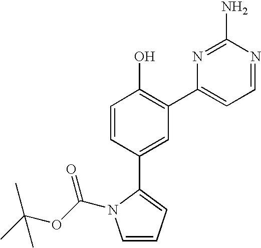 |
| 96 | 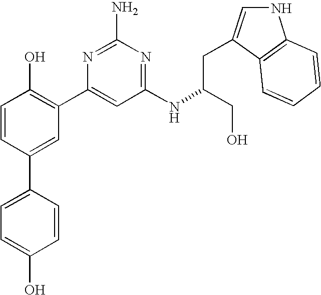 |
| 97 | 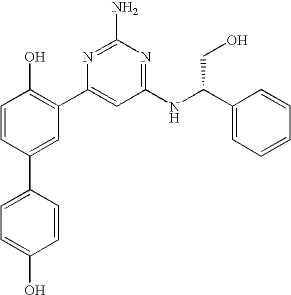 |
| 98 | 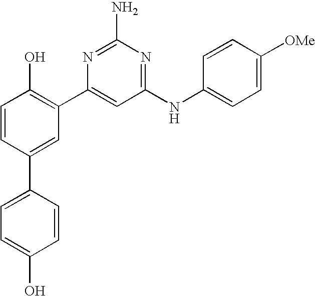 |
| 99 | 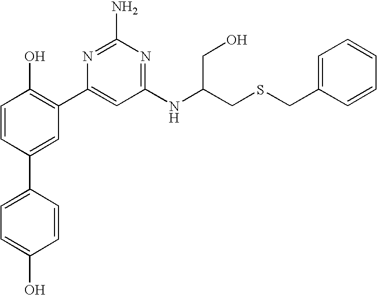 |
| 100 | 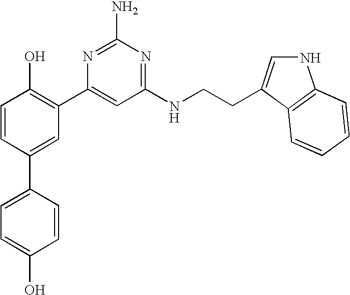 |
| 101 | 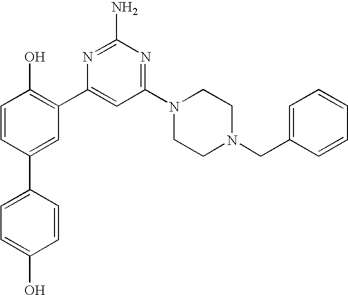 |
| 102 | 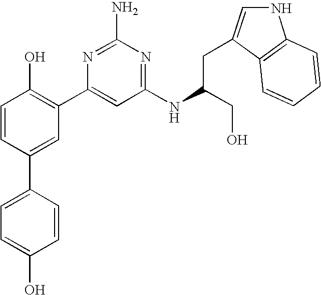 |
| 103 | 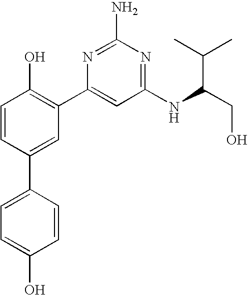 |
| 104 | 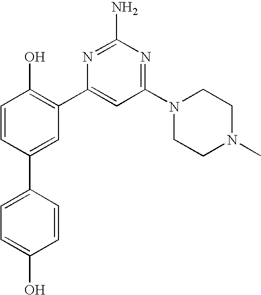 |
| 105 | 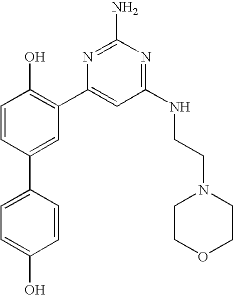 |
| 106 | 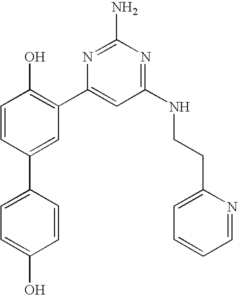 |
| 107 | 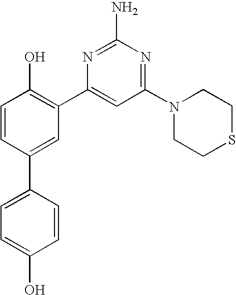 |
| 108 | 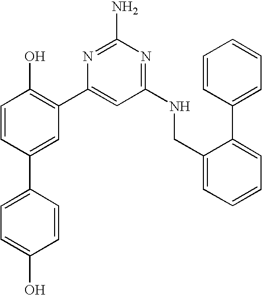 |
| 109 | 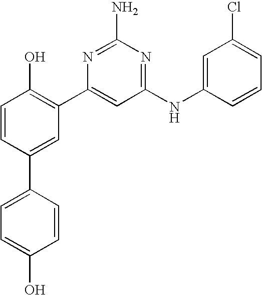 |
| 110 | 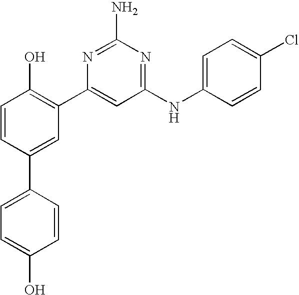 |
| 111 | 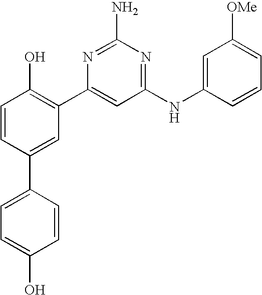 |
| 112 | 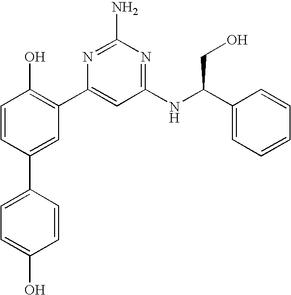 |
| 113 | 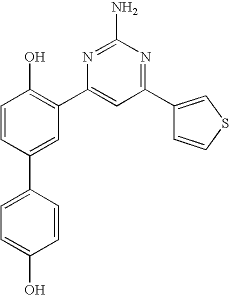 |
| 114 | 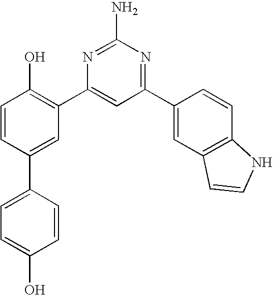 |
| 115 | 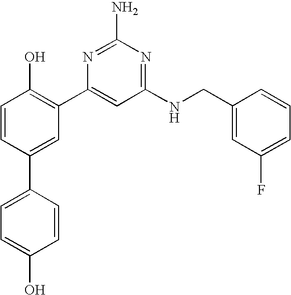 |
| 116 | 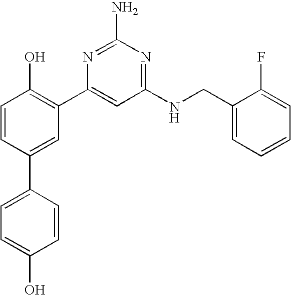 |
| 117 | 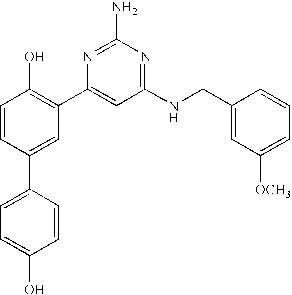 |
| 118 | 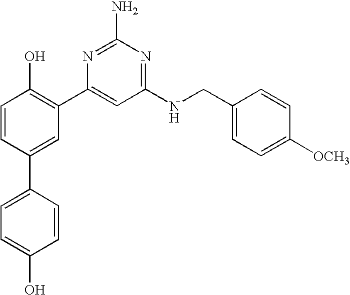 |
| 119 | 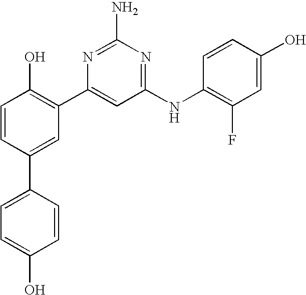 |

### II. Combinatorial Libraries

In another aspect, the invention provides a combinatorial library of at least 10 compounds that can be formed by reacting an acyl compound of Formula VII with an amine of Formula VIII,

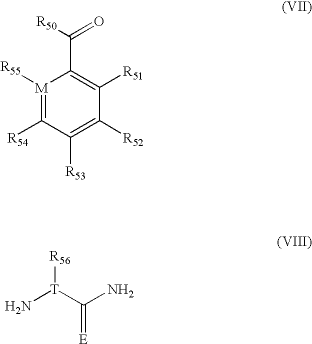

wherein

a) R₅₀ is selected from the group consisting of hydrogen, lower alkyl, aryl, alkaryl, heteroaryl, and amino;

b) R₅₁ is selected from the group consisting of hydrogen, lower alkyl, and hydroxy;

c) R₅₂ is selected from the group consisting of hydrogen, optionally substituted lower alkyl, hydroxy, lower alkoxy, halogen, and nitro;

d) R₅₃ is selected from the group consisting of hydrogen, hydroxy, optionally substituted lower alkyl, and optionally substituted lower alkoxy;

e) R₅₄ is selected from the group consisting of hydrogen, hydroxy, optionally substituted lower alkyl, optionally substituted lower alkoxy, optionally substituted aryl, optionally substituted cycloalkyl, halogen, and nitro;

f) R₅₅ is selected from the group consisting of hydrogen, lower alkyl, and hydroxy;

or R₅₃ and R₅₄, taken together along with the two ring carbons to which they are attached, or R₅₄ and R₅₅, taken together along with the two ring carbons to which they are attached, form an optionally substituted six-membered aromatic or heteroaromatic, or an optionally substituted five- or six-membered heteroaromatic ring,

g) M is selected from the group consisting of carbon and nitrogen, such that when M is nitrogen, R₅₅ does not exist;

h) T is selected from the group consisting of nitrogen and —CR₅₆—;

i) R₅₆ is selected from the group consisting of hydrogen and optionally substituted lower alkyl; and

j) E is selected from the group consisting of oxygen, sulfur, and NH;

wherein at least one of R₅₁-R₅₅ is not a hydrogen and at least one of R₅₁, R₅₂, or R₅₅ is a hydroxy.

A “combinatorial library” refers to all the compounds formed by the reaction of each compound of one dimension with a compound in some or all of the other dimensions in a multi-dimensional array of compounds. In the context of the present invention, the array may be two dimensional, where one dimension represents the acyl compounds of Formula VII and the second dimension represents the amine of Formula VIII. Each acyl compound may be reacted with each and every amine in order to form a compound of the invention. All compounds of the invention formed in this way are within the scope of the present invention. Also within the scope of the present invention are smaller combinatorial libraries formed by the reaction of some of the acyl based compounds with all of the amines, all of the acyl compounds with some of the amines, or some of the acyl compounds with some of the amines.

In certain embodiments, R₅₀ may be selected from the group consisting of (i) hydrogen, (ii) cyano, (iii) amino, (iv) hydroxycarbonyl, (v) aminocarbonyl, (vi) aminothiocarbonyl, (vii) (C₁-C₄)alkylamino, (viii) arylamino, (ix) C₁-C₈ straight-chain, branched, and cyclic saturated and unsaturated alkyl or alkenyl, (x) optionally substituted aryl and (xi) optionally substituted hereocycle.

In other embodiments, R₅₁ and R₅₅ may each independently be hydrogen or hydroxy, R₅₂ may be selected form the group consisting of hydrogen, hydroxy, fluoro, chloro, bromo, iodo, halomethyl, methoxy, and nitro, while R₅₃ may be selected form the group consisting of hydrogen, hydroxy, methoxy, and benzyloxy. In further embodiments, R₅₄ may be selected form the group consisting of hydrogen, methyl, ethyl, propyl, phenyl, cyclohexyl, hydroxy, fluoro, chloro, bromo, iodo, methoxy, and nitro.

Other embodiments of the invention relate to a combinatorial library, in which the acyl compound of Formula VII is selected from the group consisting of

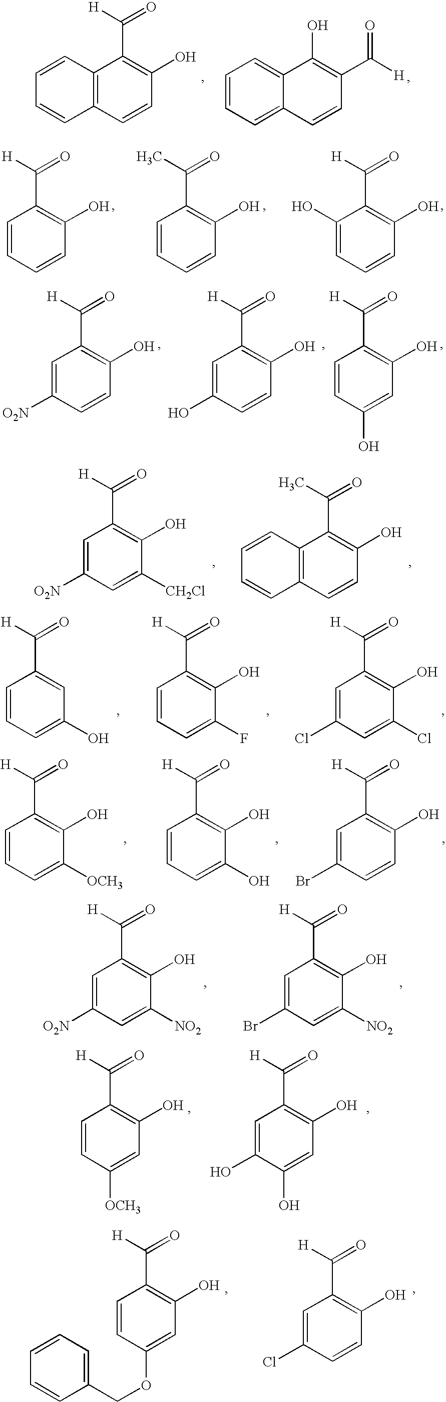

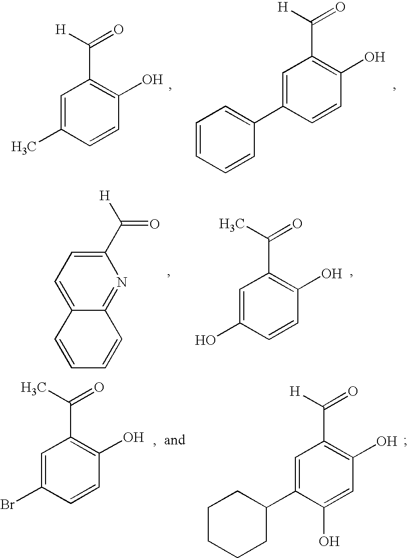

and the amine compound of Formula VIII is selected from the group consisting of

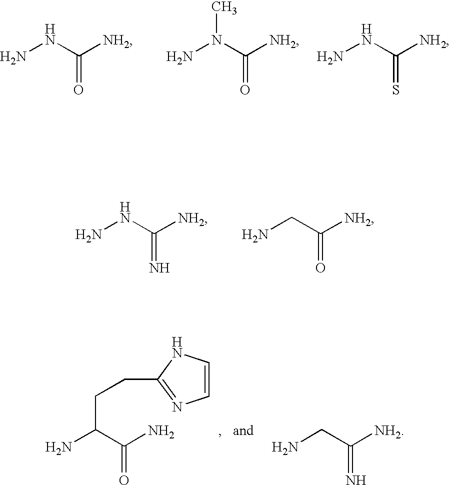

The reaction between the acyl of Formula VII and the amine of Formula VIII may be conducted under the following general conditions. Equal amounts (by volume) of 0.05 M DMSO solutions of each of the acyl compound and the amine compound are combined in combinatorial fashion, along with a catalytic amount of sulphonic acid. After 16 hours, thin-layer chromatographic analysis shows the completion of the reaction. The resulting product in DMSO can be used in an assay without further purification.

### III. Synthesis of the Compounds of the Invention

There are three synthetic strategies used to make the phenol derivative compounds of the invention, which have the general structures as follows:

Phenol

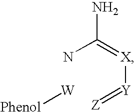

Phenol

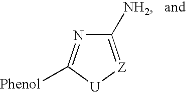

and Phenol

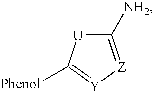

where U, W, X, Y, and Z are as defined herein. The first method is to generate boronic acid attached to the biaryl core structure as shown in Reaction Scheme 1 and Reaction Scheme 2. The second method is to construct the heterocycle ring using a functional group on the biaryl, as shown in Reaction Schemes 3-4. For the synthesis of compounds of Formula II, the aminopyrimidine ring was constructed before conducting the Suzuki reaction to introduce second aromatic ring, as shown in the Reaction Scheme 5.

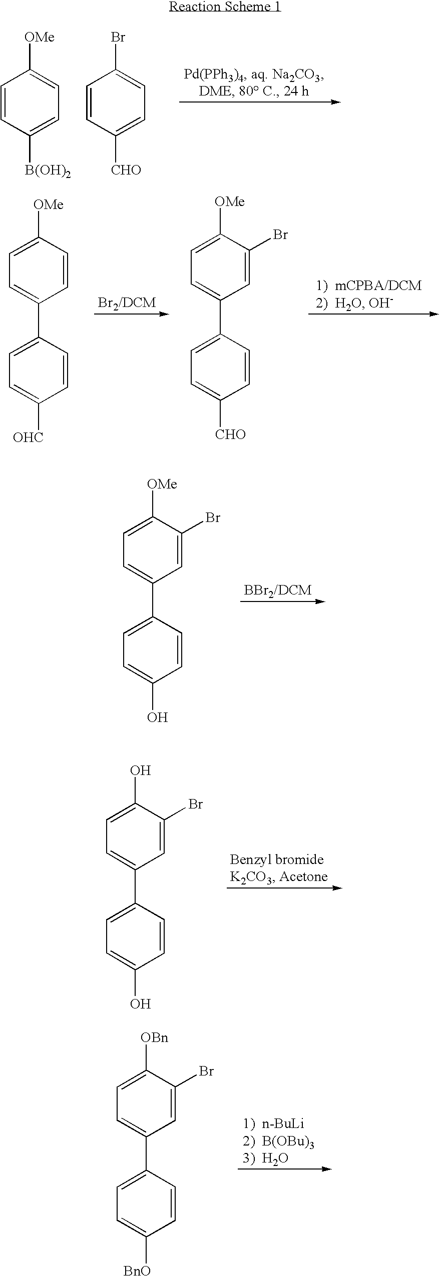

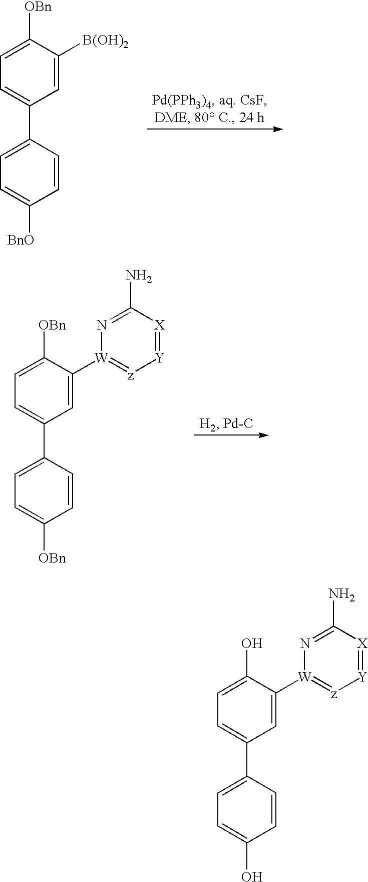

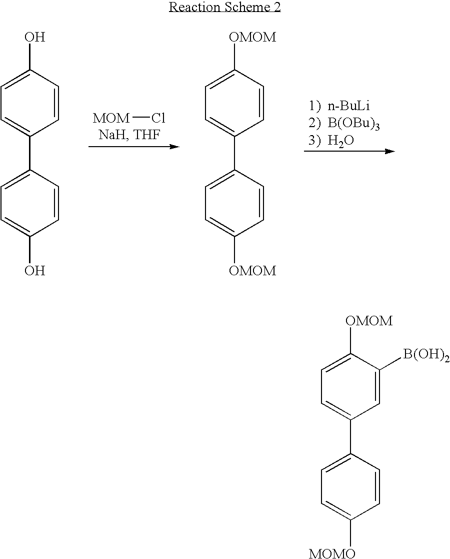

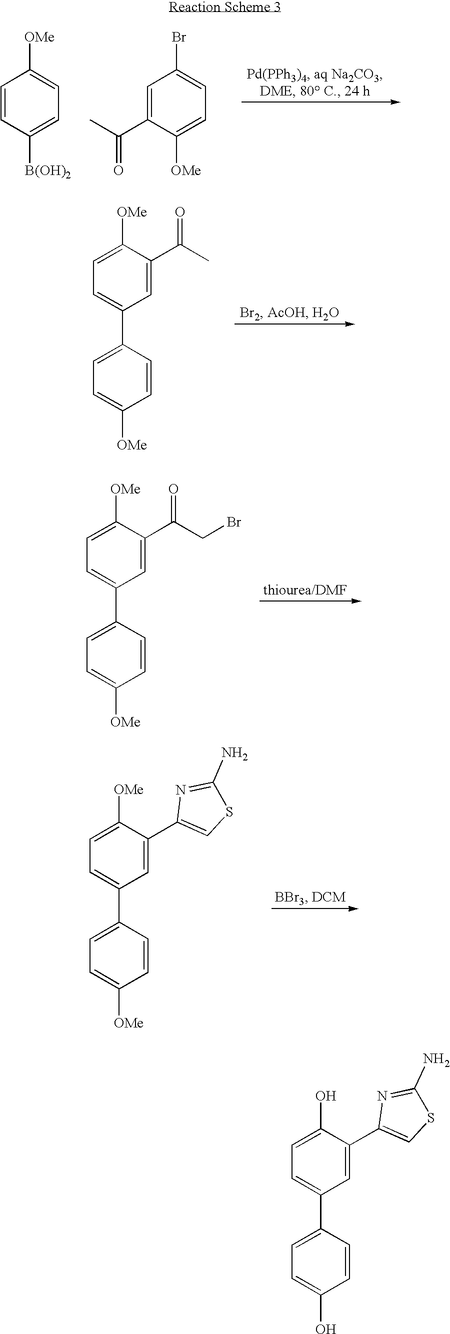

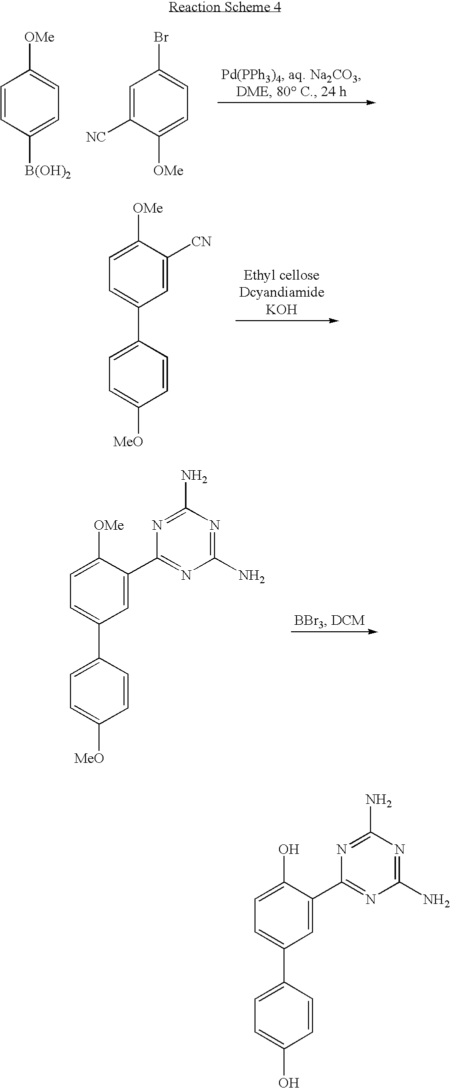

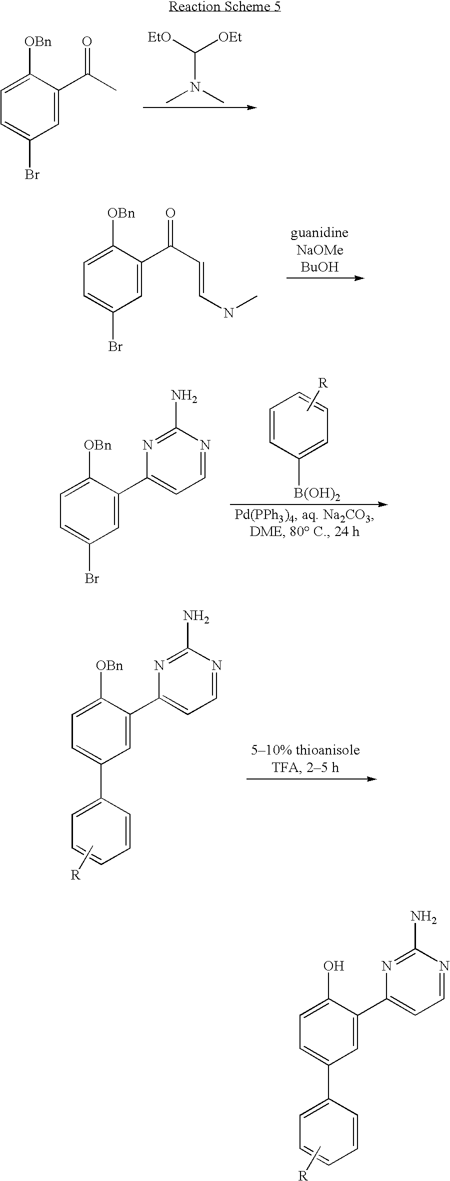

The semicarbazone/thiosemicarbazone compounds of the invention are synthesized according to the following schemes.

In Reaction Scheme 11, the salicylaldehydes are reacted with semicarbazide hydrochloride or thiosemicarbazide in the presence of catalytic amount of sulfonic acid to give high yield of semicarbazone/thiosemicarbarzone product.

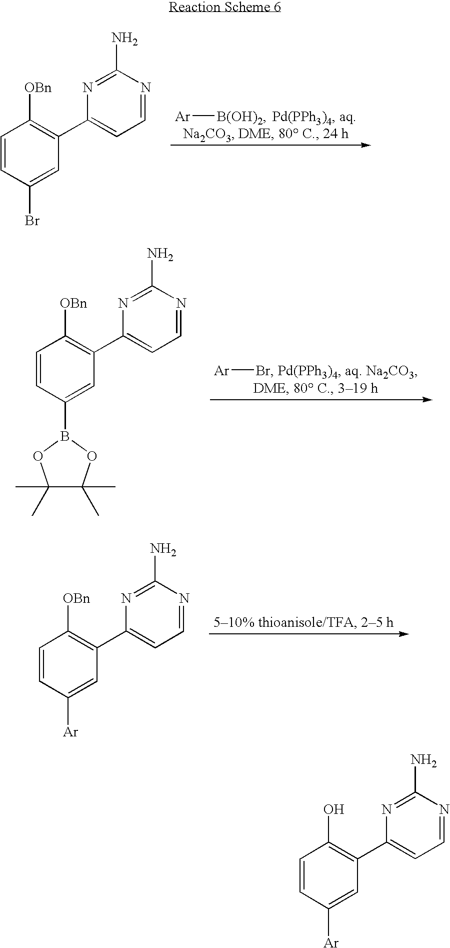

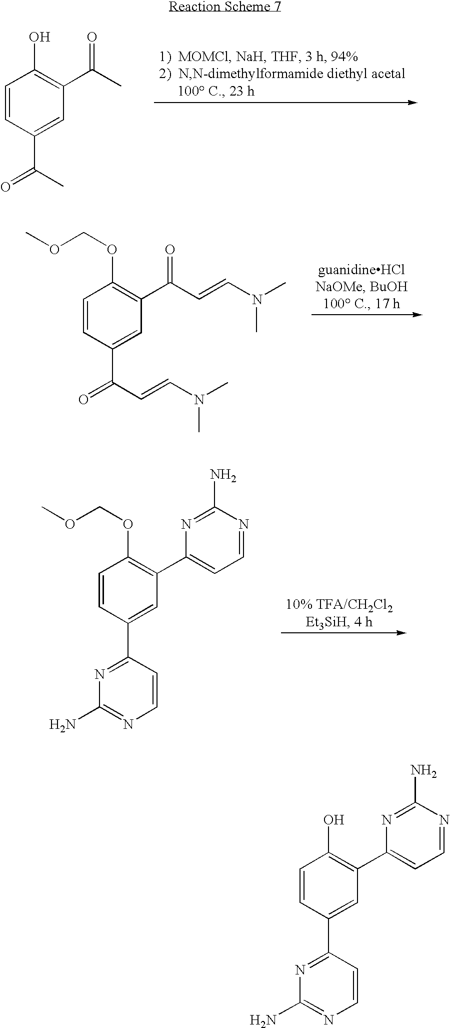

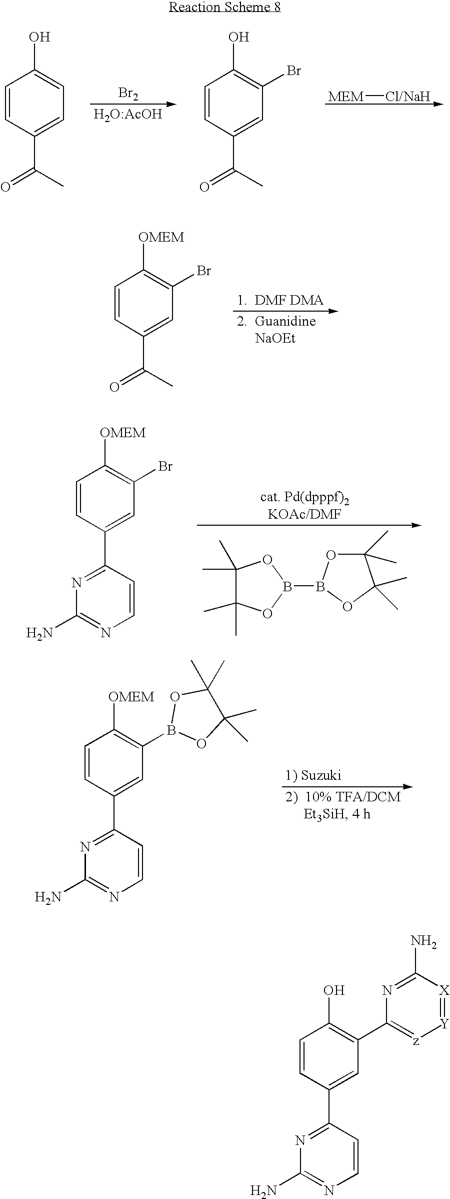

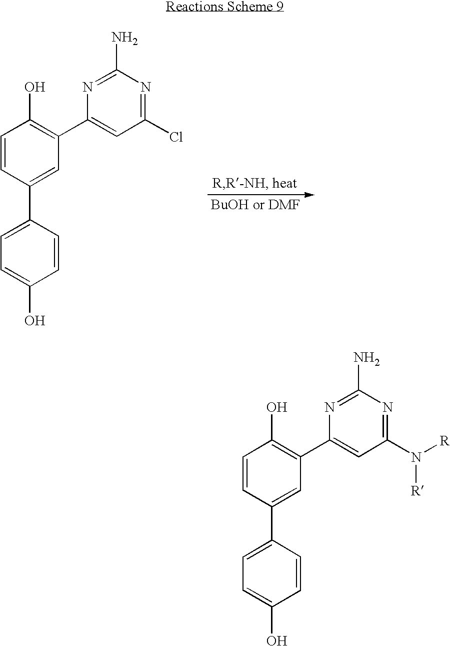

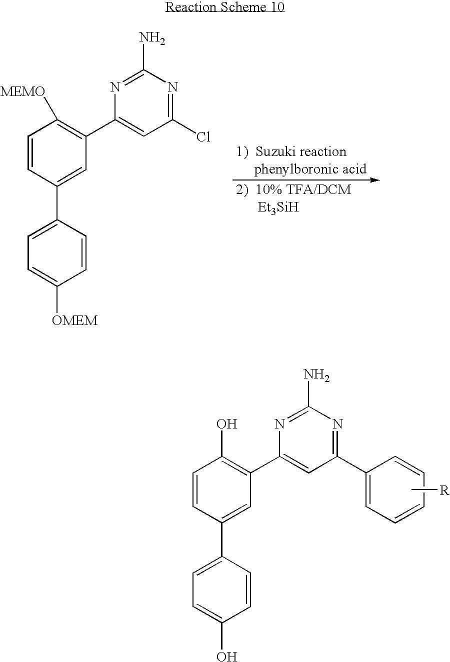

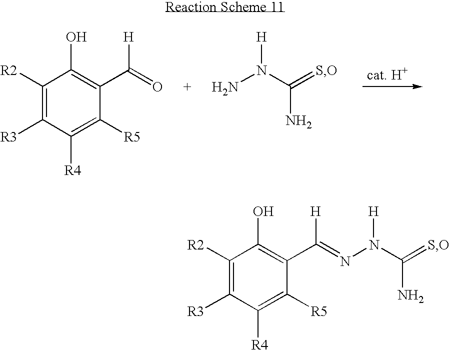

The preparation of the 5-aryl salicylaldehydes was achieved via the Suzuki reaction (Miyaura, N.; Suzuki, A.: Chem. Rev. 1995, 95, 2457-2483). In this reaction, 1 equivalent of 5-bromo salicylaldehydes, 1.05 equivalents of arylboronic, and 0.02 equivalents tetrakis (triphenylphosphine) palladium are dissolved in ethylene glycol dimethyl (DME) ether to give a final concentration of 0.2-0.3 M. To this mixture, 2.5 equivalents aqueous 2.0 M sodium carbonate is added and the mixture is refluxed under nitrogen for 2-16 hours. The resulting products were purified by column chromatography.

Following the reactions set forth in Reaction Scheme 12, to 1.05 equivalents of the resulting 5-aryl salicylaldehydes, 1 equivalent of semicarbazide or thiosemicarbazide is added and dissolved in dimethyl formamide to a final concentration of 0.5 molar. Following addition of a trace amount of camphorsulfonic acid, the reaction is stirred at room temperature for 2-18 hours. Upon completion of the reaction, as judged by thin layer chromatography, the solvent is removed in vacuo and the product is washed six times with diethyl ether and dried in vacuo.

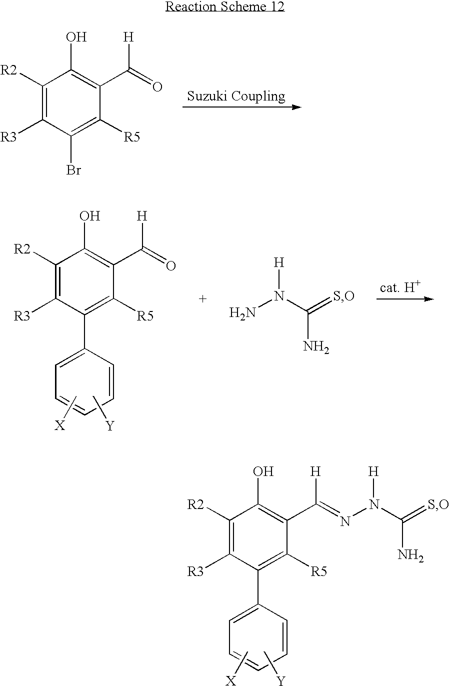

When a methoxy group is attached to the arylboronic acid, the Suzuki reaction was performed first to give methoxy substituted aryl salicyaldedehydes (Reaction Scheme 13). The methyl groups were removed using tribromoborane in DCM at −78° C. The resulting hydroxyaryl substituted salicylaldehydes are reacted with semicarbazide hydrochloride or thiosemicarbazide in the presence of catalytic amount of sulfonic acid to give high yield of semicarbazone/thiosemicarbarzone product.

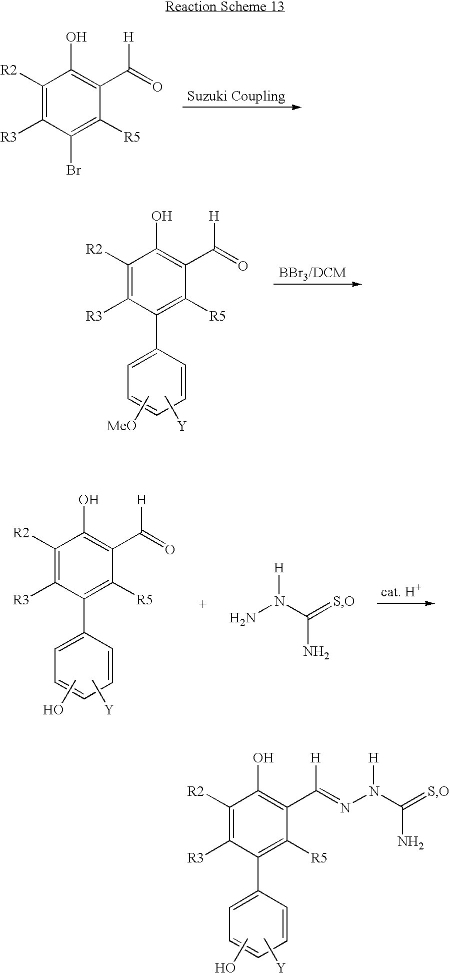

To synthesize 5-(5-pyrimidyl)salicylaldehyde thiosemicarbazone, the boronate was introduced onto the suitable protected salicylaldehyde moiety (Murata, M.; Oyama, T.; Watanabe, S and Masuda, Y. J. Org. Chem. 2000, 65, 164-168) (Reaction Scheme 14). The boronate obtained was then reacted with 5-bromo-pyrimidine via the Suzuki coupling reaction and the protected 5-(5-pyrimidyl)-salicylaldehyde was obtained in yield of 72%. The protecting groups were then removed by using tribromoborane in dichloromethane at low temperature before the semicarbazone formation reaction was performed.

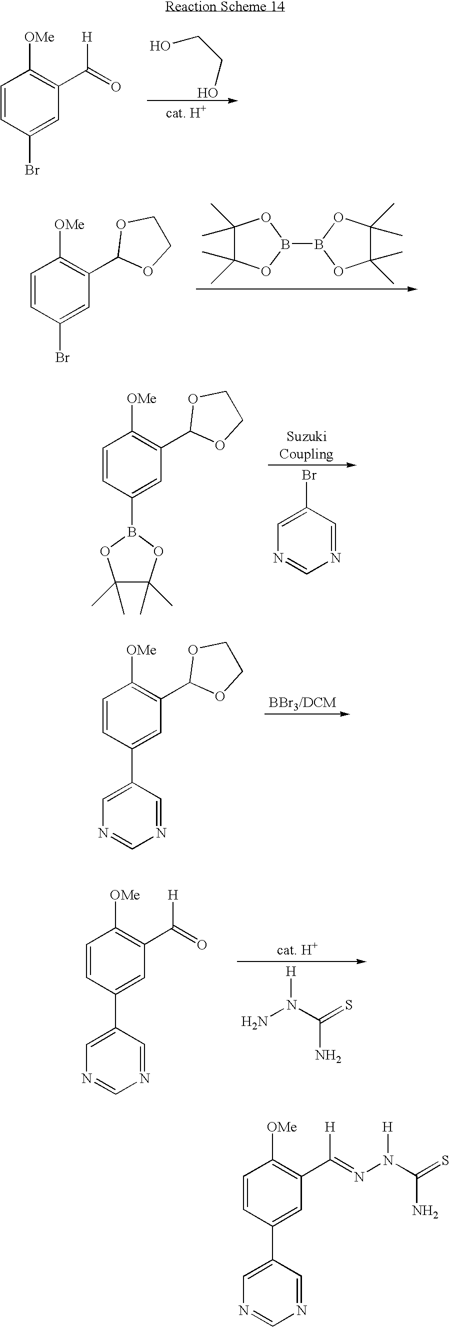

The cinnamate type of compounds (Reaction Schemes 15, 16, and 17) were obtained through Heck reaction, starting from 5-bromosalicylaldehyde and the corresponding acrylate or acrylamide (Zebovitz, T. C.; Heck, R. F. J. Org. Chem., 1997, 42, 3907-3909). The resulting aldehyde was reacted further with semicarbazide or thiosemicarbazide to give corresponding semicarbazone and thiosemicarbazone product.

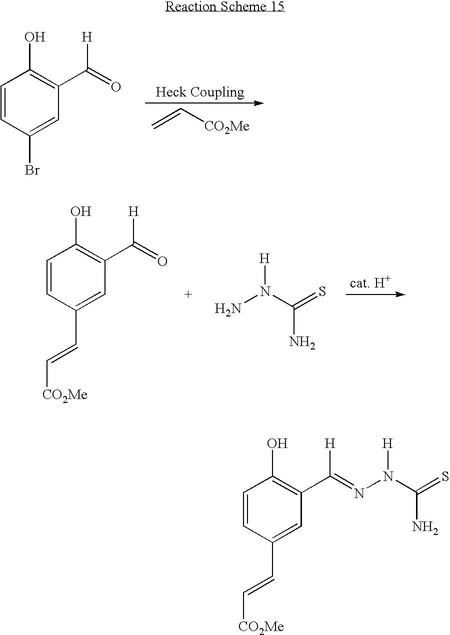

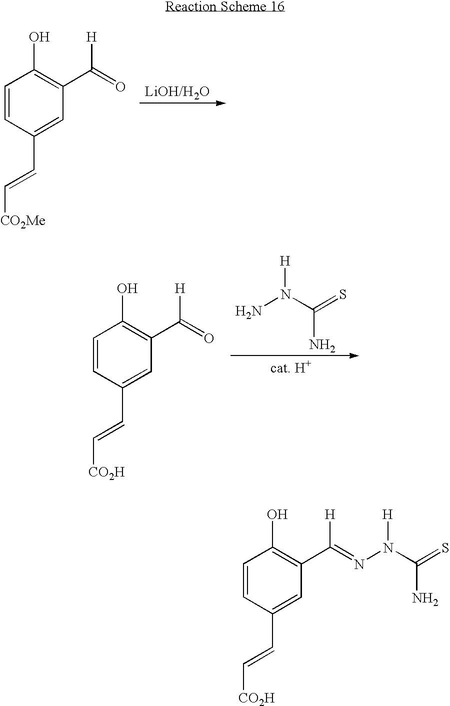

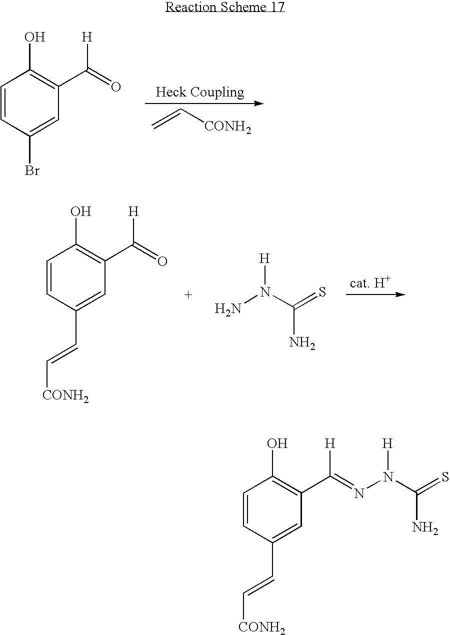

As shown in the Reaction Scheme 18, 3-(4-hydroxyphenyl)propionic acid was readily formylated at the ortho position to the hydroxy group based on a known procedure (Russel, A. and Lockhart, L. B., Org. Syn. Coll., V3, 463-464) to give 3-(3-formyl-4-hydroxyphenyl)propionic acid. This aldehyde is reacted further with thiosemicarbazide to convert to the corresponding thiosemicarbazone product.

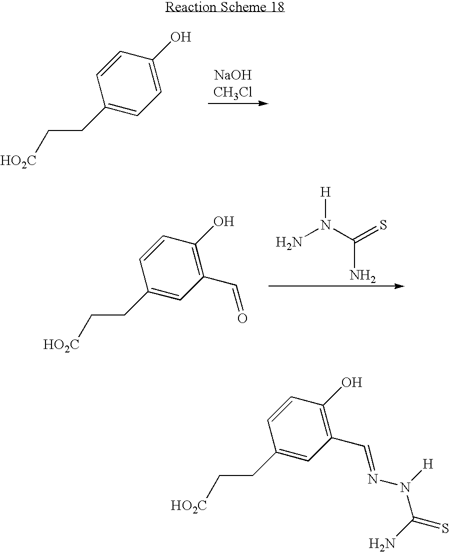

Process and methods for preparing the naphthalene dervative compounds of the invention are illustrated in the following reaction Schemes.

In Reaction Scheme 19, naphthalenes (I) are formylated with phosphorous oxochloride and N,N-dimethylformamide and the aldehydes obtained are further reacted with semicarbazide or thiosemicarbazide in the presence of catalytic amount of sulfonic acid.

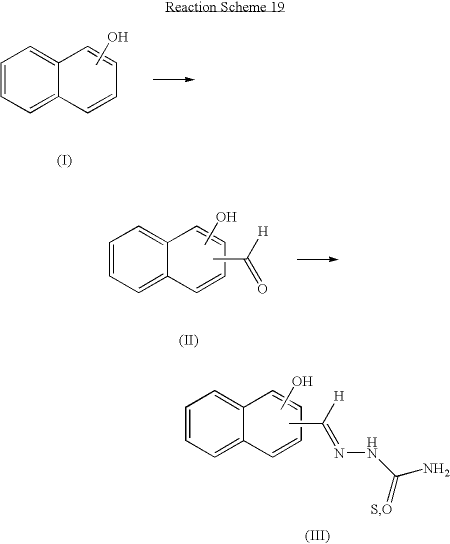

Reaction Scheme 20 provides the synthetic route to pyrimidine compound (VII). One equivalent of ketone (IV) and 15 eq. of N,N-dimethylformamide diethyl acetal are combined and heated to 100° C. overnight. The reaction mixture is concentrated in vacuo and dissolved in butanol (0.1 M) with 3 equivalents of sodium ethoxide followed by 1 equivalents of guanidine hydrochloride salt. This mixture is refluxed for 16 hours to give corresponding pyrimidine (VI). The methyl ether is then readily converted to a hydroxyl group by using tribromoborane in methylenedichloride.

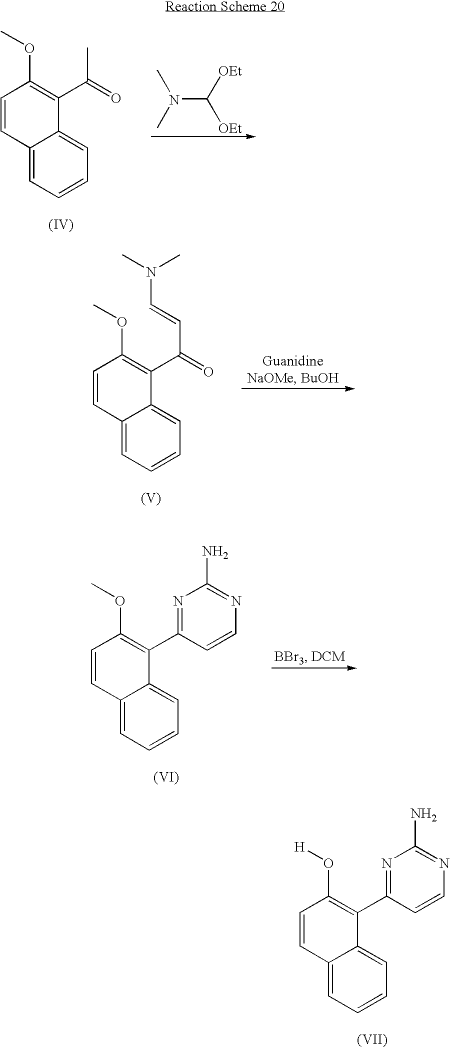

The following Reaction Schemes 21-26 represent novel methods of synthesizing some of the aromatic compounds of the present invention.

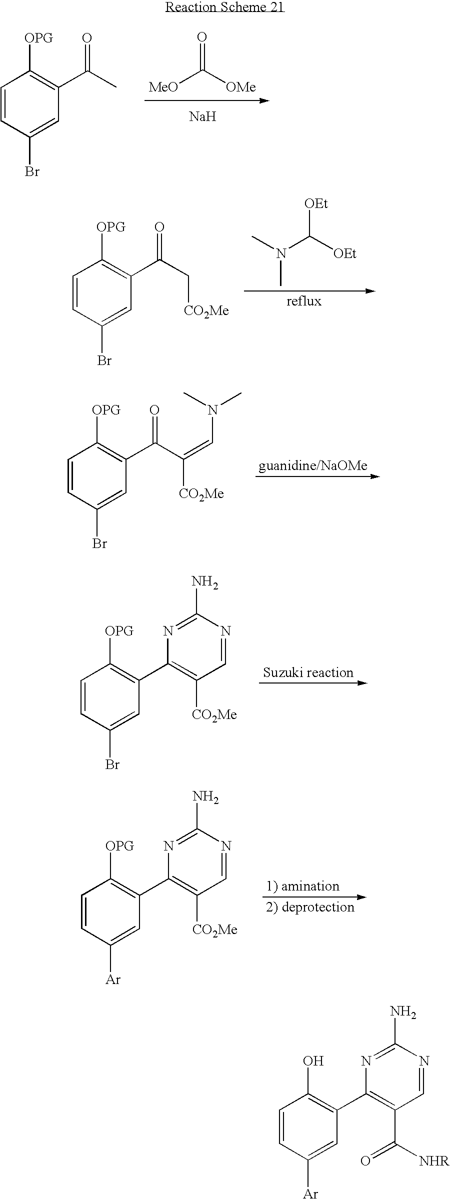

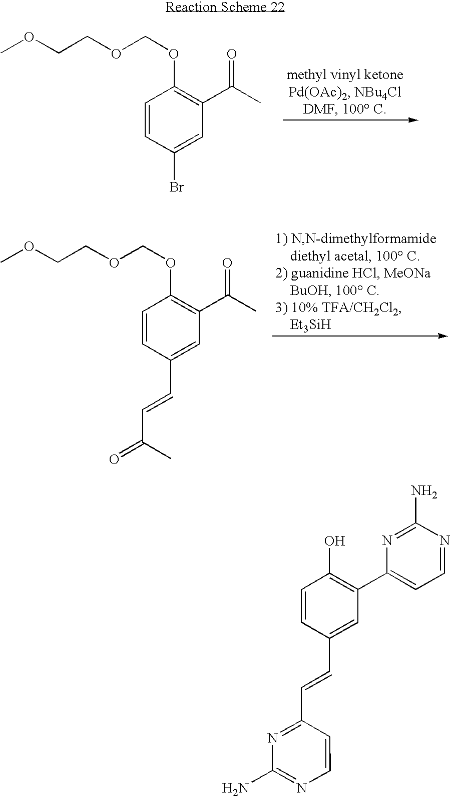

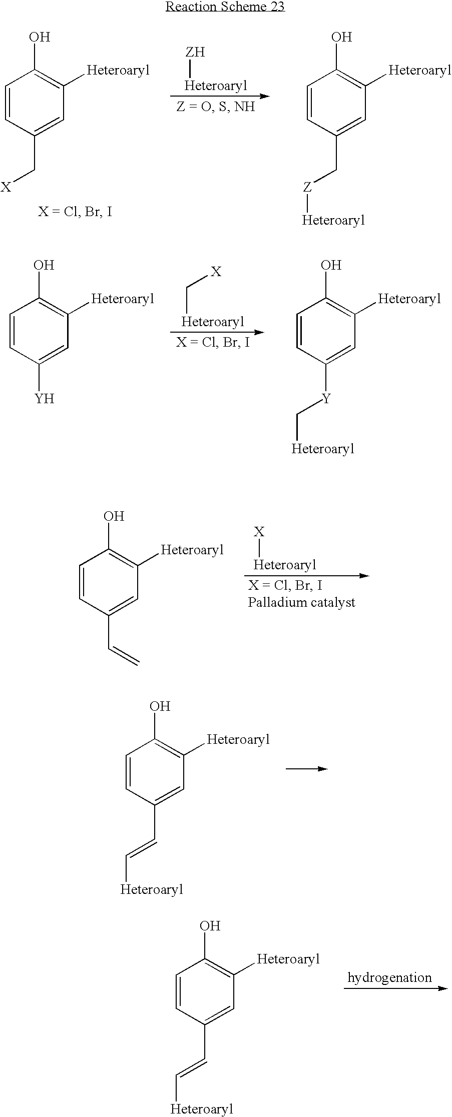

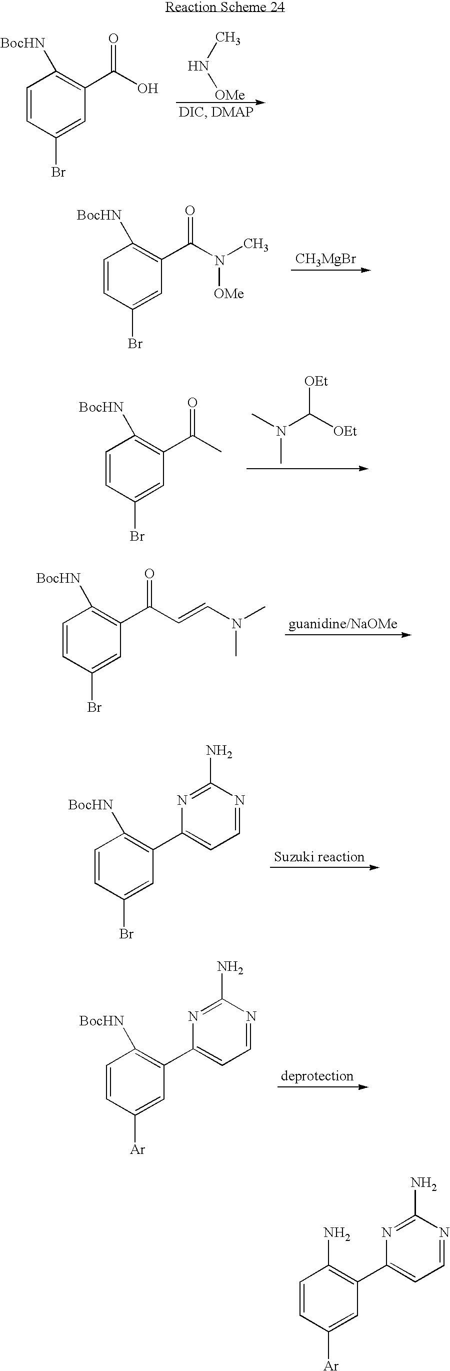

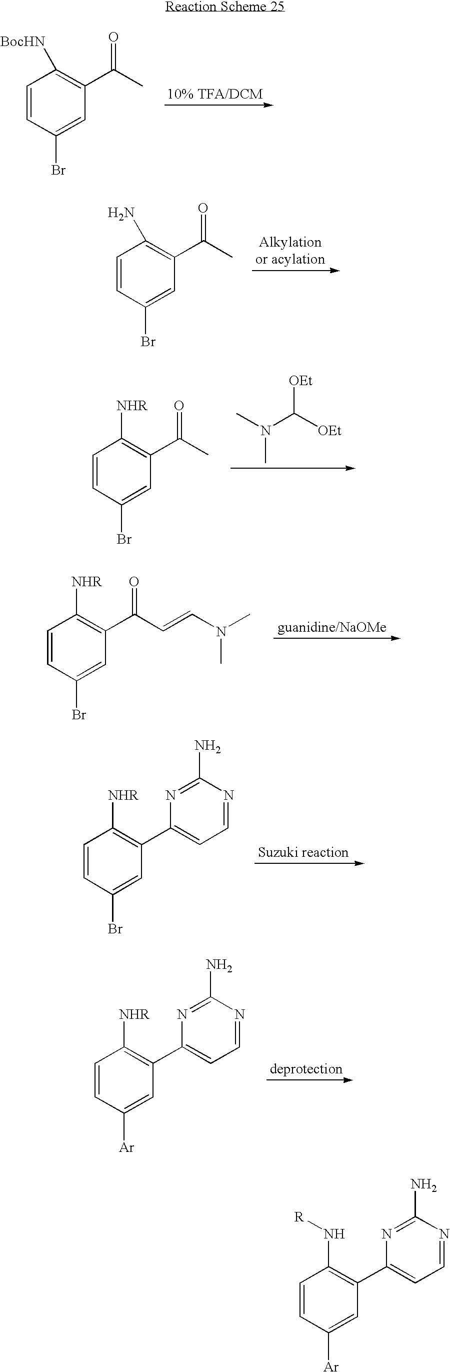

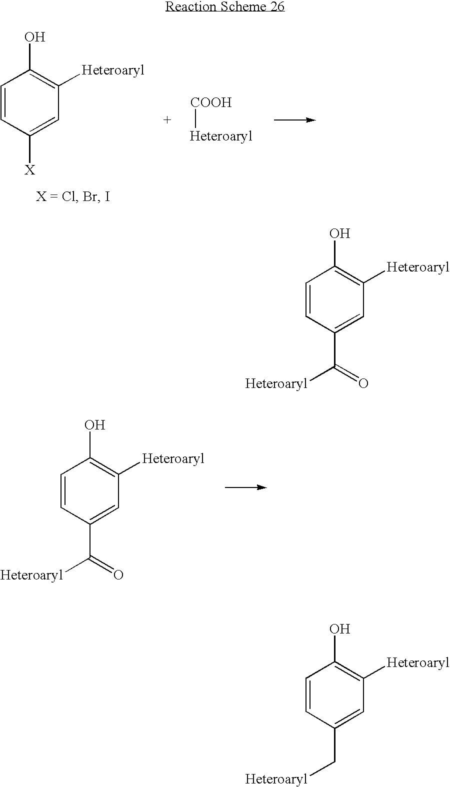

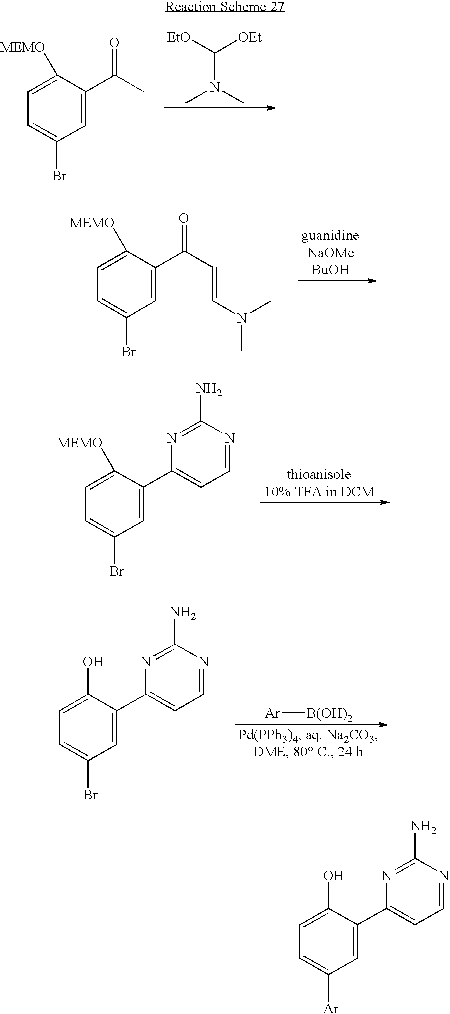

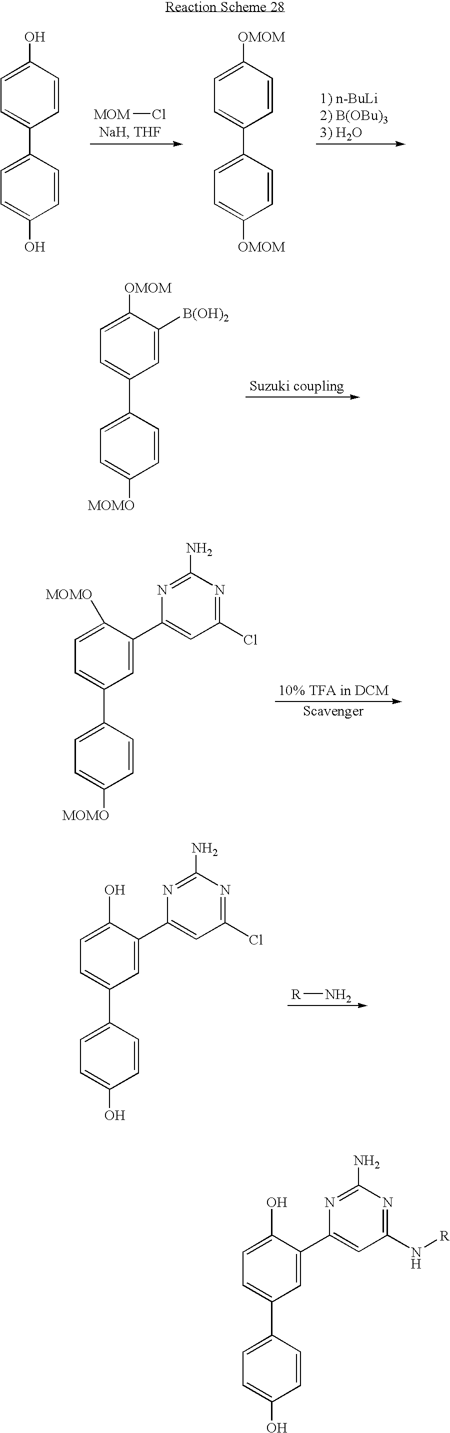

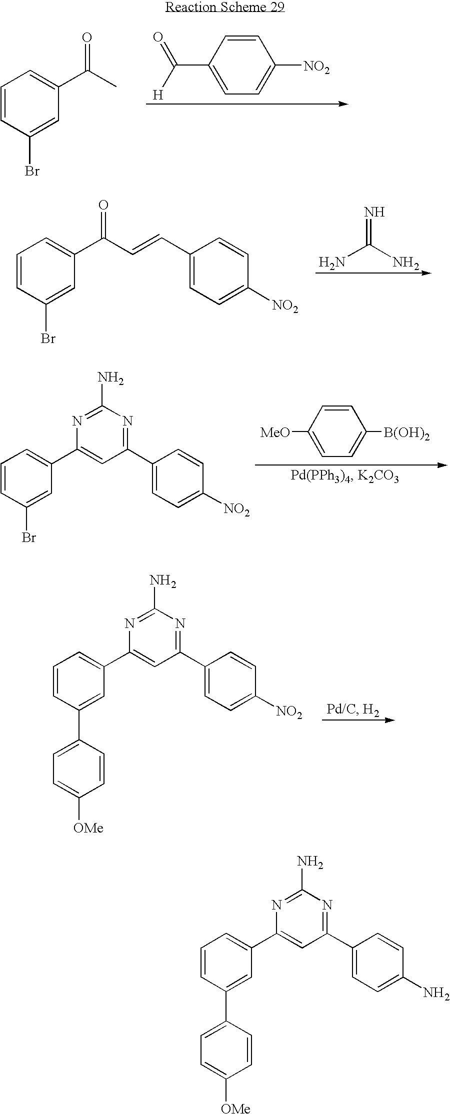

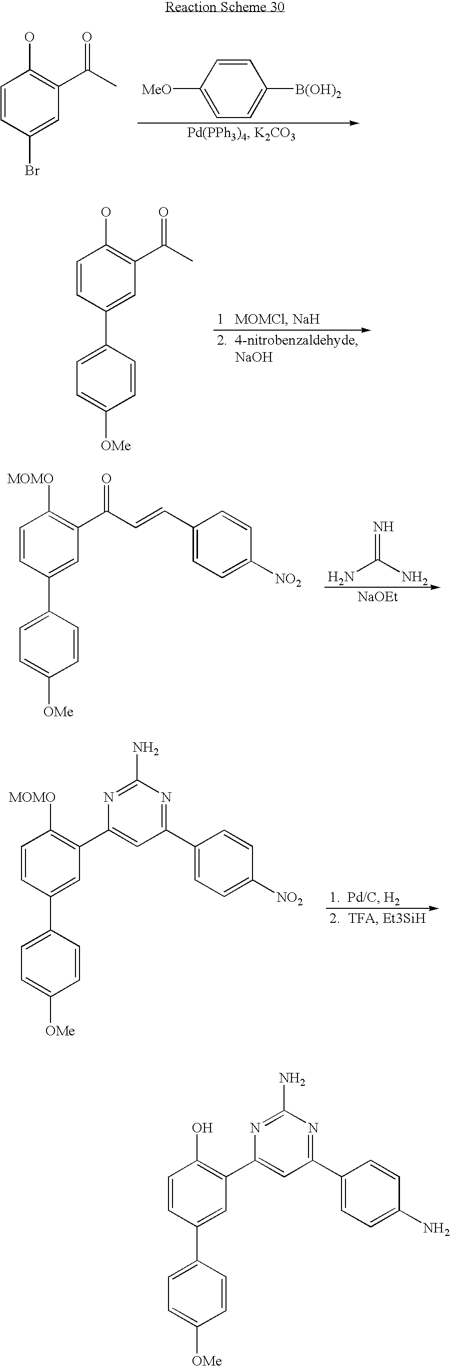

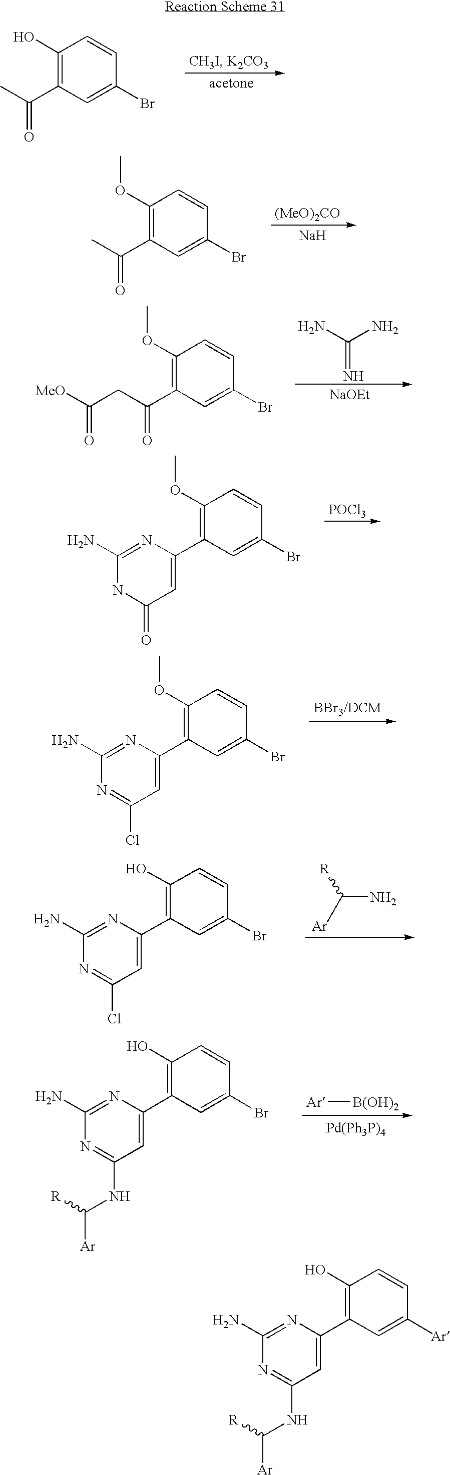

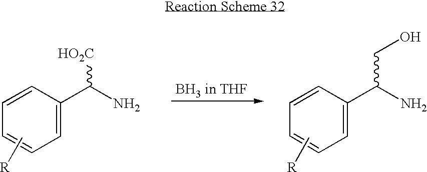

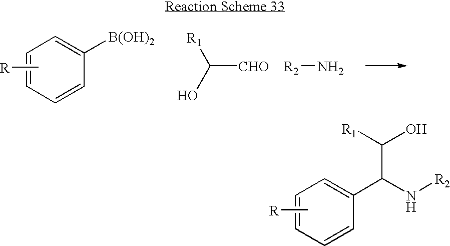

### IV. Target Diseases to be Treated

The compounds described herein are useful for treating disorders related to unregulated kinase-signal transduction, including cell proliferative disorders, fibrotic disorders and metabolic disorders. The compounds of the present invention are useful in the same manner as is described in the International Publication WO 00/56709, incorporated by reference herein in its entirety.

Cell proliferative disorders which can be treated or further studied by the present invention include cancers, blood vessel proliferative disorders and mesangial cell proliferative disorders.

Blood vessel proliferative disorders refer to angiogenic and vasculogenic disorders generally resulting in abnormal proliferation of blood vessels. The formation and spreading of blood vessels, or vasculogenesis and angiogenesis, respectively, play-important roles in a variety of physiological processes such as embryonic development, corpus luteum formation, wound healing and organ regeneration. They also play a pivotal role in cancer development. Other examples of blood vessel proliferation disorders include arthritis, where new capillary blood vessels invade the joint and destroy cartilage, and ocular diseases, like diabetic retinopathy, where new capillaries in the retina invade the vitreous, bleed and cause blindness. Conversely, disorders related to the shrinkage, contraction or closing of blood vessels, such as restenosis, are also implicated.

Fibrotic disorders refer to the abnormal formation of extracellular matrix. Examples of fibrotic disorders include hepatic cirrhosis and mesangial cell proliferative disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis. Other fibrotic disorders implicated include atherosclerosis.

Mesangial cell proliferative disorders refer to disorders brought about by abnormal proliferation of mesangial cells. Mesangial proliferative disorders include various human renal diseases, such as glomerulonephritis, diabetic nephropathy, malignant nephrosclerosis, thrombotic microangiopathy syndromes, transplant rejection, and glomerulopathies. The PDGF-R has been implicated in the maintenance of mesangial cell proliferation. Floege et al., 1993, Kidney International 43:47S-54S.

PKs have been associated with such cell proliferative disorders. For example, some members of the receptor tyrosine kinase family have been associated with the development of cancer. Some of these receptors, like the EGFR (Tuzi et al., 1991, Br. J Cancer 63:227-233; Torp et al., 1992, APMIS 100:713-719), HER2/neu (Slamon et al., 1989, Science 244:707-712) and the PDGF-R (Kumabe et al, 1992, Oncogene 7:627-633) are overexpressed in many tumors and/or persistently activated by autocrine loops. In fact, in the most common and severe cancers these receptor overexpressions (Akbasak and Suner-Akbasak et al, 1992, J. Neurol. Sci. 119-133; Dickson et al, 1992, Cancer Treatment Res. 61:249-273; Korc et al., 1992, J Clin. Invest. 90:1352-1360) and autocrine loops (Lee and Donoghue, 1992, J Cell. Biol 118:1057-1070; Korc et al, supra; Akbasak and Suner-Akbasak et al, supra) have been demonstrated. For example, the EGFR receptor has been associated with squamous cell carcinoma, astrocytoma, glioblastoma, head and neck cancer, lung cancer and bladder cancer. HER2 has been associated with breast, ovarian, gastric, lung, pancreas and bladder cancer. The PDGF-R has been associated with glioblastoma, lung, ovarian, melanoma and prostate cancer. The RK c-met has been generally associated with hepatocarcinogenesis and thus hepatocellular carcinoma. Additionally, c-met has been linked to malignant tumor formation. More specifically, the RK c-met has been associated with, among other cancers, colorectal, thyroid, pancreatic and gastric carcinoma, leukemia and lymphoma. Additionally, over-expression of the c-met gene has been detected in patients with Hodgkin's disease, Burkitt's disease, and the lymphoma. cell line. Flk has likewise been associated with a broad spectrum of tumors including, without limitation, mammary, ovarian and lung tumors as well as gliomas such as glioblastoma.

The IGF-IR, in addition to being implicated in nutritional support and in type-II diabetes, has also been associated with several types of cancers. For example, IGF-I has been implicated as an autocrine growth stimulator for several tumor types, e.g., human breast cancer carcinoma cells (Azteaga et aL, 1989, J Clin. Invest. 84:1418-1423) and small lung tumor cells (Macauley et aL, 1990, Cancer Res. 50:2511-2517). In addition, IGF-I, integrally involved in the normal growth and differentiation—of the nervous system, appears to be an autocrine stimulator of human glionias. Sandberg-Nordqvist et aL, 1993, Cancer Res. 53:2475-2478. The importance of the IGF-IR and its ligands in cell proliferation is further supported by the fact that many cell types in culture (fibroblasts, epithelial cells, smooth muscle cells, T-lymphocytes, myeloid cells, chondrocytes, osteoblasts, the stem cells of the bone marrow) are stimulated to grow by IGF-I. Goldring and Goldring, 1991, Eukaryotic Gene Expression 1:301-326. In a series of recent publications, Baserga even suggests that IGF-I-R plays a central role in the mechanisms of transformation and; as such, could be a preferred target for therapeutic interventions for a broad spectrum of human malignancies. Baserga, 1995, Cancer Res. 55:249-252; Baserga, 1994, Cell 79:927-930; Coppola et aL, 1994, MoL Cell. BioL 14:4588-4595.

Some protein kinases (PKs) have been implicated in many types of cancer including, notably, breast cancer (Cance, et al., Int. J Cancer, 54:571-77 (1993)).

The association between abnormalities in RKs and disease are not restricted to cancer, however. For example, RKs have been associated with metabolic diseases like psoriasis, diabetes mellitus, wound healing, inflammation, and neurodegenerative diseases. These diseases include, but are not limited to hypertension, depression, generalized anxiety disorder, phobias, post-traumatic stress syndrome, avoidant personality disorder, sexual dysfimction, eating disorders, obesity, chemical dependencies, cluster headache, migraine, pain, Alzheimer's disease, obsessive-compulsive disorder, panic disorder, memory disorders, Parkinson's disease, endocrine disorders, vasospasm, cerebellar ataxia, and gastrointestinal tract disorders. For example, the EGF-R is indicated in corneal and dermal wound healing. Defects in the Insulin-R and the IGF-1R are indicated in type-II diabetes mellitus. A more complete correlation between specific RKs and their therapeutic indications is set forth in Plowman et aL, 1994, DN&P 7:334-339.

Not only receptor type kinases, but also many cellular kinases (CKs) including src, abl, fps, yes, fyn, lyn, lck, blk, lick, fgr, yrk (reviewed by Bolen et al, 1992, FASEB J 6:3403-3409) are involved in the proliferative and metabolic signal transduction pathway and thus in indications of the present invention. For example, mutated src (v-src) has been demonstrated as an oncoprotein (pp60^{v-src}) in chicken. Moreover, its cellular homolog, the proto-oncogene pp60^{c-src} transmits oncogenic signals of many receptors. For example, overexpression of EGF-R or HER2/neu in tumors leads to the constitutive activation of pp6^{c-src}, which is characteristic for the malignant cell but absent from the normal cell. On the other hand, mice deficient for the expression of c-src exhibit an osteopetrotic phenotype, indicating a key participation of c-src in osteoclast fitriction and a possible involvement in related disorders. Similarly, Zap 70 is implicated in T-cell signaling.

Furthermore, the identification of CTK modulating compounds to augment or even synergizewith RIC-aimed blockers is an aspect of the present invention.

Additionally, both RKs and non-receptor type kinases have been connected to hyperimmune disorders.

Further, the compounds of the present invention are also effective in treating diseases that are related to the PYK-2 protein.

Thus it can be appreciated that the compounds and the methods of the present invention are suitable to obtain a therapeutic effect against a disease or an abnormal condition, which is selected from the group consisting of cell proliferative disease, cerebrovascular damage, autoimmune diseases, neurodegenerative disease, degenerative diseases of the musculoskeletal system.

Examples of the neurodegerative disease to be treated by the compounds and methods of the present invention include, but are not limited to AIDS related dementia, Alzheimer's disease, Parkinson's disease, Pick's disease, Huntington's disease, diffuse Lewy body disease, multiple system atrophy, amyotrophic lateral sclerosis, dementia associated with Down's syndrome, cerebrovascular dementia, canine motor neuron disease, retinitis pigmentosa, spinal muscular atrophy, myelodysplastic syndromes, stroke and reperfusion injury, aplastic anemia, ischemic injury associated with myocardial infarctions, arrythmia, atherosclerosis, toxin-induced or alcohol related diseases, hematological diseases including but not limited to chronic anemia and aplastic anemia, and cerebral degeneration.

Examples of the cerebrovascular damage to be treated by the compounds and methods of the present invention include, but are not limited to cerebrovascular dementia, stroke, cerebral ischemia, and head trauma.

Certain autoimmune diseases which may be treated by the compounds and methods of the present invention include, but are not limited to systemic lupus, erthematosus, autoimmune mediated glomerulophritis, rheumatoid arthritis, psoriasis, inflammatory bowel disease, autoimmune diabetes mellitus, and the development of AIDS in HIV-infected individuals.

The compounds and methods of the present invention may also be effective against neurodegenerative diseases, such as (without limitation) AIDS related dementia, dementias including Alzheimer's disease, Parkinson's disease, Pick's disease, Huntington's disease, diffuse Lewy body disease, multiple system atrophy, amyotrophic lateral sclerosis, dementia associated with Down's syndrome, cerebrovascular dementia, and canine motor neuron disease, retinitis pigmentosa, spinal muscular atrophy and cerebral degeneration.

It is also contemplated that the compounds and methods of the present invention are effective against degenerative diseases, which include, but are not limited to osteoporosis, arthritis, aspirin sensitive rhinosinusitis, cystic fibrosis, multiple sclerosis, kidney disease, chemotherapy induced hair loss, allopecia, and cancer pain.

The cell proliferative diseases to be treated by the compounds and methods of the present invention include, but are not limited to carcinoma, selected from the group consisting of carcinoma of breast, lung, colon, kidney, liver, prostate, stomach, esophagus, gall bladder, ovary, pancreas, cervix, bladder, thyroid, skin, and squamous cell carcinoma; hematopoietic tumors of myeloid lineage, selected from the group consisting of acute and chronic mylogenous leukemias, promyelocytic leukemia, and myelodysplastic syndrome; hematopoietic tumors of lymphoid lineage, selected from the group consisting of B-cell lymphoma, T-cell lymphoma, Hodgkin's lymphoma, non-Hodgkin's lymphoma, hairy cell lymphoma, Burkett's lymphoma, leukemia, acute lymphocytic leukemia, and acute lymphoblastic leukemia; tumors of messenchymal origin, selected from the group consisting of fibrosarcoma and rhabdomyosarcoma; tumors of the central and peripheral nervous system, selected from the group consisting of neuroblastoma, astrocytoma, glioma and schwannomas; Karposi's sarcoma, melanoma, seminoma, teratocarcinoma, xenoderoma, pigmentosum, osteosarcoma, keratoctanthoma, and thyroid follicular cancer; benign prostate hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.

### V. Target Proteins of the Invention

The present invention features a method of modulating the function of a protein kinase with a compound of the invention, comprising the step of contacting cells expressing the protein kinase with the compound.

A still further aspect of this invention is that the protein kinase whose catalytic activity is being modulated by a compound of this invention is selected from the group consisting of receptor protein tyrosine kinases, cellular tyrosine kinases and serine-threonine kinases.

It is an aspect of this invention that the receptor protein kinase whose catalytic activity is modulated by a compound of this invention is selected from the group consisting of EGF, EGF receptor, HER2, HER3, HER4, IR, IGF-1R, IRR, PDGFRα, PDGFRβ, CSFIR, C-Kit, C-fms, Flk-1R, Flk4, KDR/Flk-1, Flt-1, FGFR-1R, FGFR-2R, FGFR-3R, FGFR-4R, protein kinase C, MEK1, MAP kinase, RAF1, PI3 kinase, and weel kinase. In addition, it is an aspect of this invention that the cellular tyrosine kinase whose catalytic activity is modulated by a compound of this invention is selected from the group consisting of Src, Frk, Btk, Csk, Abl, ZAP70, Fes/Fps, Fak, Jak, Ack, Yes, Fyn, Lyn, Lck, Blk, Hck, Fgr and Yrk. Another aspect of this invention is that the serine-threonine protein kinase whose catalytic activity is modulated by a compound of this invention is selected from the group consisting of the cyclin dependent kinase (CDK) family of enzymes, including, but not limited to, CDK1 (CDC2), CDK2, CDK3, CDK4, CDK5, CDK6, CDK7, CDK8, and CDK9, and Raf. The cyclin dependent kinase may be selected from the group consisting of CDK2 and CDK5.

A protein kinase natural binding partner can bind to a protein kinase's intracellular region with high affinity. High affinity represents an equilibrium binding constant on the order of 10⁻⁶ M or less. In addition, a natural binding partner can also transiently interact with a protein kinase intracellular region and chemically modify it. Protein kinase natural binding partners are chosen from a group that includes, but is not limited to, SRC homology 2 (SH2) or 3 (SH3) domains, other phosphoryl tyrosine binding (PTB) domains, guanine nucleotide exchange factors, protein phosphatases, and other protein kinases. Methods of determining changes in interactions between protein kinases and their natural binding partners are readily available in the art.

The compounds of the invention preferably modulate the activity of the protein tyrasine kinase in vitro. These compounds preferably show positive results in one or more in vitro assays for an activity corresponding to treatment of the disease or disorder in question (such as the assays described in the Examples below).

The invention also features a method of identifying compounds that modulate the function of protein kinase, comprising the following steps: (a) contacting cells expressing the protein tyrosine kinase with the compound; and (b) monitoring an effect upon the cells. The effect upon the cells is preferably a change in cell phenotype, more preferably it is a change or an absence of a change in cell proliferation., even more preferably it is a change or absence of a change in the catalytic activity of the protein kinase, and most preferably it is a change or absence of a change in the interaction between the protein kinase with a natural binding partner, as described herein.

In a preferred embodiment, the invention features a method for identifying the compounds of the invention, comprising the following steps: (a) lysing the cells to render a lysate comprising protein tyrosine kinase; (b) adsorbing the protein tyrosine kinase to an antibody; (c)incubating the adsorbed protein tyrosine kinase with a substrate or substrates; and (d) adsorbing the substrate or substrates to a solid support or antibody; where the step of monitoring the effect on the cells comprises measuring the phosphate concentration of the substrate or substrates.

In yet another aspect, the invention features a method for treating a disease related to unregulated kinase signal transduction, where the method includes the step of administering to a subject in need thereof a therapeutically effective amount of a compound of the invention as described herein.

The invention also features a method of regulating kinase signal transduction comprising administering to a subject a therapeutically effective amount of a compound of the invention as described herein.

Furthermore, the invention features a method of preventing or treating an abnormal condition in an organism, where the abnormal condition is associated with an aberration in a signal transduction pathway characterized by an interaction between a protein kinase and a natural binding partner, where the method comprises the following steps: (a) administering a compound of the invention as described herein; and (b) promoting or disrupting the abnormal interaction. The organism may be a mammal and the abnormal condition is as enumerated here generally, and specifically in Section III above. The mammal may be selected from the group consisting of mice, rats, rabbits, guinea pigs, dogs, cats, sheep, goats, cows, primates, such as monkeys, chimpanzees, and apes, and humans.

As used herein, “PK related disorder,” “PK driven disorder,” and “abnormal PK activity” all refer to a condition characterized by inappropriate; i.e., under or, more commonly, over, PK catalytic activity, where the particular PK can be an RTK, a CTK or an STK. Inappropriate catalytic activity can arise as the result of either: (1) PK expression in cells which normally do not express PKs; (2) increased PK expression leading to unwanted cell proliferation, differentiatica and/or growth; or, (3) decreased PK expression leading to unwanted reductions in cell proliferation, differentiation and/or growth. Over-activity of a PK refers to either amplification of the gene encoding a particular PK or production of a level of PK activity which can correlate with a cell proliferation, differentiation and/or growth disorder (that is, as the level of the PK increases, the severity of one or more of the symptoms of the cellular disorder increases). Under-activity is, of course, the converse, wherein the severity of one or more symptoms of a cellular disorder increase as the level of the PK activity decreases.

The term “therapeutically effective amount” as used herein refers to that amount of the compound being administered which will relieve to some extent one or more of the symptoms of the disorder being treated. Thus, a therapeutically effective amount refers to that amount which has the effect of (1) inversing the rate of progress of a disease, or, in case of cancer reducing the size of the tumor; (2) inhibiting to some extent further progress of the disease, which in case of cancer may mean slowing to some extent, or preferably stopping tumor metastasis or tumor growth; and/or, (3) relieving to some extent (or, preferably, eliminating) one or more symptoms associated with the disease.

It is an aspect of this invention that the above-referenced protein kinase related disorder is selected from the group consisting of a receptor protein tyrosine kinase related disorder, a cellular tyrosine kinase disorder and a serine-threonine kinase related disorder.

In yet another aspect of this invention, the above referenced protein kinase related disorder is selected from the group consisting of an EGFR related disorder, a PDGFR related disorder, an IGFR related disorder and a flk related disorder.

In addition to modulating PK activity, the compounds of this invention may inhibit the activity of protein phosphatases, which are enzymes that remove phosphate groups from phosphorylated proteins. Thus the compounds disclosed herein-inay also represent a new generation of therapeutic compounds for diseases and disorders associated with abnormal phosphatase activity (such as, without limitation, diabetes, cell proliferation disorders and inflammatory disorders). The terms defined herein with respect to PKs would be understood by one skilled in the art to have the same or similar meamne with regard to phosphastases.

In another aspect, the invention provides a method of modulating a signal transduction pathway in a cells comprising the step of contacting the cell with with a compound of the invention. In an embodiment, the cells express a protein kinase and the compound modulates the function of the protein kinase.

In another aspect, the invention provides for a method of identifying an aromatic compound that modulates the function of protein kinase, comprising the following steps: a) contacting cells expressing the protein kinase with a compound of the invention; and b) monitoring an effect of the compound upon the cells.

The term “function” refers to the cellular role of a protein kinase. The protein kinase family includes members that regulate many steps in signaling cascades, including cascades controlling cell growth, migration, differentiation, gene expression, muscle contraction, glucose metabolism, cellular protein synthesis, and regulation of the cell cycle.

The term “catalytic activity”, in the context of tile invention, defines the rate at which a protein kinase phosphorylates a substrate. Catalytic activity can be measured, for example, by determining the amount of a substrate converted to a product as a function of time. Phosphorylation of a substrate occurs at the active-site of a protein kinase. The active-site is normally a cavity in which the substrate binds to the protein kinase and is phosphorylated.

The term “substrate” as used herein refers to a molecule phosphorylated by a protein kinase. The substrate is preferably a peptide and more preferably a protein.

The term “activates” refers to increasing the cellular ftmction of a protein kinase. The protein kinase function is preferably the interaction with a natural binding partner and most preferably catalytic activity.

The term “inhibit” refers to decreasing the cellular function of a protein kinase. The protein kinase function is preferably the interaction with a natural binding partner and most preferably catalytic activity.

The term “modulates” refers to altering the function of a protein kinase by increasing or decreasing the probability that a complex forms between a protein kinase and a natural binding partner. A modulator may increase the probability that such a complex forms between the protein kinase and the natural binding partner, or may increase or decrease the probability that a complex forms between the protein kinase and the natural binding partner depending on the concentration of the compound exposed to the protein kinase, or may decrease the probability that a complex forms between the protein kinase and the natural binding partner. A modulator may activate the catalytic activity of a protein kinase, or may activate or inhibit the catalytic activity of a protein kinase depending on the concentration of the compound exposed to the protein kinase, or may inhibit the catalytic activity of a protein kinase.

The term “complex” refers to an assembly of at least two molecules bound to one another. Signal transduction complexes often contain at least two protein molecules bound to one another.

The term “natural binding partner” refers to polypeptides that bind to a protein kinase in cells. Natural binding partners can play a role in propagating a signal in a protein kinase signal transduction process. A change in the interaction between a protein kinase and a natural binding partner can manifest itself as an increased or decreased probability that the interaction forms, or an increased or decreased concentration of the protein kinase/natural binding partner complex.

The term “contacting” as used herein refers to mixing a solution comprising a compound of the invention with a liquid medium bathing the cells of the methods. The solution comprising the compound may also comprise another component, such as dimethylsulfoxide (DMSO), which facilitates the uptake of the compound or compounds into the cells of the methods. The solution comprising the compound of the invention may be added to the medium bathing the cells by utilizing a delivery apparatus, such as a pipet-based device or syringe-based device.

The term “monitoring” refers to observing the effect of adding the compound to the cells of the method. The effect can be manifested in a change in cell phenotype, cell proliferation, protein kinase catalytic activity, or in the interaction between a protein kinase and a natural binding partner.

The term “effect” describes a change or an absence of a change in cell phenotype or cell proliferation. “Effect” can also describe a change or an absence of a change in the catalytic activity of the protein kinase. “Effect” can also describe a change or an absence of a change in an interaction between the protein kinase and a natural binding partner.

The term “cell phenotype” refers to the outward appearance of a cell or tissue or the function of the cell or tissue. Examples of cell phenotype are cell size (reduction or enlargement), cell proliferation (increased or decreased numbers of cells), cell differentiation (a change or absence of a change in cell shape), cell survival, apoptosis (cell death), or the utilization of a metabolic nutrient (e.g., glucose uptake). Changes or the absence of changes in cell phenotype are readily measured by techniques known in the art.

The term “antibody” refers to an antibody (e.g., a monoclonal or polyclonal antibody), or antibody fragment, having specific binding affinity to protein kinase or its fragment.

By “specific binding affinity” is meant that the antibody binds to target (protein kinase) polypeptides with greater affinity than it binds to other polypeptides under specified conditions. Antibodies having specific binding affinity to a protein kinase may be used in methods for detecting the presence and/or amount of a protein kinase in a sample by contacting the sample with the antibody under conditions such that an immunocomplex forms and detecting the presence and/or amount of the antibody conjugated to the protein kinase. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

The term “polyclonal” refers to antibodies that are heterogenous populations of antibody molecules derived from the sera of animals immunized with an antigen or an antigenic functional derivative thereof For the production of polyclonal antibodies, various host animals may be immunized by injection with the antigen.

Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art. See, for example, Kohler, et al., Nature 256:495-497 (1975), and U.S. Pat. No. 4,376,110.

The term “antibody fragment” refers to a portion of an antibody, often the hypervariable region and portions of the surrounding heavy and light chains, that displays' specific binding affinity for a particular molecule. A hypervariable region is a portion of an antibody that physically binds to the polypeptide target.

The term “Aberration”, in conjunction with a signal transduction process, refers to a protein kinase that is over- or under-expressed in an organism, mutated such that its catalytic activity is lower or higher than wild-type protein kinase activity, mutated such that it can no longer interact with a natural binding partner, is no longer modified by another protein kinase or protein phosphatase, or no longer interacts with a natural binding partner.

The term “promoting or disrupting the abnormal interaction” refers to a method that can be accomplished by administering a compound of the invention to cells or tissues in an organism. A compound can promote an interaction between a protein kinase and natural binding partners by forming favorable interactions with multiple atoms at the complex interface. Alternatively, a compound can inhibit an interaction between a protein kinase and natural binding partners by compromising favorable interactions formed between atoms at the complex interface.

“In vitro” refers to procedures performed in an artificial environment, such as, without limitation, in a test tube, in a cell, or culture medium. As used herein, “in vivo” refers to procedures performed within a living organism such as, without limitation, a mouse, rat, or rabbit.

Thus, it can be appreciated by those of skill in the art that the methods and compounds described herein can be used to obtain a therapeutic effect agains an unregulated protein kinase signal transduction which lead to a disease or an abnormal condition in an organism. In addition, the methods of the present invention lead to the treatment or prevention of a disease or an abnormal condition, where the disease or abnormal condition is associated with an aberration in a signal transduction pathway characterized by an interaction between a protein kinase and a binding partner, and where the method further comprises the steps of promoting or disrupting the abnormal interaction.

### VI. Pharmaceutical Compositions

The present invention also relates to a pharmaceutical composition comprising

a) a compound of the invention as described herein; and

b) a pharmaceutically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The compounds described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

a) Routes of Administration

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation. Furthermore, one may administer the drug in a targeted drug delivery system, for example, in a liposome coated with tumor-specific antibody. The liposomes will be targeted to and taken up selectively by the tumor.

b) Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with one or more compound of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethyl cellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacqucr solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant Polysorbate 80™, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

c) Effective Dosage.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective dose can be estimated initially from cell culture assays. For example, a dose can be formulated in animal models to achieve a circulating concentration range that includes the IC₅₀ as determined in cell culture. Such information can be used to more accurately determine useful doses in humans.

Toxicity and therapeutic efficacy of the compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient.

Dosage amount and interval may be adjusted individually to provide plasma levels of the active moiety which are sufficient to maintain the kinase modulating effects, or minimal effective concentration (MEC). The MEC will vary for each compound but can-be estimated from in vitro data; e.g., the concentration necessary to achieve 50-90%, inhibition of the kinase using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

d) Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the polynucleotide for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

### VII. Embodiments of the Invention

Some of the embodiments of the invention are set forth below. It is understood that the present invention encompasses embodiments other than those listed below.

In a first embodiment, the present invention relates to a compound of Formula I

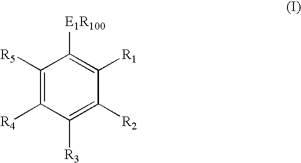

or a pharmaceutically acceptable salt, amide, ester, or prodrug thereof, where

a) R₁ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) an acyl of formula —(X₁)ₙ₁—C(O)—X₂, where

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

where X₃ is selected from the group consisting of hydrogen, alkyl, aryl, heteroaryl, amino, and amide; and

n1 is 0 or 1; and

iii) a substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that R₁ is not —C₆H₅, —C(O)H, —C(O)CH₃, —C(O)—C₆H₅, —C(O)NH₂, or —C₆H₄CH₃;

b) R₂, R₃, and R₄ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

where X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

or R₂ and R₃, taken together along with the two ring carbons to which they are attached, or R₄ and R₃, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1; and

c) R₅ is selected from the group consisting of hydrogen, lower alkyl, aryl, and heteroaryl, or R₄ and R₅, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, where

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, where X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, where

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, where

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, where

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

where X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, where

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

d) R₁₀₀ is selected from the group consisting of hydrogen, lower alkyl, lower alkene, lower alkyne, aryl, and heteroaryl;

e) E₁ is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

provided that at least one of R₁-R₅ is not selected from the group consisting of hydrogen, lower alkyl, aryl, and heteroaryl.

In a second embodiment, the invention relates to a compound of Formula II

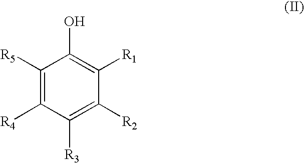

or a pharmaceutically acceptable salt, amide, ester, or prodrug thereof, wherein

a) R₁ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) an acyl of formula —(X₁)ₙ₁—C(O)—X₂, wherein

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

wherein X₃ is selected from the group consisting of hydrogen, alkyl, aryl, heteroaryl, amino, and amide; and

n1 is 0 or 1; and

iii) a substituent of formula —C(X₄)═N—NX₅—C(═E)—NX₆X₇, or of formula —C(X₄)═N—N═C(EX₅)—NX₆X₇, wherein

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that R₁ is not —C₆H₅, —C(O)H, —C(O)CH₃, —C(O)—C₆H₅, —C(O)NH₂, or —C₆H₄CH₃;

b) R₂, R₃, and R₄ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, wherein

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, wherein

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

wherein X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, wherein

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

or R₂ and R₃, taken together along with the two ring carbons to which they are attached, or R₄ and R₃, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, wherein

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alky, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, wherein X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, wherein

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, wherein

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

wherein X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, wherein

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1; and

c) R₅ is selected from the group consisting of hydrogen, lower alkyl, aryl, and heteroaryl, or R₄ and R₅, taken together along with the two ring carbons to which they are attached, form a six-membered aromatic or heteroaromatic, or a five- or six-membered heteroaromatic ring, optionally substituted with one or more substituents selected from the group consisting of

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X)═N—NX₅—C(═E)—NX₆X₇, or of formula —C(X₄)═N—N═C(EX₅)—NX₆X₇, wherein

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, wherein X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, wherein

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, wherein

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

wherein X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, wherein

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

provided that at least one of R₁-R₅ is not selected from the group consisting of hydrogen, lower alkyl, aryl, and heteroaryl.

In the third embodiment, the invention relates to the compound of the first embodiment, wherein R₁ is selected from the group consisting of

i) hydrogen;

ii) a six-membered aromatic ring, optionally substituted with one or more substituents selected from the group consisting of lower alkyl, hydroxy, and amino;

iii) a six-membered heteroaromatic ring, optionally substituted with one or more substituents selected from the group consisting of lower alkyl, hydroxy, alkoxy, amino, and nitro;

iv) a five-membered heteroaromatic ring, optionally substituted with one or more substituents selected from the group consisting of lower alkyl, hydroxy, and amino;

v) acyl of formula —(X₁)ₙ₁—C(O)—X₂, wherein

X₁ is lower alkylene or lower alkenylene;

X₂ is selected from the group consisting of hydrogen, amino, hydroxy, and —NH—X₃,

wherein X₃ is selected from the group consisting of hydrogen, lower alkyl, amino, and amide; and

n1 is 0 or 1; and

vi) substituent of formula —C(X₄)═N—NH—C(═E)—NX₆X₇, or of formula —C(X₄)═N—N═C(EX₅)—NX₆X₇, wherein

X₄ is selected from the group consisting of hydrogen, lower alkyl, aryl, and alkaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen and lower alkyl.

In the forth embodiment, the invention relates to the compound of the first embodiment, wherein R₁ is selected from the group consisting of

i) hydrogen;

ii) a phenyl, optionally substituted with one or more of hydroxy or —NH₂;

iii) a six-membered heteroaromatic ring, selected from the group consisting of pyridine, pyrazine, pyridazine, pyrimidine, and 1,3,5-triazine, each independently and optionally substituted with one or more substituents selected from the group consisting of lower alkyl, hydroxy, alkoxy, and amino;

iv) a five-membered heteroaromatic ring, selected from the group consisting of pyrrole, imidazole, 1,2,3-triazole, 1,2,4-triazole, tetrazole, thiophene, thiazole, and thiadiazole, each independently and optionally substituted with one or more substituent selected from the group consisting of hydroxy, an amide of formula —(X₂₄)ₙ₂₄—NH—C(O)—X₂₅ or —(X₂₆)ₙ₂₆—C(O)—NH—X₂₇, and —NH₂;

v) acyl of formula —C(O)—X₂, wherein X₂ is hydrogen or lower alkyl;

vi) acyl of formula —X₁—C(O)—X₂, wherein

X₁ is lower alkylene or lower alkenylene; and

X₂ is —NH—X₃, wherein X₃ is selected from the group consisting of hydrogen, amino, and amide;

vi) substituent of formula —C(X₄)═N—NH—C(═E)—NH₂, wherein

X₄ is selected from the group consisting of hydrogen, methyl, ethyl, phenyl, and —CH₂CH₂—Ph; and

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

vii) substituent of formula —C(X₄)═N—N═C(SX₅)—NH₂, wherein

X₄ is selected from the group consisting of hydrogen, methyl, ethyl, phenyl, and —CH₂CH₂—Ph; and

X₅ is hydrogen or methyl.

In the fifth embodiment, the invention relates to the compound of the first embodiment, wherein said five- or six-membered heteroaryl ring in R₁ is selected from the group consisting of optionally substituted

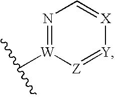

optionally substituted

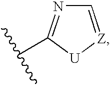

and optionally substituted

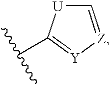

wherein W, X, Y, and Z are each independently CR or nitrogen, and U is selected from the group consisting of CR₂, oxygen, sulfur, and NR;

wherein R is selected from the group consisting of hydrogen, alkyl, and aryl.

In the sixth embodiment, the invention relates to the compound of the fifth embodiment, wherein said heteroaryl ring is selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, triazine,

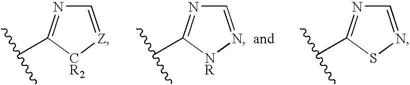

wherein R is selected from the group consisting of hydrogen, alkyl, and aryl.

In the seventh embodiment, the invention relates to the compound of the sixth embodiment, wherein said heteroaryl ring is selected from the group consisting of aminofuran, aminothiophene, aminopyrrole, aminopyrroline, aminopyrrolidine, aminooxazole, aminothiazole, aminoimidazole, aminoimidazoline, aminoimidazolidine, aminopyrazole, aminopyrazoline, aminopyrazolidine, aminoisoxazole, aminoisothiazole, aminotriazole, aminothiadiazole, aminopyran, aminopyridine, aminopiperidine, aminomorpholine, aminothiomorpholine, aminopyridazine, aminopyrimidine, aminopyrazine, aminopiperazine, aminotriazine,

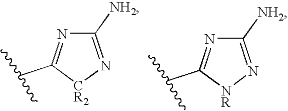

and

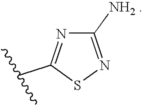

In the eighth embodiment, the invention relates to he compound of the first embodiment, wherein R₁ is selected from the group consisting of hydrogen, —C(O)—CH₃, —C(O)—NH—CH₂—C(O)—NH₂, —CH═CH—C(O)—NH₂, —CH₂CH₂—C(O)—NH—NH₂, —C(H)═N—NH—C(O)—NH₂, —C(CH₃)═N—NH—C(O)—NH₂, —C(H)═N—NH—C(S)—NH₂, —C(CH₃)═N—NH—C(S)—NH₂, —C(Ph)═N—NH—C(S)—NH₂, —C(CH₂CH₂Ph)═N—NH—C(S)—NH₂, —C(H)═N—N═C(SCH₃)—NH₂,

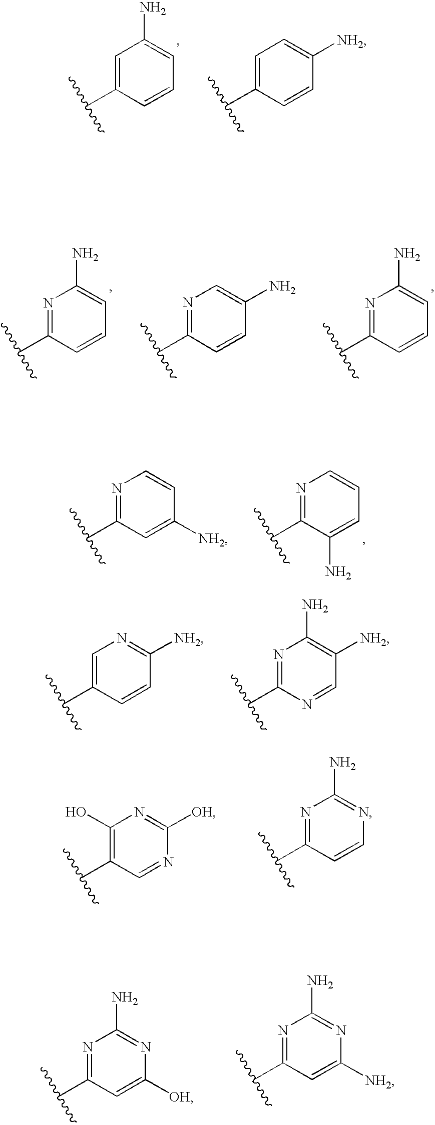

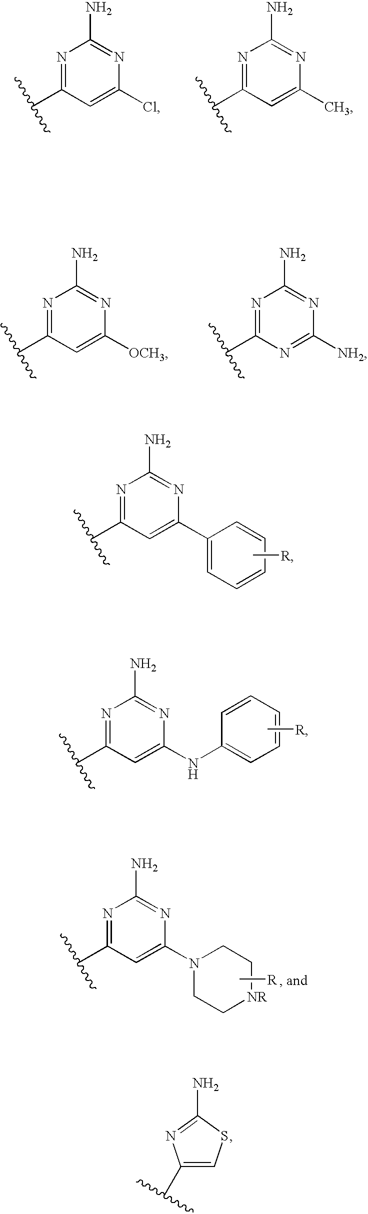

wherein R is selected from the group consisting of hydrogen, alkyl, and aryl.

In the ninth embodiment, the invention relates to the compound of the first embodiment, wherein R₃ is selected from the group consisting of

i) hydrogen;

ii) C₂-C₆ alkenylene;

iii) halogen or perhaloalkyl;

iv) an alkoxy of formula —O—X₁₄, wherein

X₁₄ is selected from the group consisting of hydrogen, lower alkyl, and aryl; and

v) a five-membered or six-membered heteroaryl ring or a six-membered aryl or heteroaryl ring, optionally substituted with one or more substituents selected from the group consisting of

A) optionally substituted C₁-C₄ straight-chain, branched, or cyclic saturated or unsaturated alkyl;

B) an alkoxy of formula —O—X_{14,} wherein X₁₄ is hydrogen or lower alkyl; and

C) halogen or perhaloalkyl;

D) cyano;

E) nitro;

F) a substituent of formula —(X₁₈)ₙ₁₈—C(O)—X₁₉, wherein

X₁₈ is lower alkylene;

X₁₉ is selected from the group consisting of hydrogen, lower alkyl, aryl, heteroaryl, hydroxy, alkoxy, amino, and —NX₂₀X₂₁,

wherein X₂₀ and X₂₁ are each independently selected from the group consisting of hydrogen, alkyl, and aryl; and

n18 is 0 or 1; and

G) a thioether or thiol of formula —S—X₂₃, wherein X₂₃ is selected from the group consisting of hydrogen, lower alkyl, aryl, and heteroaryl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, lower alkyl, aryl, hydroxy, and alkoxy; and

X₂₇ is selected from the group consisting of hydrogen, lower alkyl, aryl, and heteroaryl.

In the tenth embodiment, the invention relates to the compound of the first embodiment, wherein R₃ is selected from the group consisting of

i) hydrogen;

ii) C₂-C₆ alkenylene;

iii) halogen or perfluoroalkyl;

iv) an alkoxy of formula —O—X₁₄, wherein

X₁₄ is selected from the group consisting of hydrogen, methyl, ethyl, and propyl; and

v) a six-membered aryl ring, optionally substituted with one or more substituents selected from the group consisting of

A) methyl, ethyl, and propyl;

B) hydroxy, methoxy, ethoxy, phenoxy, hydroxymethyl, 2-hydroxyethyl, and 3-hydroxypropyl; and

C) halogen or perfluoroalkyl;

D) cyano;

E) nitro;

F) a substituent of formula —C(O)—X₁₉, wherein

X₁₉ is selected from the group consisting of hydrogen, methyl, ethyl, propyl, phenyl, hydroxy, methoxy, ethoxy, propoxy, amino, and —NX₂₀X₂₁,

wherein X₂₀ and X₂₁ are each independently selected from the group consisting of hydrogen, methyl, ethyl, propyl, and phenyl; and

G) a thioether or thiol of formula —S—X₂₃, wherein X₂₃ is selected from the group consisting of hydrogen, methyl, ethyl, propyl, and phenyl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, methyl, ethyl, propyl, phenyl, hydroxy, methoxy, and phenoxy; and

X₂₇ is selected from the group consisting of hydrogen, methyl, ethyl, propyl, and phenyl.

In the eleventh embodiment, the invention relates to the compound of the first embodiment, wherein said five-membered or six-membered heteroaryl ring or said six-membered aryl or heteroaryl ring of R₃ is selected from the group consisting of optionally substituted

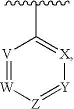

optionally substituted

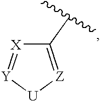

and optionally substituted

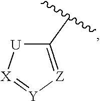

wherein V, W, X, Y and Z are each independently CR or nitrogen, and U is selected from the group consisting of CR₂, oxygen, sulfur, and NR;

wherein R is selected from the group consisting of hydrogen, alkyl, and aryl.

In the twelfth embodiment, the invention relates to the compound of the first embodiment, wherein said five-membered or six-membered heteroaryl ring or said six-membered aryl or heteroaryl ring of R₃ is selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

In the thirteenth embodiment, the invention relates to the compound of the first embodiment, wherein R₃ is selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonylphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-fluoro-4-methoxyphenyl,

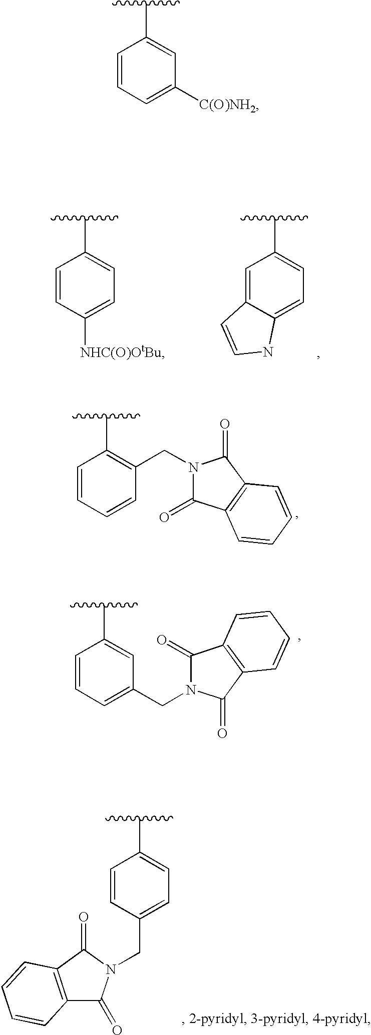

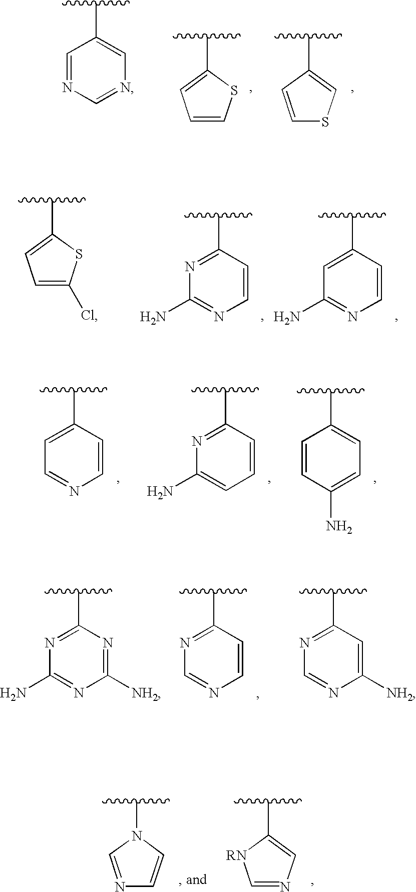

wherein R is selected from the group consisting of hydrogen, alkyl, and aryl.

In the fourteenth embodiment, the invention relates to the compound of the first embodiment, wherein R₂ and R₃, taken together along with the two ring carbons to which they are attached, or R₄ and R₃, taken together along with the two ring carbons to which they are attached, or R₄ and R₅, taken together along with the two ring carbons to which they are attached, form a six-membered aromatic or heteroaromatic, or a five- or six-membered heteroaromatic ring, optionally substituted with one or more substituents selected from the group consisting of hydrogen, hydroxy, halogens, cyano, nitro, amino, hydroxycarbonyl, aminocarbonyl, aminothiocarbonyl, lower alkoxy, phenoxy, amino-furan, amino-thiophene, amino-pyrrole, amino-pyrroline, amino-pyrrolidine, amino-oxazole, amino-thiazole, amino-imidazole, amino-imidazoline, amino-imidazolidine, amino-pyrazole, amino-pyrazoline, amino-pyrazolidine, amino-isoxazole, amino-isothiazole, aminotriazole, amino-thiadiazole, amino-pyran, amino-pyridine, amino-piperidine, amino-morpholine, amino-thiomorpholine, amino-pyridazine, amino-pyrimidine, amino-pyrazine, aminopiperazine, amino-triazine, semicarbazone, thiosemicarbazone, and amino guanidine.

In the fifteenth embodiment, the invention relates to the compound of the fourteenth embodiment, wherein said R₂ and R₃, taken together along with the rest of the compound of Formula II, or said R₄ and R₃, taken together along with the rest of the compound of Formula II, or said R₄ and R₅, taken together along with the rest of the compound of Formula II, result in the formation of an optionally substituted naphthalene.

In the sixteenth embodiment, the invention relates to the compound of the fourteenth embodiment, wherein said substituent is hydroxy.

In the seventeenth embodiment, the invention relates to a compound of Formula III

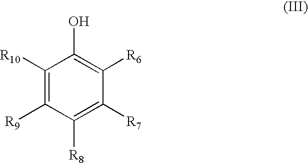

or a pharmaceutically acceptable salt, amide, ester, or prodrug thereof, wherein

a) R₆ is selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro; and

ii) acyl of formula —(X₁)ₙ₁—C(O)—X₂, wherein

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

wherein X₃ is selected from the group consisting of hydrogen, alkyl, aryl, heteroaryl, amino, and amide; and

n1 is 0 or 1;

provided that R₆ is not —C₆H₅, —C(O)H, —C(O)CH₃, —C(O)—C₆H₅, —C(O)NH₂, or —C₆H₄CH₃.

b) R₇, R₈, and R₉ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, wherein

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, wherein

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

wherein X₂₀ and X₂₁, are each independently selected from the group consisting of hydrogen, alkyl, aryl, and heteroaryl; and

n18 is 0 or 1; and

H) a thioether or thiol of formula —(X₂₂)ₙ₂₂—S—X₂₃, wherein

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl; and

c) R₁₀ is selected from the group consisting of hydrogen, lower alkyl, aryl, and heteroaryl;

provided that at least one of R₆-R₁₀ is not selected from the group consisting of hydrogen, lower alkyl, aryl, and heteroaryl.

In the eighteenth embodiment, the invention relates to the compound of the seventeenth embodiment, wherein R₆ is selected from the group consisting of

i) hydrogen;

ii) a six-membered aromatic ring, optionally substituted with one or more substituents selected from the group consisting of lower alkyl, hydroxy, and amino;

iii) a six-membered heteroaromatic ring, optionally substituted with one or more substituents selected from the group consisting of lower alkyl, hydroxy, alkoxy, amino, and nitro;

iv) a five-membered heteroaromatic ring, optionally substituted with one or more substituents selected from the group consisting of lower alkyl, hydroxy, and amino; and

v) acyl of formula —(X₁)ₙ₁—C(O)—X₂, wherein

X₁ is lower alkylene or lower alkenylene;

X₂ is selected from the group consisting of hydrogen, amino, hydroxy, and —NH—X₃,

wherein X₃ is selected from the group consisting of hydrogen, lower alkyl, amino, and amide; and

n1 is 0 or 1.

In the nineteenth embodiment, the invention relates to the compound of the seventeenth embodiment, wherein R₆ is selected from the group consisting of

i) hydrogen;

ii) a phenyl, optionally substituted with one or more of hydroxy or —NH₂;

iii) a six-membered heteroaromatic ring, selected from the group consisting of pyridine, pyrazine, pyridazine, pyrimidine, and 1,3,5-triazine, each independently and optionally substituted with one or more substituents selected from the group consisting of lower alkyl, hydroxy, alkoxy, and amino;

iv) a five-membered heteroaromatic ring, selected from the group consisting of pyrrole, imidazole, 1,2,3-triazole, 1,2,4-triazole, tetrazole, thiophene, thiazole, and thiadiazole, each independently and optionally substituted with one or more substituent selected from the group consisting of hydroxy, an amide of formula —(X₂₄)ₙ₂₄—NH—C(O)—X₂₅ or —(X₂₆)ₙ₂₆—C(O)—NH—X₂₇, and —NH₂;

v) acyl of formula —C(O)—X₂, wherein X₂ is hydrogen or lower alkyl; and

vi) acyl of formula —X₁—C(O)—X₂, wherein

X₁ is lower alkylene or lower alkenylene; and

X₂ is —NH—X₃, wherein X₃ is selected from the group consisting of hydrogen, amino, and amide.

In the twentieth embodiment, the invention relates to the compound of the seventeenth embodiment, wherein said five- or six-membered heteroaryl ring in R₆ is selected from the group consisting of optionally substituted

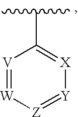

optionally substituted

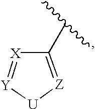

and optionally substituted

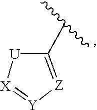

wherein V, W, X, Y and Z are each independently CR or nitrogen, and U is selected from the group consisting of CR₂, oxygen, sulfur, and NR;

wherein R is selected from the group consisting of hydrogen, alkyl, and aryl.

In the twenty first embodiment, the invention relates to the compound of the twentieth embodiment, wherein said heteroaryl ring is selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, oxadiazole

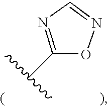

pyran, pyridine, piperidine, morpholine, thiomorpholine, pyridazine, pyrimidine, pyrazine, piperazine, and triazine.

In the twenty second embodiment, the invention relates to the compound of the twenty first embodiment, wherein said heteroaryl ring is selected from the group consisting of aminofuran, aminothiophene, aminopyrrole, aminopyrroline, aminopyrrolidine, aminooxazole, aminothiazole, aminoimidazole, aminoimidazoline, aminoimidazolidine, aminopyrazole, aminopyrazoline, aminopyrazolidine, aminoisoxazole, aminoisothiazole, aminotriazole, aminothiadiazole, aminooxadiazole, aminopyran, aminopyridine, aminopiperidine, aminomorpholine, aminothiomorpholine, aminopyridazine, aminopyrimidine, aminopyrazine, aminopiperazine, and aminotriazine.

In the twenty third embodiment, the invention relates to the compound of the seventeenth embodiment, wherein R₆ is selected from the group consisting of hydrogen, —C(O)—CH₃, —C(O)—NH—CH₂—C(O)—NH₂, —CH═CH—C(O)—NH₂, —CH₂CH₂—C(O)—NH—NH₂,

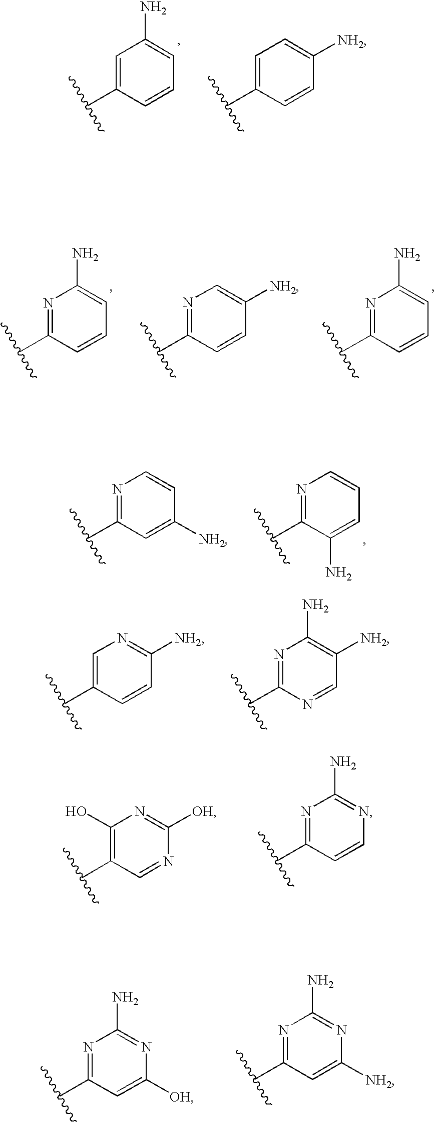

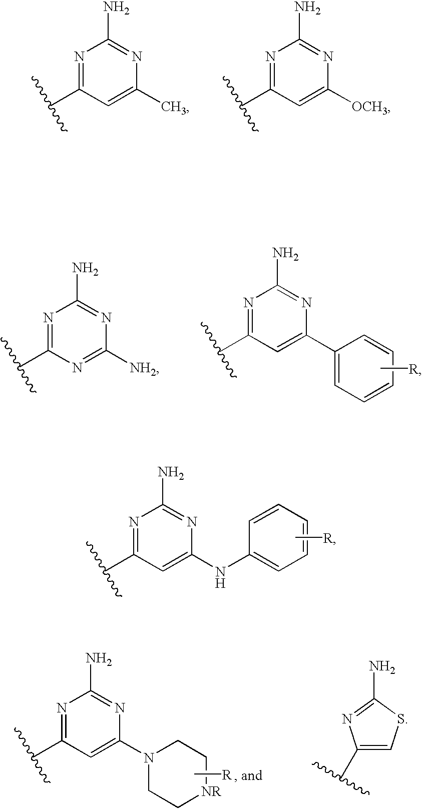

In the twenty fourth embodiment, the invention relates to the compound of the seventeenth embodiment, wherein R₈ is selected from the group consisting of

i) hydrogen;

ii) C₂-C₆ alkenylene;

iii) halogen or perhaloalkyl;

iv) an alkoxy of formula —O—X₁₄, wherein

X₁₄ is selected from the group consisting of hydrogen, lower alkyl, and aryl; and

v) a five-membered or six-membered heteroaryl ring or a six-membered aryl or heteroaryl ring, optionally substituted with one or more substituents selected from the group consisting of

A) optionally substituted C₁-C₄ straight-chain, branched, or cyclic saturated or unsaturated alkyl;

B) an alkoxy of formula —O—X₁₄, wherein X₁₄ is hydrogen or lower alkyl; and

C) halogen or perhaloalkyl;

D) cyano;

E) nitro;

F) a substituent of formula —(X₁₈)ₙ₁₈—C(O)—X₁₉, wherein

X₁₈ is lower alkylene;

X₁₉ is selected from the group consisting of hydrogen, lower alkyl, aryl, heteroaryl, hydroxy, alkoxy, amino, and —NX₂₀X₂₁,

wherein X₂₀ and X₂₁ are each independently selected from the group consisting of hydrogen, alkyl, and aryl; and

n18 is 0 or 1; and

G) a thioether or thiol of formula —S—X₂₃, wherein X₂₃ is selected from the group consisting of hydrogen, lower alkyl, aryl, and heteroaryl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, lower alkyl, aryl, hydroxy, and alkoxy; and

X₂₇ is selected from the group consisting of hydrogen, lower alkyl, aryl, and heteroaryl.

In the twenty fifth embodiment, the invention relates to the compound of the seventeenth embodiment, wherein R₈ is selected from the group consisting of

i) hydrogen;

ii) C₂-C₆ alkenylene;

iii) halogen or perfluoroalkyl;

iv) an alkoxy of formula —O—X₁₄, wherein

X₁₄ is selected from the group consisting of hydrogen, methyl, ethyl, and propyl; and

v) a six-membered aryl ring, optionally substituted with one or more substituents selected from the group consisting of

A) methyl, ethyl, and propyl;

B) hydroxy, methoxy, ethoxy, phenoxy, hydroxymethyl, 2-hydroxyethyl, and 3-hydroxypropyl; and

C) halogen or perfluoroalkyl;

D) cyano;

E) nitro;

F) a substituent of formula —C(O)—X₁₉, wherein

X₁₉ is selected from the group consisting of hydrogen, methyl, ethyl, propyl, phenyl, hydroxy, methoxy, ethoxy, propoxy, amino, and —NX₂₀X₂₁,

wherein X₂₀ and X₂₁ are each independently selected from the group consisting of hydrogen, methyl, ethyl, propyl, and phenyl; and

G) a thioether or thiol of formula —S—X₂₃, wherein X₂₃ is selected from the group consisting of hydrogen, methyl, ethyl, propyl, and phenyl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, methyl, ethyl, propyl, phenyl, hydroxy, methoxy, and phenoxy; and

X₂₇ is selected from the group consisting of hydrogen, methyl, ethyl, propyl, and phenyl.

In the twenty sixth embodiment, the invention relates to the compound of the seventeenth embodiment, wherein said five-membered or six-membered heteroaryl ring or said six-membered aryl or heteroaryl ring of R₈ is selected from the group consisting of optionally substituted

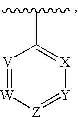

optionally substituted

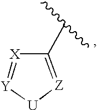

and optionally substituted

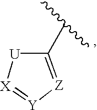

wherein V, W, X, Y and Z are each independently CR or nitrogen, and U is selected from the group consisting of CR₂, oxygen, sulfur, and NR;

wherein R is selected from the group consisting of hydrogen, alkyl, and aryl.

In the twenty seventh embodiment, the invention relates to the compound of the seventeenth embodiment, wherein said five-membered or six-membered heteroaryl ring or said six-membered aryl or heteroaryl ring of R₈ is selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

In the twenty eighth embodiment, the invention relates to the compound of the seventeenth embodiment, wherein R₈ is selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonyphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-fluoro-4-methoxyphenyl,

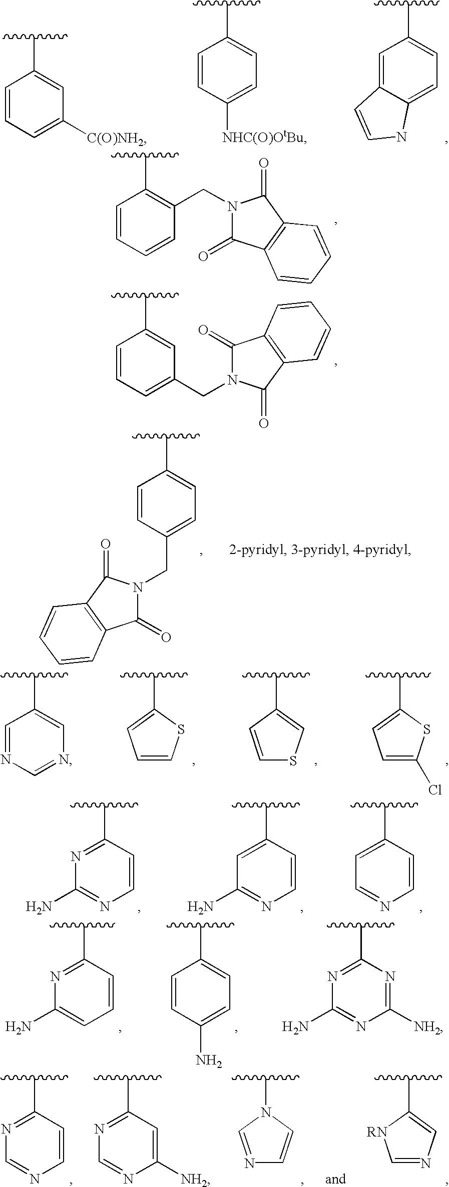

wherein R is selected from the group consisting of hydrogen, alkyl, and aryl.

In the twenty ninth embodiment, the invention relates to a compound of Formula IV

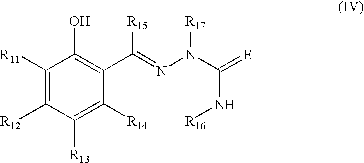

or a pharmaceutically acceptable salt, amide, ester, or prodrug thereof, wherein

a) R₁₁ is selected from the group consisting of hydrogen, lower alkyl, aryl, and heteroaryl;

b) R₁₂, R₁₃, and R₁₄, are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, wherein

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, wherein

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

wherein X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, wherein

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl;

c) R₁₅ is selected from the group consisting of hydrogen, lower alkyl, aryl, alkaryl, heteroaryl, and amino;

d) R₁₆ and R₁₇ are each independently selected from the group consisting of hydrogen, lower alkyl, aryl, and heteroaryl; and

e) E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl.

In the thirtieth embodiment, the invention relates to the compound of the twenty ninth embodiment, wherein R₁₃ is selected from the group consisting of

i) hydrogen;

ii) C₂-C₆ alkenylene;

iii) halogen or perhaloalkyl;

iv) an alkoxy of formula —O—X₁₄, wherein

X₁₄ is selected from the group consisting of hydrogen, lower alkyl, and aryl; and

v) a five-membered or six-membered heteroaryl ring or a six-membered aryl or heteroaryl ring, optionally substituted with one or more substituents selected from the group consisting of

A) optionally substituted C₁-C₄ straight-chain, branched, or cyclic saturated or unsaturated alkyl;

B) an alkoxy of formula —O—X₁₄, wherein X₁₄ is hydrogen or lower alkyl; and

C) halogen or perhaloalkyl;

D) cyano;

E) nitro;

F) a substituent of formula —(X₁₈)ₙ₁₈—C(O)—X₁₉, wherein

X₁₈ is lower alkylene;

X₁₉ is selected from the group consisting of hydrogen, lower alkyl, aryl, heteroaryl, hydroxy, alkoxy, amino, and —NX₂₀X₂₁,

wherein X₂₀ and X₂₁ are each independently selected from the group consisting of hydrogen, alkyl, and aryl; and

n18 is 0 or 1; and

G) a thioether or thiol of formula —S—X₂₃, wherein X₂₃ is selected from the group consisting of hydrogen, lower alkyl, aryl, and heteroaryl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, lower alkyl, aryl, hydroxy, and alkoxy; and

X₂₇ is selected from the group consisting of hydrogen, lower alkyl, aryl, and heteroaryl.

In the thirty first embodiment, the invention relates to the compound of the twenty ninth embodiment, wherein R₁₃ is selected from the group consisting of

i) hydrogen;

ii) C₂-C₆ alkenylene;

iii) halogen or perfluoroalkyl;

iv) an alkoxy of formula —O—X₁₄, wherein

X₁₄ is selected from the group consisting of hydrogen, methyl, ethyl, and propyl; and

v) a six-membered aryl ring, optionally substituted with one or more substituents selected from the group consisting of

A) methyl, ethyl, and propyl;

B) hydroxy, methoxy, ethoxy, phenoxy, hydroxymethyl, 2-hydroxyethyl, and 3-hydroxypropyl; and

C) halogen or perfluoroalkyl;

D) cyano;

E) nitro;

F) a substituent of formula —C(O)—X₁₉, wherein

X₁₉ is selected from the group consisting of hydrogen, methyl, ethyl, propyl, phenyl, hydroxy, methoxy, ethoxy, propoxy, amino, and —NX₂₀X₂₁,

wherein X₂₀ and X₂₁ are each independently selected from the group consisting of hydrogen, methyl, ethyl, propyl, and phenyl; and

G) a thioether or thiol of formula —S—X₂₃, wherein X₂₃ is selected from the group consisting of hydrogen, methyl, ethyl, propyl, and phenyl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, methyl, ethyl, propyl, phenyl, hydroxy, methoxy, and phenoxy; and

X₂₇ is selected from the group consisting of hydrogen, methyl, ethyl, propyl, and phenyl.

In the thirty second embodiment, the invention relates to the compound of the twenty ninth embodiment, wherein said five-membered or six-membered heteroaryl ring or said six-membered aryl or heteroaryl ring of R₁₃ is selected from the group consisting of optionally substituted

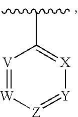

optionally substituted

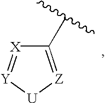

and optionally substituted

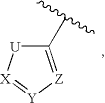

wherein V, W, X, Y and Z are each independently CR or nitrogen, and U is selected from the group consisting of CR₂, oxygen, sulfur, and NR;

wherein R is selected from the group consisting of hydrogen, alkyl, and aryl.

In the thirty third embodiment, the invention relates to the compound of the twenty ninth embodiment, wherein said five-membered or six-membered heteroaryl ring or said six-membered aryl or heteroaryl ring of R₁₃ is selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

In the thirty fourth embodiment, the invention relates to the compound of the twenty ninth embodiment, wherein R₁₃ is selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonylphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-fluoro-4-methoxyphenyl,

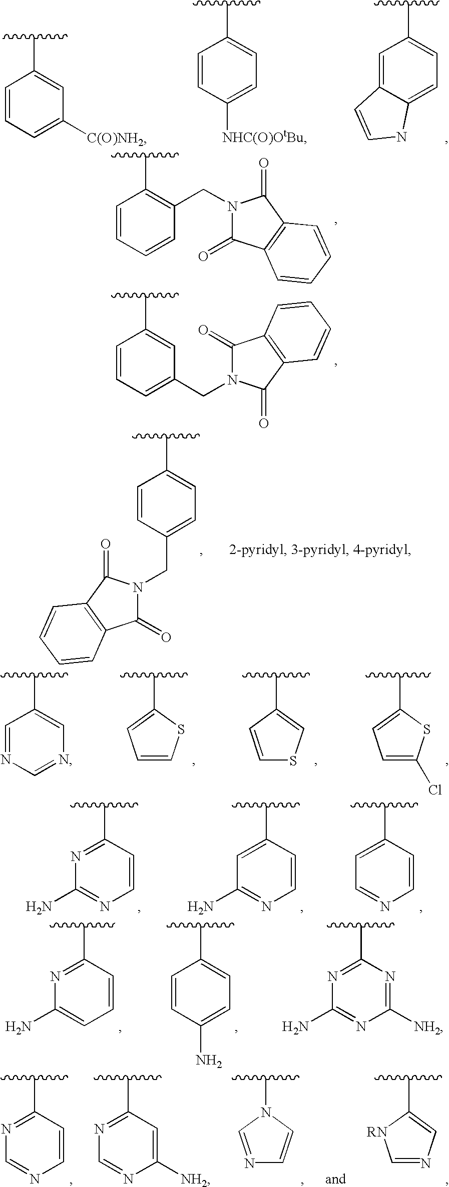

wherein R is selected from the group consisting of hydrogen, alkyl, and aryl.

In the thirty fifth embodiment, the invention relates to the compound of the twenty ninth embodiment, wherein said R₁₁, R₁₂, R₁₃, and R₁₄ is each independently selected from the group consisting of (i) hydrogen, (ii) hydroxyl, (iii) halogens, (iv) cyano, (v) nitro, (vi) amino, (vii) hydroxycarbonyl, (viii) aminocarbonyl, (ix) aminothiocarbonyl, (x) lower alkoxy, (xi) phenoxy, (xii) (C₁-C₄)alkylamino, (xiii) arylamino, (xiv) C₁-C₈ straight-chain, branched, and cyclic saturated and unsaturated alkyl or alkenyl, (xv) optionally substituted aryl and (xvi) optionally substituted hereocycle.

In the thirty sixth embodiment, the invention relates to the compound of the twenty ninth embodiment, wherein said R₁₅ is selected from the group consisting of (i) hydrogen, (ii) cyano, (iii) amino, (iv) hydroxycarbonyl, (v) aminocarbonyl, (vi) aminothiocarbonyl, (vii) (C₁-C₄)alkylamino, (viii) arylamino, (ix) C₁-C₈ straight-chain, branched, and cyclic saturated and unsaturated alkyl or alkenyl, (x) optionally substituted aryl and (xi) optionally substituted hereocycle.

In the thirty seventh embodiment, the invention relates to the compound of the twenty ninth embodiment, wherein said R₁₆ is selected from the group consisting of (i) hydrogen, (ii) amino, (iii) hydroxycarbonyl, (iv) aminocarbonyl, (v) aminothiocarbonyl, (vi) (C₁-C₄)alkylamino, (vii) arylamino, (viii) C₁-C₈ straight-chain, branched, and cyclic saturated and unsaturated alkyl or alkenyl, (ix) optionally substituted aryl and (x) optionally substituted hereocycle.

In the thirty eighth embodiment, the invention relates to the compound of the twenty ninth embodiment, wherein said R₁₇ is selected from the group consisting of (i) hydrogen, (ii) (C₁-C₄)alkylamino, (iii) arylamino, (iv) C₁-C₈ straight-chain, branched, and cyclic saturated and unsaturated alkyl, (v) optionally substituted aryl and (vi) optionally substituted hereocycle.

In the thirty ninth embodiment, the invention relates to the compound of set forth in the thirty fifth through thirty eighth embodiments, wherein said heterocyle is selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, triazine, and benzimidazole.

In the fortieth embodiment, the invention relates to a compound of Formula V or of Formula VI

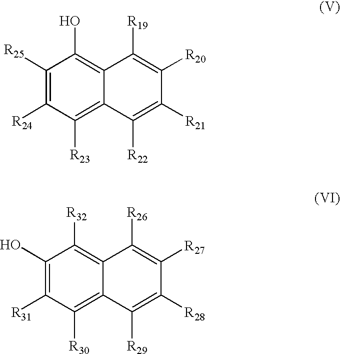

or a pharmaceutically acceptable salt, amide, ester, or prodrug thereof, wherein

a) R₁₉-R₂₂ and R₂₆-R₂₉ are each independently selected from the group consisting of:

i) hydrogen;

ii) optionally substituted C₁-C₈ straight-chain, branched, or cyclic saturated or unsaturated alkyl;

iii) optionally substituted aryl;

iv) optionally substituted heterocyle;

v) substituent of formula —C(X₄)═N—NX₅—C(═E)—NX₆X₇, or of formula —C(X₄)═N—N═C(EX₅)—NX₆X₇, wherein

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

vi) an amino guanidine of formula —NX₈—NX₉—C(═NX₁₀)—NX₁₁X₁₂, wherein X₈, X₉, X₁₀, X₁₁, and X₁₂ are each independently selected from the group consisting of hydrogen, lower alkyl, aryl, and heteroaryl;

vii) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1;

viii) halogen or perhaloalkyl;

ix) cyano;

x) nitro;

xi) an amino of formula —(X₁₅)ₙ₁₅—NX₁₆X₁₇, wherein

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

xii) a substituent of formula —(X₁₈)ₙ₁₈—C(═E)—X₁₉, wherein

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, amino, and —NX₂₀X₂₁,

wherein X₂₀ and X₂₁ are each independently selected from the group consisting of hydrogen, alkyl, aryl, and heteroaryl; and

n18 is 0 or 1; and

xiii) a thioether or thiol of formula —(X₂₂)ₙ₂₂—S—X₂₃, wherein

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

b) R₂₃ and R₃₀ are each independently selected from the group consisting of

i) hydrogen;

ii) lower alkyl;

iii) lower alkylene;

iv) halogen or perhaloalkyl;

v) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1; and

vi) a five-membered or six-membered heteroaryl ring or a six-membered aryl or heteroaryl ring, optionally substituted with one or more substituents selected from the group consisting of

A) optionally substituted C₁-C₈ straight-chain, branched, or cyclic saturated or unsaturated alkyl;

B) an alkoxy of formula —(X₁₃)ₙ₁₃—O—X₁₄, wherein

X₁₃ is selected from the group consisting of lower alkylene, lower alkenylene, lower alkynylene, aryl, and heteroaryl;

X₁₄ is selected from the group consisting of hydrogen, lower alkyl, aryl, and heteroaryl; and

n13 is 0 or 1

C) halogen or perhaloalkyl;

D) cyano;

E) nitro;

F) an amino of formula —(X₁₅)ₙ₁₅—NX₁₆X₁₇, wherein

X₁₅ is selected from the group consisting of lower alkylene, lower alkenylene, lower alkynylene, aryl, and heteroaryl;

X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and

n15 is 0 or 1;

G) a substituent of formula —(X₁₈)ₙ₁₈—C(═E)—X₁₉, wherein

X₁₈ is selected from the group consisting of lower alkylene, lower alkenylene, lower alkynylene, aryl, and heteroaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;

X₁₉ is selected from the group consisting of hydrogen, lower alkyl, aryl, heteroaryl, hydroxy, alkoxy, amino, and —NX₂₀X₂₁,

wherein X₂₀ and X₂₁ are each independently selected from the group consisting of hydrogen, alkyl, aryl, and heteroaryl; and

n18 is 0 or 1; and

H) a thioether or thiol of formula —(X₂₂)ₙ₂₂—S—X₂₃, wherein

X₂₂ is selected from the group consisting of lower alkylene, lower alkenylene, lower alkynylene, aryl, and heteroaryl;

X₂₃ is selected from the group consisting of hydrogen, lower alkyl, aryl, and heteroaryl; and

n22 is 0 or 1;

I) an amide of formula —(X₂₄)ₙ₂₄—NH—C(O)—X₂₅ or —(X₂₆)ₙ₂₆—C(O)—NH—X₂₇

X₂₄ and X₂₆ are each independently selected from the group consisting of lower alkylene, lower alkenylene, lower alkynylene, aryl, and heteroaryl;

X₂₅ is selected from the group consisting of hydrogen, lower alkyl, lower alkenyl, aryl, heteroaryl, hydroxy, alkoxy, and amide; and

X₂₇ is selected from the group consisting of hydrogen, lower alkyl, lower alkenyl, aryl, and heteroaryl; and

c) R₂₄, R₂₅, R₃₁ and R₃₂ are each independently selected from the group consisting of

i) a six-membered aromatic or heteroaromatic, or a five- or six-membered heteroaromatic ring, optionally substituted with one or more substituents selected from the group consisting of lower alkyl, hydroxy, alkoxy, amino, and nitro;

ii) acyl of formula —(X₁)ₙ₁—C(O)—X₂, wherein

X₁ is selected from the group consisting of lower alkylene, lower alkenylene, lower alkynylene, aryl, and heteroaryl;

X₂ is selected from the group consisting of hydrogen, lower alkyl, aryl, heteroaryl, hydroxy, alkoxy, amino, and —NH—X₃,

wherein X₃ is selected from the group consisting of hydrogen, alkyl, aryl, heteroaryl, amino, and amide; and

n1 is 0 or 1; and

iii) substituent of formula —C(X₄)═N—NX₅—C(═E)—NX₆X₇, or of formula —C(X₄)═N—N═C(EX₅)—NX₆X₇, wherein

X₄ is selected from the group consisting of hydrogen, lower alkyl, aryl, alkaryl, heteroaryl, and amino;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen, lower alkyl, aryl, and heteroaryl;

provided that none of R₂₄, R₂₅, R₃₁ or R₃₂ is —C₆H₅, —C(O)H, —C(O)CH₃, —C(O)—C₆H₅, —C(O)NH₂, or —C₆H₄CH₃.

In the forty first embodiment, the invention relates to the compound of the fortieth embodiment, wherein R₂₄, R₂₅, R₃₁ and R₃₂ are each independently selected from the group consisting of

i) hydrogen;

ii) a six-membered aromatic ring, optionally substituted with one or more substituents selected from the group consisting of lower alkyl, hydroxy, and amino;

iii) a six-membered heteroaromatic ring, optionally substituted with one or more substituents selected from the group consisting of lower alkyl, hydroxy, alkoxy, amino, and nitro;

iv) a five-membered heteroaromatic ring, optionally substituted with one or more substituents selected from the group consisting of lower alkyl, hydroxy, and amino;

v) acyl of formula —(X₁)ₙ₁—C(O)—X₂, wherein

X₁ is lower alkylene or lower alkenylene;

X₂ is selected from the group consisting of hydrogen, amino, hydroxy, and —NH—X₃,

wherein X₃ is selected from the group consisting of hydrogen, lower alkyl, amino, and amide; and

n1 is 0 or 1; and

vi) substituent of formula —C(X₄)═N—NH—C(═E)—NX₆X₇, or of formula —C(X₄)═N—N═C(EX₅)—NX₆X₇, wherein

X₄ is selected from the group consisting of hydrogen, lower alkyl, aryl, and alkaryl;

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

X₅, X₆, and X₇ are each independently selected from the group consisting of hydrogen and lower alkyl.

In the forty second embodiment, the invention relates to the compound of the fortieth embodiment, wherein R₂₄, R₂₅, R₃₁ and R₃₂ are each independently selected from the group consisting of

i) hydrogen;

ii) a phenyl, optionally substituted with one or more of hydroxy or —NH₂;

iii) a six-membered heteroaromatic ring, selected from the group consisting of pyridine, pyrazine, pyridazine, pyrimidine, and 1,3,5-triazine, each independently and optionally substituted with one or more substituents selected from the group consisting of lower alkyl, hydroxy, alkoxy, and amino;

iv) a five-membered heteroaromatic ring, selected from the group consisting of pyrrole, imidazole, 1,2,3-triazole, 1,2,4-triazole, tetrazole, thiophene, thiazole, and thiadiazole, each independently and optionally substituted with one or more substituent selected from the group consisting of hydroxy, an amide of formula —(X₂₄)ₙ₂₄—NH—C(O)—X₂₅ or —(X₂₆)ₙ₂₆—C(O)—NH—X₂₇, and —NH₂;

v) acyl of formula —C(O)—X₂, wherein X₂ is hydrogen or lower alkyl;

vi) acyl of formula —X₁—C(O)—X₂, wherein

X₁ is lower alkylene or lower alkenylene; and

X₂ is —NH—X₃, wherein X₃ is selected from the group consisting of hydrogen, amino, and amide;

vi) substituent of formula —C(X₄)═N—NH—C(═E)—NH₂, wherein

X₄ is selected from the group consisting of hydrogen, methyl, ethyl, phenyl, and —CH₂CH₂—Ph; and

E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl; and

vii) substituent of formula —C(X₄)═N—N═C(SX₅)—NH₂, wherein

X₄ is selected from the group consisting of hydrogen, methyl, ethyl, phenyl, and —CH₂CH₂—Ph; and

X₅ is hydrogen or methyl.

In the forty third embodiment, the invention relates to the compound of the fortieth embodiment, wherein said five- or six-membered heteroaryl ring in R₂₄, R₂₅, R₃₁ and R₃₂ is selected from the group consisting of optionally substituted

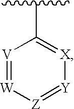

optionally substituted

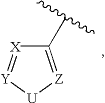

and optionally substituted

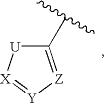

wherein V, W, X, Y and Z are each independently CR or nitrogen, and U is selected from the group consisting of CR₂, oxygen, sulfur, and NR;

wherein R is selected from the group consisting of hydrogen, alkyl, and aryl.

In the forty fourth embodiment, the invention relates to the compound of the forty third embodiment, wherein said heteroaryl ring is selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, oxadiazole, pyran, pyridine, piperidine, morpholine, thiomorpholine, pyridazine, pyrimidine, pyrazine, piperazine, and triazine.

In the forty fifth embodiment, the invention relates to the compound of the forty fourth embodiment, wherein said heteroaryl ring is selected from the group consisting of aminofuran, aminothiophene, aminopyrrole, aminopyrroline, aminopyrrolidine, aminooxazole, aminothiazole, aminoimidazole, aminoimidazoline, aminoimidazolidine, aminopyrazole, aminopyrazoline, aminopyrazolidine, aminoisoxazole, aminoisothiazole, aminotriazole, aminothiadiazole, aminooxadiazole, aminopyran, aminopyridine, aminopiperidine, aminomorpholine, aminothiomorpholine, aminopyridazine, aminopyrimidine, aminopyrazine, aminopiperazine, and aminotriazine.

In the forty sixth embodiment, the invention relates to the compound of the fortieth embodiment, wherein R₂₄, R₂₅, R₃₁ and R₃₂ are each independently selected from the group consisting of hydrogen, —C(O)—CH₃, —C(O)—NH—CH₂—C(O)—NH₂, —CH═CH—C(O)—NH₂, —CH₂CH₂—C(O)—NH—NH₂, —C(H)═N—NH—C(O)—NH₂, —C(CH₃)═N—NH—C(O)—NH₂, —C(H)═N—NH—C(S)—NH₂, —C(CH₃)═N—NH—C(S)—NH₂,—C(Ph)═N—NH—C(S)—NH₂, —C(CH₂CH₂Ph)═N—NH—C(S)—NH₂, —C(H)═N—N═C(SCH₃)—NH₂,

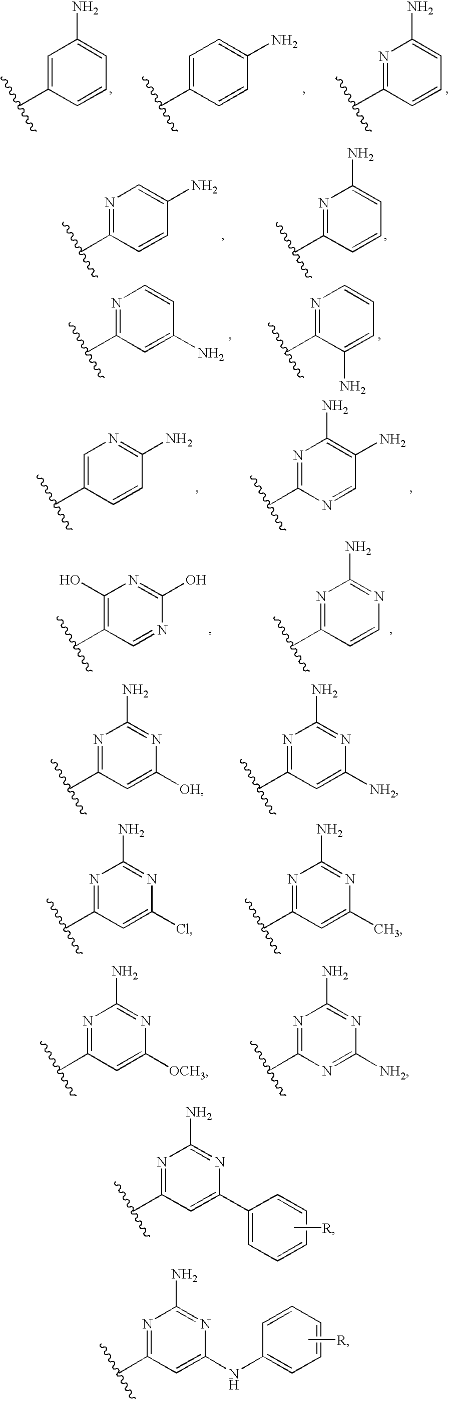

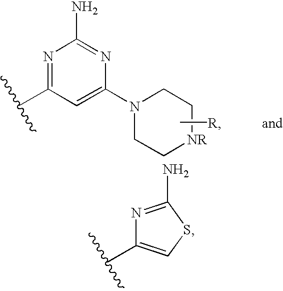

wherein R is selected from the group consisting of hydrogen, alkyl, and aryl.

In the forty seventh embodiment, the invention relates to the compound of the fortieth embodiment, wherein R₂₃ and R₃₀ are each independently selected from the group consisting of

i) hydrogen;

ii) C₂-C₆ alkenylene;

iii) halogen or perhaloalkyl;

iv) an alkoxy of formula —O—X₁₄, wherein

X₁₄ is selected from the group consisting of hydrogen, lower alkyl, and aryl; and

v) a five-membered or six-membered heteroaryl ring or a six-membered aryl or heteroaryl ring, optionally substituted with one or more substituents selected from the group consisting of

A) optionally substituted C₁-C₄ straight-chain, branched, or cyclic saturated or unsaturated alkyl;

B) an alkoxy of formula —O—X₁₄, wherein X₁₄ is hydrogen or lower alkyl; and

C) halogen or perhaloalkyl;

D) cyano;

E) nitro;

F) a substituent of formula —(X₁₈)ₙ₁₈—C(O)—X₁₉, wherein

X₁₈ is lower alkylene;

X₁₉ is selected from the group consisting of hydrogen, lower alkyl, aryl, heteroaryl, hydroxy, alkoxy, amino, and —NX₂₀X₂₁,

wherein X₂₀ and X₂₁ are each independently selected from the group consisting of hydrogen, alkyl, and aryl; and

n18 is 0 or 1; and

G) a thioether or thiol of formula —S—X₂₃, wherein X₂₃ is selected from the group consisting of hydrogen, lower alkyl, aryl, and heteroaryl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, lower alkyl, aryl, hydroxy, and alkoxy; and

X₂₇ is selected from the group consisting of hydrogen, lower alkyl, aryl, and heteroaryl.

In the forty eighth embodiment, the invention relates to the compound of the fortieth embodiment, wherein R₂₃ and R₃₀ are each independently selected from the group consisting of

i) hydrogen;

ii) C₂-C₆ alkenylene;

iii) halogen or perfluoroalkyl;

iv) an alkoxy of formula —O—X₁₄, wherein

X₁₄ is selected from the group consisting of hydrogen, methyl, ethyl, and propyl; and

v) a six-membered aryl ring, optionally substituted with one or more substituents selected from the group consisting of

A) methyl, ethyl, and propyl;

B) hydroxy, methoxy, ethoxy, phenoxy, hydroxymethyl, 2-hydroxyethyl, and 3-hydroxypropyl; and

C) halogen or perfluoroalkyl;

D) cyano;

E) nitro;

F) a substituent of formula —C(O)—X₁₉, wherein

X₁₉ is selected from the group consisting of hydrogen, methyl, ethyl, propyl, phenyl, hydroxy, methoxy, ethoxy, propoxy, amino, and —NX₂₀X₂₁,

wherein X₂₀ and X₂₁ are each independently selected from the group consisting of hydrogen, methyl, ethyl, propyl, and phenyl; and

G) a thioether or thiol of formula —S—X₂₃, wherein X₂₃ is selected from the group consisting of hydrogen, methyl, ethyl, propyl, and phenyl; and

H) an amide of formula —(X₂₄)ₙ₂₄—NH—C(O)—X₂₅ or —(X₂₆)ₙ₂₆—C(O)—NH—X₂₇

X₂₄ and X₂₆ are each independently lower alkylene;

X₂₅ is selected from the group consisting of hydrogen, methyl, ethyl, propyl, phenyl, hydroxy, methoxy, and phenoxy; and

X₂₇ is selected from the group consisting of hydrogen, methyl, ethyl, propyl, and phenyl.

In the forty ninth embodiment, the invention relates to the compound of the fortieth embodiment, wherein said five-membered or six-membered heteroaryl ring or said six-membered aryl or heteroaryl ring of R₂₃ and R₃₀ is selected from the group consisting of optionally substituted

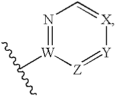

optionally substituted

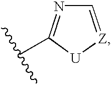

and optionally substituted

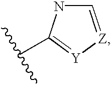

wherein W, X, Y, and Z are each independently CR or nitrogen, and U is selected from the group consisting of CR₂, oxygen, sulfur, and NR; wherein R is selected from the group consisting of hydrogen, alkyl, and aryl.

In the fiftieth embodiment, the invention relates to the compound of the fortieth embodiment, wherein said five-membered or six-membered heteroaryl ring or said six-membered aryl or heteroaryl ring of R₂₃ and R₃₀ is selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

In the fifty first embodiment, the invention relates to the compound of the fortieth embodiment, wherein R₂₃ and R₃₀ are each independently selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonylphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-fluoro-4-methoxyphenyl,

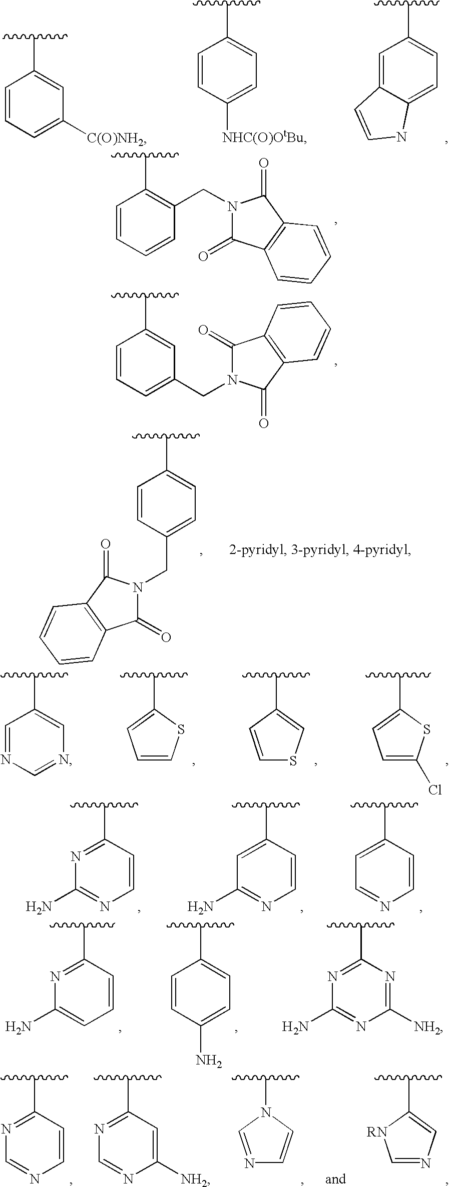

wherein R is selected from the group consisting of hydrogen, alkyl, and aryl.

In the fifty second embodiment, the invention relates to a compound selected from the group consisting of the compounds set forth in Table 1, or a pharmaceutically acceptable salt, ester, amide, or prodrug thereof.

In the fifty third embodiment, the invention relates to a compound of any one of first, second, seventeenth, twentyeigth, fortieth, or fifty second embodiments, wherein said compound is capable of inhibiting the catalytic activity of a protein kinase.

In the fifty fourth embodiment, the invention relates to the compound of the fifty third embodiment, wherein said protein kinase is selected from the group consisting of a receptor protein tyrosine kinase, a cellular tyrosine kinase, and a serine-threonine kinase.

In the fifty fifth embodiment, the invention relates to the compound of the fifty third embodiment, wherein said protein kinase is a cyclin dependent kinase.

In the fifty sixth embodiment, the invention relates to the compound of the fifty fifth embodiment, wherein said cyclin dependent kinase is selected from the group consisting of CDK1 (CDC2), CDK2, CDK3, CDK4, CDK5, CDK6, CDK7, CDK8, and CDK9.

In the fifty seventh embodiment, the invention relates to the compound of the fifty fifth embodiment, wherein said cyclin dependent kinase is selected from the group consisting of CDK2 and CDK5.

In the fifty eighth embodiment, the invention relates to the compound of the fifty fourth embodiment, whrein said protein kinases is selected from the group consisting of protein kinase C, MEK1, MAP kinase, EGF receptor, PDGF receptor, IGF receptor, her2, raf1, PI3 kinase, weel kinase, Src, and Abl.

In the fifty ninth embodiment, the invention relates to a method for the modulation of the catalytic activity of a protein kinase comprising contacting said protein kinase with a compound of any one of first, second, seventeenth, twenty eighth, fortieth, or fifty second embodiments.

In the sixtieth embodiment, the invention relates to a method of modulating a signal transduction pathway in a cells comprising the step of contacting said cell with said compound with a compound according to any one of first, second, seventeenth, twenty eighth, fortieth, or fifty second embodiments.

In the sixty first embodiment, the invention relates to the method of the sixtieth embodiment, wherein said cells express a protein kinase and wherein said compound modulates the function of said protein kinase.

In the sixty second embodiment, the invention relates to a method of identifying an aromatic compound that modulates the function of protein kinase, comprising the following steps:

a) contacting cells expressing said protein kinase with a compound of any one of first, second, seventeenth, twenty eighth, fortieth, or fifty second embodiments; and

b) monitoring an effect of said compound upon said cells.

In the sixty third embodiment, the invention relates to the method of the sixty second embodiment, wherein said effect is selected from the group consisting of a change in cell phenotype, a change in cell proliferation, a change in the catalytic activity of said protein kinase, and a change in the interaction between said protein kinase and a binding partner.

In the sixty fourth embodiment, the invention relates to a method of regulating an unregulated protein kinase signal transduction comprising administering to a subject a therapeutically effective amount of a compound according to any one of first, second, seventeenth, twenty eighth, fortieth, or fifty second embodiments.

In the sixty fifth embodiment, the invention relates to the method of the sixty fourth embodiment, wherein unregulated protein kinase signal transduction leads to a disease or an abnormal condition in an organism and said method leads to the treatment or prevention of said disease or abnormal condition;

wherein said disease or abnormal condition is associated with an aberration in a signal transduction pathway characterized by an interaction between a protein kinase and a binding partner, and

wherein said method further comprises the steps of promoting or disrupting said abnormal interaction.

In the sixty sixth embodiment, the invention relates to the method of the sixty fifth embodiment, wherein said disease or abnormal condition is selected from the group consisting of cell proliferative disease, cerebrovascular damage, autoimmune diseases, neurodegenerative disease, degenerative diseases of the musculoskeletal system,.

In the sixty seventh embodiment, the invention relates to the method of the sixty sixth embodiment,, wherein said neurodegerative disease is selected from the group consisting of AIDS related dementia, Alzheimer's disease, Parkinson's disease, Pick's disease, Huntington's disease, diffuse Lewy body disease, multiple system atrophy, amyotrophic lateral sclerosis, dementia associated with Down's syndrome, cerebrovascular dementia, canine motor neuron disease, retinitis pigmentosa, spinal muscular atrophy, myelodysplastic syndromes, stroke and reperfusion injury, aplastic anemia, ischemic injury associated with myocardial infarctions, arrythmia, atherosclerosis, toxin-induced or alcohol related diseases, hematological diseases including but not limited to chronic anemia and aplastic anemia, and cerebral degeneration.

In the sixty eighth embodiment, the invention relates to the method of the sixty sixth embodiment,, wherein said cerebrovascular damageis selected from the group consisting of cerebrovascular dementia, stroke, cerebral ischemia, and head trauma.

In the sixty ninth embodiment, the invention relates to the method of the sixty sixth embodiment,, wherein said autoimmune disease is selected from the group consisting of systemic lupus, erthematosus, autoimmune mediated glomerulophritis, rheumatoid arthritis, psoriasis, inflammatory bowel disease, autoimmune diabetes mellitus, and the development of AIDS in HIV-infected individuals.

In the seventieth embodiment, the invention relates to the method of the sixty sixth embodiment,, wherein said neurodegenerative disease is selected from the group consisting of AIDS related dementia, dementias including Alzheimer's disease, Parkinson's disease, Pick's disease, Huntington's disease, diffuse Lewy body disease, multiple system atrophy, amyotrophic lateral sclerosis, dementia associated with Down's syndrome, cerebrovascular dementia, and canine motor neuron disease, retinitis pigmentosa, spinal muscular atrophy and cerebral degeneration.

In the seventy first embodiment, the invention relates to the method of the sixty sixth embodiment,, wherein said degenerative disease is selected from the group consisting of osteoporosis, arthritis, aspirin sensitive rhinosinusitis, cystic fibrosis, multiple sclerosis, kidney disease, chemotherapy induced hair loss, allopecia, and cancer pain.

In the seventy second embodiment, the invention relates to the method of the sixty sixth embodiment,, wherein said cell proliferative disease is selected from the group consisting of

carcinoma, selected from the group consisting of carcinoma of breast, lung, colon, kidney, liver, prostate, stomach, esophagus, gall bladder, ovary, pancreas, cervix, bladder, thyroid, skin, and squamous cell carcinoma;

hematopoietic tumors of myeloid lineage, selected from the group consisting of acute and chronic mylogenous leukemias, promyelocytic leukemia, and myelodysplastic syndrome;

hematopoietic tumors of lymphoid lineage, selected from the group consisting of B-cell lymphoma, T-cell lymphoma, Hodgkin's lymphoma, non-Hodgkin's lymphoma, hairy cell lymphoma, Burkett's lymphoma, leukemia, acute lymphocytic leukemia, and acute lymphoblastic leukemia;

Tumors of messenchymal origin, selected from the group consisting of fibrosarcoma and rhabdomyosarcoma;

Tumors of the central and peripheral nervous system, selected from the group consisting of neuroblastoma, astrocytoma, glioma and schwannomas;

Karposi's sarcoma, melanoma, seminoma, teratocarcinoma, xenoderoma, pigmentosum, osteosarcoma, keratoctanthoma, and thyroid follicular cancer.

In the seventy third embodiment, the invention relates to the method of the sixty sixth embodiment,, wherein said cell proliferative disease is selected from the group consisting of benign prostate hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.

In the seventy fourth embodiment, the invention relates to a pharmaceutical composition comprising

i) a physiologically acceptable carrier, diluent, or excipient, or a combination thereof; and

ii) a compound according to any one of first, second, seventeenth, twenty eighth, fortieth, or fifty second embodiments.

### EXAMPLES

The examples below are non-limiting and are merely representative of various aspects of the invention.

### Synthesis of the Compounds of the Invention

### Example 1

### General Procedures

### General Procedure A: Semicarbazone/Thiosemicarbazone Formation Formation (Mishra, B., Ali, R., J. Indian Chem. Soc., 1989, 66, 813-814)

One equivalent of the aldehyde and 1.05 equivalents thiosemicarbazide or semicarbazide hydrochloride are dissolved in N,N-dimethylformamide to a final concentration of 0.5 M. A trace amount of camphorsulfonic acid is added and the reaction is stirred at room temperature for 2-18 hours. Following completion of the reaction, as judged by thin layer chromatography, the solvent is removed in vacuo and the product is washed six a times with diethyl ether and dried in vacuo. The reaction yield ranged 90-100%.

### General Procedure B: Biphenyl Aldehydes and Ketones

These compounds were made via the Suzuki reaction (Miyaura, N.; Suzuki, A., Chem. Rev. 1995, 95, 2457-2483). In this reaction, 1 equivalent of arylbromide, 1.05 equivalents of arylboronic acid, and 0.02 equivalents of tetrakis (triphenylphosphine) palladium are dissolved in ethylene glycol dimethyl ether to give a final concentration of 0.2-0.3 M. To this mixture, 2.5 equivalents aqueous 2.0 M sodium carbonate is added and the mixture is heated to reflux under nitrogen for 2-16 hours. The mixture is then cooled to room temperature and partitioned between dichloromethane and water. The organic layer is backwashed with saturated sodium chloride solution, dried over sodium sulfate, filtered and concentrated. The resulting product was purified by column chromatography.

### General Procedure C: Demethylation (Vickery, E. H., Pahler, L. F., Eisenbraun, E. J., J. Org. Chem., 1979, 44, 4444)

One equivalent of methoxy substituted phenol is dissolved in dichloromethane (˜0.04 M) and stirred at −78° C. under nitrogen. To this solution, 10 equivalents of boron tribromide is added slowly. The reaction mixture is stirred at −78° C. for 15 minutes and then at room temperature for 8 hours. The reaction mixture is cooled to 0° C. and one forth volume diethyl ether is carefully added, followed by saturated sodium bicarbonate solution until the mixture is basic as judged by litmus paper. The resulting mixture is extracted with 6 portions of 4:1 dichloromethane:methanol. The combined organic layers are dried over sodium sulfate, filtered and concentrated. The product is purified by preparative thin layer chromatography.

### General Procedure D: Heck Reaction (Zebovitz, T. C.; Heck, R. F. J. Org. Chem, 1997, 42, 3907-3909)

A mixture of 0.0011 g (0.005 mmol) of palladium acetate, 0.026 g (0.1 mmol) of tri-o-toluylphosphine, 1.52 g (10 mmol) of arylbromide, 11 mmol of acrylate/acrylamide and 21 mmol of triethylamine was heated at 100° C. for 15 hours. After cooling, the reaction mixture was made acidic with 0.1 M citric acid solution and extracted several times with ethyl acetate. The combined organic layers are dried over sodium sulfate, filtered and concentrated. The product was purified by column chromatography.

### General Procedure E: Vilsmeier Formylation(Krishna-Rao, G. S., J. Org. Chem, 1981, 46, 5371)

The corresponding hydroxyl naphthalene is combined with 5 equivalent of N,N-dimethylformamide and the mixture is treated with 1.5 equivalent of POCl₃. The solid formed is dissolved and extracted with ethyl acetate and water. The organic phase is washed with sodium bicarbonate (10% aq.) and dried over sodium sulfate. Removal of the solvent gives the crude product, which is then purified on a silica-gel flash column using methylenechloride-methanol (1%) to give the desired aldehyde.

### General Procedure F

One equivalent of the aldehyde obtained from General Procedure E and 1.05 equivalents thiosemicarbazide or semicarbazide hydrochloride are dissolved in N,N-dimethylformamide to a final concentration of 0.5 M. A trace amount of camphorsulfonic acid is added and the reaction is stirred at room temperature for 2-18 hours. Following completion of the reaction, as judged by thin layer chromatography, the solvent is removed in vacuo and the product is washed six times with diethyl ether and dried in vacuo.

### General Procedure G: Suzuki Reaction

Biphenyl aldehydes and ketones were made via the Suzuki reaction (Miyaura, N.; Suzuki, A. Chem. Rev. 1995, 95, 2457-2483). In this reaction, 1 equivalent of aryl bromide, 1.05 equivalents of boronic acid or boronate, and 0.02 equivalents Tetrakis (triphenylphosphine) palladium are dissolved in ethylene glycol dimethyl ether to give a final concentration of 0.2-0.3 M. To this mixture, 2.5 equivalents of aqueous 2.0 M sodium carbonate are added and the mixture is heated to reflux under nitrogen for 2-16 hours. The mixture is then cooled to room temperature and partitioned between dichloromethane and water. The organic layer is backwashed with saturated sodium chloride solution, dried over sodium sulfate, filtered and concentrated. The resulting product was purified by column chromatography to give 30-95% yield.

### General Procedure H: Suzuki Reaction

4,4′-Dibenzyloxy-3-biphenylboronic acid or 4,4′-dimethoxymethylether-3-biphenyl-boronic acid (1 mmol), corresponding arylchloride or aryl bromide (1 mmol), Pd(PPh₃)₄ (0.03 mmol) and cesium fluoride (3 mmol, 1 M solution in degassed water) were dissolved in ethylene glycol dimethyl ether (30 mL) and heated to reflux overnight under nitrogen. The mixture was cooled, poured into cold water and extracted by CH₂Cl₂ (3×50 mL). The combined organic layers were washed with water and saturated sodium chloride solution, dried over Na₂SO₄ and the solvent was evaporated. The residue was purified by column chromatography over silica gel (15×1 cm) eluting by hexane:ethylacetate gradient to get white products.

### General Procedure I: Hydrogenation

A solution of dibenzyloxybiphenyl product obtained from General Procedure H in THF:MeOH (1:3) was stirred at room temperature under hydrogen in presence of 10% Pd/C for 4 h. The catalyst was filtered off through celite and the filtrate was evaporated to dryness. The residue was purified by column chromatography over silica gel (3×20 cm) eluting by CH₂Cl₂:MeOH (9:1).

### General Procedure J: Cleavage of Methoxymethylether (MOM) or Methoxyethylether (MEM) (Corey, E. J., Danheiser, R. L., J. Am. Chem. Soc, 1978, 100, 8031)

One equivalent of disubstituted methoxymethyl phenol is dissolved in dichloromethane (˜0.04 M) and stirred. To this solution, 8 equivalents of triethylsilane is added followed by addition of trifluoroacetic acid (TFA) (10%). The reaction mixture was stirred at room temperature for 5 hours, and then evaporated to dryness. The resulting solid mixture is treated with sat. sodium bicarbonate solution and extracted with 4:1 dichloromethane:methanol several times. The combined organic layers were dried over sodium sulfate, filtered and concentrated. The product is purified by chromatography.

### General Procedure K: Amination

Chloropyrimidine derivative (1 mmol) was heated with 1.3 equivalents of amine and 4 equivalents of triethylamine in 5 mL of n-buthanol at 120° C. overnight. The solvent is then removed under reduced pressure. The resulting mixture was treated with TFA, following General Procedure J without further purification.

### General Procedure L: Deprotection of Methoxy Groups

One equivalent of methoxy substituted phenol is dissolved in dichloromethane (˜0.04 M) and stirred at −78° C. under nitrogen. To this solution, 10 equivalents boron tribromide is added slowly. The reaction mixture is stirred at −78° C. for 15 minutes and then at room temperature for 8 hours. The reaction mixture is cooled to 0° C. and one forth volume diethyl ether is carefully added followed by saturated sodium bicarbonate solution until the mixture is basic as judged by litmus paper. The resulting mixture is extracted with 6 portions of 4:1 dichloromethane:methanol. The combined organic layers are dried over sodium sulfate, filtered and concentrated. The product is purified by preparative thin layer chromatography.

### General Procedure M: Suzuki Reaction Starting from 4-[4-(2-methoxy-ethoxymethoxy)-3-(4,4,5,5-tetramethyl-[1,3,2]-dioxaborolan-2-yl-phenyl]-pyrimidin-2-ylamine (Reaction Scheme 8)

### 1-(3-Bromo-4-hydroxy-phenyl)-ethanone

A solution of 4-hydroxyacetophenone (13.6 g, 0.1 mol) in 65% acetic acid (100 mL) was treated with bromine (5.1 mL, 0.1 mol) dissolved in 80% acetic acid (50 mL) at room temperature. After addition of bromine the solution was further stirred at room temperature for 30 min and poured into 200 mL of cold water. The side product formed was filtered off and the filtrate was further diluted with 1 L of cold water. The white precipitate formed was filtered, washed several times with water and dried under vacuum. Yield: (10.4 g, 35%).

¹H NMR(CDCl₃) δ2.57 (s, 3H), 6.46 (s, 1H), 7.08 (d, 1H), 7.85 (d, 1H), 8.21 (s, 1H).

MS 216 (M+H), C₈H₇O₂Br

### 1-3-Bromo-4-(2-methoxy-ethoxymethoxy)-phenyl)-ethanone

A solution of 1-(3-Bromo-4-hydroxy-phenyl)-ethanone (15.5 g, 72.1 mmol) in anhydrous ether (100 mL) was added slowly to a suspension of NaH (3.46 g of 60% in mineral oil, 86.5 mmol) in ether (50 mL) under nitrogen at room temperature. The suspension was stirred for 2 h then treated with MOM-Cl (2-methoxyethoxymethyl chloride, 9.86 mL, 86.5 mmol) and further stirred for 1 h at room temperature under nitrogen. The solution was cooled to 0° C., treated with cold water and extracted by ether. The combined organic phase were washed subsequently with satd. NaHCO₃ solution, water and brine then dried over Na₂SO₄ and concentrated under vacuum to get yellow oil (21.64 g, 99%).

¹H NMR(CDCl₃) δ2.56 (s, 3H), 2.96 (s, 3H), 3.56 (t, 2H), 3.82 (t, 2H), 4.42 (s, 2H), 7.25 (d, 1H), 7.86 (d, 1H), 8.17 (s, 1H).

MS 304 (M+H), C₁₂H₁₅O₄Br

### 4-[3-Bromo-4-(2-methoxy-ethoxymethoxy)-phenyl]-pyrimidin-2-ylamine

A solution of 1-[3-Bromo-4-(2-methoxy-ethoxymethoxy)-phenyl]-ethanone (21.6 g, 71.28 mmol) in N,N′-dimethylformamide diethylacetal (183 ml, 1.069 mmol) was heated to reflux for 24 h and evaporated under vacuum to get a red oil. This oil was dissolved in ethanol (180 mL) and treated with sodium ethoxide (19.40 g, 285.28 mmol) and guanidine hydrochloride (10.21 g, 107 mmol) then heated to reflux overnight. The cooled solution was diluted with water, extracted by CH₂Cl₂ and the combined organic phase was concentrated to get a red viscous oil. This crude product was further purified by flash chromatography eluting with 0-5% MeOH in CH₂Cl₂ to get pale yellow crystals (12.5 g, 49%).

¹H NMR(d₆-DMSO) δ3.21 (s, 3H), 3.39 (t, 2H), 3.77 (t, 2H), 5.43 (s, 2H), 6.70 (s, 2H), 7.14 (d, 1H), 7.33 (d, 1H), 8.03 (dd, 1H), 8.26 (d, 1H), 8.35 (s, 1H).

MS 355 (M+H), C₁₄H₁₆O₃N₃Br

### 4-[4-(2-methoxy-ethoxymethoxy)-3-(4,4,5,5-tetramethyl-[1,3,2]-dioxaborolan-2-yl-phenyl]-pyrimidin-2-ylamine

A mixture of 4-[3-Bromo-4-(2-methoxy-ethoxymethoxy)-phenyl]-pyrimidin-2-ylamine (6.2 g, 17.65 mmol), bis-(pinacolato) diboron (4.93 g, 19.42 mmol), potassium acetate (5.197 g, 52.95 mmol) and Pd(dppf)Cl₂ in anhydrous DMF (125 mL) was heated overnight at 80° C. under nitrogen. The cooled solution was partitioned between CH₂Cl₂ and water. The combined organic phase were washed subsequently with water and brine then dried over Na₂SO₄ and concentrated. The crude product was purified by flash chromatography to get yellow oil (which was solidified after few days on keeping at room temperature.

¹H NMR(d₆-DMSO) δ1.29 (s, 12H), 3.21 (s, 3H), 3.47 (t, 2H), 3.78 (t, 2H), 5.30 (s, 2H), 6.26 (s, 2H), 7.04 (d, 1H), 7.16 (d, 1H), 8.08 (dd, 1H), 8.26 (d, 1H), 8.29 (s, 1H).

MS 402 (M+H), C₂₀H₂₈O₅N₃B.

### Suzuki Reaction

A solution of 4-[4-(2-methoxy-ethoxymethoxy)-3-(4,4,5,5-tetramethyl-[1,3,2]-dioxaborolan-2-yl-phenyl]-pyrimidin-2-ylamine (1 mmol), heterocyclic arylbromide or chloride (1.5 mmol), 2 N aq. Na₂CO₃ (2.5 mL) and Pd(PPh₃)₄ (40 mg) in DME was heated to reflux overnight. The solution was cooled, diluted with water and extracted by CH₂Cl₂. The combined organic phase was washed subsequently by water and brine then dried over Na₂SO₄ and concentrated. The crude product was purified by flash chromatography eluting with 0-3% MeOH in CH₂Cl₂ to get a white solid.

### Example 2

### 5-Bromosalicylaldehyde Thiosemicarbazone

The title compound is made from 5-bromosalicylaldehyde and thiosemicarbazide, following General Procedure A. Yield was quantitative.

¹H NMR (d₆-DMSO): δ6.78 (d, 1H), 7.42 (dd, 1H), 8.144 (s, 1H), 8.26 (s, 1H), 10.16 (s, 1H), 11.36 (s, 1H).

MS 297 (M+Na), C₈H₈BrN₃OS.

### Example 3

### 5-Bromosalicylaldehyde Semicarbazone Hydrochloride

The title compound is made from 5-bromosalicylaldehyde and semicarbazide hydrochloride, following General Procedure A. Yield was quantitative.

¹H NMR (d₆-DMSO): δ6.78 (d, 1H), 7.42 (dd, 1H), 8.144 (s, 1H), 8.26 (s, 1H), 10.16 (s, 1H), 11.36 (s, 1H).

MS 297 (M+Na), C₈H₈BrN₃OS.

### Example 4

### 5-Phenylsalicylaldehyde Thiosemicarbazone

5-Phenylsalicylaldehyde is made from 5-bromosalicylaldehyde and phenyl boronic acid, following Generalprocedure B. Yield was 85%.

¹H NMR (CDCl₃): δ7.08 (d, 1H), 7.26 (t, 1H), 7.52 (m, 2H), 7.68 (m, 2H), 7.82 (d, 2H), 10.04 (s, 1H), 11.12(s, 1H).

The title compound is made from 5-phenylsalicylaldehyde and thiosemicarbazide, following General Procedure A. Yield was quantitative.

¹H NMR (d₆-DMSO): δ6.84 (d, 1H), 7.28 (t, 1H), 7.40 (t, 2H), 7.52 (d, 2H), 7.68 (d, 2H), 8.04 (d, 2H), 8.18 (s, 1H), 8.32 (s, 1H), 10.22 (s, 1H), 11.42(s, 1H).

MS 272 (M+H), C₁₄H₁₃N₃OS.

### Example 5

### 5-Phenylsalicylaldehyde Semicarbazone Hydrochloride

The title compound is made from 5-phenylsalicylaldehyde and semicarbazide hydrochloride, following General Procedure A. Yield was quantitative.

¹H NMR (d₆-DMSO): δ6.50 (br. 2H), 6.94 (d, 1H), 7.28 (t, 1H), 7.38 (t, 2H), 7.48 (d, 2H), 7.66 (d, 2H), 8.07 (s, 1H), 8.18 (s, 1H), 10.22 (s, 1H), 11.22(s, 1H).

MS 256 (M+H), C₁₄H₁₃N₃O₂.

### Example 6

### 5-(3-Methoxyphenyl)salicylaldehyde Thiosemicarbazone

5-(3-Methoxyphenyl)salicylaldehyde is made from 5-bromosalicylaldehyde and 3-methoxyphenyl boronic acid, using General Procedure B.

The title compound is made from 5-(3-methoxyphenyl)salicylaldehyde and thiosemicarbazide, following General Procedure A.

¹H NMR (d₆-DMSO) δ3.81.(s, 3H) 6.87 (d, 1H) 6.94 (d, 1H) 7.18 (s, 1H) 7.25 (d, 1H) 7.33 (t, 1H) 7.54 (d, 1H) 8.14 (s, 2H) 8.22 (s, 1H) 8.41 (s, 1H), 10.11 (s, 1H) 11.41 (s, 1H).

MS (ESI+) 324 (M+Na) C₁₅H₁₅N₃O₂S

### Example 7

### 5-(3-Cyanophenyl)salicylaldehyde Thiosemicarbazone

5-(3-Cyanophenyl)salicylaldehyde is made from 5-bromosalicylaldehyde and 3-cyanophenyl boronic acid, using General Procedure B.

The title compound is made from 5-(3-cyanophenyl)salicylaldehyde and thiosemicarbazide, following General Procedure A.

¹H NMR (d₆-DMSO) δ6.98 (d, 1H) 7.62-7.65 (m, 2H) 7.76 (d, 1H) 8.05 (d, 1H) 8.16 (s, 1H) 8.19(s, 1H) 8.22 (s, 1H) 8.35 (s, 1H) 8.41 (s, 1H) 10.28 (s, 1H) 11.45 (s, 1H).

MS (ESI+) 319 (M+Na) C₁₅H₁₂N₄OS.

### Example 8

### 5-(3-Hydroxymethylphenyl)salicylaldehyde Thiosemicarbazone

5-(3-Hydroxymethylphenyl)salicylaldehyde is made from 5-bromosalicylaldehyde and 3-hydroxymethylphenyl boronic acid, following General Procedure B.

The title compound is made from 5-(3-hydroxymethylphenyl)salicylaldehyde and thiosemicarbazide, following General Procedure A.

¹H NMR (d₆-DMSO) δ4.55 (s, 2H) 5.22 (t, 1H) 6.96 (d, 1H) 7.26 (d, 1H) 7.38 (t, 1H) 7.53 (dd, 1H) 7.55 (d, 1H) 7.58 (s, 1H) 8.10 (s, 1H) 8.16 (s, 1H), (s, 1H) 8.42 (s, 1H) 10.09 (s, 1H) 11.42 (s, 1H).

MS (ESI+) 324 (M+Na) C₁₅H₁₅N₃O₂S.

### Example 9

### 5-(3-Hydroxymethylphenyl)salicylaldehyde Semicarbazone Hydrochloride

5-(3-Hydroxymethylphenyl)salicylaldehyde is made from 5-bromo-salicylaldehyde and 3-hydroxymethylphenyl boronic acid, following General Procedure B.

The title compound is made from 5-(3-hydroxymethylphenyl)salicylaldehyde and semicarbazone hydrochloride, following General Procedure A.

¹H NMR (d₆-DMSO): δ4.56(s, 2H), 6.40 (br. 2H), 6.94 (d, 1H), 7.23 (d, 1H), 7.37 (t, 1H), 7.45 (d, 1H), 7.50 (d, 1H), 7.54 (s, 1H), 8.02 (s, 1H), 8.18 (s, 1H), 10.22 (2s, 2H).

MS 286 (M+H), 308 (M+Na), C₁₅H₁₅N₃O₃.

### Example 10

### 5-(4-Hydroxymethylphenyl)salicylaldehyde Thiosemicarbazone

5-(4-Hydroxymethylphenyl)salicylaldehyde is made from 5-bromosalicylaldehyde and 4-hydroxymethylphenyl boronic acid, following General Procedure B.

The title compound is made from 5-(4-hydroxymethylphenyl)salicylaldehyde and thiosemicarbazide, following General Procedure A. Yield was quantitative.

¹H NMR (d₆-DMSO) δ4.52 (d, 2H) 5.19 (t, 1H) 6.94 (d, 1H) 7.36 (d, 2H) 7.54 (dd, 1H) 7.65 (d, 2H) 8.13 (s, 1H) 8.16 (s, 1H) 8.23 (s, 1H) 8.42 (s, 1H) 10.06 (s, 1H) 11.40 (s, 1H).

MS (ESI+) 324 (M+Na) C₁₅H₁₅N₃O₂S.

### Example 11

### 5-(4-Nitrophenyl)salicylaldehyde Thiosemicarbazone

5-(4-Nitrophenyl)salicylaldehyde is made from 5-bromosalicylaldehyde and 4-nitrophenyl boronic acid, following General Procedure B.

The title compound is made from 5-(4-nitrophenyl)salicylaldehyde and thiosemicarbazide, following General Procedure A.

¹H NMR (d₆-DMSO) δ7.01 (d, 1H), 7.71(dd, 1H), 8.01 (d, 2H), 8.20(s, 1H) 8.24 (s, 1H), 8.26 (d, 2H), 8.42 (s, 1H), 8.43 (s, 1H), 10.43(s, 1H) 11.46 (s, 1H).

### Example 12

### 5-(3-Methoxycarbonylphenyl)salicylaldehyde Thiosemicarbazone

5-(3-Methoxyphenyl)salicylaldehyde is made from 5-bromosalicylaldehyde and 4-methoxycarbonylphenyl boronic acid, following General Procedure B.

The title compound is made from 5-(3-methoxycarbonylphenyl)-salicylaldehyde and thiosemicarbazide, following General Procedure A.

¹H NMR (d₆-DMSO) δ3.88 (s, 3H), 6.99 (d, 1H), 7.55-7.60 (m, 2H), 7.90 (d, 1H), 7.96 (d, 1H), 8.16 (s, 1H), 8.17 (s, 2H), 8.29 (s, 1H), 8.42 (s, 1H), 10.19 (s, 1H), 11.42 (s, 1H).

MS (ESI+) 352 (M+Na) C₁₆H₁₅N₃O₃S.

### Example 13

### 5-(3-Methoxycarbonylphenyl)salicylaldehyde Semicarbazone Hydrochloride

5-(3-Methoxyphenyl)salicylaldehyde is made from 5-bromosalicylaldehyde and 4-methoxycarbonylphenyl boronic acid, following General Procedure B.

The title compound is made from 5-(3-methoxycarbonylphenyl)-salicylaldehyde and semicarbazide hydrochloride, following General Procedure A.

¹H NMR (d₆-DMSO): δ3.82 (s, 3H), 6.50 (br. 2H), 7.04 (d, 1H), 7.48 (d, 1H), 7.53 (t, 1H), 7.84 (d, 1H), 7.92 (d, 1H), 8.07 (s, 1H), 8.12 (s, 1H), 8.20 (s, 1H), 8.84 (s, 1H), 9.92 (s, 1H).

MS 314 (M+H), 336 (M+Na), C₁₆H₁₅N₃O₄.

### Example 14

### 5-(3-Fluoro-4-methoxyphenyl)salicylaldehydethiosemicarbazone

5-(3-Fluoro-4-methoxyphenyl)salicylaldehyde is made from 5-bromo-salicylaldehyde and 3-fluoro-4-methoxyphenyl boronic acid, using General Procedure B.

The title compound is made from 5-(3-fluoroxy-4-methoxyphenyl)-salicylaldehyde and thiosemicarbazide, following General Procedure A.

¹H NMR (d₆-DMSO) δ3.86 (s, 3H), 6.93 (d, 1H), 7.19 (t, 1H), 7.47 (d, 1H), 7.53 (d, 1H), 7.59 (d, 1H), 8.17 (s, 2H), 8.20 (s, 1H), 8.41 (s, 1H), 10.08 (s, 1H), 11.41 (s, 1H).

MS (APCI) 319 (M+H; M+Na) C₁₅H₁₄N₃O₂SF.

### Example 15

### 5-(3-Fluorophenyl)salicylaldehyde Thiosemicarbazone

5-(3-Fluorophenyl)salicylaldehyde is made from 5-bromosalicylaldehyde and 3-fluorophenyl boronic acid, using General Procedure B.

The title compound is made from 5-(3-fluoroxyphenyl)salicylaldehyde and thiosemicarbazide, following General Procedure A.

¹H NMR (d₆-DMSO) δ6.96 (d, 1H), 7.12 (t, 1H), 7.45 (dd, 1H), 7.54 (d, 2H), 7.59 (dd, 1H), 8.15 (s, 2H), 8.27 (s, 1H), 8.42 (s, 1H), 10.16 (s, 1H), 11.40 (s, 1H).

MS (APCI) 289 (M+Na) C₁₄H₁₂N₃OSF.

### Example 16

### 5-(4-Fluorophenyl)salicylaldehyde Thiosemicarbazone

5-(4-Fluorophenyl)salicylaldehyde is made from 5-bromosalicylaldehyde and 4-fluorophenyl boronic acid, using General Procedure B.

The title compound is made from 5-(4-fluoroxyphenyl)salicylaldehyde and thiosemicarbazide, following General Procedure A.

¹H NMR (d₆-DMSO) δ6.95 (d, 1H), 7.24 (t, 2H), 7.51 (d, 1H), 7.71 (t, 2H), 8.11 (s, 1H), 8.13 (s, 1H), 8.21 (s, 1H), 8.41 (s, 1H), 10.06 (s, 1H), 11.39 (s, 1H).

MS (APCI) 289 (M+H; M+Na) C₁₄H₁₂N₃OSF.

### Example 17

### 5-(3-Carboxyphenyl)salicylaldehyde Thiosemicarbazone

5-(3-Carboxyphenyl)salicylaldehyde is made from 5-bromosalicylaldehyde 3-methoxycarbonylphenyl boronic acid, using General Procedure B. The methyl ester is readily hydrolyzed to give free carboxlic acid using LiOH (aq.).

The title compound is made from 5-(3-carboxyphenyl)salicylaldehyde and thiosemicarbazide, using General Procedure A.

¹H NMR (d₆-DMSO) δ6.55 (m, 4H), 6.99 (d, 1H), 7.52 (d, 1H), 7.55 (t, 1H), 7.87 (d, 1H), 7.91 (d, 1H), 8.13 (s, 1H), 8.14 (s, 1H), 8.21 (s, 1H), 8.74 (s, 1H), 13.5 (s, 1H).

MS (APCI) 316 (M+H); 322 (M+Na) C₁₅H₁₃N₃O₃S.

### Example 18

### 5-(3-Carboxyphenyl)salicylaldehyde Semicarbazone Hydrochloride

5-(3-Carboxyphenyl)salicylaldehyde is made from 5-bromosalicylaldehyde 3-methoxycarbonylphenyl boronic acid, using General Procedure B. The methyl ester is readily hydrolyzed to give free carboxlic acid using LiOH (aq.).

The title compound is made from 5-(3-carboxyphenyl)salicylaldehyde and semicarbazide hydrochloride, using General Procedure A.

¹H NMR (d₆-DMSO) δ6.55 (m, 4H), 6.99 (d, 1H), 7.52 (d, 1H), 7.55 (t, 1H), 7.87 (d, 1H), 7.91 (d, 1H), 8.13 (s, 1H), 8.14 (s, 1H), 8.21 (s, 1H), 8.74 (s, 1H), 12.0 13.5 (s, 1H).

MS (APCI) 300 (M+H); 322 (M+Na) C₁₅H₁₃N₃O₄.

### Example 19

### 5-(4-Carboxyphenyl)salicylaldehyde Thiosemicarbazone

5-(4-Carboxyphenyl)salicylaldehyde is made from 5-bromosalicylaldehyde and 4-carboxyphenyl boronic acid, using General Procedure B.

The title compound is made from 5-(4-carboxyphenyl)salicylaldehyde and thiosemicarbazide, using General Procedure A.

¹H NMR (d₆-DMSO) δ7.00 (d, 1H), 7.63 (d, 1H), 7.84 (d, 2H), 7.98 (d, 2H), 8.15 (s, 2H), 8.34 (s, 1H), 8.43 (s, 1H), 10.24 (s, 1H), 11.41 (s, 1H), 12.90 (s, 1H).

MS (APCI) 315 (M+H; M+Na) C₁₄H₁₃N₃O₃S.

### Example 20

### 5-(3-Hydroxyphenyl)salicylaldehyde Thiosemicarbazone

5-(3-Hydroxyphenyl)salicylaldehyde is made from 5-bromosalicylaldehyde and 3-methoxyphenyl boronic acid, using General Procedure B, followed by demethoxylation to release free hydroxy group, using General Procedure C.

The title compound is made from 5-(3-hydroxyphenyl)salicylaldehyde and thiosemicarbazide, following General Procedure A.

¹H NMR (d₆-DMSO) 67 6.70 (d, 1H), 6.94 (d, 1H), 7.01 (s, 1H), 7.08 (d, 1H), 7.20 (t, 1H), 7.45 (d, 1H), 8.09 (d, 2H), 8.14 (s, 1H), 8.42 (s, 1H), 9.39 (s, 1H), 10.02 (s, 1H), 11.37 (s, 1H).

MS (APCI) 287 (M+H) C₁₄H₁₃N₃O₂S.

### Example 21

### 5-(4-Hydroxyphenyl)salicylaldehyde Thiosemicarbazone

5-(4-Hydroxyphenyl)salicylaldehyde is made from 5-bromosalicylaldehyde and 4-methoxyphenyl boronic acid, using General Procedure B, followed by demethoxylation to release free hydroxy group, using General Procedure C.

The title compound is made from 5-(4-hydroxyphenyl)salicylaldehyde and thiosemicarbazide, following General Procedure A.

¹H NMR (d₆-DMSO) δ, 6.80 (d, 2H), 6.90 (d, 1H), 7.43-7.49 (m, 3H), 8.06 (s, 1H), 8.10 (s, 2H), 8.40 (s, 1H), 9.40 (s, 1H), 9.89 (s, 1H), 11.36 (s, 1H).

MS (APCI) 287 (M+H) C₁₄H₁₃N₃O₂S.

### Example 22

### 5-(4-Hydroxyphenyl)salicylaldehyde Semicarbazone Hydrochloride

5-(4-Hydroxyphenyl)salicylaldehyde is made from 5-bromosalicylaldehyde and 4-methoxyphenyl boronic acid, using General Procedure B, followed by demethoxylation to release free hydroxy group, using General Procedure C.

The title compound is made from 5-(4-hydroxyphenyl)salicylaldehyde and thiosemicarbazide, following General Procedure A.

¹H NMR (d₆-DMSO) δ6.47 (s, 2H), 6.81 (d, 2H), 6.89 (d, 1H), 7.40 (d, 1H), 7.47 (d, 2H), 7.95 (s, 1H), 8.18 (s, 1H), 9.41 (s, 1H), 9.93 (s, 1H), 10.19 (s, 1H).

MS (APCI) 271 (M+Na) C₁₄H₁₃N₃O₃.

### Example 23

### 5-(3-Fluoro-4-hydroxyphenyl)salicylaldehyde Thiosemicarbazone

5-(3-Fluoror-4-hydroxyphenyl)salicylaldehyde is made from 5-bromosalicylaldehyde and 3-fluoror-4-methoxyphenyl boronic acid, using General Procedure B, followed by demethoxylation to release free hydroxy group, using General Procedure C.

The title compound is made from 5-(3-fluoro-4-hydroxyphenyl)-salicylaldehyde and thiosemicarbazide, using General Procedure A.

¹H NMR (d₆-DMSO) δ6.91 (d, 1H), 6.98 (t, 1H), 7.32 (d, 1H), 7.49 (s, 1H), 7.51 (s, 1H), 8.16 (s, 3H), 8.40 (s, 1H), 9.85 (s, 1H), 10.02 (s, 1H), 11.40 (s, 1H).

MS (APCI) 305 (M+H; M+Na) C₁₄H₁₂N₃O₂SF.

### Example 24

### 5-(3-Fluoro-4-hydroxyphenyl)salicylaldehyde Semicarbazone Hydrochloride

5-(3-Fluoror-4-hydroxyphenyl)salicylaldehyde is made from 5-bromo-salicylaldehyde and 3-fluoror-4-methoxyphenyl boronic acid, using General Procedure B, followed by demethoxylation to release free hydroxy group, using General Procedure C.

The title compound is made from 5-(3-fluoro-4-hydroxyphenyl)-salicylaldehyde and semicarbazide hydrochloride, using General Procedure A.

¹H NMR (d₆-DMSO) δ6.57 (d, 2H), 6.88 (d, 1H), 6.98 (t, 1H), 7.31 (d, 1H), 7.42 (d, 1H), 7.46 (d, 1H), 7.99 (s, 1H), 8.16 (s, 1H), 9.84 (br., 2H), 10.05 (s, 1H) 10.20 (s, 1H).

MS (APCI) 305 (M+H; M+Na) C₁₄H₁₂N₃O₂SF.

### Example 25

### 5-(3,4-Dihydroxyphenyl)salicylaldehyde Thiosemicarbazone

5-(3,4-Dihydroxyphenyl)salicylaldehyde is made from 5-bromo-salicylaldehyde and 3,4-dimethoxyphenyl boronic acid, using General Procedure B, followed by demethoxylation to release free hydroxy group, using General Procedure C.

The title compound is made from 5-(3,4-dihydroxyphenyl)salicylaldehyde and thiosemicarbazide, using General Procedure A.

¹H NMR (d₆-DMSO) δ6.77 (d, 1H), 6.89 (d, 1H), 6.93 (d, 1H), 7.01 (s, 1H), 7.37 (d, 1H), 8.06 (s, 2H), 8.12 (s, 1H), 8.40 (s, 1H), 8.90 (s, 1H), 8.94 (s, 1H), 9.90 (s, 1H), 11.38 (s, 1H).

MS (APCI) 304 (M+H); 326 (M+Na) C₁₄H₁₃N₃O₃S.

### Example 26

### 5-(3,4-Dihydroxyphenyl)salicylaldehyde Semicarbazone Hydrochloride

5-(3,4-Dihydroxyphenyl)salicylaldehyde is made from 5-bromo-salicylaldehyde and 3,4-dimethoxyphenyl boronic acid, using General Procedure B, followed by demethoxylation to release free hydroxy group, using General Procedure C.

The title compound is made from 5-(3,4-dihydroxyphenyl)salicylaldehyde and semicarbazide hydrochloride, using General Procedure A.

¹H NMR (d₆-DMSO) δ6.47 (s, 2H), 6.81 (d, 2H), 6.89 (d, 1H), 7.40 (d, 1H), 7.47 (d, 2H), 7.95 (s, 1H), 8.18 (s, 1H), 9.41 (s, 1H), 9.93 (s, 1H), 10.19 (s, 1H).

MS (APCI) 298 (M+Na) C₁₄H₁₃N₃O₃.

### Example 27

### 5-(2,4-Dihydroxyphenyl)salicylaldehyde Thiosemicarbazone

5-(2,4-Dihydroxyphenyl)salicylaldehyde is made from 5-bromo-salicylaldehyde and 2,4-dimethoxyphenyl boronic acid, using General Procedure B, followed by demethoxylation to release free hydroxy group, using General Procedure C.

The title compound is made from 5-(2,4-dihydroxyphenyl)salicylaldehyde and thiosemicarbazide, using General Procedure A.

¹H NMR (d₆-DMSO) δ5.76 (s, 1H), 6.28 (d, 1H), 6.37 (s, 1H), 6.85 (d, 1H), 7.04 (d, 1H), 7.36 (d, 1H), 7.86 (s, 1H), 7.88 (s, 1H), 8.07 (s, 1H), 8.40 (s, 1H), 9.24 (s, 1H), 9.77 (s, 1H), 11.35 (s, 1H).

MS 304 (M+H), C₁₄H₁₃N₃O₃S.

### Example 28

### 4-Hydroxy-5-(4-hydroxyphenyl)salicylaldehyde Thiosemicarbazone

4-Hydroxy-5-(4-hydroxyphenyl)salicylaldehyde is made from 5-bromo-2,4-dimethoxybenzaldehyde and 4-methoxyphenyl boronic acid, using General Procedure B followed by demethoxylation to release free hydroxy group, using General Procedure C.

The title compound is made from 4-Hydroxy-5-(4-hydroxyphenyl)-salicylaldehyde and thiosemicarbazide, using General Procedure A.

¹H NMR (d₆-DMSO) δ4.47 (s, 1H), 6.44 (s, 1H), 6.72 (d, 2H), 7.29 (d, 2H), 7.67 (s, 1H), 7.95 (s, 1H), 8.26 (s, 1H), 9.26 (s, 1H), 9.76 (d, 1H), 11.15 (s, 1H).

MS (APCI) 304 (M+H); 326 (M+Na) C₁₄H₁₃N₃O₃S.

### Example 29

### 4-Hydroxy-5-(4-hydroxyphenyl)salicylaldehyde Semicarbazone Hydrochloride

5-(3,4-Dihydroxyphenyl)salicylaldehyde is made from 5-bromosalicyl-aldehyde and 3,4-dimethoxyphenyl boronic acid, using General Procedure B, followed by demethoxylation to release free hydroxy group, using General Procedure C.

The title compound is made from 5-(3,4-dihydroxyphenyl)salicylaldehyde and semicarbazide hydrochloride, using General Procedure A.

¹H NMR (d₆-DMSO) δ6.27(s, 2H), 6.44 (d, 1H), 6.73 (d, 2H), 7.29 (d, 2H), 7.49 (s, 1H), 8.03 (s, 1H), 8.63 (s, 2H), 9.27 (s, 1H), 9.93 (s, 1H).

MS (APCI) 288 (M+H); 310 (M+Na) C₁₄H₁₃N₃O₄.

### Example 30

### 5-(4-Pyridyl)salicylaldehyde Thiosemicarbazone

The 5-(4-pyridyl)salicylaldehyde_was obtained in a yield of 43% according to General Procedure B using 4-pyridyl boronic acid and 5-bromo salicylaldehyde as starting matrial.

The title compound is made from 5-(4-pyridyl)salicylaldehyde and thiosemi-carbazide, using General Procedure A.

¹H NMR (d₆-DMSO) δ6.98 (d, 1H), 7.52 (d, 1H), 7.82 (d, 1H), 8.08 (bs, 2H), 8.42 (s, 1H), 8.46 (s, 1H), 8.62 (d, 2H), 10.35 (s, 1H), 11.42 (s, 1H).

MS 273 (M+H), C₁₃H₁₂N₄OS.

### Example 31

### 5-(3-Pyridyl)salicylaldehyde Thiosemicarbazone

The 5-(3-pyridyl)salicylaldehyde_was obtained in a yield of 40% according to General Procedure B using 3-pyridyl boronic acid and 5-bromo salicylaldehyde as starting matrial.

The title compound is made from 5-(3-pyridyl)salicylaldehyde and thiosemi-carbazide, using General Procedure A.

¹H NMR (d₆-DMSO) δ7.01 (d, 1H), 7.45 (t, 1H), 7.53 (d, 1H), 8.08 (d, 1H), 8.18 (d, 1H), 8.22 (s, 1H), 8.32 (s, 1H), 8.42 (s, 1H), 8.53 (s, 1H), 8.95 (s, 1H), 10.25 (s, 1H), 11.42 (s, 1H).

MS 273 (M+H), C₁₃H₁₂N₄OS.

### Example 32

### 5-(5-Pyrimidyl)salicylaldehyde Thiosemicarbazone

To the solution of 5-bromo-o-anisaldehyde (26 g) in benzene(300 mL) was added ethylene glycol (8.03 mL) and p-toluenesulfonic acid monohydrate (10 mg). The reaction mixture was heated to reflux for 6 hours and water was removed. The resulting reaction mixture was washed with 1 N NaOH, dried over MgSO₄, and concentrated. The residue was purified by silica gel column chromatography to give 25 g of the acetal product.

The (2-bromo-6-methoxy)phenyl-1,3-dioxolane (2 g), obtained from above, along with triethylamine (3.34 mL), and pinacolborane (1.7 mL) was dissolved in 1,4-dioxane. [1.1′-Bis(diphenylphosphino)ferrocene]dichloropallaium was added under nitrogen. The reaction mixture was stirred at 100° C. for 12 hours, then extracted with dichloromethane, washed with water, and dried over MgSO₄, and concentrated. The residue was purified by silica gel column chromatography to give 830 mg of the boronate compound.

The boronate compound obtained above was then reacted with 5-bromopyrimidine, using General Procedure B. A yield of 78% was obtained.

The title compound was obtained in a yield of 94% according to General Procedure A.

¹H NMR (DMSO-d₆) δ7.02 (d, 1H), 7.07 (d, 1H), 8.09 (d, 2H), 8.40 (s, 1H), 8.42 (s, 1H), 9.12 (s, 1H), 9.16 (s, 1H), 9.18 (s, 1H), 10.36 (s, 1H), 11.45 (s, 1H).

MS 274 (M+H) C₁₂H₁₁N₅OS.

### Example 33

### 5-(2-Thienyl)salicylaldehyde Thiosemicarbazone

5-(2-Thienyl)salicylaldehyde is made from 5-bromosalicylaldehyde and 2-thienyl boronic acid, following General Procedure B.

The title compound is made from 5-(2-thienyl)salicylaldehyde and thiosemicarbazide, following General Procedure A.

¹H NMR (d₆-DMSO) δ6.91 (d, 1H), 7.09 (d, 1H), 7.44-7.48 (m, 3H), 8.08 (s, 1H), 8.17 (d, 2H), 8.39 (s, 1H), 10.13 (s, 1H), 11.40 (s, 1H).

MS (APCI) 278 (M+H); 300 (M+Na) C₁₂H₁₁N₃OS₂.

### Example 34

### 5-(3-Thienyl)salicylaldehyde Thiosemicarbazone

5-(3-Thienyl)salicylaldehyde is made from 5-bromosalicylaldehyde and 3-thienyl boronic acid, following General Procedure B.

The title compound is made from 5-(3-thienyl)salicylaldehyde and thiosemicarbazide, following General Procedure A.

¹H NMR (d₆-DMSO) δ6.90 (d, 1H), 7.59 (m, 3H), 7.80 (s, 1H), 8.09 (s, 1H), 8.17 (s, 1H), 8.24 (d, 1H), 8.40 (s, 1H), 10.00 (s, 1H), 11.39 (s, 1H).

MS (APCI) 278 (M+H); 300 (M+Na) C₁₂H₁₁N₃OS₂.

### Example 35

### 5-[2-(5-Chloro-thienyl)]salicylaldehyde Thiosemicarbazone

5-[2-(5-Chlorothienyl)]salicylaldehyde is made from 5-bromosalicyl aldehyde and 5-chloro-2-thienyl boronic acid, following General Procedure B.

The title compound is made from 5-[2-(5-chlorothienyl)]salicylaldehyde and thiosemicarbazide, following General Procedure A.

¹H NMR (d₆-DMSO) δ6.91 (d, 1H), 7.11 (s, 1H), 7.44-7.48 (m, 3H), 8.11-8.17 (m, 3H), 8.37 (s, 2H), 10.23 (s, 1H), 11.41 (s, 1H).

MS (APCI) 335 (M+Na) C₁₂H₁₀ClN₃OS₂.

### Example 36

### 5-(5-Indolyl)salicylaldehyde Thiosemicarbazone

5-(5-Indolyl)salicylaldehyde is made from 5-bromosalicylaldehyde and 5-indolyl boronic acid, using General Procedure B.

The title compound is made from 5-(4-fluoroxyphenyl)salicylaldehyde and thiosemicarbazide, following General Procedure A.

¹H NMR (d₆-DMSO) δ6.32 (d, 1H), 6.90 (d, 1H), 7.34 (d, 1H), 7.38 (s, 1H), 7.40 (d, 1H), 7.54 (d, 1H), 7.82 (d, 1H), 8.15 (bs, 2H), 8.23 (s, 1H), 8.48 (s, 1H), 9.91 (s, 1H), 11.07 (s, 1H), 11.38 (s, 1H).

MS 311 (M+H), C₁₆H₁₄N₄OS.

### Example 37

### Methyl (3-formyl-4-hydroxy)cinnamate, Thiosemicarbazone

Methyl (3-formyl-4-hydroxy)cinnamate is made, using General Procedure E, from 5-bromosalicylaldehyde and methyl acrylate in 90%.

The title compound is made from methyl (3-formyl-4-hydroxy)cinnamate and thiosemicarbazide, following General Procedure A.

¹H NMR (d₆-DMSO) δ3.69 (s, 3H), 6.56 (d, 1H), 6.87 (d, 1H), 7.55-7.60 (m, 2H), 8.13 (d, 2H), 8.33 (d, 2H), 10.05 (s, 1H), 11.38 (s, 1H).

MS 302 (M+Na), C₁₂H₁₃N₃O₃S.

### Example 38

### 3-Formyl-4-hydroxycinnamic Acid, Thiosemicarbazone

Methyl (3-formyl-4-hydroxy)cinnamate is hydrolyzed by using aq. LiOH (0.5 M).

The title compound is made from Methyl (3-formyl-4-hydroxy)cinnamate and thiosemicarbazide, following General Procedure A.

¹H NMR (DMSO-d₆) δ6.44 (d, 1H), 6.87 (d, 1H), 7.50 (d, 1H), 7.53 (d, 1H), 8.13 (d, 2H), 8.32 (d, 2H), 8.61 (s, 1H), 10.43 (s, 1H), 11.38 (s, 1H).

MS 288 (M+Na), C₁₁H₁₁N₃O₃S.

### Example 39

### 3-Formyl-4-hydroxycinnamide, Thiosemicarbazone

3-Formyl-4-hydroxycinnamide is made, using General Procedure E, from 5-bromosalicylaldehyde and acryl amide in 70%.

The title compound is made from methyl 3-formyl-4-hydroxycinnamide and thiosemicarbazide, following General Procedure A.

¹H NMR (d₆-DMSO) δ6.46 (d, 1H), 6.88 (d, 1H), 6.93 (s,1H), 7.37 (d, 1H), 7.43 (d, 1H), 7.92 (s, 1H), 8.17 (d, 2H), 8.33 (d, 1H), 10.03 (s, 1H), 11.39 (s, 1H).

MS 287 (M+Na), C₁₁H₁₂N₄O₂S.

### Example 40

### 3-(3-Formyl-4-hydroxyphenyl)propionic Acid, Thiosemicarbazone

To a solution of 3-(4-hydroxyphenyl)propionic acid (1 g) in CHCl₃ (5 mL) is added 4 N NaOH (4 mL). After stirring for 6 h at 80° C., the mixture was extracted with water, dried over MgSO₄, and concentrated. The residue was purified by silica gel column chromatography to give 850 mg of the aldehyde.

The title compound 3-(3-formyl-4-hydroxyphenyl)propionic acid, thiosemicarbazone is made from 3-(3-formyl-4-hydroxyphenyl)propionic acid and thiosemicarbazide, following General Procedure B.

¹H NMR (d₆-DMSO) δ2.45 (t, 2H), 2.68 (t, 2H), 6.73 (d, 1H), 7.03 (d, 1H), 7.75 (s, 1H), 7.89 (s, 1H), 8.12 (s, 1H), 8.32 (s, 1H), 9.65 (s, 1H), 11.32 (s, 1H), 12.12 (bs, 1H).

MS 268 (M+H), C₁₁H₁₃N₃O₃S.

### Example 41

### 2-[(2-Hydroxy-1-naphthyl)methylidene]hydrazine-1-carbamide Hydrochloride

Preparation method: General Procedure F.

The starting material 2-hydroxy-1-naphthaldehyde is obtained from commercial source. The other starting material is semicarbazide hydrochloride.

¹H NMR (d₆-DMSO): δ7.20 (d, 1H), 7.36 (t, 1H), 7.62 (t, 1H), 7.80 (d, 1H), 7.85 (d, 1H), 7.84 (d, 1H), 8.28 (s, 1H), 8.46 (s, 1H), 8.60 (s, 1H), 9.05 (s, 1H).

MS 230 (M+H), C₁₂H₁₁N₃O₂.

### Example 42

### 2-[(2-Hydroxy-1-naphthyl)methylidene]hydrazine-1-carbothioamide

Preparation method: General Procedure E.

The starting material 2-hydroxy-1-naphthaldehyde is obtained from commercial source. The other starting material is thiosemicarbazide.

¹H NMR (d₆-DMSO): δ7.18 (d, 1H), 7.36 (t, 1H), 7.58 (t, 1H), 7.80 (d, 1H), 7.85 (d, 1H), 7.88 (d, 1H), 8.18 (s, 1H), 8.48 (s, 1H), 8.62 (s, 1H), 9.05 (s, 1H).

MS 246 (M+H), C₁₂H₁₁N₃OS.

### Example 43

### 2-[(2,7-Dihydroxy-1-naphthyl)methylidene]hydrazine-1-carbothioamide

The starting material for example 3 (2,7-dihydroxy-3-naphthaldehyde) is prepared followed General Procedure E from 2,7-dihydroxy-naphthalene with a yield of 40%. Another formylated product (2,7-dihydroxy-1-naphthaldehyde) is isolated with a yield of 20%, which is used for the synthesis of the Example 44.

2,7-dihydroxy-3-naphthaldehyde: ¹H NMR (d₆-DMSO): δ6.92 (m, 2H), 7.56 (d, 1H), 7.94 (s, 1H), 8.28 (s, 1H), 9,90(s, 1H), 10.7 (s, 1H), 11.36 (s, 1H).

The compound of Example 43 was prepared followed General Procedure F with thiosemicarbazide. Yield was quantative.

¹H NMR (d₆-DMSO): δ6.85 (dd, 1H), 6.92 (s, 1H), 7.52 (s, 1H), 7.64 (d, 1H), 7.68 (d, 1H), 8.94 (s, 1H), 9.90 (s, 1H), 10.36 (d, 1H), 11.36(s, 1H).

MS 262 (M+H), C₁₂H₁₁N₃O₂S.

### Example 44

### 2-[(2,7-Dihydroxy-1-naphthyl)methylidene]hydrazine-1-carbothioamide

The starting material for example 4 (2,7-dihydroxy-1-naphthaldehyde) is prepared followed General Procedure E from 2,7-dihydroxy-naphthalene with a yield of 20%.

2,7-dihydroxy-1-naphthaldehyde: ¹H NMR (d₆-DMSO): δ7.04-7.12 (m, 3H), 7.46 (d, 1H), 7.74 (d, 1H), 7.82 (d, 1H), 8.56 (s, 1H), 10.7 (s, 1H).

Another formylated product (2,7-dihydroxy-3-naphthaldehyde) is also isolated with a yield of 40%, which is used for the synthesis of the Example 43.

The compound of Example 44 was prepared followed General Procedure F with thiosemicarbazide. Yield was quantative.

¹H NMR (d₆-DMSO): δ7.50-7.52 (m, 4H), 7.66-7.92 (m, 4H), 9.26 (s, 1H), 9.56 (d, 1H), 9.82 (s, 1H).

MS 262 (M+H), C₁₂H₁₁N₃O₂S.

### Example 45

### 2-[(2,6-Dihydroxy-1-naphthyl)methylidene]hydrazine-1-carbothioamide

The starting material (2,6-dihydroxy-1-naphthaldehyde) is prepared followed General Procedure E from 2,6-dihydroxy-naphthalene with a yield of 30%.

2,6-dihydroxy-1-naphthaldehyde: ¹H-NMR (d₆-DMSO): δ7.04-7.12 (m, 3H), 7.46 (d, 1H), 7.74 (d, 1H), 7.82 (d, 1H), 8.56 (s, 1H), 10.7 (s, 1H).

The compound of Example 45 was prepared following General Procedure F with thiosemicarbazide. Yield was quantative.

¹H-NMR (d₆-DMSO): δ6.78 (d, 1H), 6.86 (s, 1H), 7.48 (s, 1H), 7.56 (d, 1H), 7.88 (m, 1H), 9.20 (s, 2H), 9.46 (d, 2H), 9.82 (s, 2H).

MS 262 (M+H), C₁₂H₁₁N₃O₂S.

### Example 46

### 2-Amino-4-[1-(2-hydroxynaphthyl)]pyrimidine

One equivalent of 2′-methoxy-1′-naphthone and 15 eq. of N,N-dimethylformamide diethyl acetal are combined and heated to 120° C. overnight. The reaction mixture is concentrated in vacuo and dissolved in butanol (0.1 M) with 3 equivalents of sodium ethoxide followed by 1.1 equivalents of guanidine hydrochloride. This mixture is heated to 120° C. overnight. The reaction mixture was extracted with dichloromethane, washed with water and then dried over sodium sulfate. The resulting 2-Amino-4-[1-(2-methoxynaphthyl)]pyrimidine was obtained after purification on a silica gel column.

The methoxy product is then dissolved in dichloromethane (˜0.04 molar) and stirred at −78° C. under nitrogen. To this solution, 10 equivalents boron tribromide is added slowly. The reaction mixture is stirred at −78° C. for 15 minutes and then at room temperature for 8 hours. The reaction mixture is cooled to 0° C. and one forth volume diethyl ether is carefully added followed by saturated sodium bicarbonate solution until the mixture is basic as judged by litmus paper. The resulting mixture is extracted with 6 portions of 4:1 dichloromethane:methanol. The combined organic layers are dried over sodium sulfate, filtered and concentrated. The product is purified by preparative thin layer chromatography.

¹H-NMR (d₆-DMSO): δ5.18 (s, 2H), 5.29 (s, 1H), 7.19 (s, 1H), 7.22 (d, 1H), 7.25 (s, 1H), 7.35 (t, 1H), 7.47 (t, 1H), 7.79 (d, 1H), 7.81 (d, 1H), 8.16 (s, 1H), 8.43 (d, 1H), 12.20 (s, 1H).

MS 238 (M+H), C₁₄H₁₁N₃O.

### Example 47

### 3-(2-Amino-6-methyl-pyrimidin-4-yl)-biphenyl-4,4′-diol

4′-methoxy-biphenyl-4-carboxyaldehyde was made accoding to General Procedure G from 4-bromobenzaldehyde and 4-methoxyphenyl boronic acid in 95% yield.

22.08 g (104 mmol) of 4′-methoxy-biphenyl-4-carboxyaldehyde was dissolved in 300 mL of methylenechloride and stirred at 0° C. 5.36 mL of bromine dissolved in 100 mL of methylenechloride was added dropwise. The reaction mixture stirred overnight and concentrated. The residue was redisolved in methylenechloride and basified with sodium bicarbonate. The organic layer was washed further with water and dried over sodium sulfate. The crude product was purified by column chromatography (1:1 hexane/ethyl acetate) to give desired product in 58% yield.

10.92 g (37.5 mmol) of 3′-bromo-4′-methoxy-biphenyl-4-carboxyaldehyde was dissolved in 100 mL of methylenechloride and stirred at 0° C. 14.6 g (65 mmol; 1.7 equivalent) 77% pure 3-chlorobenzcarboperoxoic acid was added. It was stirred at roomtemperature for 18 hours. The reaction mixture was cooled to 0° C. and treated with saturated sodium bicarbonate solution until no bubble were observed. It was partitioned between saturated sodium bicarbonate solution and two portion of methylenechloride and methanol (2:1) and 100 mL of 1 N lithium hydroxide were added. It was stirred for 2 hours at roomtemperature. The reaction mixture was extracted with methylenechlorid. The organic layer was washed with bicarbonate and dried over sodium sulfate, filtered and concentrated to give 8.72 g of the 3′-bromo-4-hydroxy-4′-methoxy-biphenyl in 80% yield.

The methoxy group on 3′-bromo-4-hydroxy-4′-methoxy-biphenyl was removed according to General Procedure F to give 3-bromo-4,4′-biphenol in 80% yield.

6.2 g of anhydrous potassium carbonate was added to a solution of 3-bromo-4,4′-biphenol (2 g, 7.5 mmol) and benzylbromide (1.8 mL, 15 mmol) in acetone. The mixture was refluxed for 4 h and cooled. The acetone was evaporated under vacuum, water was added to the residue and acidified slowly by cold 10% HCl. The resulted solids were filtered, washed by H₂O and dried overnight in vacuum to get white solids (96%).

¹H NMR (d₆-DMSO) δ5.14 (s, 2H), 5.24 (s, 2H), 6.65 (d, 2H), 7.22 (d, 1H), 7.33 (m, 2H), 7.40 (m, 4H), 7.45 (d, 2H), 7.48 (d, 2H), 7.57 (m, 3H), 7.82 (d, 1H).

MS 446 (M+H), C₂₆H₂₁O₂Br.

4,4′-Dibenzyloxy-3-biphenylboronic acid was obtained as follows:

To a solution of 3-bromo-4,4′-dibenzyloxybiphenol (2.88 g, 6.5 mmol) in dry THF (30 mL) was added BuLi (7.15 mmol) dropwisely at −78° C. under N₂ and further stirred at −78° C. for 30 min. A solution of B(BuO)₃ (20 mmol) in 20 mL THF was added slowly at the same temperature and further stirred at −78° C. for 6 h. The mixture was allowed to warn up and further stirred overnight at room temperature. Then it was cooled to −0° C., treated with 1N NaOH (10 mL) and water (50 mL) and extracted with ether (3×100 mL). The combined organic layer was washed by water, saturated sodium chloride solution, dried over Na₂SO₄ and evaporated. The residue was purified by column chromatography over silica gel (30×4 cm) eluting by CH₂Cl₂ to get white solids (27%).

¹H NMR (d₆-DMSO) δ5.17 (s, 2H), 5.20 (s, 2H), 7.06 (d, 2H), 7.11 (d, 1H), 7.36 (d, 2H), 7.41 (m, 4H), 7.46 (d, 2H), 7.53 (m, 4H), 7.58 (d, 1H), 7.78 (d, 1H), 7.81 (s, 2H).

MS 410 (M+H), C₂₆H₂₃O₄B.

The starting material 4-(4,4′-Bis-benzyloxy-biphenyl-3-yl)-6-methyl-pyrimidin-2-ylamine for the title compound was obtained as white solids (yield 84%) according to General Procedure H by using 2-amino-4-chloro-6-methylpyrimidine.

¹H NMR (d₆-DMSO) δ2.21 (s, 3H), 5.15 (s, 2H), 5.24 (s, 2H), 6.48 (s, 2H), 6.51 (s, 1H), 6.97 (s, 1H), 7.08 (m, 3H), 7.28 (d, 1H), 7.35 (m, 3H), 7.41 (d, 2H), 7.48 (m, 2H), 7.56 (d, 2H), 7.63 (m, 2H), 8.30 (s, 1H).

MS 461 (M+), C₃₀H₂₇N₃O₂.

The title compound was obtained as yellow solids (yield 69%) according to General Procedure I by using 3-(2-Amino-6-methyl-pyrimidin-4-yl)-biphenyl-4,4′-diol.

¹H NMR(d₆-DMSO) δ2.23 (s, 3H), 6.83 (d, 2H), 6.93 (d, 2H), 7.04 (s, 2H), 7.33 (s, 1H), 7.52 (d, 1H), 7.56 (d, 1H), 8.06 (d, 1H), 9.45 (s, 1H), 14.00 (s, 1H).

MS 282 (M+H), C₁₆H₁₅N₃O₂

### Example 48

### 3-(5-Amino-pyridin-2-yl)-biphenyl-4,4′-diol

The starting material 6-(4,4′-Bis-benzyloxy-biphenyl-3-yl)-pyridin-3-ylamine for the title compound was obtained as pale yellow solids (yield 43%) according to General Procedure B by using 5-amino-2-bromopyridine.

¹H NMR(d₆-DMSO) δ5.14 (s, 2H), 5.20 (s, 2H), 5.39 (s, 2H), 6.91 (d, 1H), 7.08 (d, 2H), 7.20 (d, 1H), 7.32 (m, 2H), 7.37-7.41 (m, 4H), 7.44-7.50 (m, 5H), 7.56 (d, 2H), 7.69 (d, 1H), 7.92 (d, 1H), 8.04 (s, 1H).

MS 459 (M+H), C₃₁H₂₆N₂O₂

The title compound was obtained as yellow solids (yield 62%) according to General Procedure C by using 6-(4,4′-Bis-benzyloxy-biphenyl-3-yl)-pyridin-2-ylamine.

¹H NMR(d₆-DMSO) δ5.75 (s, 2H), 6.82 (d, 2H), 6.86 (d, 1H), 7.18 (d, 1H), 7.34 (d, 1H), 7.48 (d, 2H), 7.92 (s, 2H), 8.05 (d, 1H), 9.48 (s, 1H), 14.19 (s, 1H).

MS 279 (M+H), C₁₇H₁₄N₂O₂

### Example 49

### 3-(6-Amino-pyridin-2-yl)-biphenyl-4,4′-diol

The starting material 6-(4,4′-Bis-benzyloxy-biphenyl-3-yl)-pyridin-2-ylamine for the title compound was obtained as white solids (yield 54%) according to General Procedure H by using 2-amino-6-bromopyridine.

¹H NMR(d₆-DMSO) δ5.14 (s, 2H), 5.21 (s, 2H), 5.91 (s, 2H), 6.39 (d, 2H), 7.09 (d, 2H), 7.12 (d, 1H), 7.22 (d, 1H), 7.33 (d, 1H), 7.37-7.41 (m, 5H), 7.44-7.47 (m, 5H), 7.55 (d, 2H), 7.93 (d, 1H).

MS 459 (M+H), C₃₁H₂₆N₂O₂.

The title compound was obtained as yellow solids (yield 62%) according to General Procedure I by using 6-(4,4′-Bis-benzyloxy-biphenyl-3-yl)-pyridin-2-ylamine.

¹H NMR(d₆-DMSO) δ6.46 (d, 1H), 6.60 (s, 2H), 6.79 (d, 2H), 6.82 (d, 1H), 7.35 (d, 1H), 7.483(d, 1H), 7.48 (d, 2H), 7.57 (d, 1H), 7.97 (d, 1H), 9.44 (s, 1H), 14.47 (s, 1H).

MS 279 (M+H), C₁₇H₁₄N₂O₂.

### Example 50

### 3-(2-Amino-6-methoxy-pyrimidin-4-yl)-biphenyl-4,4′-diol

The starting material 4-(4,4′-Bis-benzyloxy-biphenyl-3-yl)-6-methoxy-pyrimidin-2-ylamine for the title compound was obtained as white solids (yield 75%) according to General Procedure H by using 2-amino-4-chloro-6-methoxypyrimidine.

¹H NMR(d₆-DMSO) δ3.87 (s, 3H), 5.10 (s, 2H), 5.18 (s, 2H), 6.76 (s, 2H), 6.88 (s, 1H), 6.97 (s, 1H), 7.06 (m, 3H), 7.28 (d, 1H), 7.35 (m, 3H), 7.41 (d, 2H), 7.48 (m, 2H), 7.56 (d, 2H), 8.02 (m, 2H), 8.05 (s, 1H).

MS 490 (M+H), C₃₁H₂₇N₃O₃.

The title compound was obtained as yellow solids (yield 69%) according to General Procedure I by using 3-(2-Amino-6-methoxy-pyrimidin-4-yl)-biphenyl-4,4′-diol.

¹H NMR(d₆-DMSO) δ3.88 (s, 3H), 6.82 (d, 2H), 6.86 (s, 1H), 6.89 (d, 1H), 7.13 (s, 2H), 7.52 (d, 2H), 7.54 (d, 1H), 8.00 (s, 1H), 9.41 (s, 1H), 14.09 (s, 1H).

MS 310 (M+H), C₁₇H₁₅N₃O₃.

### Example 51

### 3-(4,5-Diamino-pyrimidin-2-yl)-biphenyl-4,4′-diol

The starting material 2-(4,4′-Bis-benzyloxy-biphenyl-3-yl)-pyrimidine-4,5-diamine for the title compound was obtained as white solids (yield 31%) according to General Procedure H by using 2-chloro-4,5-diaminopyrimidine.

¹H NMR(d₆-DMSO) δ4.78 (s, 2H), 5.13 (s, 4H), 6.30 (s, 2H), 7.06 (d, 2H), 7.12 (d, 1H), 7.28 (d, 1H), 7.33 (d, 2H), 7.35 (s, 1H), 7.40 (d, 2H), 7.46-7.53 (m, 4H), 7.53-7.55 (m, 3H), 7.64 (d, 1H), 7.70 (s, 1H).

MS 475 (M+H), C₃₀H₂₆N₄O₂.

The title compound was obtained as yellow solids (yield 56%) according to General Procedure I by using 2-(4,4′-Bis-benzyloxy-biphenyl-3-yl)-pyrimidine-4,5-diamine.

¹H NMR(d₆-DMSO) δ5.06 (d, 2H), 6.75 (d, 2H), 6.83 (d, 1H), 6.88 (s, 2H), 7.39 (s, 1H), 7.40 (d, 2H), 7.66 (s, 1H), 8.36 (s, 1H), 9.42 (s, 1H), 14.15 (s, 1H).

MS 295 (M+H), C₁₆H₁₄N₄O₂.

### Example 52

### 3-(2,6-Diamino-pyrimidin-4-yl)-biphenyl-4,4′-diol

The starting material 6-(4,4′-Bis-benzyloxy-biphenyl-3-yl)-pyrimidine-2,4-diamine for the title compound was obtained as white solids (yield 31%) according to General Procedure H by using 2-chloro-4,5-diaminopyrimidine.

¹H NMR(d₆-DMSO) δ4.62 (s, 2H), 4.86 (s, 2H), 5.10 (s, 2H), 5.16 (s, 2H), 6.48 (s, 1H), 7.01 (d, 2H), 7.06 (d, 1H), 7.26 (s, 1H), 7.31 (m, 2H), 7.34-7.39 (m, 3H), 7.36 (d, 2H), 7.41 (s, 1H), 7.42 (d, 1H), 7.46 (d, 1H), 7.52 (d, 2H), 8.02 (d, 1H).

MS 475 (M+H), C₃₀H₂₆N₄O₂.

The title compound was obtained as yellow solids (yield 86%) according to General Procedure I by using 6-(4,4′-Bis-benzyloxy-biphenyl-3-yl)-pyrimidine-2,4-diamine.

¹H NMR(d₆-DMSO) δ6.37 (s, 1H), 6.48 (s, 2H), 6.57 (s, 2H), 6.83 (d, 2H), 6.85 (d, 1H), 7.41 (d, 2H), 7.47 (d, 2H), 7.74 (s, 1H), 9.44 (s, 1H), 14.77 (s, 1H).

MS 295 (M+H), C₁₆H₁₄N₄O₂.

### Example 53

### 3-(2-Amino-pyrimidin-4-yl)-3′-fluoro-biphenyl-4,4′-diol

The starting material 4-(4,4′-Bis-benzyloxy-3′-fluoro-biphenyl-3-yl)-pyrimidin-2-ylamine for the title compound was obtained as white solids according to General Procedure H by using 2-chloro-4,5-diaminopyrimidine.

The title compound was obtained as yellow solids according to General Procedure I by using 4-(4,4′-Bis-benzyloxy-3′-fluoro-biphenyl-3-yl)-pyrimidin-2-ylamine.

¹H NMR (d₆-DMSO) δ6.95 (dd, 1H) 7.16 (s, 2H) 7.35 (d, 1H) 7.49 (dd, 1H) 7.55 (d, 1H) 7.62 (d, 1H) 8.01 (s, 1H) 8.10 (s, 1H) 8.39 (dd, 1H) 9.95 (s, 1H) 13.89 (s, 1H).

MS (ESI+) 321 (M+Na) C₁₆H₁₃N₃O₂F.

### Example 54

### 3-(2-Amino-6-chloro-pyrimidin-4-yl)-biphenyl-4,4′-diol

A solution of 4,4′-biphenol (1 mmol) in dry DMF (5 mL) was added dropwise to a suspension of NaH (2.5 mmol, 60% in mineral oil) in DMF (5 mL) under nitrogen by maintaining the temperature below 70° C. After being stirred for 2 h at room temperature, chloromethyl methyl ether was added dropwisely by maintaining the temperature below 50° C. under nitrogen. The mixture was stirred 3 h at room temperature and quenched with cold water (10 mL). The aqueous layer was further extracted by ether (3×10 mL). The combined organic layer was washed by 1 N NaOH, water and saturated NaCl, then dried over Na₂SO₄ and evaporated to get white solids 4,4′-Dimethoxymethyletherbiphenol (98%).

¹H NMR(CDCl3) δ3.48 (s, 6H), 5.20 (s, 4H), 7.09 (d, 4H), 7.47 (d, 4H).

MS 275 (M+H), C₁₆H₁₈O₄.

A solution of n-BuLi (7.3 mmol, 2 M in cyclohexane) was added to a solution of TMEDA (7.3 mmol) in anhydrous ether (10 mL) at −40° C. under nitrogen and stirred for 30 min at same temperature. The solution was cooled to −78° C. and a solution of 4,4′-dimethoxy-methyletherbiphenol (7.3 mmol) in 20 mL ether was added slowly by maintaining the temperature below −70° C. After stirring at −78° C. for 2 h, the mixture was allowed to come at room temperature slowly over 5 h. The solution was again cooled to −78° C. and treated with trimethylborate (21.9 mmol). The mixture was allowed to come at room temperature and stirred overnight. The mixture was cooled to 0° C. and treated with cold water (10 mL) and further stirred for 30 min. Finally, the organic layer was separated and the aqueous layer was extracted by ether (3×30 mL). The combined organic phase were washed by water and saturated NaCl solution, then dried over Na₂SO₄ and evaporated. The residue was purified by column chromatography over silica gel (3×20 cm) eluted by 1% MeOH in CH₂Cl₂ to get the title compound as colorless liquid of 4,4′-Dimethoxymethylether-3-biphenylboronic acid in 52% yield.

¹H NMR(CDCl₃) δ3.38 (s, 3H), 3.40 (s, 3H), 5.21 (s, 2H), 5.26 (s, 2H), 7.04 (d, 2H), 7.12 (d, 1H), 7.52 (d, 2H), 7.57 (dd, 1H), 7.75 (d, 1H), 7.88 (s, 2H).

MS 318 (M+H), C₁₆H₁₉BO₆.

The starting material 4-(4,4′-Bis-methoxymethoxy-biphenyl-3-yl)-6-chloro-pyrimidin-2-ylamine for the title compound was obtained as white solids according to General Procedure H by using 2-amino-4,6-dichloropyrimidine.

The title compound was obtained as yellow solids according to General Procedure J by using 4-(4,4′-Bis-methoxymethoxy-biphenyl-3-yl)-6-chloro-pyrimidin-2-ylamine.

¹H NMR (d₆-DMSO) δ6.83 (d, 2H) 6.98 (d, 1H) 7.55-7.65 (m, 6H) 8.13 (s, 1H) 9.48 (s, 1H) 13.38 (s, 1H).

MS (ESI+) 314 (M+H) C₁₆H₁₂N₃O₂Cl.

### Example 55

### 3-[2-Amino-6-(2-hydroxy-ethylamino)-pyrimidin-4-yl]-biphenyl-4,4′-diol

The title compound is prepared according to General Procedure E from 2-aminoethanol and 4-(4,4′-Bis-methoxymethoxy-biphenyl-3-yl)-6-chloro-pyrimidin-2-ylamine followed by deprotection step according to General Procedure J.

¹H NMR (d₆-DMSO) δ3.52 (m, 2H), 4.77 (s, 1H), 5.15 (m, 2H), 6.51 (s, 1H), 6.87 (d, 1H), 7.08 (d, 2H), 7.35 (d, 1H), 7.39-7.42 (m, 2H), 7.46-7.48 (m, 2H), 7.50 (d, 1H), 7.54 (s, 1H), 7.55 (s, 1H), 14.85 (s 1H).

MS (ESI+) 339 (M+H) C₁₈H₁₈N₄O₃.

### Example 56

### 3-[2-Amino-6-(2,3-dihydroxy-propylamino)-pyrimidin-4-yl]-biphenyl-4,4′-diol

The title compound is prepared according to General Procedure K from 2,3-dihydroxylethylamine and 4-(4,4′-Bis-methoxymethoxy-biphenyl-3-yl)-6-chloro-pyrimidin-2-ylamine followed by deprotection step according to General Procedure J.

¹H NMR (d₆-DMSO) δ3.23 (d, 2H) 3.49 (d, 2H) 3.61 (s, 1H) 4.66 (s, 1H) 4.91 (s, 1H) 6.52-6.62 (m, 3H) 6.82-6.85 (m, 3H) 7.06 (s, 1H) 7.43-7.47 (m, 3H) 7.76 (s, 1H) 9.53 (s, 1H) 14.76 (s, 1H).

MS (ESI+) 369 (M+H) C₁₉H₂₀N₄O₄.

### Example 57

### 3-(2-Amino-pyrimidin-4-yl)-biphenyl-4,4′-diol

One equivalent of 5′-bromo-2′-methoxy-acetophenone and 15 eq. of N,N-dimethylformamide diethyl acetal are combined and heated to 120° C. overnight. The reaction mixture is concentrated in vacuo and dissolved in butanol (0.1 M) with 3 equivalents of sodium ethoxide followed by 1.1 equivalents of guanidine hydrochloride. This mixture is heated to 120° C. overnight. The reaction mixture was extracted with dichloromethane, washed with water and then dried over sodium sulfate. The resulting 4-(2-methoxy-5-bromo-phenyl)-pyrimidin-2-ylamine was obtained after purification on a silica gel column. The Suzuki reaction is then performed followed General Procedure G with 4-methoxyphenylboronic acid to give 4-(4,4′-Dimethoxy-biphenyl-3-yl)-pyrimidin-2-ylamine in 90% yield.

The title compound was obtained as yellow solids according to General Procedure L by using 4-(4,4′-Dimethoxy-biphenyl-3-yl)-pyrimidin-2-ylamine.

¹H-NMR (d₆-DMSO): δ6.85 (d, 2H), 6.92 (d, 1H), 7.19 (s, 2H), 7.42 (d, 1H), 7.48 (d, 2H), 7.57 (d, 1H), 8.09 (d, 1H), 8.32 (d, 1H), 9.43 (d, 1H), 13.70 (s, 1H).

MS 280 (M+H), C₁₆H₁₃N₃O₂.

### Example 58

### 3-(4,6-Diamino-[1,3,5]triazin-2-yl)-biphenyl-4,4′-diol

One equivalent of 5-bromo-2-methoxy-benznitrile (1 mmol) was reacted with 4-methoxyphenylboronic acid (1.05 mmol), following General Procedure G, to give 4,4′-dimethoxy-3-cyanobiphenol in 90% yield. Five equivalents of dicyandiamide is dissolved in methyl cellosolve with five equivalents of potassium hydroxide. To this solution, one equivalent of 4,4′-dimethoxy-3-cyanobiphenol is added to a final concentration of 0.5 M. This solution is heated to 100° C. with stirring overnight or until the reaction is judged complete by thin layer chromatography. The precipitated product is filtered and dried in vacuo. (Simons, J K and Saxton, M R Organic Synthesis 1963, 78.) The starting material 6-(4,4′-Dimethoxy-biphenyl-3-yl)-[1,3,5]triazine-2,4-diamine for the title compound is obtained in 78% yield.

The title compound was obtained as yellow solids according to General Procedure L by using 6-(4,4′-Dimethoxy-biphenyl-3-yl)-[1,3,5]triazine-2,4-diamine.

¹H NMR (CDCl₃) δ7.06 (d, 1H), 7.23 (bs, 1H), 7.31 (d, 1H), 7.43 (d, 1H), 7.57 (d, 1H), 7.63 (s, 1H), 7.83 (d, 1H), 8.41 (s, 1H), 8.46 (d, 1H), 9.57 (s, 1H).

MS 282 (M+H), C₁₅H₁₁N₃O₃.

### Example 59

### 5-[3-(2-Amino-pyrimidin-4-yl)-4-hydroxy-phenyl]-furan-2-carbaldehyde

One equivalent of 5′-bromo-2′-benzyloxy-acetophenone and 15 eq. of N,N-dimethyl-formamide diethyl acetal are combined and heated to 120° C. overnight. The reaction mixture is concentrated in vacuo and dissolved in butanol (0.1 M) with 3 equivalents of sodium ethoxide followed by 1.1 equivalents of guanidine hydrochloride. This mixture is heated to 120° C. overnight. The reaction mixture was extracted with dichloromethane, washed with water, and then dried over sodium sulfate. The resulting 4-(2-benzyloxy-5-bromo-phenyl)-pyrimidin-2-ylamine was obtained after purification on a silica gel column. The Suzuki reaction is then performed following General Procedure G with 2-furfurylboronic acid to give 5-[3-(2-Amino-pyrimidin-4-yl)-4-benzyloxy-phenyl]-furan-2-carbaldehyde in 60% yield.

The title compound was obtained as yellow solids according to General Procedure I by using 5-[3-(2-Amino-pyrimidin-4-yl)-4-benzyloxy-phenyl]-furan-2-carbaldehyde.

¹H NMR (d₆-DMSO) δ6.81 (d, 2H), 6.88 (d, 1H), 7.01 (s, 2H), 7.26 (s, 2H), 7.39 (d, 2H), 7.56 (d, 1H), 8.38 (d, 1H), 9.44 (s, 1H), 13.86 (s, 1H).

MS (ESI+) 296 (M+H) C₁₅H₁₃N₅O₂.

### Example 60

### 2-(2-Amino-pyrimidin-4-yl)-4-(1H-indol-5-yl)-phenol

The 4-(2-benzyloxy-5-bromo-phenyl)-pyrimidin-2-ylamine obtained in Example 59 was used in the Suzuki reaction, following General Procedure G, with 5-indolylboronic acid to give 4-[2-Benzyloxy-5-(1H-indol-5-yl)-phenyl]-pyrimidin-2-ylamine 85% yield.

The benzyl group is then removed according to General Procedure I to give the title compound in 90% yield.

¹H NMR (d₆-DMSO) δ6.46 (s, 1H), 6.98 (d, 1H), 7.16, (s, 2H), 7.36 (s, 1H), 7.41-7.46 (m, 3H), 7.66 (d, 1H), 7.84 (s, 1H), 8.14 (s, 1H), 8.38 (d, 2H), 11.10 (s, 1H), 13.76 (s, 1H).

MS (ESI+) 303 (M+H) C₁₈H₁₄N₄O.

### Example 61

### 3-(2-Amino-thiazol-4-yl)-biphenyl-4,4′-diol

The synthesis of the title compound is achieved by dissolving 1 equivalent of ketone in glacial acetic acid to a final concentration of 0.2 M. To this solution, 1 equivalent of bromine dissolved in glacial acetic acid (1 M) is carefully added. The resulting solution is stirred at room temperature for 1 hour. The solution is partitioned twice between dichloromethane and water. The combined organic layers are washed with water, dried over sodium sulfate, filtered and concentrated. The resulting compound is redissolved in dioxane (0.2 M) and 1 equivalent of thiourea is added. This mixture is stirred at room temperature for 18 hours. The reaction mixture is partitioned between dichloromethane and saturated sodium bicarbonate solution, dried over sodium sulfate, filtered and concentrated. The final product is purified by silica gel chromatography. The methyl groups are then cleaved according to General Procedure L.

¹H NMR (d₆-DMSO) δ6.81 (d, 2H), 6.86 (d, 1H), 7.24 (s, 1H), 7.33-7.35 (dd, 1H), 7.44 (s, 1H), 7.45 (m, 3H), 7.88 (d, 1H), 9.41 (s, 1H), 11.89 (s, 1H).

MS (APCI) 307 (M+Na) C₁₅H₁₂N₂O₂S.

### Example 62

### 2-(2-Amino-pyrimidin-4-yl)-4-pyridin-4-yl-phenol

The title compound was obtained as a yellow solid from 4-(2-benzyloxy-5-bromo-phenyl)-pyrimidin-2-ylamine (see Example 59) via the Suzuki reaction with 4-pyridylboronic acid as described in General Procedure G, followed by deprotection using TFA with 5-10% thioanisole (Reaction Scheme 5).

¹H NMR (DMSOd-₆) δ7.05 (d, J=8.6 Hz, 1 H), 7.22 (br s, 2 H), 7.56 (d, J=5.2 Hz, 1 H), 7.80 (d, J=5.0 Hz, 2 H), 7.84 (d, J=8.6 Hz, 1 H), 8.37 (s, 1 H), 8.43 (d, J=5.2 Hz, 1 H), 8.60 (d, J=5.0 Hz, 2 H), 14.2 (br s, 1 H).

ESI: m/z 265.40 (M+H⁺, C₁₅H₁₂N₄O requires 265.11).

### Example 63

### 2-(2-Amino-pyrimidin-4-yl)-4-pyridin-3-yl-phenol

The title compound was obtained as a yellow solid from 4-(2-benzyloxy-5-bromo-phenyl)-pyrimidin-2-ylamine (see Example 59) via the Suzuki reaction with 3-pyridylboronic acid as described in General Procedure G, followed by deprotection using TFA with 5-10% thioanisole (Reaction Scheme 5).

¹H NMR (DMSO-d₆) δ7.04 (d, J=8.5 Hz, 1 H), 7.20 (br s, 2 H), 7.46 (dd, J=4.5, 7.5 Hz, 1 H), 7.54 (d, J=5.3 Hz, 1 H), 7.75 (d, J=8.5 Hz, 1 H), 8.13 (d, J=7.5 Hz, 1 H), 8.28 (s, 1 H), 8.41 (d, J=5.3 Hz, 1 H), 8.53 (d, J=4.5 Hz, 1 H), 8.96 (s, 1 H), 14.1 (br s, 1 H).

ESI: m/z 265.41 (M+H⁺, C₁₅H₁₂N₄O requires 265.11).

### Example 64

### 4-(6-Amino-pyridin-2-yl)-2-(2-amino-pyrimidin-4-yl)-phenol

A mixture of 4-(2-benzyloxy-5-bromo-phenyl)-pyrimidin-2-ylamine (see Example 59) (17.65 mmol), bis-(pinacolato) diboron (19.42 mmol), potassium acetate (52.95 mmol) and Pd(dppf)Cl₂ in anhydrous DMF (125 mL) was heated overnight at 80° C. under nitrogen. The cooled solution was partitioned between CH₂Cl₂ and water. The combined organic phase were washed subsequently with water and brine then dried over Na₂SO₄ and concentrated. The crude product was purified by flash chromatography to get the desired boronate.

The title compound was obtained as a yellow solid from this boronate via the Suzuki reaction with 2-amino-6-bromopyridine as described in General Procedure G, followed by deprotection using TFA with 5-10% thioanisole (Reaction Scheme 6).

¹H NMR (DMSO-d₆) δ5.96 (s, 2 H), 6.37 (d, J=7.7 Hz, 1 H), 6.96 (d, J=8.5 Hz, 1 H), 7.12 (d, J=7.7 Hz, 1 H), 7.19 (br s, 2 H), 7.34 (d, J=5.2 Hz, 1 H), 7.43 (t, J=7.7 Hz, 1 H), 8.00 (d, J=8.5 Hz, 1 H), 8.41 (d, J=5.2 Hz, 1 H), 8.49 (s, 1 H), 1.40 (br s, 1 H).

ESI: m/z 280.50 (M+H⁺, C₁₅H₁₃N₅O requires 280.12).

### Example 65

### 4-(6-Amino-pyridin-3-yl)-2-(2-amino-pyrimidin-4-yl)-phenol

The title compound was obtained as a yellow solid from the boronate (see Example 64) via the Suzuki reaction with 2-amino-5-bromopyridine as described in General Procedure G, followed by deprotection using TFA with 5-10% thioanisole (Reaction Scheme 6).

¹H NMR (DMSO-d₆) δ5.97 (s, 2 H), 6.51 (d, J=8.5 Hz, 1 H), 6.95 (d, J=8.5 Hz, 1 H), 7.16 (br s, 2 H), 7.46 (d, J=5.4 Hz, 1 H), 7.56 (d, J=8.5 Hz, 1 H), 7.74 (d, J=8.5 Hz, 1 H), 8.05 (s, 1 H), 8.27 (s, 1H), 8.38 (d, J=5.4 Hz, 1 H), 13.8 (br s, 1 H).

ESI: m/z 280.49 (M+H⁺, C₁₅H₁₃N₅O requires 280.12).

### Example 66

### 2-(2-Amino-pyrimidin-4-yl)-4-(2-amino-pyrimidin-5-yl)-phenol

The title compound was obtained as a yellow solid from the boronate (see Example 64) via the Suzuki reaction with 2-amino-5-bromopyrimidine as described in General Procedure G, followed by the deprotection using TFA with 5-10% thioanisole (Reaction Scheme 6).

¹H NMR (DMSO-d₆) δ6.71 (br s, 2 H), 6.97 (d, J=8.5 Hz, 1 H), 7.19 (br s, 2 H), 7.51 (d, J=5.2 Hz, 1 H), 7.61 (d, J=8.5 Hz, 1 H), 8.13 (s, 1 H), 8.40 (d, J=5.2 Hz, 1 H), 8.61 (s, 1 H), 13.9 (s, 1 H).

ESI: m/z 281.60 (M+H⁺, C₁₄H₁₂N₆O requires 281.12).

### Example 67

### 4-(2-Amino-pyridin-4-yl)-2-(2-amino-pyrimidin-4-yl)-phenol

The title compound was obtained as a yellow solid from the boronate (see Example 64) via the Suzuki reaction with 2-amino-4-bromopyridine as described in General Procedure G, followed by deprotection using TFA with 5-10% thioanisole (Reaction Scheme 6).

¹H NMR (DMSO-d₆) δ5.90 (s, 2 H), 6.73 (s, 1 H), 6.87 (d, J=5.1 Hz, 1 H), 7.01 (d, J=8.5 Hz, 1 H), 7.21 (br s, 2 H), 7.43 (d, J=5.2 Hz, 1 H), 7.64 (d, J=8.5 Hz, 1 H), 7.94 (d, J=5.1 Hz, 1 H), 8.16 (s, 1 H), 8.40 (d, J=5.2 Hz, 1 H), 14.0 (s, 1 H).

ESI: m/z 280.41 (M+H⁺, C₁₅H₁₃N₅O requires 280.12).

### Example 68

### 2,4-Bis-(2-amino-pyrimidin-4-yl)-phenol

A solution of 6-hydroxy-2,4-diacetophenone in anhydrous THF was treated with MOM-Cl/NaH and further stirred for 1 h at room temperature under nitrogen. The resulting mixture was then worked up upon extraction and dried. A solution of the MOM protected acetophenone (Reaction Scheme 7) in N,N′-dimethylformamide diethylacetal (15 eq) was heated to reflux for 24 h and evaporated under vacuum to get a yellow oil. This oil was dissolved in ethanol and treated with sodium ethoxide and guanidine hydrochloride then heated to reflux overnight. The cooled solution was diluted with water, extracted by CH₂Cl₂ and the combined organic phase was concentrated to get red viscous oil. This crude product was further purified by flash chromatography eluting with 0-5% MeOH in CH₂Cl₂ to get pale yellow-orange crystals. The MOM group was then removed using TFA as described in the General Procedure J.

¹H NMR (DMSO-d₆) δ6.60 (s, 2 H), 7.00 (s, 1 H), 7.22 (br s, 3 H), 7.40 (s, 1 H), 8.01 (s, 1 H), 8.26 (s, 1 H), 8.42 (s, 1 H), 8.60 (s, 1 H), 14.30 (br s, 1 H).

ESI: m/z 281.51 (M+H⁺, C₁₄H₁₂N₆O requires 281.12).

### Example 69

### 2-(2-Amino-pyrimidin-4-yl)-4-(1H-pyrrol-2-yl)-phenol

The title compound was obtained as a yellow solid from 4-(2-benzyloxy-5-bromo-phenyl)-pyrimidin-2-ylamine (see Example 59) via the Suzuki reaction with N-Boc protected 2-pyrrolylboronic acid as described in General Procedure G, followed by deprotection using TFA with 5-10% thioanisole (Reaction Scheme 5).

¹H NMR (acetone-d₆) δ6.15 (s, 1 H), 6.48 (s, 1 H), 6.63 (br s, 2 H), 6.84 (s, 1 H), 6.92 (d, J=8.5 Hz, 1 H), 7.41 (d, J=5.4 Hz, 1 H), 7.65 (d, J=8.5 Hz, 1 H), 8.18 (s, 1 H), 8.43 (d, J=5.4 Hz, 1 H), 10.52 (br s, 1 H), 13.6 (s, 1 H).

ESI: m/z 253.49 (M+H⁺, C₁₄H₁₂N₄O requires 253.11).

### Example 70

### 5-[3-(2-Amino-pyrimidin-4-yl)-4-hydroxy-phenyl]-1H-pyrimidine-2,4-dione

The title compound was obtained as a yellow solid from 4-(2-benzyloxy-5-bromo-phenyl)-pyrimidin-2-ylamine (see Example 59) via the Suzuki reaction with 2,4-dibenzyloxy-5-pyrimidylboronic acid as described in General Procedure G, followed by deprotection using hydrogenation with paaladium on charcol and TFA with 5-10% thioanisole (Reaction Scheme 5).

¹H NMR (DMSO-d₆) δ6.88 (d, J=8.5 Hz, 1 H), 7.15 (br s, 2 H), 7.27 (d, J=5.2 Hz, 1 H), 7.56 (d, J=8.5 Hz, 1 H), 7.70 (s, 1 H), 8.04 (s, 1 H), 8.37 (d, J=5.2 Hz, 1 H), 11.00 (bump), 13.80 (bump).

ESI: m/z 298.47 (M+H⁺, C₁₄H₁₁N₅O₃ requires 298.09).

### Example 71

### 2-(2-Aminopyridin-6-yl)-4-(2-aminopyrimidin-4-yl)-phenol

Following the General Procedure M, 4-[4-(2-methoxy-ethoxymethoxy)-3-(4,4,5,5-tetramethyl-[1,3,2]-dioxaborolan-2-yl-phenyl]-pyrimidin-2-ylamine was reacted with 2-amino-6-bromopyridine to give 4-[3-(2-Aminopyridin-6-yl)-4-(2-Methoxy-ethoxymethoxy)-phenyl]-pyrimidin-2-ylamine (54% yield), which was then converted to the title compound after treatment with TFA and triethylsilane in CH₂Cl₂ at room temperature. Yie1d: 47%.

¹H NMR(d₆-DMSO) 6.50 (d, 1H), 6.57 (s, 2H), 6.63 (s, 2H) 6.93 (d, 1H), 7.20 (d, 1H), 7.34 (d, 1H), 7.61 (t, 1H), 7.98 (d, 1H), 8.25 (d, 1H), 8.56 (s, 1H), 15.10 (s, 1H).

MS 280 (M+H), C₁₅H₁₃N₅O.

### Example 72

### 2-(2-Amino-4-methyl-pyrimidin-6-yl)-4-(2-aminopyrimidin-4-yl)-phenol

Following the General Procedure M, 4-[4-(2-methoxy-ethoxymethoxy)-3-(4,4,5,5-tetramethyl-[1,3,2]-dioxaborolan-2-yl-phenyl]-pyrimidin-2-ylamine was reacted with 2-amino-6-chloro-4-methylpyrimidine to give 4-[3-(2-amino-4-methyl-pyrimidin-6-yl)-4-(2-methoxy-ethoxymethoxy)-phenyl]-pyrimidin-2-ylamine, which was then converted to the title compound after treatment with TFA and triethylsilane in CH₂Cl₂ at room temperature.

¹H NMR(d₆-DMSO) 2.35 (s, 3H), 6.67 (s, 2H), 6.99 (d, 1H), 7.20 (s, 2H), 7.24 (d, 1H), 7.33 (s, 1H), 8.09 (d, 1H), 8.30 (d, 1H), 8.58 (s, 1H), 14.54 (s, 1H).

MS 295 (M+H), C₁₅H₁₄N₆O.

### Example 73

### 2-(2-Amino-4-chloro-pyrimidin-6-yl)-4-(2-aminopyrimidin-4-yl)-phenol

Following the General Procedure M, 4-[4-(2-methoxy-ethoxymethoxy)-3-(4,4,5,5-tetramethyl-[1,3,2]-dioxaborolan-2-yl-phenyl]-pyrimidin-2-ylamine was reacted with 2-amino-4,6-dichloropyrimidine to give 4-[3-(2-amino-4-chloro-pyrimidin-6-yl)-4-(2-methoxy-ethoxymethoxy)-phenyl]-pyrimidin-2-ylamine, which was then converted to the title compound after treatment with TFA and triethylsilane in CH₂Cl₂ at room temperature.

¹H NMR(d₆-DMSO) 6.58 (s, 2H), 7.03 (d, 1H), 7.30 (s, 1H), 7.57 (s, 1H), 7.69 (s, 2H), 8.16 (d, 1H), 8.24 (d, 1H), 8.59 (s, 1H), 14.05 (s, 1H).

MS 315 (M+H), C₁₄H₁₁N₆OCl.

### Example 74

### 2-(2,4-Diamino-pyrimidin-6-yl)-4-(2-aminopyrimidin-4-yl)-phenol

Following the General Procedure M, 4-[4-(2-methoxy-ethoxymethoxy)-3-(4,4,5,5-tetramethyl-[1,3,2]-dioxaborolan-2-yl-phenyl]-pyrimidin-2-ylamine was reacted with 2,4-diamino-6-chloropyrimidine to give 4-[3-(2,4-diamino-pyrimidin-6-yl)-4-(2-methoxy-ethoxymethoxy)-phenyl]-pyrimidin-2-ylamine, which was then converted to the title compound after treatment with TFA and triethylsilane in CH₂C₁₂ at room temperature.

¹H NMR (d₆-DMSO) 6.38 (s, 1H), 6.60 (d, 4H), 6.71 (s, 2H), 6.86 (d, 1H), 7.06 (d, 1H), 7.95 (d, 1H), 8.25 (d, 1H), 8.36 (s, 1H), 15.54 (s, 1H).

MS 296 (M+H), C₁₄H₁₃N₇O.

### Example 75

### 2-(2-Amino-4-methoxy-pyrimidin-6-yl)-4-(2-aminopyrimidin-4-yl)-phenol

Following the General Procedure M, 4-[4-(2-methoxy-ethoxymethoxy)-3-(4,4,5,5-tetramethyl-[1,3,2]-dioxaborolan-2-yl-phenyl]-pyrimidin-2-ylamine was reacted with 2-amino-6-chloro-4-methoxypyrimidine to give 4-[3-(2-amino-4-methoxy-pyrimidin-6-yl)-4-(2-methoxy-ethoxymethoxy)-phenyl]-pyrimidin-2-ylamine, which was then converted to the title compound after treatment with TFA and triethylsilane in CH₂Cl₂ at room temperature.

¹H NMR (d₆-DMSO) 3.90 (s, 3H), 6.59 (s, 3H), 6.83 (d, 1H), 6.97 (d, 1H), 7.26 (d, 2H), 8.08 (d, 1H), 8.25 (d, 1H), 8.56 (s, 1H), 14.66 (s, 1H).

MS 211 (M+H), C₁₅H₁₄N₆O₂.

### Example 76

### 2-[2-Amino-4-(piperazin-1-yl)-pyrimidin-6-yl]-4-(2-aminopyrimidin-4-yl)]-phenol

A solution of 4-[2-(2-Amino-4-chloro-pyrimidin-6-yl)-4-(2-aminopyrimidin-4-yl)-phenol (see Example 73) (120 mg, 0.38 mmol), t-butyl-1-piperazinecarboxylate (78.1 mg, 0.41 mmol) and DIPEA (0.2 ml, 1.14 mmol) in DMF was heated overnight at 110° C. The solution was cooled, evaporated under vacuum to get a residue which was purified by flash chromatography eluting with 2-5% MeOH in CH₂Cl₂ to get 2-[2-Amino-4-(t-butoxycarbonylpiperazin-1-yl)-pyrimidin-6-yl]-4-(2-aminopyrimidin-4-yl)-phenol as yellow solids (60 mg, 34%).

¹H NMR(d₆-DMSO) δ1.41 (s, 12H), 3.48 (t, 4H), 3.72 (t, 4H), 6.59 (s, 2H), 6.78 (s, 1H), 6.94 (d, 1H), 7.22 (s, 2H), 7.27 (d, 1H), 8.06 (d, 1H), 8.26 (t, 1H), 8.52 (s, 1H), 15.44 (s, 1H).

MS 465 (M+H), C₂₃H₂₃N₈O₃.

The title compound was prepared from 2-[2-Amino-4-(t-butoxycarbonylpiperazin-1-yl)-pyrimidin-6-yl]-4-(2-aminopyrimidin-4-yl)-phenol, following the General procedure J.

¹H NMR(d₆-DMSO) δ2.75 (t, 4H), 3.63 (t, 4H), 6.58 (s, 2H), 6.66 (s, 1H), 6.70 (s, 1H), 6.91 (d, 1H), 7.28 (d, 1H), 8.05 (d, 1H), 8.25 (d, 1H), 8.51 (s, 1H), 15.57 (s, 1H).

MS 365 (M+H), C₁₈H₂₀N₈O.

### Example 77

### 3-[2-Amino-6-(5-hydroxy-pentylamino)-pyrimidin-4-yl]-biphenyl-4,4′-diol

The title compound was obtained upon treatment of 3-(2-Amino-6-chloro-pyrimidin-4-yl)-biphenyl-4,4′-diol (see Reaction Scheme 9 and Example 54) with 5-hydroxy-pentylamine following the General Procedure K.

¹H NMR (d₆-DMSO) δ1.38 (m, 2H) 1.47 (m, 2H) 1.52 (m, 2H) 3.31 (m, 2H) 4.43 (s, 1H) 6.44 (s, 1H) 6.60 (d, 2H) 6.84-6.88 (m, 3H) 7.15 (s, 1H) 7.45-7.49 (m, 3H) 7.76 (s, 1H) 9.49 (s, 1H) 14.80 (s, 1H)

MS (ESI+) 381 (M+H) C₂₁H₂₄N₄O₃

### Example 78

### 3-(2-Amino-6-piperazin-1-yl-pyrimidin-4-yl)-biphenyl-4,4′-diol

The title compound was obtained upon treatment of 3-(2-Amino-6-chloro-pyrimidin-4-yl)-biphenyl-4,4′-diol (see Reaction Scheme 9 and Example 54) with t-butyl-1-piperazinecarboxylate based upon the General Procedure K and followed by the deprotection as described in General Procedure J.

¹H NMR (d₆-DMSO) δ2.82 (m,4H) 3.67 (m, 4H) 6.01 (s, 2H) 6.74 (s, 1H) 6.82 (d, 2H) 6.85 (d, 1H) 7.47 (dd, 1H) 7.50 (d, 2H) 8.06 (s, 1H) 9.43 (s, 1H)

MS (ESI+) 364 (M+H) C₂₀H₂₁N₅O₂

### Example 79

### 3-[2-Amino-6-(2R-hydroxymethyl-pyrrolidin-1-yl)-pyrimidin-4-yl]-biphenyl-4,4′-diol

The title compound was obtained upon treatment of 3-(2-Amino-6-chloro-pyrimidin-4-yl)-biphenyl-4,4′-diol (see Reaction Scheme 9 and Example 54) with 2R-hydroxymethyl-pyrrolidinol following the General Procedure K.

¹H NMR (d₆-DMSO) δ1.90 (m,2H) 2.00 (m, 2H) 3.33 (m, 1H) 3.40 (m, 2H) 3.57 (m, 2H) 6.79 (d, 1H) 6.83 (d, 2H) 6.85 (d, 1H) 7.37 (d, 1H) 7.45-7.48 (m, 3H) 7.95 (s, 2H) 9.44 (s, 1H) 14.83 (s, 1H)

MS (ESI+) 379 (M+H) C₂₁H₂₂N₄O₃

### Example 80

### 3-[2-Amino-6-(2S-hydroxymethyl-pyrrolidin-1-yl)-pyrimidin-4-yl]-biphenyl-4.4′-diol

The title compound was obtained upon treatment of 3-(2-Amino-6-chloro-pyrimidin-4-yl)-biphenyl-4,4′-diol (see Reaction Scheme 9 and Example 54) with 2S-hydroxymethyl-pyrrolidinol following the General Procedure K.

¹H NMR (d₆-DMSO) δ1.90 (m,2H) 2.00 (bs, 2H) 3.17 (s, 1H) 3.39 (bs, 2H) 3.56 (bs, 2H) 4.11 (s, 1H) 6.55 (bs, 1H) 6.79 (d, 1H) 6.83 (d, 2H) 6.85 (d, 1H) 7.36 (d, 1H) 7.45-7.51 (m, 3H) 7.96 (bs, 1H) 9.44 (s, 1H) 14.81 (s, 1H)

MS (ESI+) 379 (M+H) C₂₁H₂₂N₄O₃

### Example 81

### 3-(2-Amino-6-morpholin-4-yl-pyrimidin-4-yl)-biphenyl-4,4′-diol

The title compound was obtained upon treatment of 3-(2-Amino-6-chloro-pyrimidin-4-yl)-biphenyl-4,4′-diol (see Reaction Scheme 9 and Example 54) with morpholine following the General Procedure K.

¹H NMR (d₆-DMSO) δ3.60-3.70 (m, 8H) 6.76-6.79 (m, 2H) 6.82 (d, 2H) 6.86 (d, 1H) 7.36 (d, 1H) 7.48 (dd, 1H) 7.50 (d, 2H) 8.07 (s, 1H) 9.43 (s, 1H) 14.78 (s, 1H)

MS (ESI+) 365 (M+H) C₂₀H₂₀N₄O₃

### Example 82

### 3-[2-Amino-6-(3-hydroxymethyl-piperidin-1-yl)-pyrimidin-4-yl]-biphenyl-4,4′-diol

The title compound was obtained upon treatment of 3-(2-Amino-6-chloro-pyrimidin-4-yl)-biphenyl-4,4′-diol (see Reaction Scheme 9 and Example 54) with 3-hydroxymethyl-piperidine following the General Procedure K.

¹H NMR (d₆-DMSO) δ3.17 (m, 4H) 3.34 (m, 4H) 4.12 (m, 1H) 4.59 (m, 2H) 6.55 (bs, 1H) 6.79 (d, 1H) 6.82-6.85 (m, 4H) 7.45-7.50 (m, 3H) 8.02 (s, 1H) 9.42 (s, 1H) 14.85 (s, 1H)

MS (ESI+) 393 (M+H) C₂₂H₂₄N₄O₃

### Example 83

### 3-[2-Amino-6-(2-hydroxymethyl-phenyl)-pyrimidin-4-yl]-biphenyl-4,4′-diol

The title compound was obtained from 1) the Suzuki Reaction of MEM protected 3-(2-amino-6-chloro-pyrimidin-4-yl)-biphenyl-4,4′-diol (see Reaction Scheme 10 and Example 54) with 2-hydroxymethylphenylboronic acid following the General Procedure G and 2) the deprotection process following the General Procedure J.

¹H NMR (d₆-DMSO) δ4.61 (s, 2H) 5.25 (s, 1H) 6.81 (d, 2H) 6.97 (d, 1H) 7.30 (bs, 2H) 7.41 (t, 1H) 7.49 (t, 1H) 7.53 (d, 2H) 7.58-7.62 (m, 3H) 7.68 (s, 1H) 8.18 (d, 1H) 9.15 (s, 1H) 13.91 (s, 1H)

MS (ESI+) 386 (M+H) C₂₃H₁₉N₃O₃

### Example 84

### 3-[2-Amino-6-(3-hydroxymethyl-phenyl)-pyrimidin-4-yl]-biphenyl-4,4′-diol

The title compound was obtained from 1) the Suzuki Reaction of MEM protected 3-(2-Amino-6-chloro-pyrimidin-4-yl)-biphenyl-4,4′-diol (see Reaction Scheme 10 and Example 54) with 3-hydroxymethylphenylboronic acid following the General Procedure G and 2) the deprotection process following the General Procedure J.

¹H NMR (d₆-DMSO) δ4.61 (s, 2H) 5.32 (s, 1H) 6.85 (d, 2H) 6.97 (d, 1H) 7.26 (bs, 2H) 7.50 (d, 2H) 7.59 (d, 1H) 7.93 (s, 1H) 8.17 (t, 1H) 8.23 (s, 1H) 8.30 (d, 1H) 9.52 (s, 1H) 14.05 (s, 1H)

MS (ESI+) 386 (M+H) C₂₃H₁₉N₃O₃

### Example 85

### 3-[2-Amino-6-(4-hydroxymethyl-phenyl)-pyrimidin-4-yl]-biphenyl-4,4′-diol

The title compound was obtained from 1) the Suzuki Reaction of MEM protected 3-(2-Amino-6-chloro-pyrimidin-4-yl)-biphenyl-4,4′-diol (see Reaction Scheme 10 and Example 54) with 4-hydroxymethylphenylboronic acid following the General Procedure G and 2) the deprotection process following the General Procedure J.

¹H NMR (d₆-DMSO) δ2.16 (s, 2H) 5.35 (s, 1H) 6.86 (d, 2H) 6.96 (d, 1H) 7.22 (bs, 2H) 7.48 (d, 2H) 7.56-7.58 (m, 3H) 7.94 (s, 1H) 8.28 (d, 2H) 8.31 (d, 1H) 9.58 (s, 1H) 14.08 (s, 1H)

MS (ESI+) 386 (M+H) C₂₃H₁₉N₃O₃

### Example 86

### 3-[2-Amino-6-(1R-benzyl-2-hydroxy-ethylamino)-pyrimidin-4-yl]-biphenyl-4,4′-diol

The title compound was obtained upon treatment of 3-(2-Amino-6-chloro-pyrimidin-4-yl)-biphenyl-4,4′-diol (Example 54) with D-phenylalaninol following the General Procedure K.

¹H NMR (d₆-DMSO) δ2.73-2.77 (m, 1H) 2.89-2.92 (m, 1H) 3.43 (m, 2H) 4.24 (bs, 1H) 4.87 (m, 1H) 6.49 (s, 1H) 6.55 (bs, 2H) 6.84-6.85 (m, 3H) 7.07 (d, 1H) 7.17 (t, 1H) 7.25-7.30 (m, 4H) 7.40-7.47 (m, 3H) 7.74 (s, 1H) 9.48 (s, 1H) 14.76 (s, 1H)

MS (ESI+) 429 (M+H) C₂₅H₂₄N₄O₃

### Example 87

### 3-[2-Amino-6-(1S-benzyl-2-hydroxy-ethylamino)-pyrimidin-4-yl]-biphenyl-4,4′-diol

The title compound was obtained upon treatment of 3-(2-Amino-6-chloro-pyrimidin-4-yl)-biphenyl-4,4′-diol (Example 54) with L-phenylalaninol following the General Procedure K.

¹H NMR (d₆-DMSO) δ2.76-2.77 (m, 1H) 2.89-2.92 (m, 1H) 3.43 (m, 2H) 4.25 (bs, 1H) 4.87 (m, 1H) 6.50 (s, 1H) 6.55 (bs, 2H) 6.83-6.85 (m, 3H) 7.06 (bs, 1H) 7.17 (bs, 1H) 7.27-7.30 (m, 4H) 7.43-7.47 (m, 3H) 7.74 (s, 1H) 9.48 (s, 1H) 14.76 (s, 1H)

MS (ESI+) 429 (M+H) C₂₅H₂₄N₄O₃

### Example 88

### 3-[2-Amino-6-(3-nitro-phenyl)-pyrimidin-4-yl]-biphenyl-4,4′-diol

The title compound was obtained from 1) the Suzuki Reaction of MEM protected 3-(2-Amino-6-chloro-pyrimidin-4-yl)-biphenyl-4,4′-diol (Example 54) with 3-nitrophenylboronic acid following the General Procedure G and 2) the deprotection process following the General Procedure J.

¹H NMR (d₆-DMSO) δ6.89 (d, 2H) 7.00 (d, 1H) 7.47 (s, 2H) 7.61 (d, 2H) 7.64 (dd, 1H) 7.89 (t, 1H) 8.16 (s, 1H) 8.40 (d, 1H) 8.44 (dd, 1H) 8.84 (d, 1H) 9.11 (s, 1H) 9.53 (s, 1H) 13.95 (s, 1H)

MS (ESI+) 401 (M+H) C₂₂H₁₆N₄O₄

### Example 89

### 3-[2-Amino-6-(4-nitro-phenyl)-pyrimidin-4-yl]-biphenyl-4,4′-diol

The title compound was obtained from 1) the Suzuki Reaction of MEM protected 3-(2-Amino-6-chloro-pyrimidin-4-yl)-biphenyl-4,4′-diol (Example 54) with 4-nitrophenylboronic acid following the General Procedure G and 2) the deprotection process following the General Procedure J.

¹H NMR (d₆-DMSO) δ6.86 (m, 3H) 6.99 (d, 1H) 7.56-7.58 (m, 3H) 7.61 (d, 1H) 8.10 (s, 1H) 8.35 (s, 1H) 8.40 (d, 2H) 8.56 (d, 2H) 9.53 (s, 1H) 13.87 (s, 1H)

MS (ESI+) 401 (M+H) C₂₂H₁₆N₄O₄

### Example 90

### 3-[2-Amino-6-(4-amino-phenyl)-pyrimidin-4-yl]-biphenyl-4,4′-diol

The title compound was obtained from hydrogenation of the compound of Example 89.

¹H NMR (d₆-DMSO) δ5.76 (s, 2H) 6.65 (d, 2H) 6.85 (d, 2H) 6.94 (d, 1H) 6.98 (s, 2H) 7.53-7.57 (m, 3H) 7.73 (s, 1H) 8.06 (d, 2H) 8.25 (s, 1H) 9.46 (s, 1H) 14.33 (s, 1H)

MS (ESI+) 371 (M+H) C₂₂H₁₈N₄O₂

### Example 91

### 3-[2-Amino-6-(3-amino-phenyl)-pyrimidin-4-yl]-biphenyl-4,4′-diol

The title compound was obtained from hydrogenation of the compound of Example 88.

¹H NMR (d₆-DMSO) δ5.23 (s, 2H) 6.72 (d, 1H) 6.83-6.84 (m, 3H) 6.95 (d, 1H) 7.16 (t, 1H) 7.39 (m, 2H) 7.53-7.54 (m, 4H) 7.78 (bs, 1H) 8.22 (s, 1H) 13.98 (s, 1H)

MS (ESI+) 371 (M+H) C₂₂H₁₈N₄O₂

### Example 92

### 2-(2-Amino-pyrimidin-4-yl)-4-[2-(2-amino-pyrimidin-4-yl)-vinyl]-phenol

To a mixture of NBu₄Cl (1 eq), NaHCO₃ (2.5 eq), and Pd(OAc)₂ (5 mol %) under inert atmosphere was added a solution of aryl bromide in DMF (0.1 M) and methyl vinyl ketone (1.5 eq). The reaction mixture was stirred at 100° C. for 21 h and the solvent was evaporated. The crude was taken up in EtOAc and the solution was washed with water and brine, then dried over MgSO₄, filtered and evaporated. Purification by column chromatography (50% EtOAc-Hexane) afforded pure Heck coupling product.

The Heck coupling product was then converted to enamine upon reflux in N,N′-dimethylformamide diethylacetal for 24 h. The oil obtainedwas dissolved in ethanol (180 mL) and treated with sodium ethoxide (4 eq) and guanidine hydrochloride (2 eq) then refluxed overnight. The cooled solution was diluted with water, extracted by CH₂Cl₂ and the combined organic phase was concentrated to get red viscous oil. This crude product was further purified by flash chromatography eluting with 0-5% MeOH in CH₂Cl₂ to get pale yellow crystals. The MEM group was then removed as described in the General Procedure J.

Yellow solid; ¹H NMR (DMSO-d₆) δ6.49 (s, 2 H), 6.66 (d, J=4.8 Hz, 1 H), 6.95 (d, J=8.6 Hz, 1 H), 7.02 (d, J=16.0 Hz, 1 H), 7.22 (br s, 2 H), 7.43 (d, J=5.2 Hz, 1 H), 7.69 (m, 2 H), 8.20 (d, J=4.8 Hz, 1 H), 8.23 (s, 1 H), 8.42 (d, J=5.2 Hz, 1 H), 14.2 (s, 1 H); ESI: m/z 307.08 (M+H+, C₁₆H₁₄N₆O requires 307.13).

### Example 93

### 2-[2-Amino-6-(2-hydroxy-1R-phenyl-ethylamino)-pyrimidin-4-yl]-4-(1H-indol-5-yl)-phenol

### The Synthesis of the Title Compound is Described in the Reaction Scheme 31.

### 1-(5-Bromo-2-methoxy-phenyl)-ethanone

A mixture of 1-(5-bromo-2-hydroxy-phenyl)-ethanone (10 g, 47 mmol), methyl iodide (3.5 mL, 56 mmol), and potassium carbonate (10 g, 73 mmol) in acetone (80 mL) was heated at reflux for 4 h. The reaction mixture was cooled to room temperature and the resulting white solid filtered and washed thoroughly with ethyl acetate. The filtrate was washed with water, brine, dried over MgSO₄ and filtered. The filtrate was concentrated in vacuo to provide the title compound (10 g, 94%) as a white solid.

### 3-(5-Bromo-2-methoxy-phenyl)-3-oxo-propionic acid methyl ester

A mixture of 1-(5-bromo-2-methoxy-phenyl)-ethanone (10 g, 44 mmol), and 60% sodium hydride (2.8 g, 70.0 mmol) in dimethyl carbonate (100 mL) was heated at reflux for 2 h. To the resulting brown suspension was added 0.5 M aq. citric acid until a clear solution was obtained. The clear solution was extracted with ethyl acetate. The combined organic layers were washed with brine, dried over MgSO₄ and filtered. The filtrate was concentrated in vacuo and the crude title compound was used in the next step without purification.

### 2-Amino-6-(5-bromo-2-methoxy-phenyl)-3H-pyrimidin-4-one

A mixture of the crude 3-(5-bromo-2-methoxy-phenyl)-3-oxo-propionic acid methyl ester, guanidine hydrochloride (4.6 g, 48 mmol), and sodium ethoxide (9.84 g, 145 mmol) in ethanol (100 mL) was heated at reflux for 18 h. The mixture was cooled to room temperature and diluted with water. The mixture was extracted with ethyl acetate and the combined organic layers washed with brine, dried over MgSO₄ and filtered. The filtrate was concentrated in vacuo and the crude product triturated in Et₂O. The resulting solid was filtered to afford the title compound (6.0 g, 46%) as a white solid.

### 4-(5-Bromo-2-methoxy-phenyl)-6-chloro-pyrimidin-2-ylamine

2-Amino-6-(5-bromo-2-methoxy-phenyl)-3H-pyrimidin-4-one (6.0 g, 20 mmol) was dissolved in phosphorous oxychloride (30 mL) and the resulting solution heated at reflux for 16 h. The mixture was cooled to room temperature and poured into the ice water slowly. It was neutralized carefully with aq. sodium hydroxide and the aqueous layer extracted with ethyl acetate. The combined organic layers were washed with brine, dried over MgSO₄ and filtered. The filtrate was concentrated in vacuo and the crude product triturated in Et₂O. The resulting solid was filtered to afford the title compound (2.0 g) as a pale brown solid. Chromatography of the filtrate on silica gel (hexane/EtOAc=6/1 gradient to 3/1) afforded additional product (0.5 g, 39% overall) as a white solid.

The Title Compound:

4-(5-Bromo-2-methoxy-phenyl)-6-chloro-pyrimidin-2-ylamine was demethylated using the General Procedure C. The resulting 4-(5-Bromo-2-hydroxy-phenyl)-6-chloro-pyrimidin-2-ylamine was then reacted with D-phenylglycinol follow the General Procedure K. Finally, the title compound was prepared through Suzuki coupling described in the General Procedure H with 5-indolylboronic acid.

Yellow solid; ¹H NMR (d₆-DMSO) δ3.69 (m, 2H) 5.28 (bs, 1H) 5.66 (bs, 1H) 6.51 (bs, 2H) 6.45-7.80 (m, 17H) 11.04 (s, 1H) 11.6-12.4 (bs, 1H); ESI: m/z 438.19 (M+H⁺).

### Example 94

### 3′-Amino-3-(2-amino-pyrimidin-4-yl)-biphenyl-4-ol

The title compound was obtained as a yellow solid from the boronate (see Example 64) via the Suzuki reaction with 2-bromoaniline as described in General Procedure G, followed by deprotection using TFA with 5-10% thioanisole (Reaction Scheme 6).

¹H NMR (CDCl₃) δ3.82 (bs, 2H), 5.22 (s, 2H), 6.68 (d, 2H), 6.87 (s, 1H) 6.95 (d, 1H), 7.03 (t, 1H), 7.06 (d, 1H), 7.58 (d, 1H), 7.93 (s, 1H), 8.42 (d, 1H)

ESI: m/z 278 (M+H⁺)

### Example 95

### 2-[3-(2-Amino-pyrimidin-4-yl)-4-hydroxy-phenyl]-pyrrole-1-carboxylic Acid t-butyl Ester

The title compound was obtained as a yellow solid from 4-(2-benzyloxy-5-bromo-phenyl)-pyrimidin-2-ylamine (see Example 59) via the Suzuki reaction with N-Boc protected 2-pyrrolylboronic acid as described in General Procedure G (Reaction Scheme 5).

ESI: m/z 381 (M+H⁺)

### Example 96

### 3-{2-Amino-6-[2-hydroxy-1R-(1H-indol-3-ylmethyl)-ethylamino]-pyrimidin-4-yl}-biphenyl-4,4′-diol

The title compound was synthesized starting from D-tryptophanol and the chloropyrimidine derivative using General Procedure K.

Yellow solid; ¹H NMR (d₆-DMSO) δ2.83-2.87 (m, 1H) 3.40-3.48 (m, 4H) 4.83 (s, 1H) 6.51 (bs, 2H) 6.82-6.86 (m, 3H) 6.96-7.05 (m, 3H) 7.15 (s, 1H) 7.31 (d, 1H) 7.42 (d, 2H) 7.46 (d, 1H) 7.61 (d, 1H) 7.74 (s, 1H) 9.47 (s, 1H) 10.79 (s, 1H) 14.74 (bs, 1H); ESI: m/z 468.19 (M+H⁺).

### Example 97

### 3-[2-Amino-6-(2-hydroxy-1S-phenyl-ethylamino)-pyrimidin-4-yl]-biphenyl-4,4′-diol

The title compound was synthesized starting from L-phenylglycinol and the chloropyrimidine derivative using General Procedure K.

Yellow solid; ¹H NMR (d₆-DMSO) δ3.55 (m, 2H) 3.66 (m, 1H) 4.94 (bs, 1H) 6.81-6.85 (m, 4H) 6.97 (d, 1H) 7.30-7.34 (m, 5H) 7.56 (d, 2H) 7.62 (d, 1H) 7.64 (s, 1H) 8.12 (s, 1H) 9.48 (s, 1H) 13.36 (s, 1H) 14.71 (bs, 1H);ESI: m/z 415.17 (M+H⁺).

### Example 98

### 3-[2-Amino-6-(4-methoxy-phenylamino)-pyrimidin-4-yl]-biphenyl-4,4′-diol

The title compound was synthesized starting from p-methoxyaniline and the chloropyrimidine derivative using General Procedure K.

Yellow solid; ¹H NMR (d₆-DMSO) δ3.74 (s, 1H) 6.61 (s, 1H) 6.79-6.85 (m, 4H) 6.89 (d, 2H) 7.44 (d, 2H) 7.50 (d, 1H) 7.56 (d, 1H) 7.65 (d, 2H) 7.78 (s, 1H) 9.49 (s, 1H) 9.50 (s, 1H) 14.54 (s, 1H); ESI: m/z 401.15 (M+H⁺).

### Example 99

### 3-[2-Amino-6-(2-benzylsulfanyl-1R-hydroxymethyl-ethylamino)-pyrimidin-4-yl]-biphenyl-4,4′-diol

The title compound was synthesized starting from S-benzyl-L-cysteinol and the chloropyrimidine derivative using General Procedure K.

Yellow solid; ¹H NMR (d₆-DMSO) δ3.45-3.49 (m, 2H) 3.57 (m, 1H) 3.79 (s, 1H) 4.89 (s, 1H) 6.54 (s, 1H) 6.59 (bs, 2H) 6.82-6.87 (m, 3H) 7.23-7.32 (m, 5H)7.43 (d, 2H) 7.47 (d, 1H) 7.56 (d, 1H) 7.76 (s, 1H) 9.48 (s, 1) 14.74 (bs, 1H); ESI: m/z 475.17 (M+H⁺).

### Example 100

### 3-{2-Amino-6-[2-(1-indol-3-yl)-ethylamino]-pyrimidin-4-yl} Biphenyl-4,4′-diol

The title compound was synthesized starting from trptamine and the chloropyrimidine derivative using General Procedure K.

Yellow solid; ¹H NMR (d₆-DMSO) δ2.95 (m, 2H) 3.62 (m, 2H) 6.44 (s, 1H) 6.56 (bs, 2H) 6.82 (d, 2H) 6.85 (d, 1H) 6.98 (t, 1H) 7.06 (t, 1H) 7.19 (s, 1H) 7.34 (d, 1H) 7.42 (d, 2H) 7.46 (d, 1H) 7.58 (d, 1H) 7.74 (bs, 1H) 9.46 (s, 1H) 10.83 (s, 1H) 14.81 (s, 1H); ESI: m/z 438.18 (M+H⁺).

### Example 101

### 3-[2-Amino-6-(4-benzyl-piperazin-1-yl)-pyrimidin-4-yl]-biphenyl-4,4′-diol

The title compound was synthesized starting from 4-benzylpyperazine and the chloropyrimidine derivative using General Procedure K.

Yellow solid; ¹H NMR (d₆-DMSO) δ2.43 (m, 4H) 3.53 (s, 2H) 3.70 (m, 4H) 6.60 (bs, 2H) 6.74 (s, 1H) 6.81-6.86 (m, 3H) 7.27 (m, 1H) 7.34 (m, 4H) 7.45-7.49 (m, 3H) 8.04 (s, 1H) 9.43 (s, 1H) 14.80 (s, 1H); ESI: m/z 454.21 (M+H⁺).

### Example 102

### 3-{2-Amino-6-[2-hydroxy-1R-(1H-indol-3-ylmethyl)-ethylamino]-pyrimidin-4-yl}-biphenyl-4,4′-diol

The title compound was synthesized starting from L-tryptophanol and the chloropyrimidine derivative using General Procedure K.

Yellow solid; ¹H NMR (d₆-DMSO) δ2.82-2.84 (m, 1H) 3.41-3.48 (m, 4H) 4.83 (s, 1H) 6.50 (bs, 2H) 6.82-6.85 (m, 3H) 6.96-7.05 (m, 3H) 7.15 (s, 1H) 7.31 (d, 1H) 7.42 (d, 2H) 7.46 (d, 1H) 7.61 (d, 1H) 7.74 (s, 1H) 9.48 (s, 1H) 10.79 (s, 1H) 14.78 (bs, 1H); ESI: m/z 468.19 (M+H⁺).

### Example 103

### 3-[2-Amino-6-(1-hydroxymethyl-2-methyl-propylamino) Pyrimidin-4-yl]-biphenyl-4,4′-diol

The title compound was synthesized starting from L-valionl and the chloropyrimidine derivative using General Procedure K.

ESI: m/z 353 (M+H⁺)

### Example 104

### 3-[2-Amino-6-(4-methyl-piperazin-1-yl)-pyrimidin-4-yl]-biphenyl-4,4′-diol

The title compound was synthesized starting from p-methylpiperazine and the chloropyrimidine derivative using General Procedure K.

Yellow solid; ¹H NMR (DMSO-d₆) δ2.22 (s, 3 H), 2.36 (s, 4 H), 3.69 (s, 4 H), 6.60 (s, 3H), 6.76 (s, 1 H), 6.82-6.86 (m, 3 H), 7.46-7.50 (m, 1 H), 8.06 (s, 1 H), 9.43 (s, 1 H), 14.8 (br s, 1 H); ESI: m/z 378.18 (M+H⁺).

### Example 105

### 3-[2-Amino-6-(2-morpholin-4-yl-ethylamino)-pyrimidin-4-yl]-biphenyl-4,4′-diol

The title compound was synthesized starting from N-(2-aminoethyl)morpholine and the chloropyrimidine derivative using General Procedure K.

Yellow solid; ¹H NMR (DMSO-d₆) δ2.43-2.47 (m, 8 H), 3.44-3.59 (m, 8 H), 6.50 (br s, 1 H), 6.56 (s, 2 H), 6.84 (m, 3 H), 7.01 (s, 1 H), 7.45 (m, 3 H), 7.77 (s, 1 H), 9.46 (s, 1 H), 14.80 (br s, 1 H); ESI: m/z 452.14 (M+H⁺).

### Example 106

### 3-[2-Amino-6-(2-pyridin-2-yl-ethylamino)-pyrimidin-4-yl]-biphenyl-4,4′-diol

The title compound was synthesized starting from 2′-aminoethylpyridine and the chloropyrimidine derivative using General Procedure K.

Yellow solid; ¹H NMR (DMSO-d₆) δ3.44-3.46 (m, 2 H), 3.68 (br.s, 1 H), 6.43 (s, 1 H), 6.59 (br.s, 2 H), 6.82-6.86 (m, 3 H), 7.23-7.25 (m, 3 H), 7.71 (s, 2 H), 7.97-8.05 (m, 2 H), 8.50 (m, 1 H), 14.79 (br s, 1 H); ESI: m/z 400.10 (M+H⁺).

### Example 107

### 3-(2-Amino-6-thiomorpholin-4-yl-pyrimidin-4-yl)-biphenyl-4,4′-diol

The title compound was synthesized starting from thiomorpholine and the chloropyrimidine derivative using General Procedure K.

Yellow solid; ¹H NMR (DMSO-d₆) δ2.62 (s, 4 H), 4.03 (br s, 4 H), 6.65 (br. s, 2 H), 6.76 (s, 1 H), 6.82-7.50 (m, 4 H), 8.07 (s, 1 H), 9.48 (s, 1 H), 14.77 (s, 1 H); ESI: m/z 381.09 (M+H⁺).

### Example 108

### 3-{2-Amino-6-[(biphenyl-2-ylmethyl)-amino]-pyrimidin-4-yl}-biphenyl-4,4′-diol

The title compound was synthesized starting from 2-phenylbenzylamine and the chloropyrimidine derivative using General Procedure K.

Yellow solid; ¹H NMR (DMSO-d₆) δ4.43 (s, 2 H), 6.55 (br. s, 2 H), 6.82-6.76 (s, 3 H), 7.25-7.27 (d, 1 H), 7.35-7.65 (m, 12 H), 9.48 (s, 1 H), 14.70 (s, 1 H); ESI: m/z 461.15 (M+H⁺).

### Example 109

### 3-[2-Amino-6-(3-chloro-phenylamino)-pyrimidin-4-yl]-biphenyl-4,4′-diol

The title compound was synthesized starting from m-chloroaniline and the chloropyrimidine derivative using General Procedure K.

Yellow solid; ¹H NMR (d₆-DMSO) δ5.76 (s, 1H) 6.69 (s, 1H) 6.84 (d, 2H) 6.91 (d, 1H) 6.99-7.02 (m, 3H) 7.31 (t, 1H) 7.45 (d, 2H) 7.52 (d, 1H) 7.65 (d, 1H) 7.81 (s, 1H) 8.00 (s, 1H) 9.58 (s, 1H) 14.33 (s, 1H); ESI: m/z 405.10 (M+H⁺).

### Example 110

### 3-[2-Amino-6-(4-chloro-phenylamino)-pyrimidin-4-yl]-biphenyl-4,4′-diol

The title compound was synthesized starting from p-chloroaniline and the chloropyrimidine derivative using General Procedure K.

Yellow solid; ¹H NMR (d₆-DMSO) δ6.68 (d, 1H) 6.84 (d, 2H) 6.90-6.95 (m, 3H) 7.33 (d, 2H) 7.45 (d, 2H) 7.52 (d, 1H) 7.81 (s, 1H) 7.84 (d, 2H) 9.50 (s, 1H) 9.55 (s, 1H) 14.40 (s, 1H); ESI: m/z 405.10 (M+H⁺).

### Example 111

### 3-[2-Amino-6-(3-methoxy-phenylamino)-pyrimidin-4-yl]-biphenyl-4,4′-diol

The title compound was synthesized starting from m-methoxyaniline and the chloropyrimidine derivative using General Procedure K.

Yellow solid; ¹H NMR (d₆-DMSO) δ3.77 (s, 3H) 6.56 (s, 1H) 6.68 (d, 1H) 6.85 (d, 2H) 6.90-6.92 (m, 3H) 7.19 (t,1H) 7.27 (s, 1H) 7.45 (d, 2H) 7.50-7.53 (m, 2H) 7.80 (s, 1H) 9.39 (s, 1H) 9.50 (s, 1H) 14.45 (s, 1H); ESI: m/z 401.15 (M+H⁺).

### Example 112

### 3-[2-Amino-6-(2-hydroxy-1-phenyl-ethylamino)-pyrimidin-4-yl]-biphenyl-4,4′-diol

The title compound was synthesized starting from D-phenylglycinol and the chloropyrimidine derivative using General Procedure K.

Yellow solid; ¹H NMR (d₆-DMSO) δ3.66 (d, 2H) 4.95 (t, 1H) 5.23 (s, 1H) 6.50 (bs, 2H) 6.63 (s, 1H) 6.83-6.84 (m, 3H) 7.24 (t, 1H) 7.33 (d, 2H) 7.37-7.47 (m, 5H) 7.55 (s, 1H) 7.77 (s, 1H) 9.48 (s, 1H) 14.67 (s, 1H); ESI: m/z 415.17 (M+H⁺).

### Example 113

### 3-(2-Amino-6-thiophen-3-yl-pyrimidin-4-yl)-biphenyl-4,4′-diol

The title compound was synthesized starting from 3-thienylboronic acid and the chloropyrimidine derivative using General Procedure H.

Yellow solid; ¹H NMR (d₆-DMSO) δ6.86 (d, 2H) 6.96 (d, 1H) 7.16 (bs, 2H) 7.56-7.59 (m, 3H) 7.69 (d, 1H) 7.85 (s, 1H) 7.94 (d, 1H) 8.27 (s, 1H) 8.53 (s, 1H) 9.50 (s, 1H) 14.06 (s, 1H); ESI: m/z 362.09 (M+H⁺).

### Example 114

### 3-[2-Amino-6-(1H-indol-5-yl)-pyrimidin-4-yl]-biphenyl-4,4′-diol

The title compound was synthesized starting from 3-thienylboronic acid and the chloropyrimidine derivative using General Procedure H.

Yellow solid; ¹H NMR (d₆-DMSO) δ6.58 (s, 1H) 6.86 (d, 2H) 6.96 (d, 1H) 7.10 (bs, 2H) 7.43 (s, 1H) 7.50 (d, 1H) 7.57-7.59 (m, 3H) 7.95 (s, 1H) 8.10 (d, 1H) 8.31 (s, 1H) 8.59 (s, 1H) 9.62 (bs, 1H) 11.34 (s, 1H) 14.24 (s, 1H); ESI: m/z 395.14 (M+H⁺).

### Example 115

### 3-[2-Amino-6-(3-fluoro-benzylamino)-pyrimidin-4-yl]-biphenyl-4,4′-diol

The title compound was synthesized starting from 3-fluoro-benzylamine and the chloropyrimidine derivative using General Procedure K.

Yellow solid; ¹H NMR (d₆-DMSO) δ4.57 (s, 2H) 6.50 (s, 1H) 6.60 (bs, 2H) 6.82 (d, 2H) 6.85 (d, 1H) 7.06 (t, 1H) 7.19 (s, 1H) 7.36 (d, 1H) 7.42 (d, 2H) 7.48 (d, 1H) 7.64 (d, 1H) 7.78 (bs, 1H) 9.45 (s, 1H) 14.65 (s, 1H); ESI: m/z 403.16 (M+H⁺).

### Example 116

### 3-[2-Amino-6-(2-fluoro-benzylamino)-pyrimidin-4-yl]-biphenyl-4,4′-diol

The title compound was synthesized starting from 2-fluoro-benzylamine and the chloropyrimidine derivative using General Procedure K.

Yellow solid; ¹H NMR (d₆-DMSO) δ4.59 (s, 2H) 6.45 (s, 1H) 6.53 (bs, 2H) 6.73 (d, 2H) 6.85 (d, 1H) 7.06 (t, 1H) 7.19 (s, 1H) 7.32 (d, 1H) 7.43 (d, 2H) 7.48 (d, 1H) 7.57 (d, 1H) 7.77 (bs, 1H) 9.60 (s, 1H) 14.65 (s, 1H); ESI: m/z 403.16 (M+H⁺).

### Example 117

### 3-[2-Amino-6-(3-methoxy-benzylamino)-pyrimidin-4yl]-biphenyl-4,4′-diol

The title compound was synthesized starting from 3- methoxy-benzylamine and the chloropyrimidine derivative using General Procedure K.

Yellow solid; ¹H NMR (d₆-DMSO) δ3.73 (s, 3H) 4.53 (s, 2H) 6.50 (s, 1H) 6.60 (bs, 2H) 6.82 (d, 2H) 6.85 (d, 1H) 6.92 (bs, 2H) 7.23 (t, 1H) 7.42 (d, 2H) 7.47 (d, 1H) 7.57 (d, 1H) 7.76 (bs, 1H) 9.45 (s, 1H) 14.65 (s, 1H); ESI: m/z 415.18 (M+H⁺).

### Example 118

### 3-[2-Amino-6-(4-methoxy-benzylamino)-pyrimidin-4-yl]-biphenyl-4,4′-diol

The title compound was synthesized starting from 4-methoxy-bensylamine and the chloropyrimidine derivative using General Procedure K.

Yellow solid; ¹H NMR (d₆-DMSO) δ3.72 (s, 3H) 4.47 (s, 2H) 6.47 (s, 1H) 6.58 (bs, 2H) 6.82 (d, 2H) 6.85 (d, 1H) 6.90 (d, 2H) 7.28 (d, 2H) 7.42 (d, 2H) 7.46 (d, 1H) 7.50 (d, 1H) 7.76 (bs, 1H) 9.45 (s, 1H) 14.71 (s, 1H); ESI: m/z 415.18 (M+H⁺).

### Example 119

### 3-[2-Amino-6-(2-fluoro-4-hydroxyphenylamino)-pyrimidin-4-yl]-biphenyl-4,4′-diol

The title compound was synthesized starting from 2-fluoro-4-hydroxyaniline and the chloropyrimidine derivative using General Procedure K.

Yellow solid; ¹H NMR (d₆-DMSO) δ6.60 (d, 1H) 6.64-6.70 (m, 4H) 6.83 (d, 2H) 6.89 (d, 1H) 7.42 (d, 2H) 7.49 (d, 1H) 7.65 (d, 1H) 7.76 (s, 1H) 8.69 (s, 1H) 9.46 (s, 1H) 9.74 (s, 1H) 14.49 (s, 1H); ESI: m/z 405.16 (M+H⁺).

### Biological Assays

### Example 129

### Assay Conditions

The assay of cyclin dependent kinase activity was determined by monitoring the phosphorylation of a synthetic peptide, PKTPKKAKKLRRR. The assay mixture contained 0.1 mM cold ATP, ³³P-γ-ATP (2000-4000 cpm/μL), 0.1 mM peptide, 1 mM DDt, and 10 mM MgCl₂ in 100 mM HEPES buffer pH 7.4. The assay mixture, along with inhibitor and CDK enzyme (0.3-0.4 U/mL; either CDK5 complexed with p25, CDK2 complexed with complexed with cyclin A), was mixed together in a microtiter plate in a final volume of 40 μL, sealed and incubated for 2 hours at 30° C. Following incubation, a 15 μL aliquot was added to a Whatman P81 filtration plate containing 100 μL 0.5% phosphoric acid. This mixture was allowed to stand for 5 minutes prior to filtration on a vacuum manifold. The filter plate was washed with 5 successive additions of 300 μL 0.5% phosphoric acid. The filtration plate was air dried for 10 minutes before sealing the bottom. After addition of scintillation fluid to the sealed plate, phosphorylated peptide was quantified in a scintillation counter.

The test results are presented in the following table:

| | | CDK 2 | CDK 5 |
|---|---|---|---|
| Example | Structure | Activity* | Activity* |
| 2 | 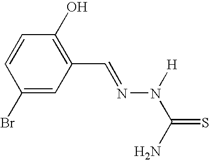 | C | B |
| 3 | 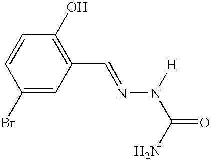 | C | B |
| 4 | 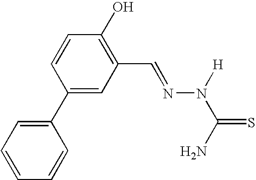 | B | A |
| 5 | 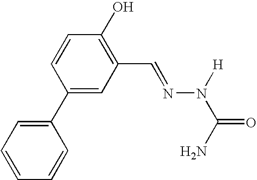 | B | A |
| 6 | 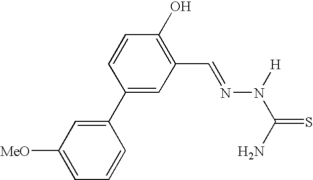 | C | C |
| 7 | 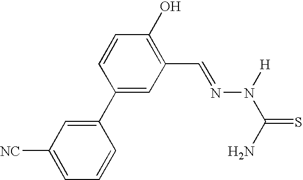 | D | C |
| 8 | 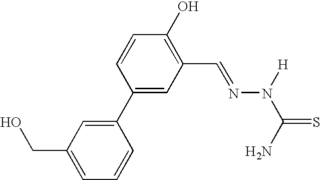 | D | D |
| 9 | 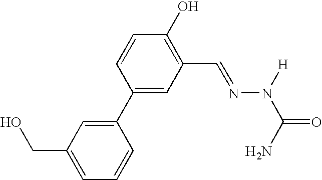 | C | B |
| 10 | 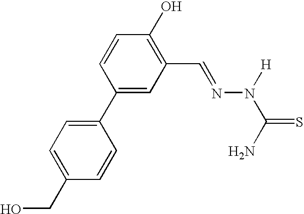 | C | B |
| 11 | 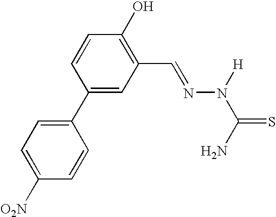 | D | C |
| 12 | 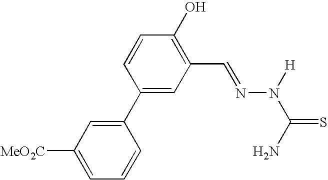 | D | B |
| 13 | 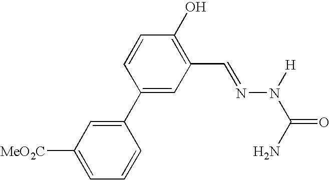 | C | D |
| 14 | 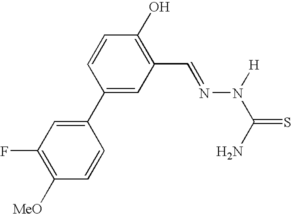 | C | B |
| 15 | 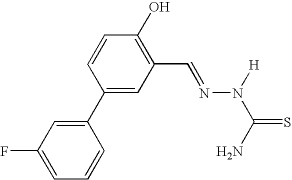 | B | B |
| 16 | 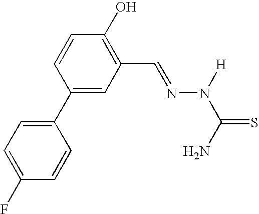 | C | B |
| 17 | 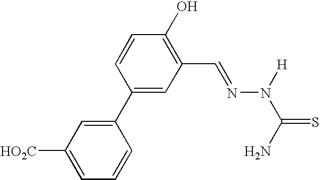 | B | C |
| 18 | 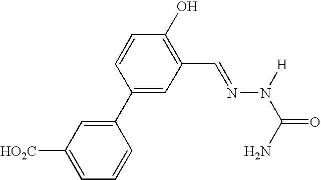 | B | A |
| 19 | 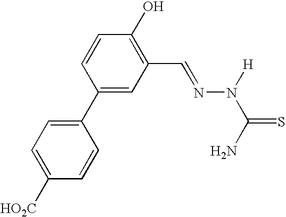 | D | A |
| 20 | 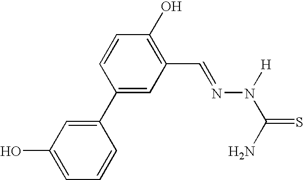 | B | A |
| 21 | 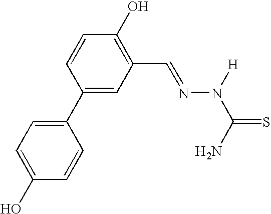 | A | A |
| 22 | 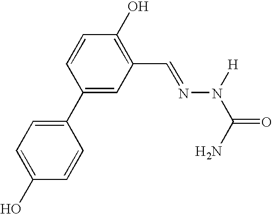 | A | A |
| 23 | 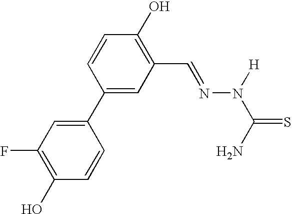 | A | A |
| 24 | 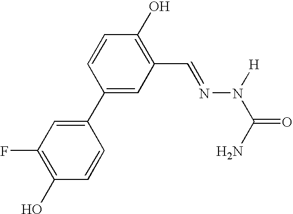 | A | A |
| 25 | 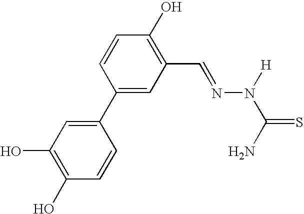 | A | A |
| 26 | 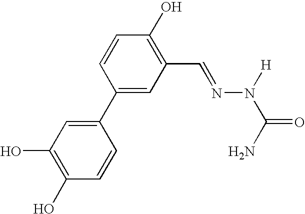 | A | A |
| 27 | 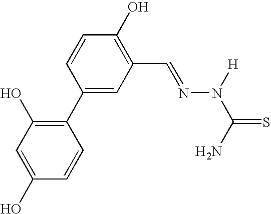 | A | A |
| 28 | 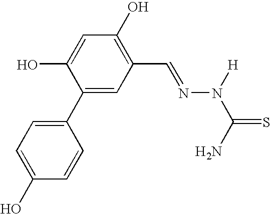 | B | C |
| 29 | 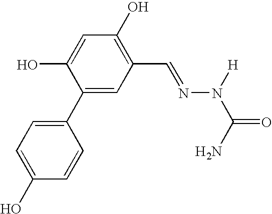 | B | D |
| 30 | 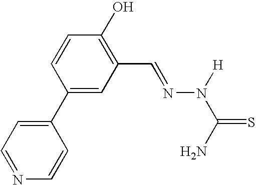 | C | A |
| 31 | 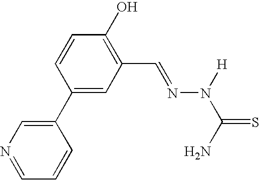 | B | A |
| 32 | 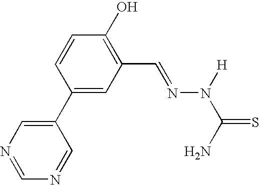 | C | C |
| 33 | 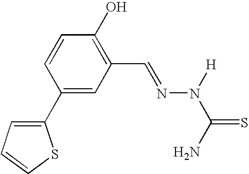 | A | B |
| 34 | 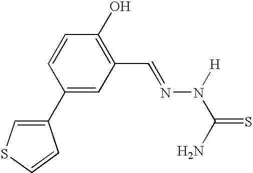 | A | B |
| 35 | 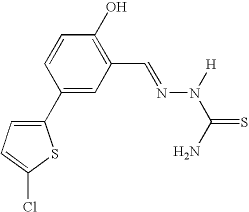 | B | D |
| 36 | 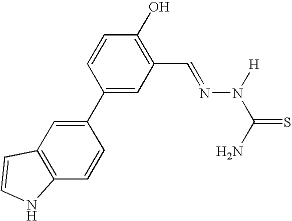 | A | A |
| 37 | 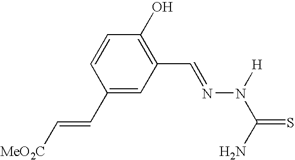 | D | B |
| 38 | 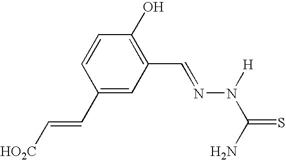 | A | A |
| 39 | 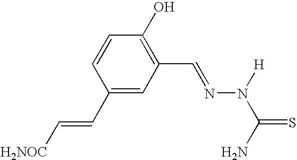 | B | D |
| 40 | 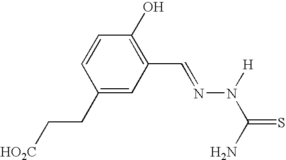 | A | A |
| 41 | 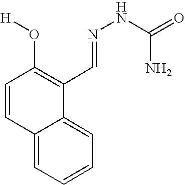 | A | A |
| 42 | 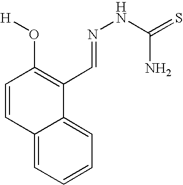 | A | A |
| 43 | 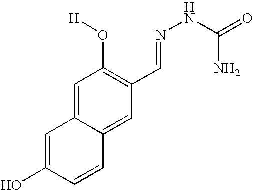 | A | A |
| 44 | 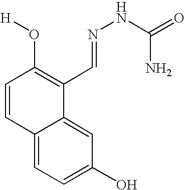 | C | B |
| 45 | 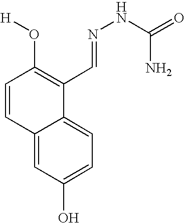 | D | C |
| 46 | 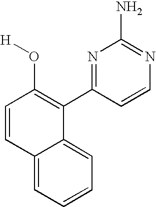 | D | C |
| 47 | 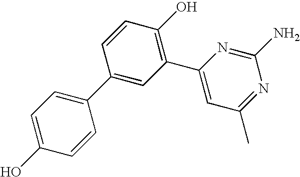 | A | A |
| 48 | 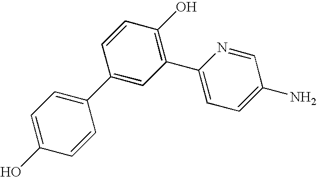 | A | A |
| 49 | 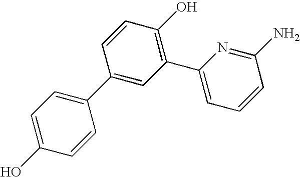 | A | A |
| 50 | 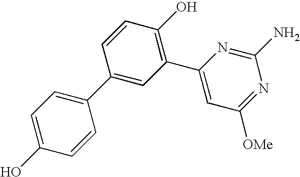 | A | A |
| 51 | 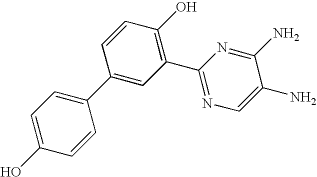 | A | A |
| 52 | 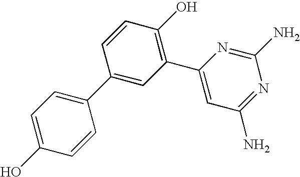 | A | A |
| 53 | 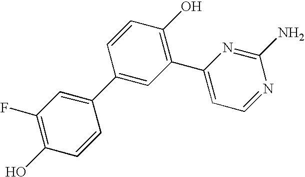 | A | A |
| 54 | 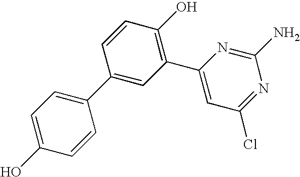 | A | A |
| 55 | 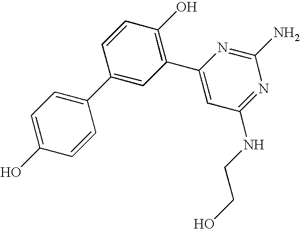 | A | A |
| 56 | 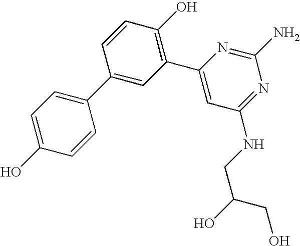 | A | A |
| 57 | 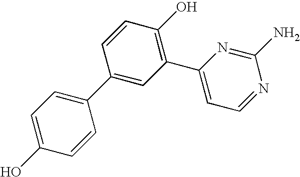 | A | A |
| 58 | 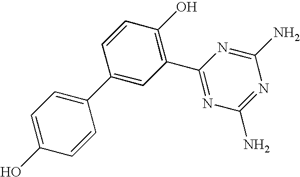 | A | A |
| 59 | 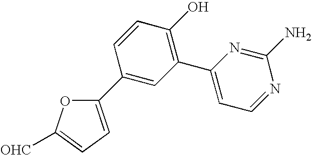 | B | B |
| 60 | 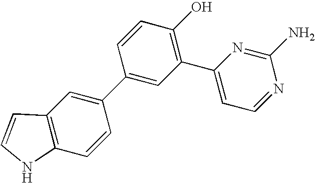 | C | A |
| 61 | 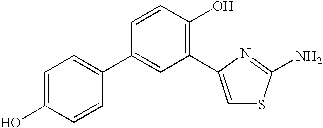 | A | A |
| 62 | 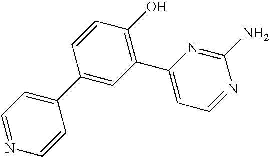 | A | A |
| 63 | 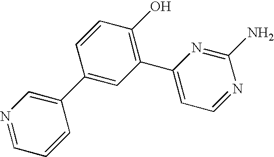 | D | A |
| 64 | 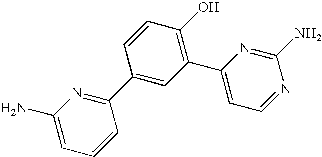 | A | A |
| 65 | 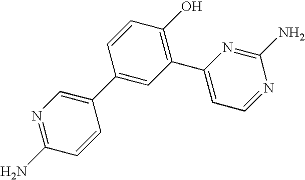 | A | B |
| 66 | 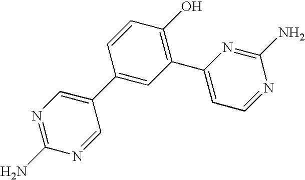 | B | B |
| 67 | 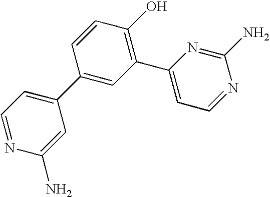 | A | NA |
| 68 | 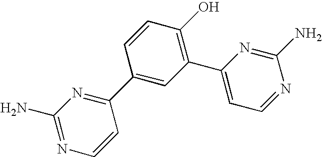 | A | A |
| 69 | 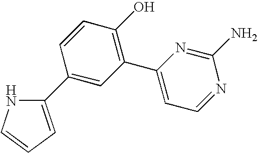 | A | A |
| 70 | 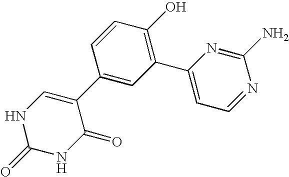 | D | C |
| 71 | 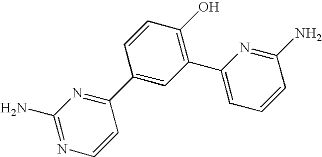 | A | A |
| 72 | 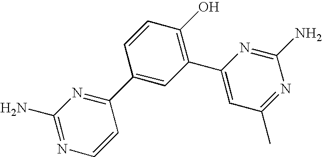 | A | NA |
| 73 | 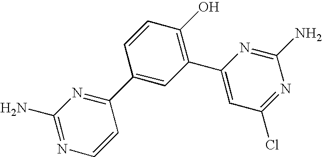 | A | NA |
| 74 | 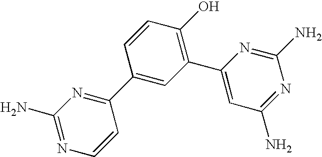 | A | NA |
| 75 | 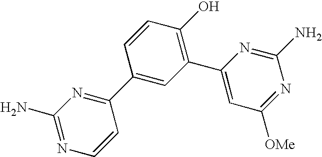 | NA | NA |
| 76 | 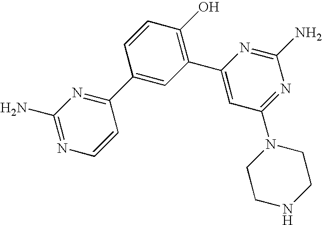 | NA | NA |
| 77 | 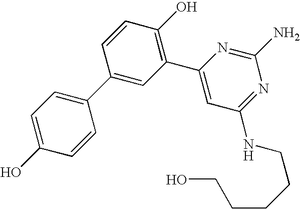 | A | A |
| 78 | 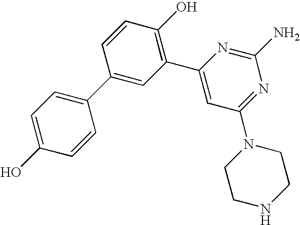 | D | A |
| 79 | 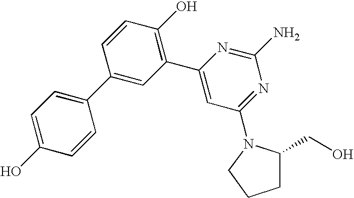 | A | A |
| 80 | 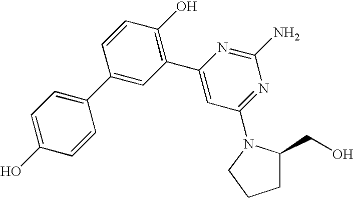 | A | A |
| 81 | 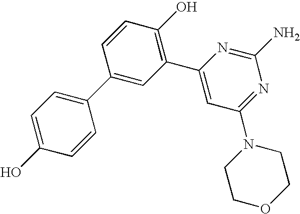 | A | A |
| 82 | 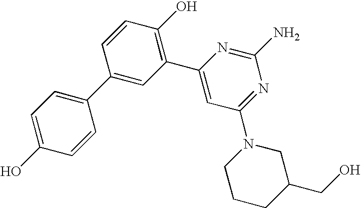 | A | A |
| 83 | 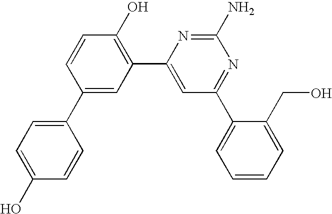 | A | A |
| 84 | 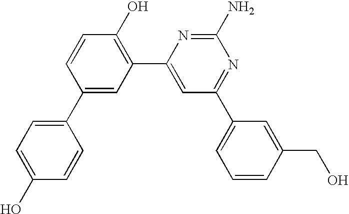 | A | A |
| 85 | 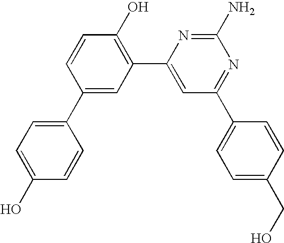 | A | A |
| 86 | 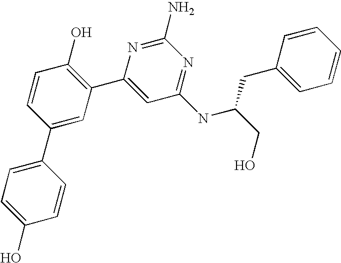 | A | A |
| 87 | 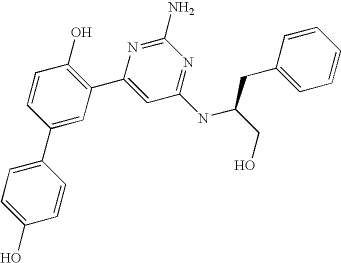 | A | A |
| 88 | 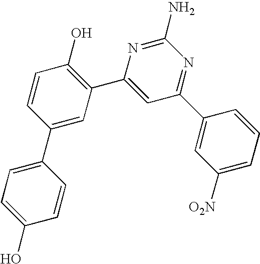 | A | A |
| 89 | 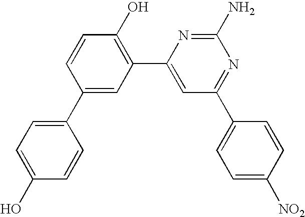 | A | A |
| 90 | 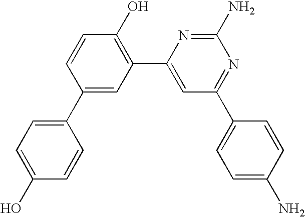 | A | A |
| 91 | 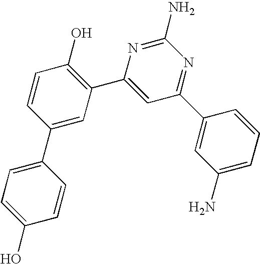 | A | A |

*: The activity of the compounds are defined as follows:
A: IC₅₀ = 0-0.5 μM
B: IC₅₀ = 0.5-2.0 μM
C: IC₅₀ = 2.0-5.0 M
D: IC₅₀ > 5.0 μM
NA: No activity data is available

### Example 130

### Specificity Studies

The assays of kinases were determined by monitoring the phosphorylation of appropriate substrates. The assay mixture contained 0.1 mM cold ATP ³³P-γ-ATP (2000-4000 cpm/μL), 0.1 mM substrate, 1 mM DTT, and 10 mM MgCl₂ in 100 mM HEPES buffer pH 7.4. The assay mixture, along with inhibitor and kinase enzyme (0.3-0.4 U/mL), was mixed together in a microtiter 96-well plate in a final volume of 40 μL, sealed and incbated for 1 hour at 30° C. Following incubation, a 15 μL aliquot was added to a Whatman P81 filtration plate containing 100 μL 0.5% phosphoric acid. This mixture was allowed to stand for 5 minutes prior to filtration on a vacuum manifold. The filter plate was washed with 5 successive additions of 300 μL 0.5% phosporic acid. The filtration plate was air dried for 10 minutes before sealing the bottom. After addition of scintillation fluid to the sealed plate, phosphorylated substrate was quantified in a scintillation counter.

3-(2-Amino-pyrimidin-4-yl)-biphenyl-4,4′-diol proved to be highly specific for CDK5 and CDK2. It showed little inhibition against a panel of protein kinases, as set forth in the following table.

| | % Inhibition at 10 | |
|---|---|---|
| Enzyme | μM BMI0000869 | Assay reference |
| Protein Kinase A (bovine heart) | NI | Chijiwa et al. (1990) |
| Protein Kinase C (rat brain) | NI | Humble et al. (1985) |
| Protein Kinase C-α (human) | 12 | Martiny-Baron et al. (1993) |
| Protein Kinase C-β1 (human) | NI | Martiny-Baron et al. (1993) |
| Protein Kinase C-β2 (human) | 49 | Martiny-Baron et al. (1993) |
| Protein Kinase C-γ (rabbit brain) | NI | Humble et al. (1985) |
| Protein Kinase C-γ (human) | 25 | Humble et al. (1985) |
| EGF-Tyrosine Kinase (human) | 25 | Carpenter et al. (1979) |
| Protein kinase p56^{Ick} (bovine thymus) | 29 | Cheng et al. (1992) |
| Protein kinase p55^{fyn} (bovine thymus) | 83 | Cheng et al. (1992) |
| MAP kinase, ERK 42 (rat) | NI | Robbins et al. (1993) |

Chijiwa et al. J Biol Chem. 1990 Mar 25; 265(9): 5267-72.
Martiny-Baron et al. J Biol Chem. 1993 May 5; 268(13): 9194-7.
Carpenter et al. J Biol Chem. 1979 Jun 10; 254(11): 4884-91.
Cheng et al. J Biol Chem. 1992 May 5; 267(13): 9248-56.
Robbins et al. J Biol Chem. 1993 Mar 5; 268(7): 5097-106.
Humble E et al Arch Biochem Biophys 1985 Aug 15; 241(1): 225-31

### Example 131

### In Vitro Tumor Cell Efficacy Test

Inhibition of cell growth was measured using cell viability assay, which is based on the use of a protein-binding dye, sulforhodamine B (SRB). The different cancer cell lines were seeded in 96-well plates at 2000 cells/100 μL per well in RPMI media with 10% FBS. Next day, the cells were treated with compounds as designed doses and incubated for 72 hours at 37° C. in the presence of 5% CO₂. Then the cells were fixed with 10% ice cold trichloroacetic acid (TCA) and incubated for an hour at 4° C. After washing the plates with tap water 4-5 times, the plates were dried in the air. Then the cells were stained with 0.4% SRB for 10 minutes, washed with 1% acetic acid 4-5 times and dried in the air. After adding 100 μL of 10 mM unbeffered Tris base to solubilize bound stain, optical densities were measured at 574 mm using Victor™ (1420 Multi-label Counter, Wallace) for the analysis. The values of IC₅₀ (the concentration of 50% growth inhibition) were calculated from the dose-reponse curve fitting.

3-(2-Amino-pyrimidin-4-yl)-biphenyl-4,4′-diol was also tested for in vitro tumor cell efficacy against several cancers.

| Cell Line | log (GI₅₀)(M) | Log (LC₅₀)(M) |
|---|---|---|
| Leukemia | | |
| CCRF-CEM | −6.7 | −4.1 |
| HL-60(TB) | −6.9 | −4.1 |
| K-562 | −6.4 | >−4 |
| MOLT-4 | −6.6 | >−4 |
| RPMI-8226 | −6.7 | >−4 |
| SR | −8.0 | >−4 |
| Non-Small Cell Lung Cancer | | |
| A549/ATCC | −6.5 | >−4 |
| EKVX | −6.6 | >−4 |
| HOP-62 | −7.1 | −5.1 |
| HOP-92 | −6.4 | >−4 |
| NCI-H23 | −6.6 | >−4 |
| NCI-H322M | −6.5 | >−4 |
| NCI-H460 | −6.6 | >−4 |
| NCI-H522 | −6.7 | >−4 |
| Colon Cancer | | |
| COLO 205 | −5.1 | >−4 |
| HCC-2998 | −5.9 | >−4 |
| HCT-116 | −6.8 | >−4 |
| HCT-15 | −6.9 | >−4 |
| HT29 | −5.5 | >−4 |
| KM12 | −6.9 | >−4 |
| SW-620 | −6.5 | >−4 |
| CNS Cancer | | |
| SF-268 | −6.6 | >−4 |
| SF-295 | −6.6 | >−4 |
| SF-539 | −6.7 | >−4 |
| SNB-19 | −6.7 | −4.7 |
| SNB-75 | −5.7 | >−4 |
| U251 | −6.7 | >−4 |
| Melanoma | | |
| LOX IMVI | −6.7 | >−4 |
| MALME-3M | −6.3 | >−4 |
| M14 | −6.4 | >−4 |
| SK-MEL-2 | −6.7 | −5.5 |
| SK-MEL-28 | −6.6 | >−4 |
| SK-MEL-5 | −7.0 | −4.7 |
| UACC-257 | −5.9 | >−4 |
| UACC-62 | −6.7 | −4.9 |
| Ovarian Cancer | | |
| IGROV1 | −6.6 | >−4 |
| OVCAR-3 | −6.6 | >−4 |
| OVCAR-4 | −6.5 | >−4 |
| OVCAR-5 | −5.7 | >−4 |
| OVCAR-8 | −6.7 | >−4 |
| SK-OV-3 | −6.3 | >−4 |
| Renal Cancer | | |
| 786-0 | −6.6 | >−4 |
| A498 | −5.8 | >−4 |
| ACHN | −6.6 | >−4 |
| CAKI-1 | −6.4 | >−4 |
| RXF 393 | −6.0 | −5.2 |
| SN 12C | −6.6 | >−4 |
| TK-10 | −5.7 | >−4 |
| UO-31 | −6.6 | >−4 |
| Prostate Cancer | | |
| PC-3 | −6.6 | >−4 |
| DU-145 | −6.1 | >−4 |
| Breast Cancer | | |
| MCF7 | −6.6 | >−4 |
| NCI/ADR-RES | −6.5 | >−4 |
| MDA-MB-231/ATCC | −6.6 | >−4 |
| HS 578T | −6.4 | >−4 |
| MDA-MB-435 | −6.6 | >−4 |
| MDA-N | −6.6 | >−4 |
| BT-549 | −6.6 | >−4 |
| T-47D | −5.7 | −4.3 |
| Mean | −6.5 | >−4 |
| St. Dev. | 0.4 | |

### Example 132

### Assay Conditions for EGFR2 (KDR)

Enzyme assays for IC₅₀ determinations were performed in 96-well titer plates, in a total reaction volume of 50 μL. Each reaction contained 1.5 μL of appropriate dilutions of inhibitor, 50 mM Hepes, pH 7.4, 1 mM DTT, 10 mM magnesium chloride, 2 mM phospho-poly(E₄Y) (Sigma Chemical Company), 500 μM unlabeled ATP, 0.0025 μM [³³P]ATP (Amersham Pharmacia Biotech), and 50 nM KDR. All components except KDR were added to the wells, and the reaction was started by the addition of the enzyme. The reactions were mixed and incubated at room temperature for 1 hr, after which the reactions were quenched by the addition of 50 μL of 8% phosphoric acid. At this point, 90 μL aliquots of the samples were applied to positively charged DEAE 96-well filter plates (Millipore Corporation MADENOB10). The plates were incubated for 30 min at room temperature. The samples were aspirated and the wells were washed 3 times with 200 μL of 0.5% phosphoric acid. To each well, 50 μL of scintillation cocktail was added and counted for 60 sec in a TopCount microplate scintillation counter (Packard Instrument Company). The concentration of a compound that inhibited the activity of KDR by 50% (IC₅₀) was determined based its inhibition curve.

The test results are presented in the following table:

| Example | Structure | KDR: IC₅₀ (nM) |
|---|---|---|
| 22 | 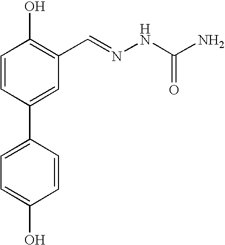 | B |
| 23 | 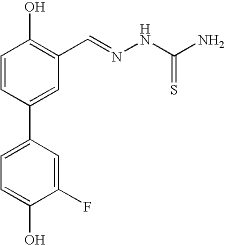 | B |
| 24 | 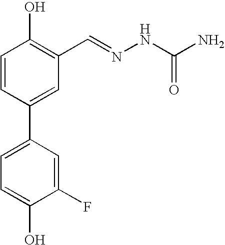 | B |
| 25 | 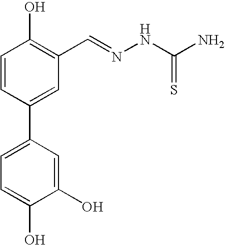 | A |
| 26 | 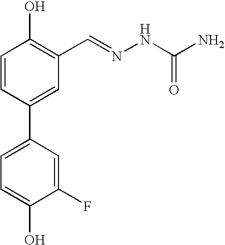 | A |
| 36 | 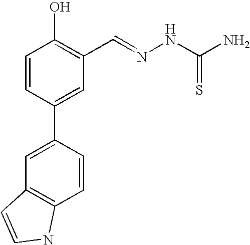 | C |
| 57 | 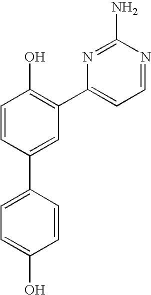 | A |
| 58 | 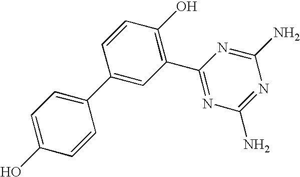 | A |
| 59 | 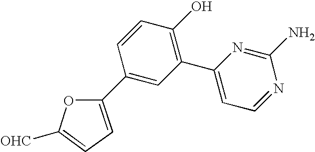 | A |
| 60 | 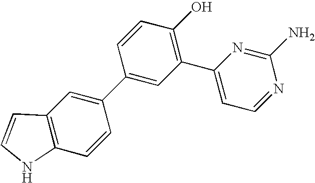 | A |
| 61 | 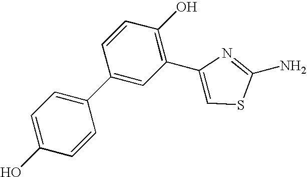 | B |
| 67 | 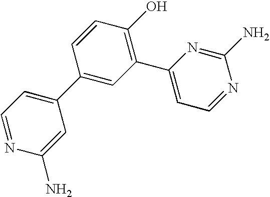 | A |
| 79 | 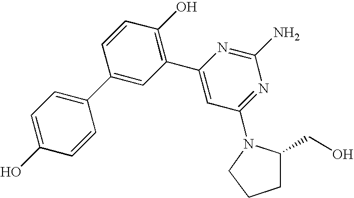 | B |
| 80 | 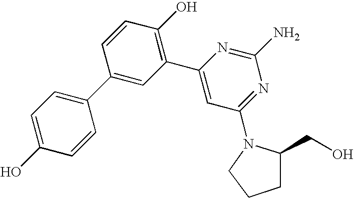 | B |
| 81 | 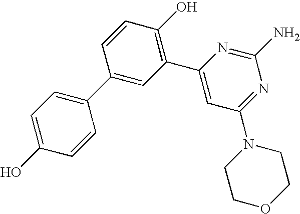 | B |
| 83 | 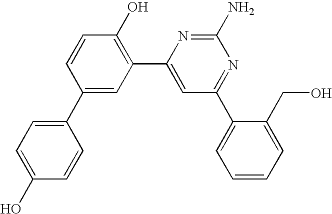 | A |
| 84 | 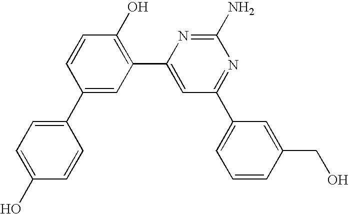 | A |
| 85 | 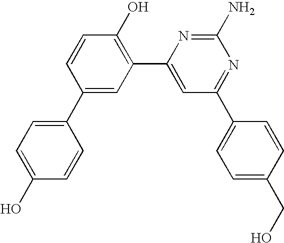 | A |
| 86 | 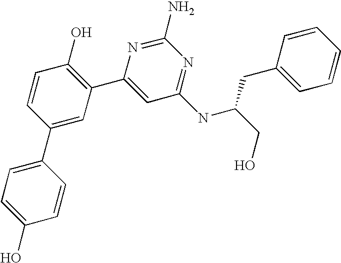 | B |
| 87 | 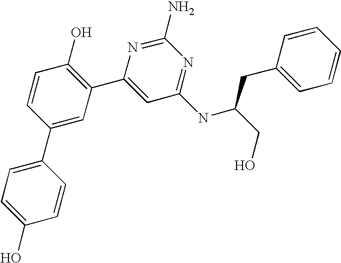 | B |
| 88 | 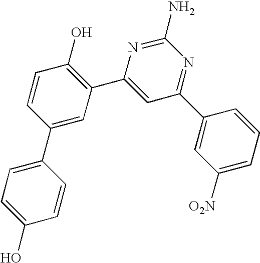 | B |
| 89 | 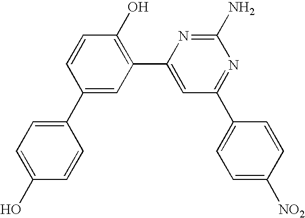 | B |
| 90 | 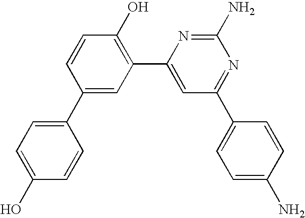 | A |
| 91 | 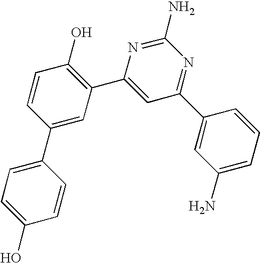 | A |
| 92 | 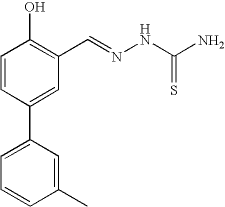 | B |
| 93 | 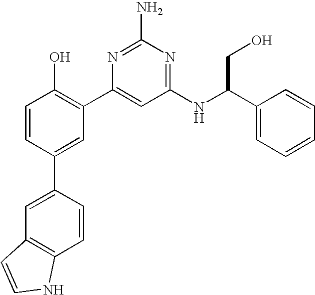 | A |
| 94 | 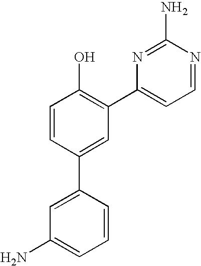 | B |
| 95 | 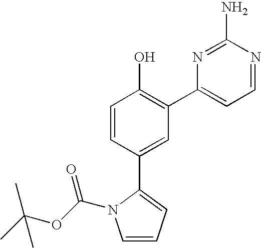 | B |
| 96 | 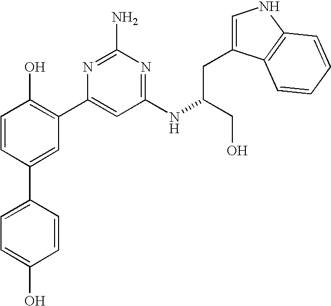 | B |
| 97 | 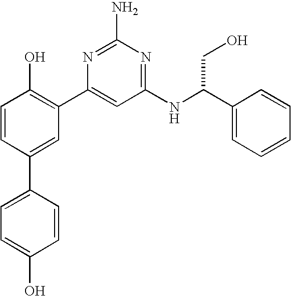 | A |
| 98 | 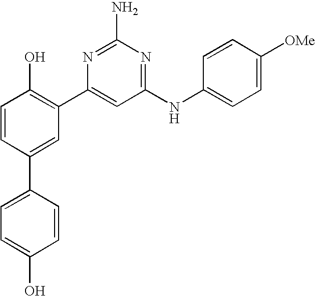 | A |
| 99 | 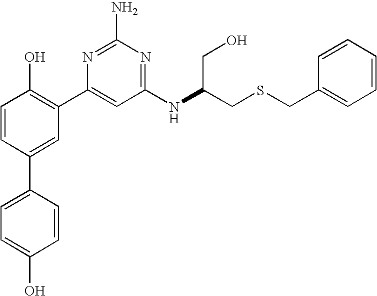 | A |
| 100 | 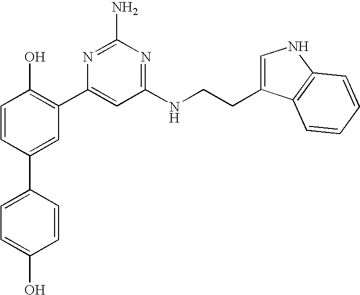 | A |
| 101 | 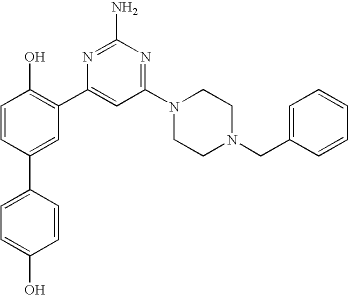 | B |
| 102 | 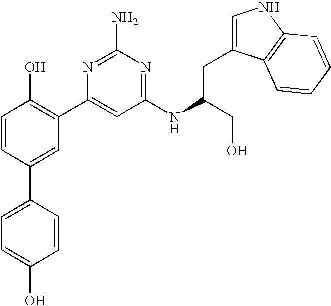 | B |
| 103 | 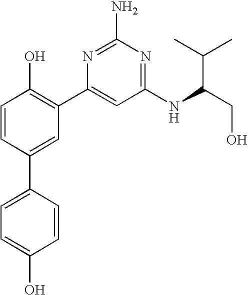 | B |
| 104 | 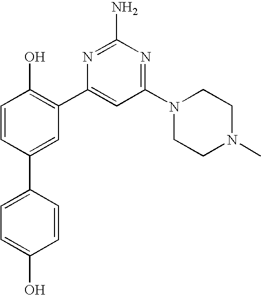 | B |
| 105 | 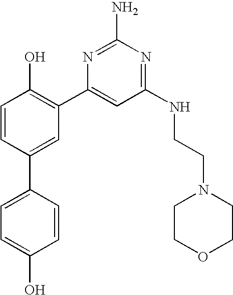 | B |
| 106 | 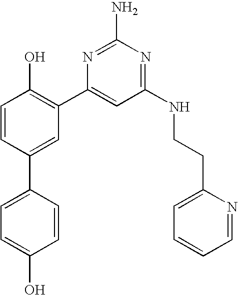 | A |
| 107 | 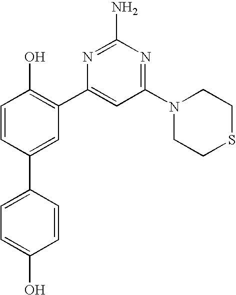 | B |
| 108 | 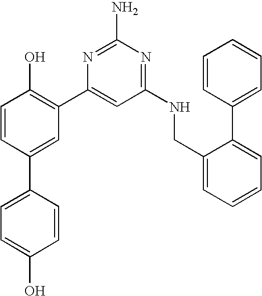 | A |
| 109 | 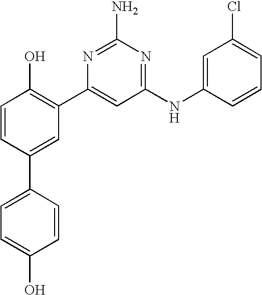 | A |
| 110 | 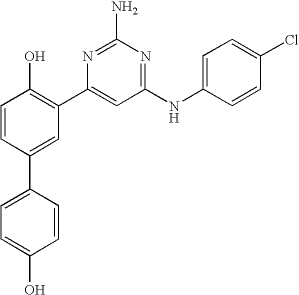 | A |
| 111 | 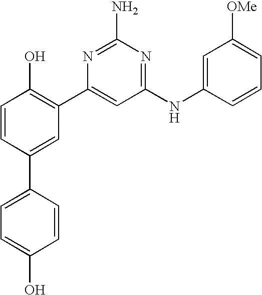 | A |
| 112 | 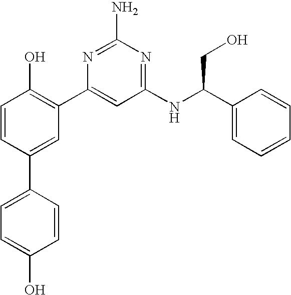 | A |
| 113 | 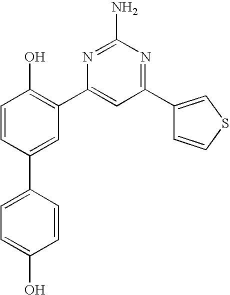 | B |
| 114 | 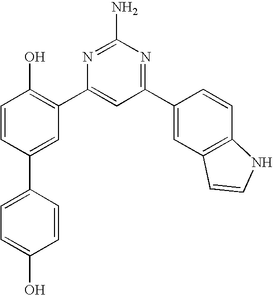 | A |
| 115 | 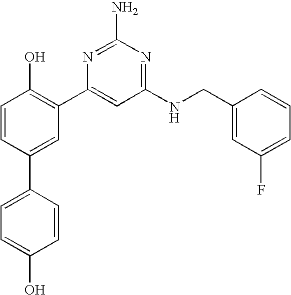 | B |
| 116 | 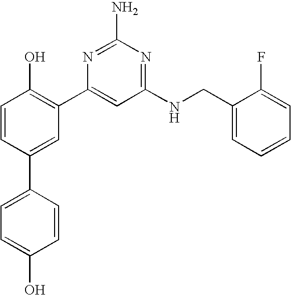 | A |
| 117 | 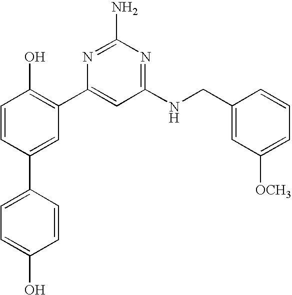 | A |
| 118 | 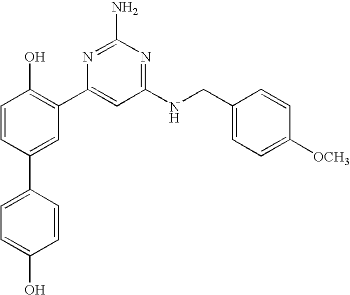 | A |
| 119 | 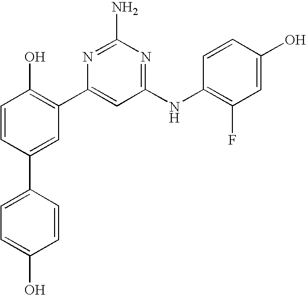 | B |

*: The activity of the compounds are defined as follows:
A: IC₅₀ = 0-1 μM
B: IC₅₀ = 1-20 μM
C: IC₅₀ = 20-50 μM
D: IC₅₀ >50 μM
NA: No activity data is available

### Conclusion

Thus, those of skill in the art appreciate that various compounds, methods and pharmacological compositions of the present invention can modulate protein kinase activity and are useful as therapeutic agents against CDK-related disorders. The protein kinase activity of any particular compound can be readily ascertained, e.g., as set forth in Example 100.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments and are exemplary and are not intended as limitations on the scope of the invention. Changes therein and other uses will occur to those skilled in the art which are encompassed within the spirit of the invention and are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically required herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof.

It is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

What is claimed is:

1. A compound of Formula IV 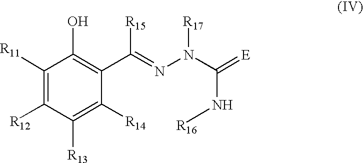
or a pharmaceutically acceptable salt, amide, ester, or prodrug thereof, wherein
a) R₁₁ is selected from the group consisting of hydrogen, lower alkyl, aryl, and heteroaryl;
b) R₁₂, R₁₃, and R₁₄, are each independently selected from the group consisting of
i) hydrogen;
ii) lower alkyl;
iii) lower alkylene;
iv) halogen or perhaloalkyl;
v) an alkoxy of formula —(X₁₃)ₙ₁₃—O—X₁₄, wherein
X₁₃ is selected from the group consisting of lower alkylene, lower alkenylene, lower alkynylene, aryl, and heteroaryl;
X₁₄ is selected from the group consisting of hydrogen, lower alkyl, aryl, and heteroaryl; and
n13 is 0 or 1; and
vi) a five-membered or six-membered heteroaryl ring or a six-membered aryl or heteroaryl ring, optionally substituted with one or more substituents selected from the group consisting of
A) optionally substituted C₁-C₈ straight-chain, branched, or cyclic saturated or unsaturated alkyl;
B) an alkoxy of formula —(X₁₃)ₙ₁₃—O—X₁₄, wherein
X₁₃ is selected from the group consisting of lower alkylene, lower alkenylene, lower alkynylene, aryl, and heteroaryl;
X₁₄ is selected from the group consisting of hydrogen, lower alkyl, aryl, and heteroaryl; and
n13 is 0 or 1
C) halogen or perhaloalkyl;
D) cyano;
E) nitro;
F) an amino of formula —(X₁₅)ₙ₁₅—NX₁₆X₁₇, wherein
X₁₅ is selected from the group consisting of lower alkylene, lower alkenylene, lower alkynylene, aryl, and heteroaryl;
X₁₆ and X₁₇ are each independently selected from the group consisting of hydrogen, lower alkyl, aryl, and heteroaryl; or X₁₆ and X₁₇, taken together with the nitrogen to which they are attached, form a five-membered or six-membered heteroaromatic or heteroaliphatic ring; and
n15 is 0 or 1;
G) a substituent of formula —(X₁₈)ₙ₁₈—C(═E)—X₁₉, wherein
X₁₈ is selected from the group consisting of lower alkylene, lower alkenylene, lower alkynylene, aryl, and heteroaryl;
E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl;
X₁₉ is selected from the group consisting of hydrogen, lower alkyl, aryl, heteroaryl, hydroxy, alkoxy, amino, and —NX₂₀X₂₁,
wherein X₂₀ and X₂₁ are each independently selected from the group consisting of hydrogen, alkyl, aryl, and heteroaryl; and
n18 is 0 or 1; and
H) a thioether or thiol of formula —(X₂₂)ₙ₂₂—S—X₂₃, wherein
X₂₂ is selected from the group consisting of lower alkylene, lower alkenylene, lower alkynylene, aryl, and heteroaryl;
X₂₃ is selected from the group consisting of hydrogen, lower alkyl, aryl, and heteroaryl; and
n22 is 0 or 1;
I) an amide of formula —(X₂₄)ₙ₂₄—NH—C(O)—X₂₅ or —(X₂₆)ₙ₂₆—C(O)—NH—X₂₇
X₂₄ and X₂₆ are each independently selected from the group consisting of lower alkylene, lower alkenylene, lower alkynylene, aryl, and heteroaryl;
X₂₅ is selected from the group consisting of hydrogen, lower alkyl, lower alkenyl, aryl, heteroaryl, hydroxy, alkoxy, and amide; and
X₂₇ is selected from the group consisting of hydrogen, lower alkyl, lower alkenyl, aryl, and heteroaryl;
c) R₁₅ is selected from the group consisting of hydrogen, lower alkyl, aryl, alkaryl, heteroaryl, and amino;
d) R₁₆ and R₁₇ are each independently selected from the group consisting of hydrogen, lower alkyl, aryl, and heteroaryl; and
e) E is selected from the group consisting of oxygen, sulfur, and —NR₁₀₁—, wherein R₁₀₁ is selected from the group consisting of hydrogen hydrogen, lower alkyl, lower alkene, lower alkyne, aryl, and heteroaryl.

2. The compound of claim 1, wherein R₁₃ is selected from the group consisting of
i) hydrogen;
ii) C₂-C₆ alkenylene;
iii) halogen or perhaloalkyl;
iv) an alkoxy of formula —O—X₁₄, wherein
X₁₄ is selected from the group consisting of hydrogen, lower alkyl, and aryl; and
v) a five-membered or six-membered heteroaryl ring or a six-membered aryl or heteroaryl ring, optionally substituted with one or more substituents selected from the group consisting of
A) optionally substituted C₁-C₄ straight-chain, branched, or cyclic saturated or unsaturated alkyl;
B) an alkoxy of formula —O—X₁₄, wherein X₁₄ is hydrogen or lower alkyl; and
C) halogen or perhaloalkyl;
D) cyano;
E) nitro;
F) a substituent of formula —(X₁₈)ₙ₁₈—C(O)—X₁₉, wherein
X₁₈ is lower alkylene;
X₁₉ is selected from the group consisting of hydrogen, lower alkyl, aryl, heteroaryl, hydroxy, alkoxy, amino, and —NX₂₀X₂₁,
wherein X₂₀ and X₂₁ are each independently selected from the group consisting of hydrogen, alkyl, and aryl; and
n18 is 0 or 1; and
G) a thioether or thiol of formula —S—X₂₃, wherein X₂₃ is selected from the group consisting of hydrogen, lower alkyl, aryl, and heteroaryl; and
H) an amide of formula —(X₂₄)ₙ₂₄—NH—C(O)—X₂₅ or —(X₂₆)ₙ₂₆—C(O)—NH—X₂₇
X₂₄ and X₂₆ are each independently lower alkylene;
X₂₅ is selected from the group consisting of hydrogen, lower alkyl, aryl, hydroxy, and alkoxy; and
X₂₇ is selected from the group consisting of hydrogen, lower alkyl, aryl, and heteroaryl.

3. The compound of claim 1, wherein R₁₃ is selected from the group consisting of
i) hydrogen;
ii) C₂-C₆ alkenylene;
iii) halogen or perfluoroalkyl;
iv) an alkoxy of formula —O—X₁₄, wherein
X₁₄ is selected from the group consisting of hydrogen, methyl, ethyl, and propyl; and
v) a six-membered aryl ring, optionally substituted with one or more substituents selected from the group consisting of
A) methyl, ethyl, and propyl;
B) hydroxy, methoxy, ethoxy, phenoxy, hydroxymethyl, 2-hydroxyethyl, and 3-hydroxypropyl; and
C) halogen or perfluoroalkyl;
D) cyano;
E) nitro;
F) a substituent of formula —C(O)—X₁₉, wherein
X₁₉ is selected from the group consisting of hydrogen, methyl, ethyl, propyl, phenyl, hydroxy, methoxy, ethoxy, propoxy, amino, and —NX₂₀X₂₁,
wherein X₂₀ and X₂₁ are each independently selected from the group consisting of hydrogen, methyl, ethyl, propyl, and phenyl; and
G) a thioether or thiol of formula —S—X₂₃, wherein X₂₃ is selected from the group consisting of hydrogen, methyl, ethyl, propyl, and phenyl; and
H) an amide of formula —(X₂₄)ₙ₂₄—NH—C(O)—X₂₅ or —(X₂₆)ₙ₂₆—C(O)—NH—X₂₇
X₂₄ and X₂₆ are each independently lower alkylene;
X₂₅ is selected from the group consisting of hydrogen, methyl, ethyl, propyl, phenyl, hydroxy, methoxy, and phenoxy; and
X₂₇ is selected from the group consisting of hydrogen, methyl, ethyl, propyl, and phenyl.

4. The compound of claim 1, wherein said five-membered or six-membered heteroaryl ring or said six-membered aryl or heteroaryl ring of R₁₃ is selected from the group consisting of optionally substituted 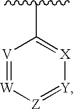
optionally substituted 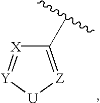
and optionally substituted 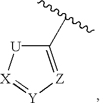
wherein V, W, X, Y and Z are each independently CR or nitrogen, and U is selected from the group consisting of CR₂, oxygen, sulfur, and NR;
wherein R is selected from the group consisting of hydrogen, alkyl, and aryl.

5. The compound of claim 1, wherein said five-membered or six-membered heteroaryl ring or the six-membered aryl or heteroaryl ring of R₁₃ is selected from the group consisting of phenyl, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine.

6. The compound of claim 1, wherein R₁₃ is selected from the group consisting of hydrogen, chloro, bromo, hydroxy, —CH═CH—CH₂CH₂CH₂CH₃, —CH═CH—C(O)—OH, —CH═CH—C(O)—OCH₃, —CH═CH—C(O)—NH₂, —CH₂CH(NH₂)COOH, phenyl, —O—CH₂-phenyl, 2-methylphenyl, 3-methylphenyl, 2-methylthiophenyl, 3-methylthiophenyl, 4-methylthiophenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2-hydroxymethylphenyl, 3-hydroxymethylphenyl, 4-hydroxymethylphenyl, 2-fluorophenyl, 3-fluorophenyl, 4-fluorophenyl, 2-cyanophenyl, 3-cyanophenyl, 4-cyanophenyl, 2-nitrophenyl, 3-nitrophenyl, 4-nitrophenyl, 2-hydroxycarbonylphenyl, 3-hydroxycarbonylphenyl, 4-hydroxycarbonylphenyl, 2-methoxycarbonylphenyl, 3-methoxycarbonylphenyl, 4-methoxycarbonylphenyl, 2-trifluoromethylphenyl, 4-benzyloxyphenyl, 2-phenoxyphenyl, 2,4-dihydroxyphenyl, 3,4-dihydroxyphenyl, 2-hydroxy-4-methoxyphenyl, 4-hydroxy-2-methoxyphenyl, 2,4-dimethoxyphenyl, 3,4-dimethoxyphenyl, 3-fluoro-4-hydroxyphenyl, 3-fluoro-4-methoxyphenyl, 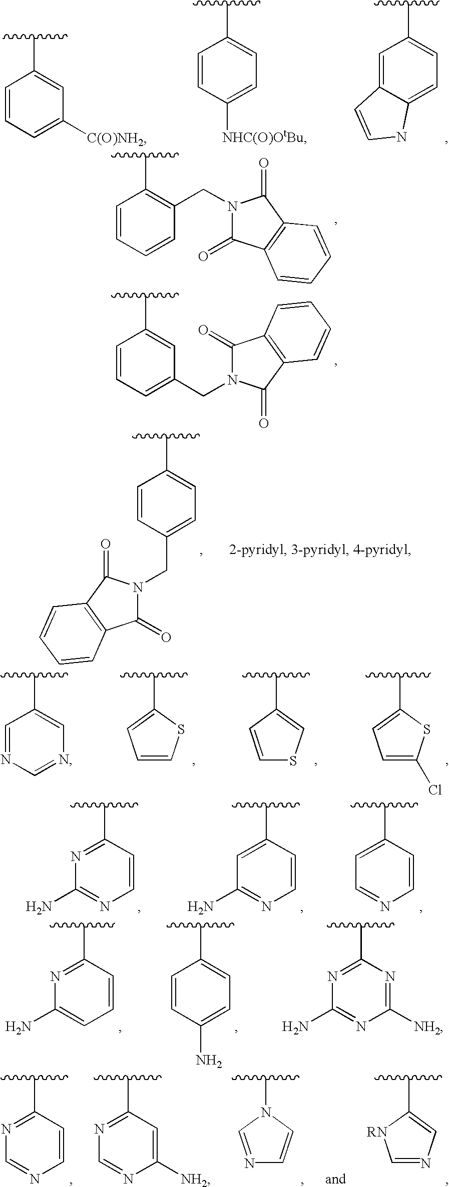
wherein R is selected from the group consisting of hydrogen, alkyl, and aryl.

7. The compound of claim 1, wherein said R₁₁, R₁₂, R₁₃, and R₁₄ is each independently selected from the group consisting of (i) hydrogen, (ii) hydroxyl, (iii) halogens, (iv) cyano, (v) nitro, (vi) amino, (vii) hydroxycarbonyl, (viii) aminocarbonyl, (ix) aminothiocarbonyl, (x) lower alkoxy, (xi) phenoxy, (xii) (C₁-C₄)alkylamino, (xiii) arylamino, (xiv) C₁-C₈ straight-chain, branched, and cyclic saturated and unsaturated alkyl or alkenyl, (xv) optionally substituted aryl and (xvi) optionally substituted hereocycle.

8. The compound of claim 1, wherein said R₁₅ is selected from the group consisting of (i) hydrogen, (ii) cyano, (iii) amino, (iv) hydroxycarbonyl, (v) aminocarbonyl, (vi) aminothiocarbonyl, (vii) (C₁-C₄)alkylamino, (viii) arylamino, (ix) C₁-C₈ straight-chain, branched, and cyclic saturated and unsaturated alkyl or alkenyl, (x) optionally substituted aryl and (xi) optionally substituted hereocycle.

9. The compound of claim 1, wherein said R₁₆ is selected from the group consisting of (i) hydrogen, (ii) amino, (iii) hydroxycarbonyl, (iv) aminocarbonyl, (v) aminothiocarbonyl, (vi) (C₁-C₄)alkylamino, (vii) arylamino, (vii) C₁-C₈ straight-chain, branched, and cyclic saturated and unsaturated alkyl or alkenyl, (ix) optionally substituted aryl and (x) optionally substituted hereocycle.

10. The compound of claim 1, wherein said R₁₇ is selected from the group consisting of (i) hydrogen, (ii) (C₁-C₄)alkylamino, (iii) arylamino, (iv) C₁-C₈ straight-chain, branched, and cyclic saturated and unsaturated alkyl, (v) optionally substituted aryl and (vi) optionally substituted hereocycle.

11. The compound of any one of claims 7-10, wherein said heterocycle is selected from the group consisting of furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, triazine, and benzimidazole.

12. A compound selected from the group consisting of 5-bromosalicylaldehyde thiosemicarbazone; 5-bromosalicylaldehyde semicarbazone hydrochloride; 5-phenylsalicylaldehyde thiosemicarbazone; 5-phenylsalicylaldehyde semicarbazone hydrochloride; 5-(3-methoxyphenyl)salicylaldehyde thiosemicarbazone; 5-(3-cyanophenyl)salicylaldehyde thiosemi-carbazone; 5-(3-hydroxymethylphenyl)salicylaldehyde thiosemicarbazone; 5-(3-hydroxymethyl-phenyl)salicylaldehyde semicarbazone hydrochloride; 5-(4-hydroxymethylphenyl)salicyl-aldehyde thiosemicarbazone; 5-(4-nitrophenyl)salicylaldehyde thiosemicarbazone; 5-(3-methoxycarbonylphenyl)salicylaldehyde thiosemicarbazone; 5-(3-methoxycarbonylphenyl)-salicylaldehyde semicarbazone hydrochloride; 5-(3-fluoro-4-methoxyphenyl)salicylaldehyde thiosemicarbazone; 5-(3-fluorophenyl)salicylaldehyde thiosemicarbazone; 5-(4-fluorophenyl)-salicylaldehyde thiosemicarbazone; 5-(3-carboxyphenyl)salicylaldehyde thiosemicarbazone; 5-(3-carboxyphenyl)salicylaldehyde semicarbazone hydrochloride; 5-(4-carboxyphenyl)salicyl-aldehyde thiosemicarbazone; 5-(3-hydroxyphenyl)salicylaldehyde thiosemicarbazone; 5-(4-hydroxyphenyl)salicylaldehyde thiosemicarbazone; 5-(4-hydroxyphenyl)salicylaldehyde semi-carbazone hydrochloride; 5-(3-fluoro-4-hydroxyphenyl)salicylaldehyde thiosemicarbazone; 5-(3-fluoro-4-hydroxyphenyl)salicylaldehyde semicarbazone hydrochloride; 5-(3,4-dihydroxy-phenylsalicylaldehyde thiosemicarbazone; 5-(3,4-dihydroxyphenyl)salicylaldehyde semicarbazone hydrochloride; 5-(2,4-dihydroxyphenyl)salicylaldehyde thiosemicarbazone; 4-hydroxy-5-(4-hydroxyphenyl)salicylaldehyde thiosemicarbazone; 4-hydroxy-5-(4-hydroxy-phenyl)salicylaldehyde semicarbazone hydrochloride; 5-(4-pyridyl)salicylaldehyde thiosemi-carbazone; 5-(3-pyridyl)salicylaldehyde thiosemicarbazone; 5-(5-pyrimidyl)salicylaldehyde thiosemicarbazone; 5-(2-thienyl)salicylaldehyde thiosemicarbazone; 5-(3-thienyl)salicylaldehyde thiosemicarbazone; 5-[2-(5-chloro-thienyl)]salicylaldehyde thiosemicarbazone; 5-(5-indolyl)-salicylaldehyde thiosemicarbazone; methyl (3-formyl-4-hydroxy)cinnamate, thiosemicarbazone; 3-formyl-4-hydroxycinnamic acid, thiosemicarbazone; 3-formyl-4-hydroxycinnamide, thiosemi-carbazone; 3-(3-formyl-4-hydroxyphenyl)propionic acid, thiosemicarbazone; 2-[(2-hydroxy-1-naphthyl)methylidene]hydrazine-1-carbamide hydrochloride; 2-[(2-hydroxy-1-naphthyl)-methylidene]hydrazine-1-carbothioamide; 2-[(2,7-dihydroxy-1-naphthyl)methylidene]hydrazine-1-carbothioamide; 2-[(2,7-dihydroxy-1-naphthyl)methylidene]hydrazine-1-carbothioamide; 2-[(2,6-dihydroxy-1-naphthyl)methylidene]hydrazine-1-carbothioamide; 2-amino-4-[1-(2-hydroxy-naphthyl)]pyrimidine; 3-(2-amino-pyrimidin-4-yl)-biphenyl-4,4′-diol; 2-(2-amino-pyrimidin-4-yl)-4-[2-(2-amino-pyrimidin-4-yl)-vinyl]-phenol.
